SEQUENCE LISTING (157 sequences)

SEQ ID NO: 1 - protein, 10 aa, Artificial sequence
  synthetic construct
GFSLSTYSMS

SEQ ID NO: 2 - protein, 16 aa, Artificial sequence
  synthetic construct
FIGSRGDTYYASWAKG

SEQ ID NO: 3 - protein, 14 aa, Artificial sequence
  synthetic construct
ERDPYGGGAYPPHL

SEQ ID NO: 4 - protein, 13 aa, Artificial sequence
  synthetic construct
QSSHSVYSDNDLA

SEQ ID NO: 5 - protein, 7 aa, Artificial sequence
  synthetic construct
QASKLAS

SEQ ID NO: 6 - protein, 12 aa, Artificial sequence
  synthetic construct
LGGYDDESDTYG

SEQ ID NO: 7 - protein, 122 aa, Artificial sequence
  synthetic construct
QVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSS

SEQ ID NO: 8 - protein, 112 aa, Artificial sequence
  synthetic construct
AIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 9 - protein, 122 aa, Artificial sequence
  synthetic construct
QVQLQQWGAGLLKPSETLSLTCAVYGFSLSTYSMSWIRQPPGKGLEWIGFIGSRGDTYYASWAKGRVTISRDTSKNQVSLKLSSVTAADTAVYYCARERDPYGGGAYPPHLWGRGTLVTVSS

SEQ ID NO: 10 - protein, 112 aa, Artificial sequence; identical to SEQ ID NO: 8
  synthetic construct

SEQ ID NO: 11 - protein, 10 aa, Artificial sequence
  synthetic construct
GFNIKDTYMH

SEQ ID NO: 12 - protein, 17 aa, Artificial sequence
  synthetic construct
RIDPANGNSKYVPKFQG

SEQ ID NO: 13 - protein, 12 aa, Artificial sequence
  synthetic construct
FGYYVSDYAMAY

SEQ ID NO: 14 - protein, 15 aa, Artificial sequence
  synthetic construct
RAGESVDIFGVGFLH

SEQ ID NO: 15 - protein, 7 aa, Artificial sequence
  synthetic construct
RASNRAT

SEQ ID NO: 16 - protein, 9 aa, Artificial sequence
  synthetic construct
QQTNEDPYT

SEQ ID NO: 17 - protein, 121 aa, Artificial sequence
  synthetic construct
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSS

SEQ ID NO: 18 - protein, 111 aa, Artificial sequence
  synthetic construct
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIK

SEQ ID NO: 19 - protein, 10 aa, Artificial sequence
  synthetic construct
GYTFTEFGMN

SEQ ID NO: 20 - protein, 17 aa, Artificial sequence
  synthetic construct
WINTKTGEATYVEEFKG

SEQ ID NO: 21 - protein, 12 aa, Artificial sequence
  synthetic construct
WDFAYYVEAMDY

SEQ ID NO: 22 - protein, 11 aa, Artificial sequence
  synthetic construct
KASAAVGTYVA

SEQ ID NO: 23 - protein, 7 aa, Artificial sequence
  synthetic construct
SASYRKR

SEQ ID NO: 24 - protein, 10 aa, Artificial sequence
  synthetic construct
HQYYTYPLFT

SEQ ID NO: 25 - protein, 121 aa, Artificial sequence
  synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 26 - protein, 108 aa, Artificial sequence
  synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 27 - protein, 450 aa, Artificial sequence
  synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 450 aa, Artificial sequence
  synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 29 - protein, 450 aa, Artificial sequence; identical to SEQ ID NO: 28
  synthetic construct

SEQ ID NO: 30 - protein, 450 aa, Artificial sequence; identical to SEQ ID NO: 27
  synthetic construct

SEQ ID NO: 31 - protein, 20 aa, Artificial sequence
  synthetic construct
GGGGSGGGGSGGGGSGGGGS

SEQ ID NO: 32 - protein, 591 aa, Artificial sequence
  synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSS

SEQ ID NO: 33 - protein, 581 aa, Artificial sequence
  synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 34 - protein, 215 aa, Artificial sequence
  synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 5 aa, Artificial sequence
  synthetic construct
DYGVH

SEQ ID NO: 36 - protein, 16 aa, Artificial sequence
  synthetic construct
VIWSGGGTAYNTALIS

SEQ ID NO: 37 - protein, 11 aa, Artificial sequence
  synthetic construct
RGSYPYNYFDA

SEQ ID NO: 38 - protein, 14 aa, Artificial sequence
  synthetic construct
GSSTGAVTASNYAN

SEQ ID NO: 39 - protein, 7 aa, Artificial sequence
  synthetic construct
GHNNRPP

SEQ ID NO: 40 - protein, 9 aa, Artificial sequence
  synthetic construct
ALWYSDHWV

SEQ ID NO: 41 - protein, 119 aa, Artificial sequence
  synthetic construct
HVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGQGTTVTVSS

SEQ ID NO: 42 - protein, 109 aa, Artificial sequence
  synthetic construct
QAVVIQESALTTPPGETVTLTCGSSTGAVTASNYANWVQEKPDHLFTGLIGGHNNRPPGVPARFSGSLIGDKAALTIAGTQTEDEAIYFCALWYSDHWVIGGGTKLTVL

SEQ ID NO: 43 - protein, 5 aa, Artificial sequence
  synthetic construct
DYYMN

SEQ ID NO: 44 - protein, 19 aa, Artificial sequence
  synthetic construct
FIGNKANAYTTEYSASVKG

SEQ ID NO: 45 - protein, 10 aa, Artificial sequence
  synthetic construct
DRGLRFYFDY

SEQ ID NO: 46 - protein, 10 aa, Artificial sequence
  synthetic construct
RASSSVTYIH

SEQ ID NO: 47 - protein, 7 aa, Artificial sequence
  synthetic construct
ATSNLAS

SEQ ID NO: 48 - protein, 9 aa, Artificial sequence
  synthetic construct
QHWSSKPPT

SEQ ID NO: 49 - protein, 121 aa, Artificial sequence
  synthetic construct
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSS

SEQ ID NO: 50 - protein, 106 aa, Artificial sequence
  synthetic construct
EIVLTQSPATLSLSPGERATLSCRASSSVTYIHWYQQKPGQAPRSWIYATSNLASGIPARFSGSGSGTDFTLTISSLEPEDFAVYYCQHWSSKPPTFGQGTKLEIK

SEQ ID NO: 51 - protein, 450 aa, Artificial sequence
  synthetic construct
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 52 - protein, 591 aa, Artificial sequence
  synthetic construct
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSS

SEQ ID NO: 53 - protein, 449 aa, Artificial sequence
  synthetic construct
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 54 - protein, 213 aa, Artificial sequence
  synthetic construct
EIVLTQSPATLSLSPGERATLSCRASSSVTYIHWYQQKPGQAPRSWIYATSNLASGIPARFSGSGSGTDFTLTISSLEPEDFAVYYCQHWSSKPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 450 aa, Artificial sequence
  synthetic construct
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 581 aa, Artificial sequence
  synthetic construct
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 57 - protein, 449 aa, Artificial sequence
  synthetic construct
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 58 - protein, 213 aa, Artificial sequence; identical to SEQ ID NO: 54
  synthetic construct

SEQ ID NO: 59 - protein, 10 aa, Artificial sequence
  synthetic construct
GGTFSYYAIS

SEQ ID NO: 60 - protein, 17 aa, Artificial sequence
  synthetic construct
GILPAFGAANYAQKFQG

SEQ ID NO: 61 - protein, 11 aa, Artificial sequence
  synthetic construct
LPPLPGAGLDY

SEQ ID NO: 62 - protein, 11 aa, Artificial sequence
  synthetic construct
RASQSISSWLA

SEQ ID NO: 63 - protein, 7 aa, Artificial sequence
  synthetic construct
DASSLES

SEQ ID NO: 64 - protein, 9 aa, Artificial sequence
  synthetic construct
QQNTQYPMT

SEQ ID NO: 65 - protein, 120 aa, Artificial sequence
  synthetic construct
QVQLVQSGAEVKKPGSSVKVSCKASGGTFSYYAISWVRQAPGQGLEWMGGILPAFGAANYAQKFQGRVTITADKSTSTAYMELSSLRSEDTAVYYCARLPPLPGAGLDYWGQGTTVTVSS

SEQ ID NO: 66 - protein, 107 aa, Artificial sequence
  synthetic construct
DIQMTQSPSTLSASVGDRVTITCRASQSISSWLAWYQQKPGKAPKLLIYDASSLESGVPSRFSGSGSGTEFTLTISSLQPDDFATYYCQQNTQYPMTFGQGTKVEIK

SEQ ID NO: 67 - protein, 10 aa, Artificial sequence
  synthetic construct
GFTFSKYAMA

SEQ ID NO: 68 - protein, 17 aa, Artificial sequence
  synthetic construct
SISTGGVNTYYADSVKG

SEQ ID NO: 69 - protein, 8 aa, Artificial sequence
  synthetic construct
HTGDYFDY

SEQ ID NO: 70 - protein, 15 aa, Artificial sequence
  synthetic construct
RASQSVSISGINLMN

SEQ ID NO: 71 - protein, 7 aa, Artificial sequence
  synthetic construct
HASILAS

SEQ ID NO: 72 - protein, 9 aa, Artificial sequence
  synthetic construct
QQTRESPLT

SEQ ID NO: 73 - protein, 117 aa, Artificial sequence
  synthetic construct
EVQLLESGGGLVQPGGSLRLSCAASGFTFSKYAMAWVRQAPGKGLEWVASISTGGVNTYYADSVKGRFTISRDNSKNTLYLQMNSLRAEDTAVYYCATHTGDYFDYWGQGTMVTVSS

SEQ ID NO: 74 - protein, 111 aa, Artificial sequence
  synthetic construct
EIVLTQSPGTLSLSPGERATLSCRASQSVSISGINLMNWYQQKPGQQPKLLIYHASILASGIPDRFSGSGSGTDFTLTISRLEPEDFAVYYCQQTRESPLTFGQGTRLEIK

SEQ ID NO: 75 - protein, 10 aa, Artificial sequence
  synthetic construct
GFTFSSYAMS

SEQ ID NO: 76 - protein, 17 aa, Artificial sequence
  synthetic construct
AIIGSGASTYYADSVKG

SEQ ID NO: 77 - protein, 8 aa, Artificial sequence
  synthetic construct
GWFGGFNY

SEQ ID NO: 78 - protein, 12 aa, Artificial sequence
  synthetic construct
RASQSVTSSYLA

SEQ ID NO: 79 - protein, 7 aa, Artificial sequence
  synthetic construct
VGSRRAT

SEQ ID NO: 80 - protein, 9 aa, Artificial sequence
  synthetic construct
QQGIMLPPT

SEQ ID NO: 81 - protein, 117 aa, Artificial sequence
  synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 82 - protein, 108 aa, Artificial sequence
  synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 83 - protein, 450 aa, Artificial sequence
  synthetic construct
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 581 aa, Artificial sequence
  synthetic construct
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 85 - protein, 450 aa, Artificial sequence
  synthetic construct
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 86 - protein, 450 aa, Artificial sequence; identical to SEQ ID NO: 85
  synthetic construct

SEQ ID NO: 87 - protein, 594 aa, Artificial sequence
  synthetic construct
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSAST

SEQ ID NO: 88 - protein, 450 aa, Artificial sequence; identical to SEQ ID NO: 83
  synthetic construct

SEQ ID NO: 89 - protein, 218 aa, Artificial sequence
  synthetic construct
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 449 aa, Artificial sequence
  synthetic construct
QVQLVQSGAEVKKPGSSVKVSCKASGGTFSYYAISWVRQAPGQGLEWMGGILPAFGAANYAQKFQGRVTITADKSTSTAYMELSSLRSEDTAVYYCARLPPLPGAGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 580 aa, Artificial sequence
  synthetic construct
QVQLVQSGAEVKKPGSSVKVSCKASGGTFSYYAISWVRQAPGQGLEWMGGILPAFGAANYAQKFQGRVTITADKSTSTAYMELSSLRSEDTAVYYCARLPPLPGAGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 92 - protein, 449 aa, Artificial sequence
  synthetic construct
QVQLVQSGAEVKKPGSSVKVSCKASGGTFSYYAISWVRQAPGQGLEWMGGILPAFGAANYAQKFQGRVTITADKSTSTAYMELSSLRSEDTAVYYCARLPPLPGAGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 93 - protein, 449 aa, Artificial sequence; identical to SEQ ID NO: 92
  synthetic construct

SEQ ID NO: 94 - protein, 593 aa, Artificial sequence
  synthetic construct
QVQLVQSGAEVKKPGSSVKVSCKASGGTFSYYAISWVRQAPGQGLEWMGGILPAFGAANYAQKFQGRVTITADKSTSTAYMELSSLRSEDTAVYYCARLPPLPGAGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSAST

SEQ ID NO: 95 - protein, 449 aa, Artificial sequence; identical to SEQ ID NO: 90
  synthetic construct

SEQ ID NO: 96 - protein, 214 aa, Artificial sequence
  synthetic construct
DIQMTQSPSTLSASVGDRVTITCRASQSISSWLAWYQQKPGKAPKLLIYDASSLESGVPSRFSGSGSGTEFTLTISSLQPDDFATYYCQQNTQYPMTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 450 aa, Artificial sequence; identical to SEQ ID NO: 27
  synthetic construct

SEQ ID NO: 98 - protein, 579 aa, Artificial sequence
  synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSQAVVIQESALTTPPGETVTLTCGSSTGAVTASNYANWVQEKPDHLFTGLIGGHNNRPPGVPARFSGSLIGDKAALTIAGTQTEDEAIYFCALWYSDHWVIGGGTKLTVL

SEQ ID NO: 99 - protein, 450 aa, Artificial sequence; identical to SEQ ID NO: 28
  synthetic construct

SEQ ID NO: 100 - protein, 450 aa, Artificial sequence; identical to SEQ ID NO: 28
  synthetic construct

SEQ ID NO: 101 - protein, 592 aa, Artificial sequence
  synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGQGTTVTVSSAST

SEQ ID NO: 102 - protein, 450 aa, Artificial sequence; identical to SEQ ID NO: 27
  synthetic construct

SEQ ID NO: 103 - protein, 215 aa, Artificial sequence; identical to SEQ ID NO: 34
  synthetic construct

SEQ ID NO: 104 - protein, 447 aa, Artificial sequence
  synthetic construct
EVQLLESGGGLVQPGGSLRLSCAASGFTFSKYAMAWVRQAPGKGLEWVASISTGGVNTYYADSVKGRFTISRDNSKNTLYLQMNSLRAEDTAVYYCATHTGDYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 359 aa, Artificial sequence
  synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSSIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 106 - protein, 369 aa, Artificial sequence
  synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSA

SEQ ID NO: 107 - protein, 218 aa, Artificial sequence
  synthetic construct
EIVLTQSPGTLSLSPGERATLSCRASQSVSISGINLMNWYQQKPGQQPKLLIYHASILASGIPDRFSGSGSGTDFTLTISRLEPEDFAVYYCQQTRESPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 447 aa, Artificial sequence
  synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 359 aa, Artificial sequence; identical to SEQ ID NO: 105
  synthetic construct

SEQ ID NO: 110 - protein, 369 aa, Artificial sequence; identical to SEQ ID NO: 106
  synthetic construct

SEQ ID NO: 111 - protein, 215 aa, Artificial sequence
  synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 451 aa, Artificial sequence
  synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 359 aa, Artificial sequence
  synthetic construct
SIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIKGGGGSGGGGSGGGGSGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 114 - protein, 369 aa, Artificial sequence
  synthetic construct
GVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSGGGGSGGGGSGGGGSGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 115 - protein, 215 aa, Artificial sequence; identical to SEQ ID NO: 34
  synthetic construct

SEQ ID NO: 116 - protein, 5 aa, Artificial sequence
  synthetic construct
DSYMH

SEQ ID NO: 117 - protein, 17 aa, Artificial sequence
  synthetic construct
WIDPENGDTEYAPKFQG

SEQ ID NO: 118 - protein, 17 aa, Artificial sequence
  synthetic construct
WIDPENGGTNYAQKFQG

SEQ ID NO: 119 - protein, 11 aa, Artificial sequence
  synthetic construct
GTPTGPYYFDY

SEQ ID NO: 120 - protein, 10 aa, Artificial sequence
  synthetic construct
SASSSVSYMH

SEQ ID NO: 121 - protein, 10 aa, Artificial sequence
  synthetic construct
RASSSVSYMH

SEQ ID NO: 122 - protein, 10 aa, Artificial sequence
  synthetic construct
RASQSISSYM

SEQ ID NO: 123 - protein, 7 aa, Artificial sequence
  synthetic construct
STSNLAS

SEQ ID NO: 124 - protein, 7 aa, Artificial sequence
  synthetic construct
YTSNLAS

SEQ ID NO: 125 - protein, 7 aa, Artificial sequence
  synthetic construct
STSSLQS

SEQ ID NO: 126 - protein, 9 aa, Artificial sequence
  synthetic construct
QQRSSYPLT

SEQ ID NO: 127 - protein, 120 aa, Artificial sequence
  synthetic construct
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSS

SEQ ID NO: 128 - protein, 106 aa, Artificial sequence
  synthetic construct
ENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELK

SEQ ID NO: 129 - protein, 120 aa, Artificial sequence
  synthetic construct
QVQLVQSGAEVKKPGASVKVSCKASGFNIKDSYMHWVRQAPGQGLEWMGWIDPENGDTEYAPKFQGRVTMTTDTSISTAYMELSRLRSDDTAVYYCNEGTPTGPYYFDYWGQGTLVTVSS

SEQ ID NO: 130 - protein, 120 aa, Artificial sequence
  synthetic construct
QVQLVQSGAEVKKPGASVKVSCKASGYTFKDSYMHWVRQAPGQGLEWMGWIDPENGDTEYAPKFQGRVTMTTDTSISTAYMELSRLRSDDTAVYYCNEGTPTGPYYFDYWGQGTLVTVSS

SEQ ID NO: 131 - protein, 120 aa, Artificial sequence
  synthetic construct
QVQLVQSGAEVKKPGASVKVSCKASGFNIKDSYMHWVRQAPGQGLEWMGWIDPENGGTNYAQKFQGRVTMTTDTSISTAYMELSRLRSDDTAVYYCNEGTPTGPYYFDYWGQGTLVTVSS

SEQ ID NO: 132 - protein, 120 aa, Artificial sequence
  synthetic construct
QVQLVQSGAEVKKPGASVKVSCKASGFNIKDSYMHWVRQAPGQGLEWMGWIDPENGDTEYAPKFQGRVTMTTDTSISTAYMELSRLRSDDTAVYYCARGTPTGPYYFDYWGQGTLVTVSS

SEQ ID NO: 133 - protein, 120 aa, Artificial sequence
  synthetic construct
QVQLVQSGAEVKKPGASVKVSCKASGFNIKDSYMHWVRQAPGQGLEWMGWIDPENGDTEYAPKFQGRVTMTRDTSISTAYMELSRLRSDDTAVYYCNEGTPTGPYYFDYWGQGTLVTVSS

SEQ ID NO: 134 - protein, 120 aa, Artificial sequence
  synthetic construct
QVQLVQSGAEVKKPGSSVKVSCKASGFNIKDSYMHWVRQAPGQGLEWMGWIDPENGDTEYAPKFQGRVTITTDESTSTAYMELSSLRSEDTAVYYCNEGTPTGPYYFDYWGQGTLVTVSS

SEQ ID NO: 135 - protein, 106 aa, Artificial sequence
  synthetic construct
DIQMTQSPSSLSASVGDRVTITCRASSSVSYMHWYQQKPGKAPKLLIYSTSNLASGVPSRFSGSGSGTDFTLTISSLQPEDFATYYCQQRSSYPLTFGGGTKLEIK

SEQ ID NO: 136 - protein, 106 aa, Artificial sequence
  synthetic construct
EIQMTQSPSSLSASVGDRVTITCRASSSVSYMHWYQQKPGKAPKLLIYSTSNLASGVPSRFSGSGSGTDFTLTISSLQPEDFATYYCQQRSSYPLTFGGGTKLEIK

SEQ ID NO: 137 - protein, 107 aa, Artificial sequence
  synthetic construct
EIQMTQSPSSLSASVGDRVTITCRASQSISSYMHWYQQKPGKAPKLLIYSTSNLASGVPSRFSGSGSGTDFTLTISSLQPEDFATYYCQQRSSYPLTFGGGTKLEIK

SEQ ID NO: 138 - protein, 106 aa, Artificial sequence
  synthetic construct
EIQMTQSPSSLSASVGDRVTITCRASSSVPYMHWYQQKPGKAPKLLIYSTSNLASGVPSRFSGSGSGTDFTLTISSVQPEDFATYYCQQRSSYPLTFGGGTKLEIK

SEQ ID NO: 139 - protein, 106 aa, Artificial sequence
  synthetic construct
EIQMTQSPSSLSASVGDRVTITCRASSSVPYMHWLQQKPGKAPKLLIYSTSNLASGVPSRFSGSGSGTDFTLTISSVQPEDFATYYCQQRSSYPLTFGGGTKLEIK

SEQ ID NO: 140 - protein, 106 aa, Artificial sequence
  synthetic construct
EIQMTQSPSSLSASVGDRVTITCRASSSVPYMHWLQQKPGKAPKLLIYSTSSLQSGVPSRFSGSGSGTDFTLTISSVQPEDFATYYCQQRSSYPLTFGGGTKLEIK

SEQ ID NO: 141 - protein, 88 aa, Artificial sequence
  synthetic construct
PKPFITSNNSNPVEDEDAVALTCEPEIQNTTYLWWVNNQSLPVSPRLQLSNDNRTLTLLSVTRNDVGPYECGIQNKLSVDHSDPVILN

SEQ ID NO: 142 - protein, 88 aa, Artificial sequence
  synthetic construct
PKPSISSNNSKPVEDKDAVAFTCEPETQDATYLWWVNNQSLPVSPRLQLSNGNRTLTLFNVTRNDTASYKCETQNPVSARRSDSVILN

SEQ ID NO: 143 - protein, 122 aa, Artificial Sequence
  synthetic construct
VTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSA

SEQ ID NO: 144 - protein, 592 aa, Artificial Sequence
  synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSA

SEQ ID NO: 145 - protein, 21 aa, Artificial Sequence
  synthetic construct
GGGGSGGGGSGGGGSGGSGGS

SEQ ID NO: 146 - protein, 21 aa, Artificial Sequence
  synthetic construct
GGGGSGGGGSGGGGSGGSGGG

SEQ ID NO: 147 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 148 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 149 - protein, 218 aa, Artificial Sequence; identical to SEQ ID NO: 89
  synthetic construct

SEQ ID NO: 150 - protein, 591 aa, Artificial Sequence
  synthetic construct
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSS

SEQ ID NO: 151 - protein, 10 aa, Artificial Sequence
  synthetic construct
GFTFTDYYMN

SEQ ID NO: 152 - protein, 20 aa, Artificial Sequence
  synthetic construct
GGGGSGGGGSGGGGSGGSGG

SEQ ID NO: 153 - protein, 594 aa, Artificial Sequence
  synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSAST

SEQ ID NO: 154 - protein, 215 aa, Artificial Sequence
  synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 155 - protein, 583 aa, Artificial Sequence
  synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSGGGGSQSVEESGGRLVTPGTPLTLTCTVSGFSLSTYSMSWVRQAPGKGLEWIGFIGSRGDTYYASWAKGRFTVSRTSTTVDLKITSPTTEDTATYFCARERDPYGGGAYPPHLWGPGTLVTVSS

SEQ ID NO: 156 - protein, 575 aa, Artificial Sequence
  synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGGGGGSGGGGSGGGGSGGGGSAVLTQTPSPVSPAVGGTVTISCQSSHSVYSDNDLAWYQQKLGQPPKLLIYQASKLASGVSSRFSGSGSGTQFTLTISGVQSDDAATYYCLGGYDDESDTYGFGGGTEVVVK

SEQ ID NO: 157 - protein, 581 aa, Artificial Sequence; identical to SEQ ID NO: 84
  synthetic construct

(19) United States
(12) Patent Application Publication
(10) Pub. No.: US 2024/0173442 A1
(43) Pub. Date: May 30, 2024

(54) COMBINATION THERAPY

(71) Applicant: Hoffmann-La Roche Inc., Little Falls, NJ (US)

(72) Inventors: Sara COLOMBETTI, Schlieren (CH); Sofia FROST, Schlieren (CH)

(21) Appl. No.: 18/271,928

(22) PCT Filed: Jan. 11, 2022
(86) PCT No.: PCT/EP2022/050453

§ 371 Date: Jul. 12, 2023

(30) Foreign Application Priority Data
Jan. 13, 2021 (EP) 21151407.0
Jul. 9, 2021 (EP) 21184780.1

(51) Int. Cl. A61K 51/10; A61K 39/395; A61P 35/00

(52) U.S. Cl. CPC A61K 51/1048; A61K 39/3955; A61K 51/1096; A61P 35/00

(57) ABSTRACT

The present invention relates to combination therapies including the use of pre-targeted radioimmunotherapy (PRIT).

### FIELD OF THE INVENTION

The present invention relates to combination therapies for use in the treatment of cancer.

### BACKGROUND

Monoclonal antibodies have been developed to target drugs to cancer cells. By conjugating a toxic agent to an antibody which binds to a tumour-associated antigen, there is the potential to provide more specific tumour killing with less damage to surrounding tissues.

In pre-targeted radioimmunotherapy (PRIT), use is made of an antibody construct which has affinity for the tumour-associated antigen on the one hand and for a radiolabelled compound on the other. In a first step, the antibody is administered and localises to tumour. Subsequently, the radiolabelled compound is administered. Because the radiolabelled compound is small, it can be delivered quickly to the tumour and is fast-clearing, which reduces radiation exposure outside of the tumour (Goldenberg et al Theranostics 2012, 2(5), 523-540). A similar procedure can also be used for imaging. Pre-targeting can make use of a bispecific antibody or systems using avidin-biotin, although the latter has the disadvantage that avidin/streptavidin is immunogenic.

The present invention relates to combination therapies including the use of PRIT.

### SUMMARY

The present invention relates to a combination therapy involving the use of i) a multispecific antibody or split multispecific antibody, said multispecific antibody or split multispecific antibody having a binding site for a radiolabelled compound and a binding site for a target antigen; ii) a CD40 agonist and iii) an immune checkpoint inhibitor.

In one aspect, the present invention relates to a pharmaceutical product comprising A) as a first component a composition comprising as an active ingredient a multispecific antibody or a split multispecific antibody having a binding site for a radiolabelled compound and a binding site for a target antigen; B) as a second component a composition comprising as an active ingredient a CD40 agonist; and C) as a third component a composition comprising as an active ingredient an immune checkpoint inhibitor, preferably a PD-L1 inhibitor, for the combined, simultaneous or sequential, treatment of a proliferative disease, preferably cancer. In some embodiments, the pharmaceutical product may further comprise the radiolabelled compound.

In another aspect the present invention provides a kit comprising the pharmaceutical product as disclosed herein together with instructions to use it.

In another aspect, the present invention relates to a multispecific antibody or a split multispecific antibody having a binding site for a radiolabelled compound and a binding site for a target antigen, for use in a method of treating a proliferative disease such as cancer, wherein the treatment comprises administering the multispecific antibody or split multispecific antibody, and wherein the treatment further comprises administering i) the radiolabelled compound, ii) an CD40 agonist and iii) an immune checkpoint inhibitor.

In another aspect, the present invention relates to a CD40 agonist for use in a method of treating a proliferative disease such as cancer, wherein the treatment further comprises administering i) a multispecific antibody or split multispecific antibody having a binding site for a radiolabelled compound and a binding site for a target antigen; ii) the radiolabelled compound, and iii) an immune checkpoint inhibitor.

In another aspect the present invention relates to an immune checkpoint inhibitor for use in a method of treating a proliferative disease such as cancer, wherein the treatment further comprises administering i) a multispecific antibody or split multispecific antibody having a binding site for a radiolabelled compound and a binding site for a target antigen; ii) the radiolabelled compound and iii) a CD40 agonist.

In another aspect, the present invention relates to i) a multispecific antibody or a split multispecific antibody having a binding site for a radiolabelled compound and a binding site for a target antigen; ii) the radiolabelled compound; iii) a CD40 agonist and iv) an immune checkpoint inhibitor for use in combination in a method of treating a proliferative disease such as cancer.

In another aspect, the invention relates to a method of treating a proliferative disease such as cancer, comprising administering to a patient i) a multispecific antibody or split multispecific antibody having a binding site for a radiolabelled compound and a binding site for a target antigen; ii) a radiolabelled compound; iii) a CD40 agonist; and iv) an immune checkpoint inhibitor.

In another aspect, the invention relates to a method of treating a proliferative disease such as cancer in a subject, comprising:
i) a radioimmunotherapy treatment comprising administering to the subject a multispecific antibody or split multispecific antibody, said multispecific antibody or split multispecific antibody having a binding site for a radiolabelled compound and a binding site for a target antigen, and further comprising administering to the subject the radiolabelled compound; and
ii) an immunotherapy treatment comprising administering to the subject a CD40 agonist and an immune checkpoint inhibitor.

The radiolabelled compound is administered to the patient after the multispecific antibody or the split multispecific antibody. The multispecific antibody or split multispecific antibody binds to the target antigen. The radiolabelled compound then binds to the multispecific antibody or split multispecific antibody, and is thus localised to the target cell.

The anti-CD40 antibody and immune checkpoint inhibitor can be administered simultaneously or sequentially, in either order. They may be administered before or after the administration of the multispecific antibody/split multispecific antibody and the radiolabelled compound. Preferably, they are administered after the multispecific antibody/split multispecific antibody and the radiolabelled compound.

In one embodiment, a cycle of the treatment comprises a first step of pre-targeted radioimmunotherapy comprising administering the multispecific antibody or split multispecific antibody and then administering the radiolabelled compound, and a second step of immunotherapy comprising administering a CD40 agonist and an immune checkpoint inhibitor, wherein the anti-CD40 antibody and the immune checkpoint inhibitor are administered simultaneously or sequentially in either order.

The treatment may comprise one cycle, or may comprise multiple cycles, e.g., 2, 3, 4, 5, or 6 cycles.

In some embodiments, not all cycles of the treatment are the same. In some embodiments:
the first cycle comprises a first step of pre-targeted radioimmunotherapy comprising administering the multispecific antibody or split multispecific antibody and then administering the radiolabelled compound, and a second step of immunotherapy comprising administering a CD40 agonist and an immune checkpoint inhibitor, wherein the anti-CD40 antibody and the immune checkpoint inhibitor are administered simultaneously or sequentially in either order; and
one or more subsequent cycles comprises a first step of pre-targeted radioimmunotherapy comprising administering the multispecific antibody or split multispecific antibody and then administering the radiolabelled compound, and a second step of immunotherapy comprising administering an immune checkpoint inhibitor.

For instance, there may be 1, 2, 3, 4 or 5 subsequent cycles as described above.

The multispecific antibody or split multispecific antibody may be a bispecific antibody or split bispecific antibody.

In one embodiment, the multispecific antibody (e.g., bispecific antibody) may be an antibody comprising at least one binding site for the target antigen, and at least one binding site for a radiolabelled compound, e.g., a Pb-DOTAM chelate. Exemplary antibodies are described in WO2019/201959.

A split multispecific antibody is comprised of two different parts, referred to herein as hemibodies. Each hemibody comprises an antigen binding moiety capable of binding to the target antigen. The antigen binding site for the radiolabelled compound is split across the two hemibodies such that a functional antigen binding site is formed only when the two hemibodies are associated. Thus, the split antibody comprises:
i) a first hemibody that binds to the target antigen (i.e., comprises an antigen binding moiety capable of binding to the target antigen), and which further comprises a VH domain of an antigen binding site for a radiolabelled compound, but which does not comprise a VL domain of an antigen binding site for the radiolabelled compound; and
ii) a second hemibody that binds to the target antigen (i.e., comprises an antigen binding moiety capable of binding to the target antigen), and which further comprises a VL domain of an antigen binding site for the radiolabelled compound, but which does not comprise a VH domain of the antigen binding site for the radiolabelled compound,
wherein said VH domain of the first hemibody and said VL domain of the second hemibody are together capable of forming a functional antigen binding site for the radiolabelled compound.

Neither the first nor the second hemibody comprise, on their own, a functional antigen binding site for a radiolabelled compound. The first hemibody has only a VH domain from the functional binding site for the radiolabelled compound, and not the VL domain. The second hemibody has only the VL domain, and not the VH domain.

A functional antigen binding site for the radiolabelled compound is formed when the VH and VL domains of the first and second hemibodies are associated. This may occur, for example, when the first and second antibodies are bound to the same individual target cell or to adjacent cells.

The terms “hemibodies”, “demibodies”, SPLITs, and “single domain split antibodies” may be used interchangeably. Exemplary hemibodies/demibodies are described in co-pending application PCT/EP2020/069561.

When the treatment makes use of a single antibody molecule comprising at least one binding site for the target antigen and at least one binding site for a radiolabelled compound, (i.e., does not make use of split antibodies), the treatment may also comprise administration of a clearing agent. The clearing agent is administered after the multispecific antibody and before the radiolabelled compound. The clearing agent binds to the antigen binding site for the radiolabelled compound. The clearing agent blocks the antigen binding site for the radiolabelled compound, preventing circulating antibody from binding to the chelated radionuclide. Alternatively or additionally, the clearing agent may increase the rate of clearance of antibody from the body. The “clearing agent” may alternatively be referred to as a “blocking agent”: these terms can be substituted for each other in the discussion that follows. The clearing agent may be conjugated to a clearing moiety as discussed further herein.

When the treatment makes use of hemibodies, it is not required that the treatment comprises a clearing step. That is, in some embodiments, the method does not comprise a step of administering a clearing agent or a blocking agent between the administration of the first and second hemibodies and the administration of radiolabelled compound (i.e., after the administration of the hemibodies but before administration of the radiolabelled compound).

In another embodiment, no agent is administered between the administration of the first and second hemibodies and the administration of radiolabelled compound, other than optionally a radiosensitizer and/or a chemotherapeutic agent. In another embodiment, no agent is administered between the administration of the first and second hemibodies and the administration of radiolabelled compound.

In some embodiments, the combination therapy results in one or more advantages compared to treatment with the pre-targeted radioimmunotherapy alone and/or with the immunotherapy alone. The reference treatment with the pre-targeted radioimmunotherapy alone involves administering the same pre-targeted radioimmunotherapy treatment as in the combination therapy (i.e., the same compounds, dose, administration times, number of cycles) but without the immunotherapy (i.e., without administration of the CD40 agonist or immune checkpoint inhibitor). The reference treatment with the immunotherapy alone involves administering the same immunotherapy treatment as in the combination therapy (i.e., the same CD40 agonist and immune checkpoint inhibitor compounds, dose, administration times, number of cycles) but without the pre-targeted radioimmunotherapy (i.e., without administration of the multispecific/split multispecific antibody, radiolabelled compound and clearing agent, as applicable). In some embodiments, the combination therapy results in a slower rate of tumour growth than the pre-targeted radioimmunotherapy alone and/or the immunotherapy alone. In some embodiments, the combination therapy results in an increased likelihood of patient/subject survival than treatment with the pre-targeted radioimmunotherapy alone and/or with the immunotherapy alone. In some embodiments, the combination therapy results in an increased frequency of activated intratumoral CD8 T cells (e.g., as measured by upregulation of 41BB expression), and/or an increased frequency of activated plasmacytoid DCs (pDCs) and classical DCs (cDCs) in tumor, spleen and draining lymphnodes (DLNs) (e.g., as measured by upregulation of CD86 expression), and/or increased frequency of T cells in total immune cells than treatment with the pre-targeted radioimmunotherapy alone and/or with the immunotherapy alone. In some embodiments, the combination therapy results in an enhanced immune memory response or reduced likelihood of tumour recurrence than treatment with the pre-targeted radioimmunotherapy alone and/or with the immunotherapy alone.

In some embodiments, the cancer is refractory to the immune checkpoint inhibitor.

In some embodiments, it may be preferred that the target antigen is CEA.

In some embodiments, it may be preferred that the radiolabelled compound is Pb-DOTAM.

### BRIEF DESCRIPTION OF THE FIGURES

FIG. 1 shows the schematic structure of a target antigen (TA)-DOTAM bispecific antibody (TA-DOTAM BsAb), and exemplary TA-split-DOTAM-VH/VL antibodies.

FIG. 2 is a schematic diagram showing the assembly of a split-VH/VL DOTAM binder on tumour cells. The TA-split-DOTAM-VH/VL antibodies will not significantly bind ²¹²Pb-DOTAM unless bound to tumour antigen (TA) on targeted cells, where the two domains of the DOTAM binder are assembled.

FIG. 3 shows a schematic overview of an example of the Three-Step TA-PRIT concept, involving use of a clearing agent.

FIG. 4 shows a schematic overview of an example of the Two-Step TA-PRIT concept, in which a clearing agent is not used.

FIG. 5 shows binding of split antibodies to MKN45 cells to demonstrate CEA binding competence. Detection of antibodies is done using human IgG specific secondary antibodies

FIG. 6 shows binding of split antibodies to MKN45 cells to demonstrate DOTAM binding competence. Detection of antibodies is done using Pb-DOTAM-FITC.

FIG. 7A shows an exemplary protocol for two-step PRIT with a CEA-split-DOTAM-VH/NL, carried out in in SCID mice carrying SC BxPC3 tumours (h=hours, d=days, w=weeks).

FIG. 7B shows an exemplary protocol for a three-step PRIT control, carried out in SCID mice carrying SC BxPC3 tumours (h=hours, d=days, w=weeks).

FIG. 8 shows the biodistribution of pretargeted ²¹²Pb-DOTAM in SCID mice carrying SC BxPC3 tumors, 6 hours after injection of ²¹²Pb-DOTAM, pretargeted either by CEA-split-DOTAM-VH alone, CEA-split-DOTAM-VL alone, or the two complementary antibodies combined, or using standard three-step PRIT (% ID/g±SD, n=4).

FIG. 9 shows CEA-Split-DOTAM-VH/VL pharmacokinetics after IV injection in SCID mice.

FIG. 10 shows the experimental design of protocol 158, comprising CEA-PRIT in 2 (top) or 3 steps (bottom) in SCID mice carrying SC BxPC3 tumors. *CEA split DOTAM BsAb dose adjusted to compensate for hole/hole impurities in 2/4 constructs.

FIG. 11 shows the biodistribution of pretargeted ²¹²Pb-DOTAM in SCID mice carrying SC BxPC3 tumors (6 h p.i.). The distribution is of ²¹²Pb in tumour-bearing SCID mice, 6 hours after injection of ²¹²Pb-DOTAM, pretargeted by CEA-DOTAM BsAb or bi-paratopic combinations of CEA-split-DOTAM antibodies. The radioactive content in organs and tissues is expressed as average % ID/g±SD (n=4).

FIG. 12 shows the experimental schedule of protocol 160, comprising one cycle of 3-step CEA-PRIT (top), 2-step CEA-PRIT (middle), or 1-step CEA-RIT in SCID mice carrying SC BxPC3 tumors. Biodistribution (BD) scouts were euthanized 24 hours after the radioactive injection, whereas mice in the efficacy groups were maintained and monitored carefully until the termination criteria were reached.

FIG. 13 shows biodistribution of pretargeted ²¹²Pb-DOTAM and ²¹²Pb-DOTAM-CEA-DOTAM in SCID mice carrying SC BxPC3 tumors (24 h p.i.). The distribution is of ²¹²Pb in tumor-bearing SCID mice 24 hours after injection of CEA-DOTAM-pretargeted ²¹²Pb-DOTAM or pre-incubated ²¹²Pb-DOTAM-CEA-DOTAM. The radioactive content in organs and tissues is expressed as average % ID/g±SD (n=3).

FIG. 14 shows tumor growth averages with standard error for PRIT-treated groups and control (groups A-E) in the BxPC3 model (n=10). Curves were truncated at n<5. Dotted vertical lines indicate ²¹²Pb-DOTAM administration (20 μCi) for some or all groups, according to the study design.

FIG. 15 shows individual tumor growth curves for PRIT-treated groups and control (groups A-E) in the BxPC3 model (n=10). Dotted vertical lines indicate administration of ²¹²Pb-labeled compounds (20 μCi).

FIG. 16 shows average body weight loss in mice treated with CEA-PRIT and CEA-RIT (groups A-E, n=10) in the BxPC3 model. Curves were truncated at n<5. Dotted vertical lines indicate administration of ²¹²Pb-labeled compounds for some or all groups, according to the study design.

FIG. 17 shows the experimental design of protocol 175, comprising two-step CEA-PRIT in SCID mice carrying SC BxPC3 tumors, with sacrifice and necropsy 24 hours after the ²¹²Pb-DOTAM injection. The CEA-split-DOTAM-VH-AST dose was adjusted to compensate for hole/hole impurities.

FIG. 18 shows distribution of ²¹²Pb in tumor-bearing SCID mice 24 hours after injection of ²¹²Pb-DOTAM, pretargeted by CEA-split-DOTAM-VH/VL antibodies (protocol 175). The radioactive content in organs and tissues is expressed as average % ID/g±SD (n=4).

FIG. 19 shows the experimental design of protocol 185, comprising two-step CEA-PRIT in SCID mice carrying SC BxPC3 tumors, with sacrifice and necropsy 6 hours after the ²¹²Pb-DOTAM injection. The CEA-split-DOTAM-VH-AST (CH1A1A) dose was adjusted to compensate for hole/hole impurities.

FIG. 20 shows distribution of ²¹²Pb in tumor-bearing SCID mice 6 hours after injection of ²¹²Pb-DOTAM, pretargeted by CEA-split-DOTAM-VH/VL antibodies (protocol 185). The radioactive content in organs and tissues is expressed as average % ID/g±SD (n=5).

FIG. 21 shows distribution of CEA-split-DOTAM-VH/VL pairs (VH and VL antibodies combined) in two selected SC BxPC3 tumors 7 days after injection. A and B show sections of a tumor from mouse A3, injected with CEA-split-DOTAM-VH/VL targeting T84.66, where A shows the CEA expression, and B shows the corresponding CEA-split-DOTAM-VH/VL distribution. C and D show tumor sections from mouse C5, injected with CEA-split-DOTAM-VH/VL targeting CH1A1A: C showing the CEA expression and D the corresponding CEA-split-DOTAM-VH/VL distribution.

FIG. 22 shows the experimental design of protocol 189, comprising two-step CEA-PRIT in SCID mice carrying SC BxPC3 tumors, with sacrifice and necropsy 6 hours after the ²¹²Pb-DOTAM injection. The CEA-split-DOTAM-VH-AST (CH1A1A) dose was adjusted to compensate for hole/hole impurities.

FIG. 23 shows distribution of ²¹²Pb in tumor-bearing SCID mice 6 hours after injection of ²¹²Pb-DOTAM, pretargeted by bi-paratopic pairs of CEA-split-DOTAM-VH/VL antibodies (T84.66 and CH1A1A), compared with the positive control (CH1A1A only). The radioactive content in organs and tissues is expressed as average % ID/g±SD.

FIG. 24 shows mean Flurescence Intensity (MFI) as determined by FACS for SPLIT antibodies. Binding of Pb-DOTA-FITC determined by FACS can only be shown for a co-incubation of both SPLIT antibodies with Pb-DOTA-FITC. Single SPLIT antibodies did not give rise to a significant signal.

FIG. 25A-C shows further exemplary formats of split antibodies.

FIG. 26 shows results from example 8, experiment 1, assessing binding of individual TA-split-DOTAM-VH and TA-split-DOTAM-VL antibodies to biotinylated DOTAM captured on a chip.

FIG. 27 shows results from example 8, experiment 2, assessing binding of DOTAM to individual TA-split-DOTAM-VH and TA-split-DOTAM-VL antibodies captured on a chip.

FIG. 28 shows results from example 8, experiment 3, assessing binding of DOTAM to TA-split-DOTAM-VH/VL antibodies (antibody pairs), captured on a chip.

FIG. 29 shows the study outline of protocol 119, assessing CEA-PRIT of orthotopic PancO2-huCEA-luc tumors in B6-huCEA mice (IPANC=intrapancreatic, d=days, h=hours).

FIG. 30 shows distribution of ²¹²Pb in tumor-bearing B6-huCEA mice 24 hours after injection of ²¹²Pb-DOTAM pretargeted by CEA-DOTAM (mu) (cycle 1). The radioactive content in organs and tissues is expressed as average % ID/g±standard deviation (SD; n=3).

FIG. 31 shows serum concentration of anti-CD40 and anti-PD-L1 antibodies 24 hours after IP administration (200 μg/antibody/mouse) to mice in groups B and D of protocol 119, as determined by ELISA. The graphs are showing individual values with mean±SD for each treatment cycle.

FIG. 32 shows average background-subtracted BLI signal for groups A-D in the orthotopic Panc02-huCEA-luc model, expressed as photons (P) per second per mm²±standard error of the mean (SEM; n=8). Dashed and dotted vertical lines indicate immunotherapy and ²¹²Pb-DOTAM administration (20 μCi), respectively, for some or all groups, according to the study design.

FIG. 33 shows background-subtracted BLI signal for individual mice in groups A-D in the orthotopic Panc02-huCEA-luc model, expressed as photons (P) per second per mm² (n=8). Dashed and dotted vertical lines indicate immunotherapy and ²¹²Pb-DOTAM administration (20 μCi), respectively, for some or all groups, according to the study design. The arrow indicates day 88, the last day of imaging, by which time 3 mice in group D were still alive and without signal.

FIG. 34 shows Kaplan-Meier curves showing the survival in groups A-D in the orthotopic Panc02-huCEA-luc model (n=8). Dotted and dashed vertical lines indicate ²¹²Pb-DOTAM (20 μCi) and immunotherapy administration, respectively, for some or all groups, according to the study design.

FIG. 35 shows average change in BW after the various treatments, expressed as percentage of initial BW±SEM. The dotted and dashed lines indicate ²¹²Pb-DOTAM and immunotherapy administration, respectively, depending on the treatment scheme.

FIG. 36 shows the study outline of protocol 136, assessing CEA-PRIT of SC Panc02-huCEA-luc tumors in B6-huCEA mice (d=days, h=hours).

FIG. 37 shows distribution of ²¹²Pb in tumor-bearing B6-huCEA mice 24 hours after injection of ²¹²Pb-DOTAM, pretargeted by CEA-DOTAM (mu) (cycle 1). The radioactive content in organs and tissues is expressed as average % ID/g±SD (n=3).

FIG. 38 shows serum concentration of anti-CD40 and anti-PD-L1 24 hours after IP administration (200 μg/antibody/mouse) to mice in groups B and D, as determined by ELISA. The graphs are showing individual values and the mean±SD for each treatment cycle. The asterisks (*) in the right graph indicate skewed averages due to outliers (not shown on graph; 1 data point for cycle 1 and 3 data points for cycle 3).

FIG. 39 shows tumor growth averages with standard error for groups A-D in the SC Panc02-huCEA-luc model (n=9). Curves were truncated at n<5. Dashed and dotted vertical lines indicate immunotherapy and ²¹²Pb-DOTAM administration (20 μCi), respectively, for some or all groups according to the study design.

FIG. 40 shows individual tumor growth curves for groups A-D in the SC Panc02-huCEA-luc model (n=9). Dashed and dotted vertical lines indicate administration of immunotherapy and ²¹²Pb-DOTAM (20 μCi), respectively.

FIG. 41 shows average background-subtracted BLI signal for groups A-D in the SC Panc02-huCEA-luc model, expressed as photons (P) per second per mm2±SEM (n=9). Dashed and dotted vertical lines indicate immunotherapy and ²¹²Pb-DOTAM administration (20 μCi), respectively, for some or all groups, according to the study design.

FIG. 42 shows background-subtracted BLI signal for individual mice in groups A-D in the SC Panc02-huCEA-luc model, expressed as photons (P) per second per mm² (n=9). Dashed and dotted vertical lines indicate immunotherapy and ²¹²Pb-DOTAM administration (20 μCi), respectively, for some or all groups, according to the study design.

FIG. 43 shows Kaplan-Meier curves showing the survival in groups A-D in the SC Panc02-huCEA-luc model, based on the termination criteria of tumor volume ≥3000 mm³ (n=9). Dashed and dotted vertical lines indicate immunotherapy and ²¹²Pb-DOTAM administration (20 μCi), respectively, for some or all groups, according to the study design.

FIG. 44 shows FACS analysis of DLN, spleen, and tumor samples from mice treated with 2 cycles of immunotherapy, CEA-PRIT, CEA-PRIT+immunotherapy, or no treatment, showing T cell activation. Samples were taken 24 hours after the immunotherapy injection, corresponding to 48 hours after the ²¹²Pb-DOTAM irradiation. Asterisks indicate level of significance (one-way ANOVA, p<0.05, n=4).

FIG. 45 shows FACS analysis of DLN, spleen, and tumor samples from mice treated with 2 cycles of immunotherapy, CEA-PRIT, CEA-PRIT+immunotherapy, or no treatment, showing activation of cDCs and pDCs. Markers for the pDC subpopulation: MHCII+ CD11c^{int} CD317+; markers for CD11b− cDC subpopulation (cross-presenting DCs): MHCII+ CD11c^{high} CD11b−; markers for CD11b+ cDC subpopulation: MHCII+ CD11c^{high} CD11b+. Samples were taken 24 hours after the immunotherapy injection, corresponding to 48 hours after the ²¹²Pb-DOTAM irradiation. Asterisks indicate level of significance (one-way ANOVA, p<0.05, n=4).

FIG. 46 shows FACS analysis of DLN, spleen, and tumor samples from mice treated with 2 cycles of immunotherapy, CEA-PRIT, CEA-PRIT+immunotherapy, or no treatment, showing overall T cell frequency. Samples were taken 24 hours after the immunotherapy injection, corresponding to 48 hours after the ²¹²Pb-DOTAM irradiation. Asterisks indicate level of significance (one-way ANOVA, p<0.05, n=4).

FIG. 47 shows tumor growth curves for rechallenged and naïve B6-huCEA mice in the SC Panc02-huCEA-luc model (n=5). Rechallenged mice were initially tumor-carriers, rendered tumor-free after 3 cycles of CEA-PRIT+CIT (anti-CD40+anti-PD-L1).

FIG. 48 shows FACS analysis of blood, spleen, and lymph node (LN) samples from rechallenged and naïve mice in the SC Panc02-huCEA-luc model (n=5). Rechallenged mice were initially tumor-carriers, rendered tumor-free after 3 cycles of CEA-PRIT+CIT (anti-CD40+anti-PD-L1). Asterisks indicate level of significance (unpaired t-test, p<0.05); dp=double-positive; iono=ionomycin.

FIG. 49 shows average change in BW after the various treatments, expressed as percentage of initial BW±SEM. The dotted and dashed lines indicate ²¹²Pb-DOTAM and immunotherapy administration, respectively, depending on the treatment scheme.

FIG. 50 shows the study outline of protocol 150, assessing CEA-PRIT of SC MC38-huCEA tumors in B6-huCEA mice (d=days, h=hours).

FIG. 51 shows distribution of ²¹²Pb in MC38-huCEA tumor-bearing B6-huCEA mice 24 hours after injection of ²¹²Pb-DOTAM pretargeted by CEA-DOTAM (mu) (cycle 1). The radioactive content in organs and tissues is expressed as average % ID/g±SD (n=4).

FIG. 52 shows serum concentration of anti-CD40 and anti-PD-L1 24 hours after IP administration (200 μg/antibody/mouse) to mice in groups B, C and E, as determined by ELISA. The graphs are showing individual values with mean±SD for each treatment cycle.

FIG. 53 shows tumor growth averages with standard error for groups A-E in the SC MC38-huCEA model (n=9). Curves were truncated at n<5. Dashed and dotted vertical lines indicate immunotherapy and ²¹²Pb-DOTAM administration (20 μCi), respectively, for some or all groups according to the study design. The arrow indicates immuno-PD analysis.

FIG. 54 shows individual tumor growth curves for groups A-E in the SC MC38-huCEA model (n=9). Dashed and dotted vertical lines indicate administration of immunotherapy and ²¹²Pb-DOTAM (20 μCi), respectively.

FIG. 55 shows Kaplan-Meier curves showing the survival in groups A-E in the SC MC38-huCEA model, based on the termination criteria of tumor volume ≥3000 mm³ (n=9). Symbols represent censored mice, euthanized for other reasons than tumor volume. Dashed and dotted vertical lines indicate immunotherapy and ²¹²Pb-DOTAM administration (20 μCi), respectively, for some or all groups, according to the study design.

FIG. 56 shows FACS analysis of lymph node samples from mice treated with 2 cycles of immunotherapy, CEA-PRIT, CEA-PRIT+immunotherapy, or no treatment. Samples were taken 24 hours after the immunotherapy injection, corresponding to 48 hours after the ²¹²Pb-DOTAM irradiation. Asterisks indicate level of significance (one-way ANOVA with correction for multiple comparisons [Tukey], p<0.05, n=4). MFI=mean fluorescence intensity.

FIG. 57 shows tumor growth curves for rechallenged and naïve (age-matched) B6-huCEA mice in the SC MC38-huCEA model. Rechallenged mice were initially tumor-carriers, rendered tumor-free after treatment with anti-CD40, anti-CD40+anti-PD-L1, CEA-PRIT, or CEA-PRIT+anti-CD40+anti-PD-L1.

FIG. 58 shows FACS analysis of rechallenged and naïve mice in the SC MC38-huCEA model. Rechallenged mice were initially tumor-carriers, rendered tumor-free after treatment with anti-CD40, anti-CD40+anti-PD-L1, CEA-PRIT, or CEA-PRIT+anti-CD40+anti-PD-L1. Asterisks indicate level of significance (one-way ANOVA with correction for multiple comparisons [Tukey], p<0.05, n=4); dp=double-positive.

FIG. 59 shows average change in BW after the various treatments, expressed as percentage of initial BW±SEM. The dotted and dashed lines indicate ²¹²Pb-DOTAM and immunotherapy administration, respectively, depending on the treatment scheme.

FIG. 60 shows the study outline of protocol 195, assessing SPLIT PRIT and/or CIT of SC Panc02-huCEA-Fluc tumors in huCEACAM5 mice (d=days, h=hours).

FIG. 61 shows distribution of ²¹²Pb in Panc02-huCEA-Fluc tumor-bearing huCEACAM5 mice 24 hours after injection of ²¹²Pb-DOTAM pretargeted by SPLIT CEA-PRIT. The radioactive content in organs and tissues is expressed as average % ID/g±SD (n=3).

FIG. 62 shows tumor growth averages with standard error for groups A-D in the SC Panc02-huCEA-Fluc model (n=10) (Protocol 195). Curves were truncated at n<5. Dashed and dotted vertical lines indicate immunotherapy and ²¹²Pb-DOTAM administration (20 μCi), respectively, for some or all groups according to the study design.

FIG. 63 shows individual tumor growth curves for groups A-D in the SC Panc02-huCEA-Fluc model (n=10) (protocol 195). Dashed and dotted vertical lines indicate administration of immunotherapy and ²¹²Pb-DOTAM (20 μCi), respectively.

FIG. 64 shows Kaplan-Meier curves showing the survival in groups A-D in the SC Panc02-huCEA-Fluc model (protocol 195), based on the termination criteria of tumor volume ≥2000 mm3 (n=10). Symbols represent censored mice, euthanized for other reasons than tumor volume. Dashed and dotted vertical lines indicate immunotherapy and ²¹²Pb-DOTAM administration (20 μCi), respectively, for some or all groups, according to the study design.

FIG. 65 shows tumor growth averages with standard error for rechallenged mice and naïve (age-matched) huCEACAM5 mice in the SC Panc02-huCEA-Fluc model. Rechallenged mice were initially tumor-carriers, rendered tumor-free after treatment with SPLIT CEA-PRIT+anti-CD40+anti-PD-L1. On day 13 after rechallenge, 3 mice per group were euthanized for immuno-PD analysis (data not shown).

FIG. 66 shows tumor growth curves for rechallenged and naïve (age-matched) huCEACAM5 mice in the SC Panc02-huCEA-Fluc model. Rechallenged mice were initially tumor-carriers, rendered tumor-free after treatment with SPLIT CEA-PRIT+anti-CD40+anti-PD-L1 On day 13 after rechallenge, 3 mice per group were euthanized for immuno-PD analysis (data not shown).

FIG. 67 shows the average change in BW after the various treatments, expressed as percentage of initial BW±SEM. The dotted and dashed lines indicate ²¹²Pb-DOTAM and immunotherapy administration, respectively, depending on the treatment scheme.

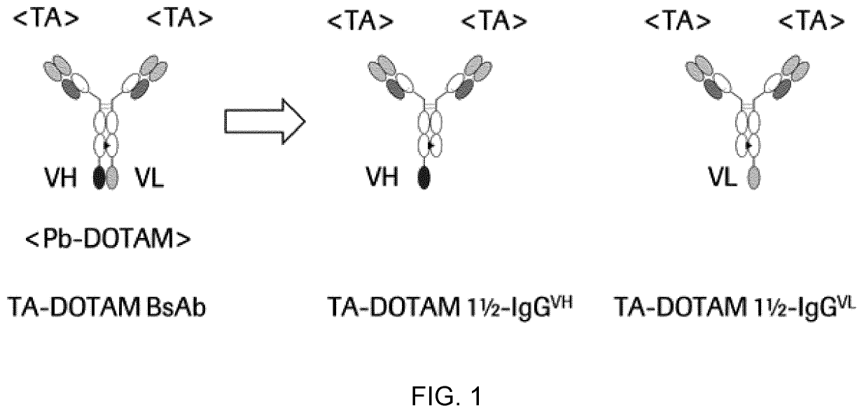

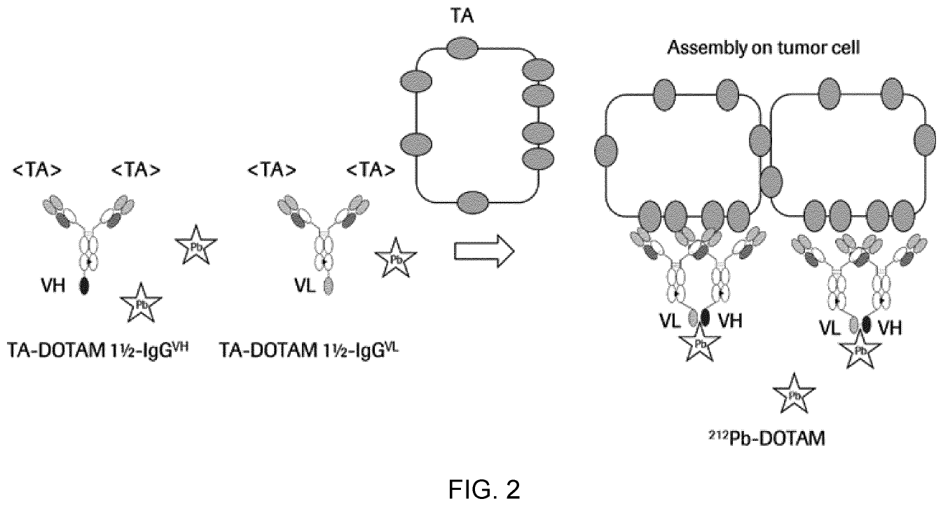

### DETAILED DESCRIPTION OF THE INVENTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary methods for measuring binding affinity are described in the following.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The term “a binding site for an antigen expressed on the surface of a target cell” or “a binding site for a target antigen” refers to a binding site that is capable of binding said antigen with sufficient affinity such that the antibody is useful as a diagnostic and/or therapeutic agent in targeting said antigen. The antibody comprising a binding site for a target antigen may comprise any binding moiety which binds to the target antigen with sufficient affinity.

In some embodiments, the antigen binding moiety may be an antibody fragment (such as a Fv, Fab, cross-Fab, Fab′, Fab′-SH, F(ab′)2; diabody; linear antibody; single-chain antibody molecule (e.g., scFv or scFab); or single domain antibody (dAbs) such as VHH). In other embodiments it may be a protein binding scaffold such as a DARPin (designed ankyrin repeat protein); affibody; Sso7d; monobody or anticalin.

In one aspect, the extent of binding of the antibody to an unrelated, non antigen protein is less than about 10% of the binding of the antibody to the antigen as measured, e.g., by surface plasmon resonance (SPR). In certain aspects, an antibody that binds to an antigen expressed on the surface of a target cell has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). An antibody is said to “specifically bind” to an antigen expressed on the surface of a target cell when the antibody has a K_{D} of 1 μM or less. In certain aspects, the antibody binds to an epitope of said antigen that is conserved among said antigen from different species.

The terms “an antigen binding site for a radiolabelled compound” or “a functional antigen binding site for a radiolabelled compound” refer to an antigen binding site capable of binding to the radiolabelled compound with sufficient affinity such that the antibody is useful as a diagnostic and/or therapeutic agent to associate the radiolabelled compound with the antibody. The antigen binding site for a radiolabelled compound preferably comprises a VH and VL domain. In one aspect, the extent of binding of the antigen binding site to an unrelated, non antigen-compound is less than about 10% of the binding of the antibody to the radiolabelled compound as measured, e.g., by surface plasmon resonance (SPR). In certain aspects, an antigen binding site that binds to a radiolabelled compound has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). It may be preferred that it has a K_{D} of 100 pM, 50 pM, 20 pM, 10 pM, 5 pM, 1 pM or less, e.g, 0.9 pM or less, 0.8 pM or less, 0.7 pM or less, 0.6 pM or less or 0.5 pM or less. For instance, the functional binding site may bind the radiolabelled compound with a K_{D} of about 1 pM-1 nM, e.g., about 1-10 pM, 1-100 pM, 5-50 pM, 100-500 pM or 500 pM-1 nM. An antigen binding site is said to “specifically bind” to a radiolabelled compound when the antigen binding site has a K_{D} of 1 μM or less.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, cross-Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005). The term “Fab fragment” refers to a protein consisting of the VH and CH1 domain of the heavy chain and the VL and CL domain of the light chain of an immunoglobulin. “Fab′ fragments” differ from Fab fragments by the addition of residues at the carboxy terminus of the CH1 domain including one or more cysteines from the antibody hinge region. For discussion of Fab and F(ab′)₂ fragments comprising salvage receptor binding epitope residues and having increased in vivo half-life, see U.S. Pat. No. 5,869,046.

As used herein, a reference to a “Fab fragment” is intended to include a cross-Fab fragment or a scFab as well as a conventional Fab fragment (i.e., one comprising a light chain comprising a VL domain and a CL domain, and a heavy chain fragment comprising a VH domain and a CH1 domain).

The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

As used herein, the term “single-chain” refers to a molecule comprising amino acid monomers linearly linked by peptide bonds. A single-chain Fab molecule is a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule.

Asymmetrical Fab arms can also be engineered by introducing charged or non-charged amino acid mutations into domain interfaces to direct correct Fab pairing. See e.g., WO 2016/172485.

A “single-chain variable fragment” or “scFv” is a fusion protein of the variable domains of the heavy (VH) and light chains (VL) of an antibody, connected by a peptide linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

A split antibody refers to an antibody in which the binding site for an antigen is split between two parts, such as two individual antibody molecules. The two parts may be referred to as “hemibodies” or “demibodies”. When the two parts are associated, a functional binding site for the antigen is formed. In the present invention, each hemibody comprises an antigen binding moiety for an antigen on the surface of a target cell, as well as either the VH or VL of an antigen binding site for a radiolabeled compound. When the two hemibodies bind to the same or adjacent target cells, a stable association may be formed between the VH and VL, thus forming a functional binding site for the radiolabeled compound. “CEA-targeted SPLIT PRIT” refers to a split antibody targeting CEA. The term “SPLIT PRIT” may also be used interchangeably with the term “TA-split-DOTAM-VH/VL”. The term “CEA-targeted SPLIT PRIT” may be used interchangeably with the term “CEA-split-DOTAM-VH/VL”.

The term “clearing agent” refers to an agent which increases the rate of clearance of an antibody from the circulation of the subject and/or which blocks the binding of an effector molecule, in particular the radiolabelled compound, to a functional binding site for that effector molecule. Generally the clearing agent binds to the antibody, e.g., specifically binds to the antibody. It may bind to the functional binding site for the effector molecule, e.g., specifically bind to the said functional binding site.

The term “clearing step” or “clearing phase” as used herein encompasses the use of an agent which increases the rate of clearance of an antibody from the circulation of the subject and/or which blocks the binding of an effector molecule. Some agents can function in both clearing and blocking.

The term “epitope” denotes the site on an antigen, either proteinaceous or nonproteinaceous, to which an antibody binds. Epitopes can be formed both from contiguous amino acid stretches (linear epitope) or comprise non-contiguous amino acids (conformational epitope), e.g., coming in spatial proximity due to the folding of the antigen, i.e. by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., NY).

Antigen Structure-based Antibody Profiling (ASAP), also known as Modification-Assisted Profiling (MAP), allows to bin a multitude of monoclonal antibodies specifically binding to an antigen based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin.

Also competitive binding can be used to easily determine whether an antibody binds to the same epitope as, or competes for binding with, a reference antibody. For example, an “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Also for example, to determine if an antibody binds to the same epitope as a reference antibody, the reference antibody is allowed to bind to the antigen under saturating conditions. After removal of the excess of the reference antibody, the ability of an antibody in question to bind to the antigen is assessed. If the antibody in question is able to bind to the antigen after saturation binding of the reference antibody, it can be concluded that the antibody in question binds to a different epitope than the reference antibody. But, if the antibody in question is not able to bind to the antigen after saturation binding of the reference antibody, then the antibody in question may bind to the same epitope as the epitope bound by the reference antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody is capable of binding to the antigen, then it can be concluded that the antibody in question and the reference antibody compete for binding to the antigen.

In some aspects, two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some aspects, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG₂ isotype. In certain aspects, the antibody is of the IgG₄ isotype with the S228P mutation in the hinge region to improve stability of IgG₄ antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., a pharmaceutical composition, refers to an amount effective, at dosages and for periods of time necessary, to achieve the desired therapeutic or prophylactic result.

The term “tandem Fab” refers to an antibody comprising two Fab fragments connected via a peptide linker/tether. In some embodiments, a tandem Fab may comprise one Fab fragment and one cross-Fab fragment, connected by a peptide linker/tether.

The term “Fc region” herein is used to define a C-terminal region of an immunoglobulin heavy chain that contains at least a portion of the constant region. The term “Fc domain” herein is used to define a C-terminal region of an immunoglobulin that contains the constant regions of two heavy chains, excluding the first constant region. Thus, Fc refers to the last two constant region immunoglobulin domains of IgA, IgD, and IgG, and the last three constant region immunoglobulin domains of IgE and IgM. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the Fc region may or may not be present. In one aspect, a heavy chain including an Fc region as specified herein, comprised in an antibody according to the invention, comprises an additional C-terminal glycine-lysine dipeptide (G446 and K447, numbering according to EU index). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991. A “subunit” of an Fc domain as used herein refers to one of the two polypeptides forming the dimeric Fc domain, i.e. a polypeptide comprising C-terminal constant regions of an immunoglobulin heavy chain, capable of stable association with the other of the two polypeptides forming the dimeric Fc domain. For example, a subunit of an IgG Fc domain comprises an IgG CH2 and an IgG CH3 constant domain.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein. As used herein, the term “full length antibody” denotes an antibody consisting of two “full length antibody heavy chains” and two “full length antibody light chains”. A “full length antibody heavy chain” may be a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in case of an antibody of the subclass IgE. Preferably the “full length antibody heavy chain” is a polypeptide consisting in N-terminal to C-terminal direction of VH, CH1, HR, CH2 and CH3. The possibility of cross-Mab formation is not intended to be excluded by the reference to “full length”—thus, the heavy chain may have the VH domain swapped for a VL domain, or the CH1 domain swapped for a CL domain. A “full length antibody light chain” may be a polypeptide consisting in N-terminal to C-terminal direction of an antibody light chain variable domain (VL), and an antibody light chain constant domain (CL), abbreviated as VL-CL. Alternatively, in the case of a cross-Mab, the VL domain may be swapped for a VH domain or the CL domain may be swapped for a CH1 domain. The antibody light chain constant domain (CL) can be κ (kappa) or γ (lambda). The two full length antibody chains are linked together via inter-polypeptide disulfide bonds between the CL domain and the CH1 domain and between the hinge regions of the full length antibody heavy chains. Examples of typical full length antibodies are natural antibodies like IgG (e.g. IgG1 and IgG2), IgM, IgA, IgD, and IgE.) Full length antibodies can be from a single species e.g. human, or they can be chimerized or humanized antibodies. The full length antibodies described herein comprise two antigen binding sites each formed by a pair of VH and VL, which may in some embodiments both specifically bind to the same antigen, or may bind to different antigens. The C-terminus of the heavy or light chain of said full length antibody denotes the last amino acid at the C-terminus of said heavy or light chain.

By “fused” is meant that the components are linked by peptide bonds, either directly or via one or more peptide linkers.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda M D (1991), vols. 1-3. In one aspect, for the VL, the subgroup is subgroup kappa I as in Kabat et al., supra. In one aspect, for the VH, the subgroup is subgroup III as in Kabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system. Instead of the above, the sequence of CDR-H1 as described herein may extend from Kabat26 to Kabat35, e.g., for the Pb-DOTAM binding variable domain.

In one aspect, CDR residues comprise those identified in the sequence tables or elsewhere in the specification.

Unless otherwise indicated, HVR/CDR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

Molecules as described herein may be “isolated”. An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www. ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

A reference to a target antigen as used herein, refers to any native target antigen from any vertebrate source, including mammals such as primates (e.g., humans) and rodents (e.g., mice and rats), unless otherwise indicated. The term encompasses “full-length”, unprocessed target antigen as well as any form of target antigen that results from processing in the cell.

The term also encompasses naturally occurring variants of the target antigen, e.g., splice variants or allelic variants. For instance, the target antigen CEA may have the amino acid sequence of human CEA, in particular Carcinoembryonic antigen-related cell adhesion molecule 5 (CEACAM5), which is shown in UniProt (www.uniprot.org) accession no. P06731 (version 119), or NCBI (www.ncbi.nlm.nih.gov/) RefSeq NP_004354.2.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “Pb” or “lead” as used herein include ions thereof, e.g., Pb(II). References to other metals also include ions thereof. Thus, the skilled reader understands that, for example, the terms lead, Pb, ²¹²Pb or ²⁰³Pb are intended to encompass ionic forms of the element, in particular, Pb(II).

### II. Compositions and Methods

### A. Radiolabelled Compounds

According to the present invention, the multispecific antibody or split multispecific antibody comprises a binding site for an effector molecule. (In a split multispecific antibody formed of a first and second hemibody, the first hemibody comprises the VH domain of the antigen binding site for the effector molecule and the second hemibody comprises the VL domain of the antigen binding site for the effector molecule, and the functional binding site is formed when the two hemibodies are associated).

Effector molecules according to the present invention are radiolabelled compounds which comprise a radioisotope, e.g., are a radiolabelled hapten.

In some embodiments, the effector molecule may comprise a chelated radioisotope.

In some embodiments, the functional binding site for the effector molecule may bind to a chelate comprising the chelator and the radioisotope. In other embodiments, the antibody may bind to a moiety which is conjugated to the chelated radioisotope, for instance, histamine-succinyl-glycine (HSG), digoxigenin, biotin or caffeine.

The chelator may be, for example, a multidentate molecule such as an aminopolycarboxylic acid or an aminopolythiocarboxylic acid, or a salt or functional variant thereof. The chelator may be, for example, bidentate or tridentate or tetradentate. Examples of suitable metal chelators include molecules comprising EDTA (Ethylenediaminetetraacetic acid, or a salt form such as CaNa₂EDTA), DTPA (Diethylenetriamine Pentaacetic Acid), DOTA (1,4,7,10-tetraazacyclododecane-1,4,7,10-tetraacetic acid), NOTA (2,2′,2″-(1,4,7-Triazanonane-1,4,7-triyl)triacetic acid), IDA (Iminodiacetic acid), MIDA ((Methylimino)diacetic acid), TTHA (3,6,9,12-Tetrakis(carboxymethyl)-3,6,9,12-tetra-azatetradecanedioic acid), TETA (2,2′,2″,2′″-(1,4,8,11-Tetraazacyclotetradecane-1,4,8,11-tetrayl)tetraacetic acid), DOTAM (1,4,7,10-Tetrakis(carbamoylmethyl)-1,4,7,10-tetraazacyclododecane), HEHA (1,4,7,10,13,16-hexaazacyclohexadecane-1,4,7,10,13,16-hexaacetic acid, available from Macrocyclics, Inc., Plano, Texas), NTA (nitrilotriacetic acid) EDDHA (ethylenediamine-N, N′-bis(2-hydroxyphenylacetic acid), BAL (2,3-dimercaptopropanol), DMSA (2,3-dimercaptosuccinic acid), DMPS (2,3-dimercapto-1-propanesulfonic acid), D-penicillamine (B-dimethylcysteine), MAG₃ (mercaptoacetyltriglycine), Hynic (6-hydrazinopridine-3-carboxylic acid), p-isothiocyanatobenzyl-desferrioxamine (e.g., labelled with zirkonium for imaging), and salts or functional variants/derivatives thereof capable of chelating the metal. In some embodiments, it may be preferred that the chelator is DOTA or DOTAM or a salt or functional variant/derivative thereof capable of chelating the metal. Thus, the chelator may be or may comprise DOTA or DOTAM with a radioisotope chelated thereto.

The radiolabelled compound may comprise or consist of functional variants or derivatives of the chelators above, together with the radionuclide. Suitable variants/derivatives have a structure that differs to a certain limited extent and retain the ability to function as a chelator (i.e. retains sufficient activity to be used for one or more of the purposes described herein). Functional variants/derivatives may also include a chelator as described above conjugated to one or more additional moieties or substituents, including, a small molecule, a polypeptide or a carbohydrate. This attachment may occur via one of the constituent carbons, for example in a backbone portion of the chelator. A suitable substituent can be, for example, a hydrocarbon group such as alkyl, alkenyl, aryl or alkynyl; a hydroxy group; an alcohol group; a halogen atom; a nitro group; a cyano group; a sulfonyl group; a thiol group; an amine group; an oxo group; a carboxy group; a thiocarboxy group; a carbonyl group; an amide group; an ester group; or a heterocycle including heteroaryl groups. The substituent may be, for example, one of those defined for group “R¹” below. A small molecule can be, for example, a dye (such as Alexa 647 or Alexa 488), biotin or a biotin moiety, or a phenyl or benzyl moiety. A polypeptide may be, for example, an oligo peptide, e.g., an oligopeptide of two or three amino acids. Exemplary carbohydrates include dextran, linear or branched polymers or co-polymers (e.g. polyalkylene, poly(ethylene-lysine), polymethacrylate, polyamino acids, poly- or oligosaccharides, dendrimers). Derivatives may also include multimers of the chelator compounds in which compounds as set out above are linked through a linker moiety. Derivatives may also include functional fragments of the above compounds, which retain the ability to chelate the metal ion.

Particular examples of derivatives include benzyl-EDTA and hydroxyethyl-thiourido-benzyl EDTA, DOTA-benzene (e.g., (S-2-(4-aminobenzyl)-1,4,7,10-tetraazacyclododecane tetraacetic acid), DOTA-biotin, and DOTA-TyrLys-DOTA.

In some embodiments of the present invention, the functional binding site for the radioligand binds to a metal chelate comprising DOTAM and a metal, e.g., lead (Pb). As mentioned above, “DOTAM” has the chemical name:
1,4,7,10-Tetrakis(carbamoylmethyl)-1,4,7,10-tetraazacyclododecane,
   which is a compound of the following formula:

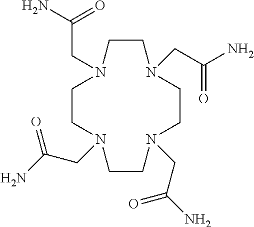 [SMILES: NC(=O)CN1CCN(CC(N)=O)CCN(CC(N)=O)CCN(CC(N)=O)CC1]

The present invention may in certain aspects and embodiments also make use of functional variants or derivatives of DOTAM incorporating a metal ion. Suitable variants/derivatives of DOTAM have a structure that differs to a certain limited extent from the structure of DOTAM and retain the ability to function (i.e. retains sufficient activity to be used for one or more of the purposes described herein). In such aspects and embodiments, the DOTAM or functional variant/derivative of DOTAM may be one of the active variants disclosed in WO 2010/099536. Suitable functional variants/derivatives may be a compound of the following formula:

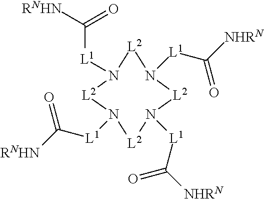

or a pharmaceutically acceptable salt thereof; wherein
   R^{N} is H, C₁₋₆ alkyl, C₁₋₆ haloalkyl, C₂₋₆ alkenyl, C₂₋₆ alkynyl, C₃₋₇ cycloalkyl, C₃₋₇ cycloalkyl-C₁₋₄alkyl, C₂₋₇ heterocycloalkyl, C₂₋₇ heterocycloalkyl-C₁₋₄ alkyl, phenyl, phenyl-C₁₋₄-alkyl, C₁₋₇ heteroaryl, and C₁₋₇ heteroaryl-C₁₋₄-alkyl; wherein C₁₋₆ alkyl, C₁₋₆ haloalkyl, C₂₋₆ alkenyl, and C₂₋₆ alkynyl are each optionally substituted by 1, 2, 3, or 4 independently selected R^{w} groups; and wherein said C₃₋₇ cycloalkyl, C₃₋₇ cycloalkyl-C₁₋₄alkyl, C₂₋₇ heterocycloalkyl, C₂₋₇ heterocycloalkyl-C₁₋₄ alkyl, phenyl, phenyl-C₁₋₄-alkyl, C₁₋₇ heteroaryl, and C₁₋₇ heteroaryl-C₁₋₄-alkyl are each optionally substituted by 1, 2, 3, or 4 independently selected R^{x} groups;
L¹ is independently C₁₋₆ alkylene, C₁₋₆ alkenylene, or C₁₋₆ alkynylene, each of which is optionally substituted by 1, 2, or 3 groups independently selected R¹ groups;
L² is C₂₋₄ straight chain alkylene, which is optionally substituted by an independently selected R¹ group; and which is optionally substituted by 1, 2, 3, or 4 groups independently selected from C₁₋₄ alkyl and or C₁₋₄ haloalkyl;
R¹ is independently selected from D¹-D²-D³, halogen, cyano, nitro, hydroxyl, C₁₋₆ alkoxy, C₁₋₆ haloalkoxy, C₁₋₆ alkylthio, C₁₋₆ alkylsulfinyl, C₁₋₆ alkylsulfonyl, amino, C₁₋₆ alkylamino, di-C₁₋₆ alkylamino, C₁₋₄ alkylcarbonyl, carboxy, C₁₋₆ alkoxycarbonyl, C₁₋₆ alkylcarbonylamino, di-C₁₋₆ alkylcarbonylamino, C₁₋₆ alkoxycarbonylamino, C₁₋₆ alkoxycarbonyl-(C₁₋₆ alkyl)amino, carbamyl, C₁₋₆ alkylcarbamyl, and di-C₁₋₆ alkylcarbamyl;
each D¹ is independently selected from C₆₋₁₀ aryl-C₁₋₄ alkyl, C₁₋₉ heteroaryl-C₁₋₄ alkyl, C₃₋₁₀ cycloalkyl-C₁₋₄ alkyl, C₂₋₉ heterocycloalkyl-C₁₋₄ alkyl, C₁₋₈ alkylene, C₁₋₈ alkenylene, and C₁₋₈ alkynylene; wherein said C₁₋₈ alkylene, C₁₋₈ alkenylene, and C₁₋₈ alkynylene are optionally substituted by 1, 2, 3, or 4 independently selected R⁴ groups; and wherein said C₆₋₁₀ aryl-C₁₋₄ alkyl, C₁₋₉ heteroaryl-C₁₋₄ alkyl, C₃₋₁₀ cycloalkyl-C₁₋₄ alkyl, C₂₋₉ heterocycloalkyl-C₁₋₄ alkyl are each optionally substituted by 1, 2, 3, or 4 independently selected R⁵ groups;
each D² is independently absent or C₁₋₂₀ straight chain alkylene, wherein from 1 to 6 non-adjacent methylene groups of said C₁₋₂₀ straight chain alkylene are each optionally replaced by an independently selected-D⁴- moiety, provided that at least one methylene unit in said C₁₋₂₀ straight chain alkylene is not optionally replaced by a-D⁴- moiety; wherein said C₁₋₂₀ straight chain alkylene is optionally substituted by one or more groups independently selected from halogen, cyano, nitro, hydroxyl, C₁₋₄ alkyl, C₁₋₄ haloalkyl, C₁₋₄ alkoxy, C₁₋₄ haloalkoxy, amino, C₁₋₄ alkylamino, di-C₁₋₄ alkylamino, C₁₋₄ alkylcarbonyl, carboxy, C₁₋₄ alkoxycarbonyl, C₁₋₄ alkylcarbonylamino, di-C₁₋₄ alkylcarbonylamino, C₁₋₄ alkoxycarbonylamino, C₁₋₄ alkoxycarbonyl-(C₁₋₄ alkyl)amino, carbamyl, C₁₋₄ alkylcarbamyl, and di-C₁₋₄ alkylcarbamyl;
each D³ is independently selected from H, halogen, cyano, nitro, hydroxyl, C₁₋₆ alkyl, C₁₋₆ haloalkyl, C₂₋₆ alkenyl, C₂₋₆ alkynyl, C₃₋₁₄ cycloalkyl, C₃₋₁₄ cycloalkyl-C₁₋₄ alkyl, C₂₋₁₄ heterocycloalkyl, C₂₋₁₄ heterocycloalkyl-C₁₋₄ alkyl, C₆₋₁₄ aryl, C₆₋₁₄ aryl-C₁₋₄ alkyl, C₁₋₁₃ heteroaryl, C₁₋₁₃ heteroaryl-C₁₋₄ alkyl; wherein said C₁₋₆ alkyl, C₁₋₆ haloalkyl, C₂₋₆ alkenyl, C₂₋₆ alkynyl are each optionally substituted by 1, 2, 3, or 4 independently selected R⁶ groups; and wherein said C₃₋₁₄ cycloalkyl, C₃₋₁₄ cycloalkyl-C₁₋₄ alkyl, C₂₋₁₄ heterocycloalkyl, C₂₋₁₄ heterocycloalkyl-C₁₋₄ alkyl, C₆₋₁₄ aryl, C₆₋₁₄ aryl-C₁₋₄ alkyl, C₁₋₁₃ heteroaryl, C₁₋₁₃ heteroaryl-C₁₋₄ alkyl are each optionally substituted by 1, 2, 3 or 4 independently selected R⁷ groups;
each D⁴ is independently selected from —O—, —S—, —NR^{a}C(═O)—, —NR^{a}C(═S)—, —NR^{b}C(═O)NR^{c}—, —NR^{b}C(═S)NR^{c}—, —S(═O)—, —S(═O)₂—, —S(═O)NR^{a}—, —C(═O)—, —C(═S)—, —C(═O)O—, —OC(═O)NR^{a}—, —OC(═S)NR^{a}—, —NR^{a}—, —NR^{b}S(═O)NR^{c}—, and NR^{b}S(═O)₂NR^{c}—;
each R⁴ and R⁶ is independently selected from halogen, cyano, nitro, hydroxyl, C₁₋₄ alkoxy, C₁₋₄ haloalkoxy, C₁₋₄ alkylthio, C₁₋₄ alkylsulfinyl, C₁₋₄ alkylsulfonyl, amino, C₁₋₄ alkylamino, di-C₁₋₄ alkylamino, C₁₋₄ alkylcarbonyl, carboxy, C₁₋₄ alkoxycarbonyl, C₁₋₄ alkylcarbonylamino, di-C₁₋₄ alkylcarbonylamino, C₁₋₄ alkoxycarbonylamino, C₁₋₄ alkoxycarbonyl-(C₁₋₄ alkyl)amino, carbamyl, C₁₋₄ alkylcarbamyl, and di-C₁₋₄ alkylcarbamyl;
each R⁵ is independently selected from halogen, cyano, cyanate, isothiocyanate, nitro, hydroxyl, C₁₋₄ alkyl, C₂₋₄ alkenyl, C₂₋₄ alkynyl, C₁₋₄ alkoxy, C₁₋₄ haloalkoxy, C₁₋₄ alkylthio, C₁₋₄ alkylsulfinyl, C₁₋₄ alkylsulfonyl, amino, C₁₋₄ alkylamino, di-C₁₋₄ alkylamino, C₁₋₄ alkylcarbonyl, carboxy, C₁₋₄ alkoxycarbonyl, C₁₋₄ alkylcarbonylamino, di-C₁₋₄ alkylcarbonylamino, C₁₋₄ alkoxycarbonylamino, C₁₋₄ alkoxycarbonyl-(C₁₋₄ alkyl)amino, carbamyl, C₁₋₄ alkylcarbamyl, and di-C₁₋₄ alkylcarbamyl;
each R⁷ is independently selected from halogen, cyano, nitro, hydroxyl, C₁₋₆ alkyl, C₂₋₆ alkenyl, C₂₋₆ alkynyl, C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄ alkyl, C₁₋₇ heteroaryl, C₁₋₇ heteroaryl-C₁₋₄ alkyl, —OR^{O}, —SR^{O}, —S(═O)R^{P}, —S(═O)₂R^{P}, —S(═O)NR^{s}R^{t}, —C(═O)R^{P}, —C(═O)OR^{P}, —C(═O)NR^{s}R^{t}, —OC(═O)R^{P}, —OC(═O)NR^{s}R^{t}, —NR^{s}R^{t}, —NR^{q}C(═O)R^{r}, —NR^{q}C(═O)OR^{r}, —NR^{q}C(═O)NR^{r}, —NR^{q}S(═O)₂R^{r}, and —NR^{P}S(═O)₂NR^{s}R^{t}; wherein said C₁₋₆ alkyl, C₂₋₆ alkenyl, C₂₋₆ alkynyl are each optionally substituted by 1, 2, 3, or 4 independently selected R′ groups; and wherein said C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄ alkyl, C₁₋₇ heteroaryl, C₁₋₇ heteroaryl-C₁₋₄ alkyl are each optionally substituted by 1, 2, 3, or 4 independently selected R″ groups;
each R^{a}, R^{b}, and R^{c} is independently selected from H, C₁₋₆ alkyl, C₁₋₆ haloalkyl, C₂₋₆ alkenyl, C₂₋₆ alkynyl, C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄ alkyl, C₁₋₇ heteroaryl, C₁₋₇ heteroaryl-C₁₋₄ alkyl; wherein said C₁₋₆ alkyl, C₁₋₆ haloalkyl, C₂₋₆ alkenyl, C₂₋₆ alkynyl are each optionally substituted by 1, 2, 3, or 4 independently selected R^{w} groups; and wherein said C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄ alkyl, C₁₋₇ heteroaryl, C₁₋₇ heteroaryl-C₁₋₄ alkyl are each optionally substituted by 1, 2, 3, or 4 independently selected R^{x} groups;
each R^{o}, R^{p}, R^{q}, R^{f}, R^{s} and R^{t} is independently selected from H, C₁₋₆ alkyl, C₁₋₆ haloalkyl, C₂₋₆ alkenyl, C₂₋₆ alkynyl, C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄ alkyl, C₁₋₇ heteroaryl, C₁₋₇ heteroaryl-C₁₋₄ alkyl; wherein said C₁₋₆ alkyl, C₁₋₆ haloalkyl, C₂₋₆ alkenyl, C₂₋₆ alkynyl are each optionally substituted by 1, 2, 3, or 4 independently selected R^{y} groups; and wherein said C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄ alkyl, C₁₋₇ heteroaryl, C₁₋₇ heteroaryl-C₁₋₄ alkyl are each optionally substituted by 1, 2, 3, or 4 independently selected R^{z} groups;
each R′, R^{w} and R^{y} is independently selected from hydroxyl, cyano, nitro, C₁₋₄ alkoxy, C₁₋₄ haloalkoxy, amino, C₁₋₄ alkylamino, and di-C₁₋₄ alkylamino; and
each R″, R^{x}, and R^{z} is independently selected from hydroxyl, halogen, cyano, nitro, C₁₋₄ alkyl, C₁₋₄ haloalkyl, C₁₋₄ alkoxy, C₁₋₄ haloalkoxy, amino, C₁₋₄ alkylamino, and di-C₁₋₄ alkylamino;
provided that the valency of each atom in the optionally substituted moieties is not exceeded.

Suitably, the functional variants/derivatives of the above formula have an affinity for an antibody of the present invention which is comparable to or greater than that of DOTAM, and have a binding strength for Pb which is comparable to or greater than that of DOTAM (“affinity” being as measured by the dissociation constant, as described above). For example, the dissociation constant of the functional/variant derivative with the antibody of the present invention or/Pb may be 1.1 times or less, 1.2 times or less, 1.3 times or less, 1.4 times or less, 1.5 times or less, or 2 times or less than the dissociation constant of DOTAM with the same antibody/Pb.

Each R^{N} may be H, C₁₋₆ alkyl, or C₁₋₆ haloalkyl; preferably H, C₁₋₄ alkyl, or C₁₋₄ haloalkyl. Most preferably, each R^{N} is H.

For DOTAM variants, it is preferred that 1, 2, 3 or most preferably each L² is C₂ alkylene. Advantageously, the C₂ alkylene variants of DOTAM can have particularly high affinity for Pb. The optional substituents for L² may be R¹, C₁₋₄ alkyl, or C₁₋₄ haloalkyl. Suitably, the optional substituents for L² may be C₁₋₄ alkyl or C₁₋₄ haloalkyl.

Optionally, each L² may be unsubstituted C₂ alkylene —CH₂CH₂—.

Each L¹ is preferably C₁₋₄ alkylene, more preferably C₁ alkylene such as —CH₂—.

The functional variant/derivative of DOTAM may be a compound of the following formula:

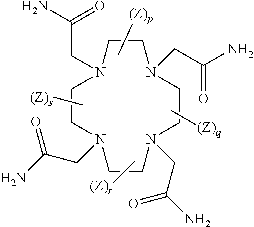

wherein each Z is independently R¹ as defined above; p, q, r, and s are 0, 1 or 2; and p+q+r+s is 1 or greater. Preferably, p, q, r, and s are 0 or 1 and/or p+q+r+s is 1. For example, the compound may have p+q+r+s=1, where Z is p-SCN-benzyl moiety—such a compound is commercially available from Macrocyclics, Inc. (Plano, Texas).

Radionuclides useful in the invention may include radioisotopes of metals, such as of lead (Pb), lutetium (Lu), or yttrium (Y).

Radionuclides particularly useful in therapeutic applications be radionuclides that are alpha or beta emitters. For instance, they may be selected from ²¹²Pb, ²¹²Bi, ²¹³Bi, ⁹⁰Y, ¹⁷⁷Lu, ²²⁵Ac, ²¹¹At, ²²⁷Th, ²²³Ra.

In some embodiments, it may be preferred that DOTAM (or salts or functional variants thereof) is chelated with Pb or Bi such as one of the Pb or Bi radioisotopes listed above. It other embodiments, it may be preferred that DOTA (or salts or functional variants thereof) is chelated with Lu or Y such as one of the Lu or Y radioisotopes listed above.

In some embodiments, the multivalent antibody or multivalent split antibody may bind to a Pb-DOTAM chelate.

In some embodiments, the multivalent antibody or multivalent split antibody may specifically bind to the radiolabelled compound. In some embodiments, it may bind to the radiolabelled compound, such as the Pb-DOTAM chelate, with a dissociation constant (K_{D}) to Pb-DOTAM and/or the target of ≤1 μM, ≤100 nM, ≤10 nM, ≤1 nM, ≤0.1 nM, ≤0.01 nM, or ≤0.001 nM (e.g. 10⁻⁷M or less, e.g. from 10⁻⁷ to 10⁻¹³, 10⁻⁸ M or less, e.g. from 10⁻⁸ M to 10⁻¹³ M, e.g., from 10⁻⁹ M to 10⁻¹³ M). It some embodiments it may be preferred that it binds with a K_{D} value of the binding affinity of 100 pM, 50 pM, 20 pM, 10 pM, 5 pM, 1 pM or less, e.g., 0.9 pM or less, 0.8 pM or less, 0.7 pM or less, 0.6 pM or less or 0.5 pM or less. For instance, the functional binding site may bind the metal chelate with a K_{D} of about 1 pM-1 nM, e.g., about 1-10 pM, 1-100 pM, 5-50 pM, 100-500 pM or 500 pM-1 nM.

### B. Exemplary Antigen Binding Sites for DOTA

In one particular embodiment of the invention, the antibody comprises a functional binding site for DOTA (or a functional derivative or variant thereof) or the first and second hemibody associate to form a functional binding site for DOTA (or a functional derivative or variant thereof), e.g., DOTA chelated with Lu or Y (e.g., ¹⁷⁷Lu or ⁹⁰Y). For instance, the functional binding site may bind the radiolabelled compound with a K_{D} of about 1 μM-1 nM, e.g., about 1-10 μM, 1-100 μM, 5-50 μM, 100-500 μM or 500 μM-1 nM.

C₈₂₅ is a known scFv with high affinity for DOTA-Bn (S-2-(4-aminobenzyl)-1,4,7,10-tetraazacyclododecane tetraacetic acid) complexed with radiometals such as ¹⁷⁷Lu and ⁹⁰Y (see for instance Cheal et al 2018, Theranostics 2018, and WO2010099536, incorporated herein by reference). The CDR sequences and the VL and VH sequences of C₈₂₅ are provided herein. In one embodiment, the heavy chain variable region forming part of the antigen binding site for the radiolabelled compound may comprise at least one, two or all three CDRs selected from (a) CDR-H1 comprising the amino acid sequence of 35; (b) CDR-H2 comprising the amino acid sequence of 36; (c) CDR-H3 comprising the amino acid sequence of 37. In an alternative embodiment, CDR-H1 may have the sequence GFSLTDYGVH. The light chain variable region forming part of the binding site for the radiolabelled compound may comprise at least one, two or all three CDRs selected from (d) CDR-L1 comprising the amino acid sequence of 38; (e) CDR-L2 comprising the amino acid sequence of 39; and (f) CDR-L3 comprising the amino acid sequence of 40.

In another embodiment, the heavy chain variable domain forming part of the functional antigen binding site for the radiolabelled compound (e.g., on the first hemibody in the case of split antibodies) comprises the amino acid sequence of SEQ ID NO: 41, or a variant thereof comprising an amino acid sequence having at least 90, 91, 92, 93, 94, 95, 96, 97, 98, or 99% identity to SEQ ID NO: 41. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but a binding site comprising that sequence retains the ability to bind to DOTA complexed with Lu or Y, preferably with an affinity as described herein. In certain embodiments, a total of 1 to 10 amino acids have been substituted, inserted and/or deleted in SEQ ID NO:41. In certain embodiments, substitutions, insertions, or deletions occur in regions outside the CDRs (i.e., in the FRs). Optionally, the antibody or first hemibody comprises the VH sequence in SEQ ID NO:41, including post-translational modifications of that sequence. In a particular embodiment, the VH comprises one, two or three CDRs selected from: (a) CDR-H1 comprising the amino acid sequence of SEQ ID NO:35 or the sequence GFSLTDYGVH, (b) CDR-H2 comprising the amino acid sequence of SEQ ID NO:36, and (c) CDR-H3 comprising the amino acid sequence of SEQ ID NO:37.

Optionally, the light chain variable domain forming part of the functional antigen binding site for the radiolabelled compound (e.g., on the second hemibody in the case of split antibodies) comprises an amino acid sequence of SEQ ID NO: 42 or a variant thereof comprising an amino acid sequence having at least 90, 91, 92, 93, 94, 95, 96, 97, 98, or 99% identity to SEQ ID NO: 42. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but a binding site comprising that sequence retains the ability to bind to DOTA complexed with Lu or Y, preferably with an affinity as described herein. In certain embodiments, a total of 1 to 10 amino acids have been substituted, inserted and/or deleted in SEQ ID NO: 42. In certain embodiments, the substitutions, insertions, or deletions occur in regions outside the CDRs (i.e., in the FRs). Optionally, the antibody or second hemibody comprises the VL sequence in SEQ ID NO:42, including post-translational modifications of that sequence. In a particular embodiment, the VL comprises one, two or three CDRs selected from (a) CDR-L1 comprising the amino acid sequence of SEQ ID NO:38; (b) CDR-L2 comprising the amino acid sequence of SEQ ID NO:39; and (c) CDR-L3 comprising the amino acid sequence of SEQ ID NO:40.

Embodiments concerned with the heavy chain variable region and the light chain variable region are explicitly contemplated in combination. Thus, the functional antigen binding site may be formed from a heavy chain variable region as defined above and a light chain variable region as defined above. In the case of a split antibody, these may be on the first and second hemibody respectively.

In any of the above embodiments, the light and heavy chain variable regions forming the binding site for the DOTA complex may be humanized. In one embodiment, the light and heavy chain variable region comprise CDRs as in any of the above embodiments, and further comprise an acceptor human framework, e.g. a human immunoglobulin framework or a human consensus framework.

In some embodiments, the heavy chain variable domain may be extended by one or more C-terminal residues such as one or more C-terminal alanine residues, or one or more residues from the N-terminus of the CH1 domain, as discussed further below.

### C. Exemplary Antigen Binding Sites for DOTAM

In another particular embodiment of the invention, the antibody comprises a functional binding site for a Pb-DOTAM chelate (Pb-DOTAM), or the first and second hemibody associate to form a functional antigen binding site for a Pb-DOTAM chelate (Pb-DOTAM).

Exemplary antigen binding sites are described in WO2019/201959, which is incorporated herein by reference in its entirety.

In certain embodiments, the functional antigen-binding site that binds to Pb-DOTAM may have one or more of the following properties:
Binds specifically to Pb-DOTAM and to Bi-DOTAM;
Is selective for Pb-DOTAM (and optionally Bi-DOTAM) as compared to other chelated metals, such as Cu-DOTAM;
Binds to Pb-DOTAM with a very high affinity;
Binds to the same epitope on Pb-DOTAM as antibodies described in WO2019/201959 e.g., PRIT-0213 or PRIT-0214 and/or has the same contact residues as said antibodies.

Radioisotopes of Pb are useful in methods of therapy. Particular radioisotopes of lead which may be of use in the present invention include ²¹²Pb.

Radionuclides which are α-particle emitters have the potential for more specific tumour cell killing with less damage to the surrounding tissue than β-emitters because of the combination of short path length and high linear energy transfer. ²¹²Bi is an α-particle emitter but its short half-life hampers its direct use. ²¹²Pb is the parental radionuclide of ²¹²Bi and can serve as an in vivo generator of ²¹²Bi, thereby effectively overcoming the short half-life of ²¹²Bi (Yong and Brechbiel, Dalton Trans. 2001 June 21; 40(23)6068-6076).

Generally, radiometals are used in chelated form. In certain aspects of the present invention, DOTAM is used as the chelating agent. DOTAM is a stable chelator of Pb(II) (Yong and Brechbiel, Dalton Trans. 2001 June 21; 40(23)6068-6076; Chappell et al Nuclear Medicine and Biology, Vol. 27, pp. 93-100, 2000). Thus, DOTAM is particularly useful in conjunction with isotopes of lead as discussed above, such as ²¹²Pb.

In some embodiments, it may be preferred that the antibodies bind Pb-DOTAM with a K_{D} value of the binding affinity of 100 pM, 50 pM, 20 pM, 10 pM, 5 pM, 1 pM or less, e.g, 0.9 pM or less, 0.8 pM or less, 0.7 pM or less, 0.6 pM or less or 0.5 pM or less. For instance, the functional binding site may bind the radiolabelled compound with a K_{D} of about 1 pM-1 nM, e.g., about 1-10 pM, 1-100 pM, 5-50 pM, 100-500 pM or 500 pM-1 nM.

In certain embodiment, the antibodies additionally bind to Bi chelated by DOTAM. In some embodiments, it may be preferred that the antibodies bind Bi-DOTAM (i.e., a chelate comprising DOTAM complexed with bismuth, also termed herein a “Bi-DOTAM chelate”) with a K_{D} value of the binding affinity of 1 nM, 500 pM, 200 pM, 100 pM, 50 pM, 10 pM or less, e.g., 9 pM, 8 pM, 7 pM, 6 pM, 5 pM or less. For instance, the functional binding site may bind a metal chelate with a K_{D} of about 1 pM-1 nM, e.g., about 1-10 pM, 1-100 pM, 5-50 pM, 100-500 pM or 500 pM-1 nM.

In some embodiments, the antibodies may bind to Bi-DOTAM and to Pb-DOTAM with a similar affinity. For instance, it may be preferred that the ratio of affinity, e.g., the ratio of K_{D} values, for Bi-DOTAM/Pb-DOTAM is in the range of 0.1-10, for example 1-10.

In one embodiment, the heavy chain variable region forming part of the antigen binding site for Pb-DOTAM (e.g., on the first hemibody in the case of split antibodies) may comprise at least one, two or all three CDRs selected from (a) CDR-H1 comprising the amino acid sequence of GFSLSTYSMS (SEQ ID NO:1); (b) CDR-H2 comprising the amino acid sequence of FIGSRGDTYYASWAKG (SEQ ID NO:2); (c) CDR-H3 comprising the amino acid sequence of ERDPYGGGAYPPHL (SEQ ID NO:3). The light chain variable region forming part of the binding site for Pb-DOTAM (e.g., on the second hemibody in the case of split antibodies) may comprise at least one, two or all three CDRs selected from (d) CDR-L1 comprising the amino acid sequence of QSSHSVYSDNDLA (SEQ ID NO:4); (e) CDR-L2 comprising the amino acid sequence of QASKLAS (SEQ ID NO:5); and (f) CDR-L3 comprising the amino acid sequence of LGGYDDESDTYG (SEQ ID NO:6).

In some embodiments, the antibodies may comprise one or more of CDR-H1, CDR-H2 and/or CDR-H3, or one or more of CDR-L1, CDR-L2 and/or CDR-L3, having substitutions as compared to the amino acid sequences of SEQ ID NOs: 1-6, respectively, e.g., 1, 2 or 3 substitutions.

In some embodiments, antibodies may share the same contact residues as the described herein: e.g., these residues may be invariant. These residues may include the following:
a) in heavy chain CDR2: Phe50, Asp56 and/or Tyr58, and optionally also Gly52 and/or Arg 54;
b) in heavy chain CDR3: Glu95, Arg96, Asp97, Pro98, Tyr99, Ala100C and/or Tyr100D and optionally also Pro100E;
c) in light chain CDR1: Tyr28 and/or Asp32;
d) in light chain CDR3: Gly91, Tyr92, Asp93, Thr95c and/or Tyr96;
e) in light chain CDR2: optionally Gln50;
all numbered according to Kabat.

For example, in some embodiments, CDR-H2 may comprise the amino acid sequence FIGSRGDTYYASWAKG (SEQ ID NO:2), or a variant thereof having up to 1, 2, or 3 substitutions in SEQ ID NO: 2, wherein these substitutions do not include Phe50, Asp56 and/or Tyr58, and optionally also do not include Gly52 and/or Arg 54, all numbered according to Kabat.

In some embodiments, CDR-H2 may be substituted at one or more positions as shown below. Here and in the substitution tables that follow, substitutions are based on the germline residues (underlined) or by amino acids which theoretically sterically fit and also occur in the crystallized repertoire at the site. In some embodiments, the residues as mentioned above may be fixed and other residues may be substituted according to the table below: in other embodiments, substitutions of any residue may be made according to the table below.

| WolfGuy | Kabat | AA | Substitution |
|---|---|---|---|
| 251 | 50 | F | Y, H |
| 252 | 51 | I | |
| 253 | 52 | G | |
| 254 | 53 | S | A, G, T, I, N |
| 288 | 54 | R | A, D, G, N, S, T, F, Y |
| 289 | 55 | G | D, S, Y, T, A, N, R, V |
| 290 | 56 | D | |
| 291 | 57 | T | K, I, A, P, S |
| 292 | 58 | Y | F, W, H |
| 293 | 59 | Y | N, F, H, L, S |
| 294 | 60 | A | G, N, S, T |
| 295 | 61 | S | A, G, N, Q, T |
| 296 | 62 | W | K, P, S, A, T, D, N, R, Q |
| 297 | 63 | A | F, L, V, M, I |
| 298 | 64 | K | N, Q, R, E |
| 299 | 65 | G | S, T, D, N, A |

Optionally, CDR-H3 may comprise the amino acid sequence ERDPYGGGAYPPHL (SEQ TD NO:3), or a variant thereof having up to 1, 2, or 3 substitutions in SEQ TD NO: 3, wherein these substitutions do not include Glu95, Arg96, Asp97, Pro98, and optionally also do not include Ala100C, Tyr100D, and/or Pro100E and/or optionally also do not include Tyr99. For instance, in some embodiments the substitutions do not include Glu95, Arg96, Asp97, Pro98, Tyr99 Ala100C and Tyr100D.

In certain embodiments, CDR-H3 may be substituted at one or more positions as shown below. In some embodiments, the residues as mentioned above may be fixed and other residues may be substituted according to the table below: in other embodiments, substitutions of any residue may be made according to the table below.

| WolfGuy | Kabat | AA | Substitution |
|---|---|---|---|
| 351 | 95 | E | |
| 352 | 96 | R | K, E |
| 353 | 97 | D | |
| 354 | 98 | P | |
| 355 | 99 | Y | F, G, S, T, D |
| 356 | 100 | G | |
| 392 | 100A | G | |
| 393 | 100B | G | |
| 394 | 100C | A | S, T |
| 395 | 100D | Y | F |
| 396 | 100E | P | |
| 397 | 100F | P | |
| 398 | 101 | H | A, T, V, D |
| 399 | 102 | L | Y, V, I, H, F |

Optionally, CDR-L1 may comprise the amino acid sequence QSSHSVYSDNDLA (SEQ ID NO:4) or a variant thereof having up to 1, 2, or 3 substitutions in SEQ ID NO: 4, wherein these substitutions do not include Tyr28 and/or Asp32 (Kabat numbering).

In certain embodiments, CDR-L1 may be substituted at one or more positions as shown below. Again, in some embodiments, the residues as mentioned above may be fixed and other residues may be substituted according to the table below: in other embodiments, substitutions of any residue may be made according to the table below.

| WolfGuy | Kabat | AA | Substitution |
|---|---|---|---|
| 551 | 24 | Q | R, K |
| 552 | 25 | S | A, G |
| 554 | 26 | S | T |
| 555 | 27 | H | Q, S, R, K |
| 556 | 27A | S | Q |
| 557 | 27B | V | I, D, N |
| 561 | 28 | Y | F |
| 562 | 29 | S | T, V |
| 571 | 30 | D | R, S, N, G |
| 572 | 31 | N | K |
| 597 | 32 | D | |
| 598 | 33 | L | I, V, M |
| 599 | 34 | A | S |

Optionally, CDR-L3 may comprise the amino acid sequence LGGYDDESDTYG (SEQ ID NO:6) or a variant thereof having up to 1, 2, or 3 substitutions in SEQ ID NO: 6, wherein these substitutions do not include Gly91, Tyr92, Asp93, Thr95c and/or Tyr96 (Kabat).

In certain embodiments, CDR-L3 may be substituted at the following positions as shown below. (Since most residues are solvent exposed and without antigen contacts, many substitutions are conceivable). Again, in some embodiments, the residues as mentioned above may be fixed and other residues may be substituted according to the table below: in other embodiments, substitutions of any residue may be made according to the table below.

| WolfGuy | Kabat | AA | Substitution |
|---|---|---|---|
| 751 | 89 | L | A, V, Q |
| 752 | 90 | G | A |
| 753 | 91 | G | |
| 754 | 92 | Y | A, D, E, F, G, H, I, |
| | | | K, L, N, Q, R, S, T, |
| | | | V |
| 755 | 93 | D | A, E, F, G, H, I, K, |
| | | | L, M, N, Q, R, S, T, |
| | | | V, W, Y |
| 756 | 94 | D | A, E, F, G, H, I, K, |
| | | | L, M, N, Q, R, S, T, |
| | | | V, W, Y |
| 794 | 95 | E | A, D, F, G, H, I, K, |
| | | | L, M, N, Q, R, S, T, |
| | | | V, W, Y |
| 795 | 95A | S | A, F, G, H, I, K, L, |
| | | | M, N, Q, R, T, V, W, |
| | | | Y |
| 796 | 95B | D | A, E, F, G, H, I, L, |
| | | | M, N, Q, S, T, V, W, |
| | | | Y |
| 797 | 95C | T | S |
| 798 | 96 | Y | F, H, R |
| 799 | 97 | G | A, E, I, K, L, M, N, |
| | | | Q, S, T, V |

The antibody may further comprise CDR-H1 or CDR-L2, optionally having the sequence of SEQ TD NO: 1 or SEQ TD NO: 5 respectively, or a variant thereof having at least 1, 2 or 3 substitutions relative thereto, optionally conservative substitutions.

Thus, the heavy chain variable domain forming part of the antigen binding site for Pb-DOTAM may comprise at least:
a) heavy chain CDR2 comprising the amino acid sequence FIGSRGDTYYASWAKG (SEQ ID NO:2), or a variant thereof having up to 1, 2, or 3 substitutions in SEQ ID NO: 2, wherein these substitutions do not include Phe50, Asp56 and/or Tyr58, and optionally also do not include Gly52 and/or Arg54;
b) heavy chain CDR3 comprising the amino acid sequence ERDPYGGGAYPPHL (SEQ ID NO:3), or a variant thereof having up to 1, 2, or 3 substitutions in SEQ ID NO: 3, wherein these substitutions do not include Glu95, Arg96, Asp97, Pro98, and optionally also do not include Ala100C, Tyr100D, and/or Pro100E and/or optionally also do not include Tyr99.

In some embodiments, the heavy chain variable domain additionally includes a heavy chain CDR1 which is optionally:
c) a heavy chain CDR1 comprising the amino acid sequence GFSLSTYSMS (SEQ ID NO:1) or a variant thereof having up to 1, 2, or 3 substitutions in SEQ ID NO: 1.

In some embodiments, the heavy chain variable domain additionally includes a C-terminal alanine (e.g. Ala114 according Kabat numbering system) to avoid the binding of pre-existing antibodies recognizing the free VH region. As reported in Holland M C et al J. Clin Immunol (2013), a free C-terminus appears to be important for binding of HAVH (human anti-VH domain) autoantibodies to VH domain antibodies, since HAVH autoantibodies do not bind to intact IgG or IgG fragments (fAb or modified VH molecules) containing the same VH framework sequences, or to VK domain antibodies. Cordy J C et al Clinical and Experimental Immunology (2015) notes the existence of a cryptic epitope at the C-terminal epitope of VH dAbs, which is not naturally accessible to HAVH antibodies in full IgG molecules.

Thus, where the antibody comprises a free VH region (not fused to any other domain at its C-terminus), the sequence may be extended by one or more C-terminal residue. The extension may prevent the binding of antibodies recognizing the free VH region. For instance, the extension may be by 1-10 residues, e.g., 1,2,3,4,5,6,7,8,9 or 10 residues. In one embodiment, the VH sequence may be extended by one or more C-terminal alanine residues. The VH sequence may also be extended by an N-terminal portion of the CH1 domain, e.g., by 1-10 residues from the N-terminus of the CH1 domain, e.g., from the human IgG1 CH1 domain. (The first ten residues of the human IgG1 CH1 domain are ASTKGPSVFP (SEQ ID NO.: 149), and so in one embodiment, from 1-10 residues may be taken from the N-terminus of this sequence). For instance, in one embodiment, the peptide sequence AST (corresponding to the first 3 residues of the IgG1 CH1 domain) is added to the C-terminus of the VH region.

In another embodiment, the light chain variable domain forming part of the antigen binding site for Pb-DOTAM comprises at least:
d) light chain CDR1 comprising the amino acid sequence QSSHSVYSDNDLA (SEQ ID NO:4) or a variant thereof having up to 1, 2, or 3 substitutions in SEQ ID NO: 4, wherein these substitutions do not include Tyr28 and Asp32;
e) light chain CDR3 comprising the amino acid sequence LGGYDDESDTYG (SEQ ID NO:6) or a variant thereof having up to 1, 2, or 3 substitutions in SEQ ID NO: 6, wherein these substitutions do not include Gly91, Tyr92, Asp93, Thr95c and Tyr96.

In some embodiments, the light chain variable domain additionally includes a light chain CDR2 which is optionally:
f) a light chain CDR2 comprising the amino acid sequence QASKLAS (SEQ ID NO: 5) or a variant thereof having at least 1, 2 or 3 substitutions in SEQ ID NO: 5, optionally not including Gln50.

In any embodiments of the present invention which include variants of a sequence comprising the CDRs as set out above (e.g., of a variable domain), the protein may be invariant in one or more of the CDR residues as set out above.

Optionally, the heavy chain variable domain forming part of the functional antigen binding site for Pb-DOTAM (e.g., on the first hemibody in the case of a split antibody) comprises an amino acid sequence selected from the group consisting of SEQ ID NO: 7 and SEQ ID NO 9, or a variant thereof comprising an amino acid sequence having at least 90, 91, 92, 93, 94, 95, 96, 97, 98, or 99% identity to SEQ ID NO: 7 or SEQ ID NO: 9. (The N-terminal amino acid in these reference sequences, shown in parentheses, may be present or absent, and in some embodiments may be retained in any variant sequence). In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but a binding site comprising that sequence retains the ability to bind to Pb-DOTAM, preferably with an affinity as described herein. The VH sequence may retain the invariant residues as set out above. In certain embodiments, a total of 1 to 10 amino acids have been substituted, inserted and/or deleted in SEQ ID NO: 7 or SEQ ID NO 9. In certain embodiments, substitutions, insertions, or deletions occur in regions outside the CDRs (i.e., in the FRs). Optionally, the antibody comprises the VH sequence in SEQ ID NO:7 or SEQ ID NO: 9 (with or without the N-terminal residue shown in parentheses), including post-translational modifications of that sequence. In a particular embodiment, the VH comprises one, two or three CDRs selected from: (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3.

In some embodiments, as mentioned above, in some variants SEQ ID NO: 7 or 9 may be extended by one or more additional C-terminal residues, e.g., by one or more alanine residues, optionally a single alanine residue. Thus, for instance, in one specific variant, the sequence of SEQ ID NO: 7 may be extended to be:

VTLKESGPVLVKPTETLTLTCTVSGFSLSTYSMSWIRQPPGKALEWLGF
IGSRGDTYYASWAKGRLTISKDTSKSQVVLTMTNMDPVDTATYYCARER
DPYGGGAYPPHLWGRGTLVTVSSA

In other embodiments, the extension may be by an N-terminal portion of the CH1 domain as described above, e.g., by 1-10 residues from the N-terminus of the CH1 domain, e.g., from the human IgG1 CH1 domain. For instance, the extension may be by the peptide sequence AST.

Optionally, the light chain variable domain forming part of the functional antigen binding site for Pb-DOTAM (e.g., on the second hemibody in the case of a split antibody) comprises an amino acid sequence of SEQ ID NO: 8, or a variant thereof comprising an amino acid sequence having at least 90, 91, 92, 93, 94, 95, 96, 97, 98, or 99% identity to SEQ ID NO: 8. (The N-terminal amino acid in this reference sequences, shown in parentheses, may be present or absent, and in some embodiments may be retained in any variant). In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Pb-DOTAM binding site comprising that sequence retains the ability to bind to Pb-DOTAM, preferably with an affinity as described herein. The VL sequence may retain the invariant residues as set out above. In certain embodiments, a total of 1 to 10 amino acids have been substituted, inserted and/or deleted in SEQ ID NO:8. In certain embodiments, the substitutions, insertions, or deletions occur in regions outside the CDRs (i.e., in the FRs). Optionally, the anti-Pb-DOTAM antibody comprises the VL sequence in SEQ ID NO:8 (with or without the N-terminal residue shown in parentheses), including post-translational modifications of that sequence. In a particular embodiment, the VL comprises one, two or three CDRs selected from (a) CDR-L1 comprising the amino acid sequence of SEQ ID NO:4; (b) CDR-L2 comprising the amino acid sequence of SEQ ID NO:5; and (c) CDR-L3 comprising the amino acid sequence of SEQ ID NO:6.

Embodiments concerned with the heavy chain variable region and the light chain variable region are explicitly contemplated in combination. Thus, the functional antigen binding site for Pb-DOTAM may be formed from a heavy chain variable region as defined above and a light chain variable region as defined above. In the case of a split antibody, these may be on the first and second hemibody respectively.

Optionally, the antigen binding site specific for the Pb-DOTAM chelate may be formed from a heavy chain variable domain comprising an amino acid sequence selected from the group consisting of SEQ ID NO: 7 or SEQ ID NO: 9 (with or without the N-terminal residue shown in parentheses), or a variant thereof as defined above (including a variant with a C-terminal extension as discussed above), and a light chain variable domain comprising an amino acid sequence of SEQ ID NO: 8 (with or without the N-terminal residue shown in parentheses), or a variant thereof as defined above. For example, the antigen binding site specific for the Pb-DOTAM chelate may comprise a heavy chain variable domain comprising the amino acid sequence of SEQ ID NO: 7 or a variant thereof, and a light chain variable domain comprising the amino acid sequence of SEQ ID NO: 8 or a variant thereof, including post-translational modifications of those sequences. In another embodiment, it may comprise a heavy chain variable domain comprising the amino acid sequence of SEQ ID NO: 9 or a variant thereof (including a variant with a C-terminal extension as discussed above) and a light chain variable domain comprising the amino acid sequence of SEQ ID NO: 8 or a variant thereof, including post-translational modifications of those sequences.

In any of the above embodiments, the light and heavy chain variable regions forming the anti-Pb-DOTAM binding site may be humanized. In one embodiment, the light and heavy chain variable region comprise CDRs as in any of the above embodiments, and further comprise an acceptor human framework, e.g. a human immunoglobulin framework or a human consensus framework. In another embodiment, the light and/or heavy chain variable regions comprise CDRs as in any of the above embodiments, and further comprises framework regions derived from vk 1 39 and/or vh 2 26. For vk 1 39, in some embodiments there may be no back mutations. For vh 2 26, the germline Ala49 residue may be backmutated to Gly49.

### D. Target Antigens

The multispecific antibody (or split multispecific antibody) used in the combination therapy binds to a target antigen. This is an antigen expressed on the surface of a target cell. It can also be referred to as a “target cell antigen”.

The treatment is preferably of a tumour or cancer.

The target antigen can be, for example, a tumour-associated antigen.

The term “tumour-associated antigen” or “tumour specific antigen” as used herein refers to any molecule (e.g., protein, peptide, lipid, carbohydrate, etc.) solely or predominantly expressed or over-expressed by tumour cells and/or cancer cells, or by other cells of the stroma of the tumour such as cancer-associated fibroblasts, such that the antigen is associated with the tumour(s) and/or cancer(s). The tumour-associated antigen can additionally be expressed by normal, non-tumour, or non-cancerous cells. However, in such cases, the expression of the tumour-associated antigen by normal, non-tumour, or non-cancerous cells is not as robust as the expression by tumour or cancer cells. In this regard, the tumour or cancer cells can over-express the antigen or express the antigen at a significantly higher level, as compared to the expression of the antigen by normal, non-tumour, or non-cancerous cells. Also, the tumour-associated antigen can additionally be expressed by cells of a different state of development or maturation. For instance, the tumour-associated antigen can be additionally expressed by cells of the embryonic or foetal stage, which cells are not normally found in an adult host. Alternatively, the tumour-associated antigen can be additionally expressed by stem cells or precursor cells, which cells are not normally found in an adult host.

The tumour-associated antigen can be an antigen expressed by any cell of any cancer or tumour, including the cancers and tumours described herein. The tumour-associated antigen may be a tumour-associated antigen of only one type of cancer or tumour, such that the tumour-associated antigen is associated with or characteristic of only one type of cancer or tumour. Alternatively, the tumour-associated antigen may be a tumour-associated antigen (e.g., may be characteristic) of more than one type of cancer or tumour. For example, the tumour-associated antigen may be expressed by both breast and prostate cancer cells and not expressed at all by normal, non-tumour, or non-cancer cells.

Exemplary tumour-associated antigens to which the antibodies of the invention may bind include, but are not limited to, Melanoma-associated Chondroitin Sulfate Proteoglycan (MCSP), mucin 1 (MUCl; tumour-associated epithelial mucin), preferentially expressed antigen of melanoma (PRAME), carcinoembryonic antigen (CEA), prostate specific membrane antigen (PSMA), PSCA, EpCAM, Trop2 (trophoblast-2, also known as EGP-1), granulocyte-macrophage colony-stimulating factor receptor (GM-CSFR), CD56, human epidermal growth factor receptor 2 (HER2/neu) (also known as erbB-2), CDS, CD7, tyrosinase related protein (TRP) I, and TRP2. In another embodiment, the tumour antigen may be selected from the group consisting of cluster of differentiation (CD) 19, CD20, CD21, CD22, CD25, CD30, CD33 (sialic acid binding Ig-like lectin 3, myeloid cell surface antigen), CD79b, CD123 (interleukin 3 receptor alpha), transferrin receptor, EGF receptor, mesothelin, cadherin, Lewis Y, Glypican-3, FAP (fibroblast activation protein alpha), GPRC5D (G Protein-Coupled Receptor Class C Group 5 Member D), PSMA (prostate specific membrane antigen), CA9=CAIX (carbonic anhydrase IX), L1 CAM (neural cell adhesion molecule L 1), endosialin, HER3 (activated conformation of epidermal growth factor receptor family member 3), Alk1/BMP9 complex (anaplastic lymphoma kinase 1/bone morphogenetic protein 9), TPBG=5T4 (trophoblast glycoprotein), ROR1 (receptor tyrosine kinase-like surface antigen), HER1 (activated conformation of epidermal growth factor receptor), and CLL1 (C-type lectin domain family 12, member A). Mesothelin is expressed in, e.g., ovarian cancer, mesothelioma, non-small cell lung cancer, lung adenocarcinoma, fallopian tube cancer, head and neck cancer, cervical cancer, and pancreatic cancer. CD22 is expressed in, e.g., hairy cell leukaemia, chronic lymphocytic leukaemia (CLL), prolymphocytic leukaemia (PLL), non-Hodgkin's lymphoma, small lymphocytic lymphoma (SLL), and acute lymphatic leukaemia (ALL). CD25 is expressed in, e.g., leukemias and lymphomas, including hairy cell leukaemia and Hodgkin's lymphoma. Lewis Y antigen is expressed in, e.g., bladder cancer, breast cancer, ovarian cancer, colorectal cancer, esophageal cancer, gastric cancer, lung cancer, and pancreatic cancer. CD33 is expressed in, e.g., acute myeloid leukaemia (AML), chronic myelomonocytic leukaemia (CML), and myeloproliferative disorders.

Exemplary antibodies that specifically bind to tumour-associated antigens include, but are not limited to, antibodies against the transferrin receptor (e.g., HB21 and variants thereof), antibodies against CD22 (e.g., RFB4 and variants thereof), antibodies against CD25 (e.g., anti-Tac and variants thereof), antibodies against mesothelin (e.g., SS 1, MORAb-009, SS, HN1, HN2, MN, MB, and variants thereof) and antibodies against Lewis Y antigen (e.g., B3 and variants thereof). In this regard, the targeting moiety (cell-binding agent) may be an antibody selected from the group consisting of B3, RFB4, SS, SS1, MN, MB, HN1, HN2, HB21, and MORAb-009, and antigen binding portions thereof. Further exemplary targeting moieties suitable for use in the inventive chimeric molecules are disclosed e.g., in U.S. Pat. No. 5,242,824 (anti-transferrin receptor); U.S. Pat. No. 5,846,535 (anti-CD25); U.S. Pat. No. 5,889,157 (anti-Lewis Y); U.S. Pat. No. 5,981,726 (anti-Lewis Y); U.S. Pat. No. 5,990,296 (anti-Lewis Y); U.S. Pat. No. 7,081,518 (anti-mesothelin); U.S. Pat. No. 7,355,012 (anti-CD22 and anti-CD25); U.S. Pat. No. 7,368,110 (anti-mesothelin); U.S. Pat. No. 7,470,775 (anti-CD30); U.S. Pat. No. 7,521,054 (anti-CD25); and U.S. Pat. No. 7,541,034 (anti-CD22); U.S. Patent Application Publication 2007/0189962 (anti-CD22); Frankel et al., Clin. Cancer Res., 6: 326-334 (2000), and Kreitman et al., AAPS Journal, 8(3): E532-E551 (2006), each of which is incorporated herein by reference.

Further antibodies have been raised to target specific tumour related antigens including: Cripto, CD30, CD19, CD33, Glycoprotein NMB, CanAg, Her2 (ErbB2/Neu), CD56 (NCAM), CD22 (Siglec2), CD33 (Siglec3), CD79, CD138, PSCA, PSMA (prostate specific membrane antigen), BCMA, CD20, CD70, E-selectin, EphB2, Melanotransferin, Muc16 and TMEFF2. Any of these, or antigen-binding fragments thereof, may be useful in the present invention, i.e., may be incorporated into the antibodies described herein.

In some embodiments of the present invention, it may be preferred that the tumour-associated antigen is carcinoembryonic antigen (CEA).

CEA is advantageous in the context of the present invention because it is relatively slowly internalized, and thus a high percentage of the antibody will remain available on the surface of the cell after initial treatment, for binding to the radionuclide. Other low internalizing targets/tumour associated antigens may also be preferred. Other examples of tumour-associated antigen include CD20 or HER2. In still further embodiments, the target may be EGP-1 (epithelial glycoprotein-1, also known as trophoblast-2), colon-specific antigen-p (CSAp) or a pancreatic mucin MUC1. See for instance Goldenberg et al 2012 (Theranostics 2(5)), which is incorporated herein by reference. This reference also describes antibodies such as Mu-9 binding to CSAp (see also Sharkey et al Cancer Res. 2003; 63: 354-63), hPAM4 binding to MUC1 (see also Gold et al Cancer Res. 2008: 68: 4819-26), valtuzumab binding to CD20 (see also Sharkey et al Cancer Res. 2008; 68: 5282-90) and hRS7 which binds to EGP-1 (see also Cubas et al Biochim Biophys Acta 2009; 1796: 309-14). Any of these or antigen-binding portions thereof may be useful in the present invention, i.e., may be incorporated into the antibodies described herein. One example of an antibody that has been raised against CEA is T84.66 (as shown in NCBI Acc No: CAA36980 for the heavy chain and CAA36979 for the light chain, or as shown in SEQ ID NO 317 and 318 of WO2016/075278) and humanized and chimeric versions thereof, such as T84.66-LCHA as described in WO2016/075278 A1 and/or WO2017/055389. Another example is CH1A1a, an anti-CEA antibody as described in WO2012/117002 and WO2014/131712, and CEA hMN-14 (see also U.S. Pat. Nos. 6,676,924 and 5,874,540). Another anti-CEA antibody is A5B7 as described in M. J. Banfield et al, Proteins 1997, 29(2), 161-171. Humanized antibodies derived from murine antibody A5B7 have been disclosed in WO 92/01059 and WO 2007/071422. See also co-pending application PCT/EP2020/067582. An example of a humanized version of A5B7 is A5H1EL1(G54A). A further exemplary antibody against CEA is MFE23 and the humanized versions thereof described in U.S. Pat. No. 7,626,011 and/or co-pending application PCT/EP2020/067582. A still further example of an antibody against CEA is 28A9. Any of these or an antigen binding fragment thereof may be useful to form a CEA-binding moiety in the present invention.

In some embodiments, the antibodies of the invention may bind specifically to the target antigen (e.g., any of the target antigens discussed herein). In some embodiments, they may bind with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁷M or less, e.g. from 10⁻⁷ to 10⁻¹³, 10⁻⁸ M or less, e.g. from 10⁻⁸ M to 10⁻¹³ M, e.g., from 10⁻⁹ M to 10⁻¹³ M).

### E. Exemplary Antigen Binding Sites for CEA

In a particular embodiment of the present invention, which may be combined with the embodiments discussed above, e.g., the radiolabelled compounds and the exemplary binding sites for DOTA/DOTAM, the target antigen bound by the multispecific antibody or by the first and/or second hemibody of the split multispecific antibody may be CEA (carcinoembryonic antigen). Antibodies that have been raised against CEA include T84.66 and humanized and chimeric versions thereof, such as T84.66-LCHA as described in WO2016/075278 A1 and/or WO2017/055389, CH1A1a, an anti-CEA antibody as described in WO2012/117002 and WO2014/131712, and CEA hMN-14 or labetuzimab (e.g., as described in U.S. Pat. Nos. 6,676,924 and 5,874,540). Another exemplary antibody against CEA is A5B7 (e.g., as described in M. J. Banfield et al, Proteins 1997, 29(2), 161-171), or a humanized antibody derived from murine A5B7 as described in WO 92/01059 and WO 2007/071422. See also co-pending application PCT/EP2020/067582. An example of a humanized version of A5B7 is A5H1EL1(G54A). A further exemplary antibody against CEA is MFE23 and the humanized versions thereof described in U.S. Pat. No. 7,626,011 and/or co-pending application PCT/EP2020/067582. A still further example of an anti-CEA antibody is 28A9. Any of these or antigen binding fragments thereof may be used to form a CEA-binding moiety in the present invention.

Optionally, the antigen-binding moiety which binds to CEA may bind with a K_{D} value of 1 nM or less, 500 μM or less, 200 μM or less, or 100 μM or less for monovalent binding.

In some embodiments, the multispecific antibody or the first and/or second hemibody of the split multispecific may bind to the CH1A1a epitope, the A5B7 epitope, the MFE23 epitope, the T84.66 epitope or the 28A9 epitope of CEA.

In some embodiments, the multispecific antibody or the first and/or second hemibody of the split multispecific binds to a CEA epitope which is not present on soluble CEA (sCEA). Soluble CEA is a part of the CEA molecule which is cleaved by GPI phospholipase and released into the blood. An example of an epitope not found on soluble CEA is the CH1A1A epitope. Optionally, in the case of split antibodies, one of the first and/or second hemibody binds to an epitope which is not present on soluble CEA, and the other binds to an epitope which is present on soluble CEA.

The epitope for CH1A1a and its parent murine antibody PR1A3 is described in WO2012/117002A1 and Durbin H. et al., Proc. Natl. Scad. Sci. USA, 91:4313-4317, 1994. An antibody which binds to the CH1A1a epitope binds to a conformational epitope within the B3 domain and the GPI anchor of the CEA molecule. In one aspect, the antibody binds to the same epitope as the CH1A1a antibody having the VH of SEQ ID NO: 25 and VL of SEQ ID NO 26 herein. The A5B7 epitope is described in co-pending application PCT/EP2020/067582. An antibody which binds to the A5B7 epitope binds to the A2 domain of CEA, i.e., to the domain comprising the amino acids of SEQ ID NO: 141:

(SEQ ID NO: 141)
PKPFITSNNSNPVEDEDAVALTCEPEIQNTTYLWWVNNQSLPVSPRLQL
SNDNRTLTLLSVTRNDVGP YECGIQNKLSVDHSDPVILN.

In one aspect, the antibody binds to the same epitope as the A5B7 antibody having the VH of SEQ ID NO: 49 and VL of SEQ ID NO: 50 herein.

In one aspect, the antibody binds to the same epitope as the T84.66 described in WO2016/075278. The antibody may bind to the same epitope as the antibody having the VH of SEQ ID NO: 17 and VL of SEQ ID NO:18 herein.

The MFE23 epitope is described in co-pending application PCT/EP2020/067582. An antibody which binds to the MFE23 epitope binds to the A1 domain of CEA, i.e., to the domain comprising the amino acids of SEQ ID NO: 142:

(SEQ ID NO: 142)
PKPSISSNNSKPVEDKDAVAFTCEPETQDATYLWWVNNQSLPVSPRLQL
SNGNRTLTLFNVTRNDTAS YKCETQNPVSARRSDSVILN.

In one aspect, the antibody may bind to the same epitope as an antibody having the VH domain of SEQ ID NO: 127 and the VL domain of SEQ ID NO: 128 herein.

In some embodiments of split multi-specific antibodies, the first and the second hemibody bind the same epitope of CEA as each other. Thus, for example, the first and the second hemibody may both bind to the CH1A1a epitope, the A5B7 epitope, the MFE23 epitope, the T84.66 epitope or the 28A9 epitope.

In some embodiments, both the first and second hemibody may have CEA binding sequences (i.e., CDRs and/or VH/VL domains) from CH1A1A; or, the first and the second hemibody may both have CEA binding sequences from A5B7 or a humanized version thereof, or, the first and the second hemibody may both have CEA binding sequences from T84.66 or a humanized version thereof, or the first and the second hemibody may both have CEA binding sequences from MFE23 or a humanized version thereof, or the first and second hemibody may both have CEA binding sequences from 28A9 or a humanized version thereof. Exemplary sequences are disclosed herein.

In other embodiments, the first and the second hemibodies bind to different epitopes of CEA. Thus, for example, i) one hemibody may bind the CH1A1A epitope and the other may bind the A5B7 epitope, the T84.66 epitope, the MFE23 epitope or the 28A9 epitope; ii) one hemibody may bind the A5B7 epitope and the other may bind the CH1A1A epitope, T84.66 epitope, MFE23 epitope or 28A9 epitope; iii) one hemibody may bind the MFE23 epitope and the other may bind the CH1A1A epitope, A5B7 epitope, T84.66 epitope or 28A9 epitope; iv) one hemibody may bind the T84.66 epitope and the other may bind the CH1A1A epitope, A5B7 epitope, MFE23 epitope or 28A9 epitope; or v) one hemibody may bind the 28A9 epitope and the other may bind the CH1A1a epitope, the A5B7 epitope, the MFE23 epitope, or the T84.66 epitope.

In some embodiments, i) one hemibody may have CEA binding sequences (i.e., CDRs or VH/VL domains) from CH1A1A and the other may have CEA binding sequences from A5B7 or a humanized version thereof, from T84.66 or a humanized version thereof, from MFE23 or a humanized version thereof, or from 28A9 or a humanized version thereof, ii) one hemibody may have CEA binding sequences from A5B7 or a humanized version thereof and the other may have CEA binding sequences from CH1A1A, from T84.66 or a humanized version thereof, from MFE23 or a humanized version thereof, or from 28A9 or a humanized version thereof, iii) one hemibody may have CEA binding sequences from MFE23 or a humanized version thereof and the other may have CEA binding sequences from CH1A1A, from A5B7 or a humanized version thereof, from T84.66 or a humanized version thereof, or from 28A9 or a humanized version thereof, iv) one hemibody may have CEA binding sequences from T84.66 or a humanized version thereof and the other may have CEA binding sequences from CH1A1A, from A5B7 or a humanized version thereof, from MFE23 or a humanized version thereof, or from 28A9 or a humanized version; v) one hemibody may have CEA-binding sequences from 28A9 or a humanized version thereof and the other may have CEA binding sequences from CH1A1A, from A5B7 or a humanized version thereof, from T84.66 or a humanized version thereof, or from MFE23 or a humanized version thereof.

In one particular embodiment, one hemibody may bind the CH1A1A epitope and the other may bind the A5B7 epitope. The first hemibody may have CEA binding sequences from the antibody CH1A1A and the second hemibody may have CEA binding sequences from A5B7 (including a humanized version thereof); or, the first hemibody may have CEA binding sequences from the antibody A5B7 (including a humanized version thereof) and the second hemibody may have CEA binding sequences from CH1A1A.

In another particular embodiment, one hemibody may bind the CH1A1A epitope and the other may bind the T84.66 epitope. The first hemibody may have CEA binding sequences from the antibody CH1A1A and the second hemibody may have CEA binding sequences from T84.66 (including a humanized version thereof); or, the first hemibody may have CEA binding sequences from the antibody T84.66 (including a humanized version thereof) and the second hemibody may have CEA binding sequences from CH1A1A. In some embodiments, a first hemibody may bind the T84.66 epitope and/or have an antigen binding site as described in (i) below, and the second hemibody may bind the CH1A1A epitope and/or have an antigen binding site as described in (ii) below.

Exemplary CEA-binding sequences i)-v) are disclosed below. These provide examples of CEA-binding sequences from i) T84.66, ii) CH1A1A, iii) A5B7, iv) 28A9 and v) MFE23 (or from humanized versions thereof).

i). In one embodiment, the antigen-binding site which binds to CEA may comprise at least one, two, three, four, five, or six CDRs selected from (a) CDR-H1 comprising the amino acid sequence of SEQ ID NO:11; (b) CDR-H2 comprising the amino acid sequence of SEQ ID NO:12; (c) CDR-H3 comprising the amino acid sequence of SEQ ID NO:13; (d) CDR-L1 comprising the amino acid sequence of SEQ ID NO:14; (e) CDR-L2 comprising the amino acid sequence of SEQ ID NO:15; and (f) CDR-L3 comprising the amino acid sequence of SEQ ID NO:16.

Optionally, the antigen-binding site which binds to CEA may comprise at least one, at least two, or all three VH CDR sequences selected from (a) CDR-H1 comprising the amino acid sequence of SEQ ID NO:11; (b) CDR-H2 comprising the amino acid sequence of SEQ ID NO:12; and (c) CDR-H3 comprising the amino acid sequence of SEQ ID NO:13.

Optionally, the antigen-binding site which binds to CEA comprises at least one, at least two, or all three VL CDRs sequences selected from (a) CDR-L1 comprising the amino acid sequence of SEQ ID NO:14; (b) CDR-L2 comprising the amino acid sequence of SEQ ID NO:15; and (c) CDR-L3 comprising the amino acid sequence of SEQ ID NO:16.

Optionally, the antigen-binding site which binds to CEA comprises (a) a VH domain comprising at least one, at least two, or all three VH CDR sequences selected from (i) CDR-H1 comprising the amino acid sequence of SEQ ID NO:11, (ii) CDR-H2 comprising the amino acid sequence of SEQ ID NO:12, and (iii) CDR-H3 comprising an amino acid sequence selected from SEQ ID NO:13; and (b) a VL domain comprising at least one, at least two, or all three VL CDR sequences selected from (i) CDR-L1 comprising the amino acid sequence of SEQ ID NO:14, (ii) CDR-L2 comprising the amino acid sequence of SEQ ID NO:15, and (c) CDR-L3 comprising the amino acid sequence of SEQ ID NO:16.

In another aspect, the antigen-binding site which binds to CEA comprises (a) CDR-H1 comprising the amino acid sequence of SEQ ID NO:11; (b) CDR-H2 comprising the amino acid sequence of SEQ ID NO:12; (c) CDR-H3 comprising the amino acid sequence of SEQ ID NO:13; (d) CDR-L1 comprising the amino acid sequence of SEQ ID NO:14; (e) CDR-L2 comprising the amino acid sequence of SEQ ID NO:15; and (f) CDR-L3 comprising the amino acid sequence of SEQ ID NO:16.

In any of the above embodiments, the multispecific antibody may be humanized. In one embodiment, the anti-CEA antigen binding site comprises CDRs as in any of the above embodiments, and further comprises an acceptor human framework, e.g. a human immunoglobulin framework or a human consensus framework.

In another embodiment, the antigen-binding site which binds to CEA comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:17. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen binding site comprising that sequence retains the ability to bind to CEA, preferably with the affinity as set out above. In certain embodiments, a total of 1 to 10 amino acids have been substituted, inserted and/or deleted in SEQ ID NO:17. In certain embodiments, substitutions, insertions, or deletions occur in regions outside the HVRs (i.e., in the FRs). Optionally, the antigen-binding site which binds to CEA comprises the VH sequence in SEQ ID NO:17, including post-translational modifications of that sequence. In a particular embodiment, the VH comprises one, two or three CDRs selected from: (a) CDR-H1 comprising the amino acid sequence of SEQ ID NO:11, (b) CDR-H2 comprising the amino acid sequence of SEQ ID NO:12, and (c) CDR-H3 comprising the amino acid sequence of SEQ ID NO:13.

In another embodiment, the antigen-binding site which binds to CEA comprises a light chain variable domain (VL) having at least 90%, 91%, 92%, 93%, 94%, 95%, 96%, 97%, 98%, 99%, or 100% sequence identity to the amino acid sequence of SEQ ID NO:18. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen-binding site comprising that sequence retains the ability to bind to CEA, preferably with the affinity set out above. In certain embodiments, a total of 1 to 10 amino acids have been substituted, inserted and/or deleted in SEQ ID NO:18. In certain embodiments, the substitutions, insertions, or deletions occur in regions outside the HVRs (i.e., in the FRs). Optionally, the antigen-binding site for CEA comprises the VL sequence in SEQ ID NO:18, including post-translational modifications of that sequence. In a particular embodiment, the VL comprises one, two or three CDRs selected from (a) CDR-L1 comprising the amino acid sequence of SEQ ID NO:14; (b) CDR-L2 comprising the amino acid sequence of SEQ ID NO:15; and (c) CDR-L3 comprising the amino acid sequence of SEQ ID NO:16.

In another embodiment, the antigen-binding site which binds to CEA comprises a VH as in any of the embodiments provided above, and a VL as in any of the embodiments provided above. In one embodiment, the antibody comprises the VH and VL sequences in SEQ ID NO:17 and SEQ ID NO:18, respectively, including post-translational modifications of those sequences.

ii). In further particular embodiment, the antigen-binding site which binds to CEA may comprise at least one, two, three, four, five, or six CDRs selected from (a)CDR-H1 comprising the amino acid sequence of SEQ ID NO:19; (b) CDR-H2 comprising the amino acid sequence of SEQ ID NO:20; (c) CDR-H3 comprising the amino acid sequence of SEQ ID NO:21; (d) CDR-L1 comprising the amino acid sequence of SEQ ID NO:22; (e) CDR-L2 comprising the amino acid sequence of SEQ ID NO:23; and (f) CDR-L3 comprising the amino acid sequence of SEQ ID NO:24.

Optionally, the antigen-binding site which binds to CEA may comprise at least one, at least two, or all three VH CDR sequences selected from (a) CDR-H1 comprising the amino acid sequence of SEQ ID NO:19; (b) CDR-H2 comprising the amino acid sequence of SEQ ID NO:20; and (c) CDR-H3 comprising the amino acid sequence of SEQ ID NO:21.

Optionally, the antigen-binding site which binds to CEA comprises at least one, at least two, or all three VL CDRs sequences selected from (a) CDR-L1 comprising the amino acid sequence of SEQ ID NO:22; (b) CDR-L2 comprising the amino acid sequence of SEQ ID NO:23; and (c) CDR-L3 comprising the amino acid sequence of SEQ ID NO:24.

Optionally, the antigen-binding site which binds to CEA comprises (a) a VH domain comprising at least one, at least two, or all three VH CDR sequences selected from (i) CDR-H1 comprising the amino acid sequence of SEQ ID NO:19, (ii) CDR-H2 comprising the amino acid sequence of SEQ ID NO:20, and (iii) CDR-H3 comprising an amino acid sequence selected from SEQ ID NO:21; and (b) a VL domain comprising at least one, at least two, or all three VL CDR sequences selected from (i) CDR-L1 comprising the amino acid sequence of SEQ ID NO:22, (ii) CDR-L2 comprising the amino acid sequence of SEQ ID NO:23, and (c) CDR-L3 comprising the amino acid sequence of SEQ ID NO:24.

In another aspect, the antigen-binding site which binds to CEA comprises (a) CDR-H1 comprising the amino acid sequence of SEQ ID NO:19; (b) CDR-H2 comprising the amino acid sequence of SEQ ID NO:20; (c) CDR-H3 comprising the amino acid sequence of SEQ ID NO:21; (d) CDR-L1 comprising the amino acid sequence of SEQ ID NO:22; (e) CDR-L2 comprising the amino acid sequence of SEQ ID NO:23; and (f) CDR-L3 comprising the amino acid sequence of SEQ ID NO:24.

In any of the above embodiments, the multispecific antibody may be humanized. In one embodiment, the anti-CEA antigen binding site comprises CDRs as in any of the above embodiments, and further comprises an acceptor human framework, e.g. a human immunoglobulin framework or a human consensus framework.

In another embodiment, the antigen-binding site which binds to CEA comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:25. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen binding site comprising that sequence retains the ability to bind to CEA, preferably with the affinity as set out above. In certain embodiments, a total of 1 to 10 amino acids have been substituted, inserted and/or deleted in SEQ ID NO:25. In certain embodiments, substitutions, insertions, or deletions occur in regions outside the HVRs (i.e., in the FRs). Optionally, the antigen-binding site which binds to CEA comprises the VH sequence in SEQ ID NO:25, including post-translational modifications of that sequence. In a particular embodiment, the VH comprises one, two or three CDRs selected from: (a) CDR-H1 comprising the amino acid sequence of SEQ ID NO:19, (b) CDR-H2 comprising the amino acid sequence of SEQ ID NO:20, and (c) CDR-H3 comprising the amino acid sequence of SEQ ID NO:21.

In another embodiment, the antigen-binding site which binds to CEA comprises a light chain variable domain (VL) having at least 90%, 91%, 92%, 93%, 94%, 95%, 96%, 97%, 98%, 99%, or 100% sequence identity to the amino acid sequence of SEQ ID NO:26. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen-binding site comprising that sequence retains the ability to bind to CEA, preferably with the affinity set out above. In certain embodiments, a total of 1 to 10 amino acids have been substituted, inserted and/or deleted in SEQ ID NO:26. In certain embodiments, the substitutions, insertions, or deletions occur in regions outside the HVRs (i.e., in the FRs). Optionally, the antigen-binding site for CEA comprises the VL sequence in SEQ ID NO:26, including post-translational modifications of that sequence. In a particular embodiment, the VL comprises one, two or three CDRs selected from (a) CDR-L1 comprising the amino acid sequence of SEQ ID NO:22; (b) CDR-L2 comprising the amino acid sequence of SEQ ID NO:23; and (c) CDR-L3 comprising the amino acid sequence of SEQ ID NO:24.

In another embodiment, the antigen-binding site which binds to CEA comprises a VH as in any of the embodiments provided above, and a VL as in any of the embodiments provided above. In one embodiment, the antibody comprises the VH and VL sequences in SEQ ID NO:25 and SEQ ID NO:26, respectively, including post-translational modifications of those sequences.

iii) In further particular embodiment, the antigen-binding site which binds to CEA may comprise at least one, two, three, four, five, or six CDRs selected from (a) CDR-H1 comprising the amino acid sequence of SEQ ID NO:43; (b) CDR-H2 comprising the amino acid sequence of SEQ ID NO:44; (c) CDR-H3 comprising the amino acid sequence of SEQ ID NO:45; (d) CDR-L1 comprising the amino acid sequence of SEQ ID NO:46; (e) CDR-L2 comprising the amino acid sequence of SEQ ID NO:47; and (f) CDR-L3 comprising the amino acid sequence of SEQ ID NO:48. In some embodiments, CDR-H1 may have the sequence GFTFTDYYMN (SEQ ID NO.: 151).

Optionally, the antigen-binding site which binds to CEA may comprise at least one, at least two, or all three VH CDR sequences selected from (a) CDR-H1 comprising the amino acid sequence of SEQ ID NO:43; (b) CDR-H2 comprising the amino acid sequence of SEQ ID NO:44; and (c) CDR-H3 comprising the amino acid sequence of SEQ ID NO:45. In some embodiments, CDR-H1 may have the sequence GFTFTDYYMN (SEQ ID NO.: 151).

Optionally, the antigen-binding site which binds to CEA comprises at least one, at least two, or all three VL CDRs sequences selected from (a) CDR-L1 comprising the amino acid sequence of SEQ ID NO:46; (b) CDR-L2 comprising the amino acid sequence of SEQ ID NO:47; and (c) CDR-L3 comprising the amino acid sequence of SEQ ID NO:48.

Optionally, the antigen-binding site which binds to CEA comprises (a) a VH domain comprising at least one, at least two, or all three VH CDR sequences selected from (i) CDR-H1 comprising the amino acid sequence of SEQ ID NO:43, (ii) CDR-H2 comprising the amino acid sequence of SEQ ID NO:44, and (iii) CDR-H3 comprising an amino acid sequence selected from SEQ ID NO:45; and (b) a VL domain comprising at least one, at least two, or all three VL CDR sequences selected from (i) CDR-L1 comprising the amino acid sequence of SEQ ID NO:46, (ii) CDR-L2 comprising the amino acid sequence of SEQ ID NO:47, and (c) CDR-L3 comprising the amino acid sequence of SEQ ID NO48. In some embodiments, CDR-H1 may have the sequence GFTFTDYYMN (SEQ ID NO.: 151).

In another aspect, the antigen-binding site which binds to CEA comprises (a) CDR-H1 comprising the amino acid sequence of SEQ ID NO:43; (b) CDR-H2 comprising the amino acid sequence of SEQ ID NO:44; (c) CDR-H3 comprising the amino acid sequence of SEQ ID NO:45; (d) CDR-L1 comprising the amino acid sequence of SEQ ID NO:46; (e) CDR-L2 comprising the amino acid sequence of SEQ ID NO:47; and (f) CDR-L3 comprising the amino acid sequence of SEQ ID NO:48. In some embodiments, CDR-H1 may have the sequence GFTFTDYYMN (SEQ ID NO.: 151).

In any of the above embodiments, the multispecific antibody may be humanized. In one embodiment, the anti-CEA antigen binding site comprises CDRs as in any of the above embodiments, and further comprises an acceptor human framework, e.g. a human immunoglobulin framework or a human consensus framework.

In another embodiment, the antigen-binding site which binds to CEA comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:49. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen binding site comprising that sequence retains the ability to bind to CEA, preferably with the affinity as set out above. In certain embodiments, a total of 1 to 10 amino acids have been substituted, inserted and/or deleted in SEQ ID NO:49. In certain embodiments, substitutions, insertions, or deletions occur in regions outside the HVRs (i.e., in the FRs). Optionally, the antigen-binding site which binds to CEA comprises the VH sequence in SEQ ID NO:49, including post-translational modifications of that sequence. In a particular embodiment, the VH comprises one, two or three CDRs selected from: (a) CDR-H1 comprising the amino acid sequence of SEQ ID NO:43 or the sequence GFTFTDYYMN (SEQ ID NO.: 151), (b) CDR-H2 comprising the amino acid sequence of SEQ ID NO:44, and (c) CDR-H3 comprising the amino acid sequence of SEQ ID NO:45.

In another embodiment, the antigen-binding site which binds to CEA comprises a light chain variable domain (VL) having at least 90%, 91%, 92%, 93%, 94%, 95%, 96%, 97%, 98%, 99%, or 100% sequence identity to the amino acid sequence of SEQ ID NO:50. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen-binding site comprising that sequence retains the ability to bind to CEA, preferably with the affinity set out above. In certain embodiments, a total of 1 to 10 amino acids have been substituted, inserted and/or deleted in SEQ ID NO:50. In certain embodiments, the substitutions, insertions, or deletions occur in regions outside the HVRs (i.e., in the FRs). Optionally, the antigen-binding site for CEA comprises the VL sequence in SEQ ID NO:50, including post-translational modifications of that sequence. In a particular embodiment, the VL comprises one, two or three CDRs selected from (a) CDR-L1 comprising the amino acid sequence of SEQ ID NO:46; (b) CDR-L2 comprising the amino acid sequence of SEQ ID NO:47; and (c) CDR-L3 comprising the amino acid sequence of SEQ ID NO:48.

In another embodiment, the antigen-binding site which binds to CEA comprises a VH as in any of the embodiments provided above, and a VL as in any of the embodiments provided above. In one embodiment, the antibody comprises the VH and VL sequences in SEQ ID NO:49 and SEQ ID NO:50, respectively, including post-translational modifications of those sequences.

iv) In a still further particular embodiment, the antigen-binding site which binds to CEA may comprise at least one, two, three, four, five, or six CDRs selected from (a) CDR-H1 comprising the amino acid sequence of SEQ ID NO:59; (b) CDR-H2 comprising the amino acid sequence of SEQ ID NO:60; (c) CDR-H3 comprising the amino acid sequence of SEQ ID NO:61; (d) CDR-L1 comprising the amino acid sequence of SEQ ID NO:62; (e) CDR-L2 comprising the amino acid sequence of SEQ ID NO:63; and (f) CDR-L3 comprising the amino acid sequence of SEQ ID NO:64.

Optionally, the antigen-binding site which binds to CEA may comprise at least one, at least two, or all three VH CDR sequences selected from (a) CDR-H1 comprising the amino acid sequence of SEQ ID NO:59; (b) CDR-H2 comprising the amino acid sequence of SEQ ID NO:60; and (c) CDR-H3 comprising the amino acid sequence of SEQ ID NO:61.

Optionally, the antigen-binding site which binds to CEA comprises at least one, at least two, or all three VL CDRs sequences selected from (a) CDR-L1 comprising the amino acid sequence of SEQ ID NO:62; (b) CDR-L2 comprising the amino acid sequence of SEQ ID NO:63; and (c) CDR-L3 comprising the amino acid sequence of SEQ ID NO:64.

Optionally, the antigen-binding site which binds to CEA comprises (a) a VH domain comprising at least one, at least two, or all three VH CDR sequences selected from (i) CDR-H1 comprising the amino acid sequence of SEQ ID NO:59, (ii) CDR-H2 comprising the amino acid sequence of SEQ ID NO:60, and (iii) CDR-H3 comprising an amino acid sequence selected from SEQ ID NO:61; and (b) a VL domain comprising at least one, at least two, or all three VL CDR sequences selected from (i) CDR-L1 comprising the amino acid sequence of SEQ ID NO:62, (ii) CDR-L2 comprising the amino acid sequence of SEQ ID NO:63, and (c) CDR-L3 comprising the amino acid sequence of SEQ ID NO:64.

In another aspect, the antigen-binding site which binds to CEA comprises (a) CDR-H1 comprising the amino acid sequence of SEQ ID NO:59; (b) CDR-H2 comprising the amino acid sequence of SEQ ID NO:60; (c) CDR-H3 comprising the amino acid sequence of SEQ ID NO:61; (d) CDR-L1 comprising the amino acid sequence of SEQ ID NO:62; (e) CDR-L2 comprising the amino acid sequence of SEQ ID NO:63; and (f) CDR-L3 comprising the amino acid sequence of SEQ ID NO:64.

In any of the above embodiments, the multispecific antibody may be humanized. In one embodiment, the anti-CEA antigen binding site comprises CDRs as in any of the above embodiments, and further comprises an acceptor human framework, e.g. a human immunoglobulin framework or a human consensus framework.

In another embodiment, the antigen-binding site which binds to CEA comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:65. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen binding site comprising that sequence retains the ability to bind to CEA, preferably with the affinity as set out above. In certain embodiments, a total of 1 to 10 amino acids have been substituted, inserted and/or deleted in SEQ ID NO:65. In certain embodiments, substitutions, insertions, or deletions occur in regions outside the HVRs (i.e., in the FRs). Optionally, the antigen-binding site which binds to CEA comprises the VH sequence in SEQ ID NO:65, including post-translational modifications of that sequence. In a particular embodiment, the VH comprises one, two or three CDRs selected from: (a) CDR-H1 comprising the amino acid sequence of SEQ ID NO:59, (b) CDR-H2 comprising the amino acid sequence of SEQ ID NO:60, and (c) CDR-H3 comprising the amino acid sequence of SEQ ID NO:61.

In another embodiment, the antigen-binding site which binds to CEA comprises a light chain variable domain (VL) having at least 90%, 91%, 92%, 93%, 94%, 95%, 96%, 97%, 98%, 99%, or 100% sequence identity to the amino acid sequence of SEQ ID NO:66. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen-binding site comprising that sequence retains the ability to bind to CEA, preferably with the affinity set out above. In certain embodiments, a total of 1 to 10 amino acids have been substituted, inserted and/or deleted in SEQ ID NO:66. In certain embodiments, the substitutions, insertions, or deletions occur in regions outside the HVRs (i.e., in the FRs). Optionally, the antigen-binding site for CEA comprises the VL sequence in SEQ ID NO:66, including post-translational modifications of that sequence. In a particular embodiment, the VL comprises one, two or three CDRs selected from (a) CDR-L1 comprising the amino acid sequence of SEQ ID NO:62; (b) CDR-L2 comprising the amino acid sequence of SEQ ID NO:63; and (c) CDR-L3 comprising the amino acid sequence of SEQ ID NO:64.

In another embodiment, the antigen-binding site which binds to CEA comprises a VH as in any of the embodiments provided above, and a VL as in any of the embodiments provided above. In one embodiment, the antibody comprises the VH and VL sequences in SEQ ID NO:65 and SEQ ID NO:66, respectively, including post-translational modifications of those sequences.

v). In a still further particular embodiment, the antigen-binding site which binds to CEA may comprise at least one, two, three, four, five, or six CDRs selected from (a) CDR-H1 comprising the amino acid sequence of SEQ ID NO:116; (b) CDR-H2 comprising the amino acid sequence of SEQ ID NO:117 or 118; (c) CDR-H3 comprising the amino acid sequence of SEQ ID NO:119; (d) CDR-L1 comprising the amino acid sequence of SEQ ID NO:120, 121 or 122; (e) CDR-L2 comprising the amino acid sequence of SEQ ID NO:123, 124 or 125; and (f) CDR-L3 comprising the amino acid sequence of SEQ ID NO:126.

Optionally, the antigen-binding site which binds to CEA may comprise:
VH CDR sequences (a) CDR-H1 comprising the amino acid sequence of SEQ ID NO:116; (b) CDR-H2 comprising the amino acid sequence of SEQ ID NO:117 or 118; and (c) CDR-H3 comprising the amino acid sequence of SEQ ID NO:119; and/or
VL CDRs sequences (a) CDR-L1 comprising the amino acid sequence of SEQ ID NO:120, 121 or 122; (b) CDR-L2 comprising the amino acid sequence of SEQ ID NO:123, 124 or 125; and (c) CDR-L3 comprising the amino acid sequence of SEQ ID NO: 126.

In one embodiment, the antigen binding site for CEA comprises a heavy chain variable region (VH) comprise the amino acid sequence of SEQ ID NO: 127, or (more preferably) selected from SEQ ID NO: 129, 130, 131, 132, 133 or 134, and a light chain variable region (VL) comprising the amino acid sequence of SEQ ID NO: 128 or (more preferably) selected from SEQ ID NO: 135, 136, 137, 138, 139 or 140.

In any of the above embodiments, the multispecific antibody may be humanized. In one embodiment, the anti-CEA antigen binding site comprises CDRs as in any of the above embodiments, and further comprises an acceptor human framework, e.g. a human immunoglobulin framework or a human consensus framework.

In a particular aspect, the antigen binding domain capable of binding to CEA comprises:
(a) a VH domain comprising an amino acid sequence of SEQ ID NO:129 and a VL domain comprising an amino acid sequence of SEQ ID NO:139, or
(b) a VH domain comprising an amino acid sequence of SEQ ID NO:133 and a VL domain comprising an amino acid sequence of SEQ ID NO:139, or
(c) a VH domain comprising an amino acid sequence of SEQ ID NO:130 and a VL domain comprising an amino acid sequence of SEQ ID NO:139, or
(d) a VH domain comprising an amino acid sequence of SEQ ID NO:134 and a VL domain comprising an amino acid sequence of SEQ ID NO:138, or
(e) a VH domain comprising an amino acid sequence of SEQ ID NO:133 and a VL domain comprising an amino acid sequence of SEQ ID NO:138, or
(f) a VH domain comprising an amino acid sequence of SEQ ID NO:131 and a VL domain comprising an amino acid sequence of SEQ ID NO:138, or
(g) a VH domain comprising an amino acid sequence of SEQ ID NO:129 and a VL domain comprising an amino acid sequence of SEQ ID NO:138.

In another embodiment, the antigen-binding site which binds to CEA comprises a heavy chain variable domain (VH) sequence having at least 90%, 91%, 92%, 93%, 94%, 95%, 96%, 97%, 98%, 99%, or 100% sequence identity to the amino acid sequence as mentioned in a) to g) above. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen binding site comprising that sequence retains the ability to bind to CEA, preferably with the affinity as set out above. In certain embodiments, a total of 1 to 10 amino acids have been substituted, inserted and/or deleted. In certain embodiments, substitutions, insertions, or deletions occur in regions outside the HVRs (i.e., in the FRs).

In another embodiment, the antigen-binding site which binds to CEA comprises a light chain variable domain (VL) having at least 90%, 91%, 92%, 93%, 94%, 95%, 96%, 97%, 98%, 99%, or 100% sequence identity to the amino acid sequence as mentioned in a) to g) above. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen-binding site comprising that sequence retains the ability to bind to CEA, preferably with the affinity set out above. In certain embodiments, a total of 1 to 10 amino acids have been substituted, inserted and/or deleted. In certain embodiments, the substitutions, insertions, or deletions occur in regions outside the HVRs (i.e., in the FRs).

In another embodiment, the antigen-binding site which binds to CEA comprises a VH as in any of the embodiments provided above, and a VL as in any of the embodiments provided above.

### F. Exemplary Multispecific Antibodies

Various formats are possible for the multispecific antibodies used in the present invention. Exemplary formats are described in WO2019/201959, which is incorporated herein by reference, and any of the formats described therein may be applied. Specific exemplary antibodies are also described in WO2019/201959, and any of these specific antibodies may also be selected for use in the present invention.

In some embodiments, the multispecific antibody may comprise an Fc domain. The presence of an Fc region has benefits in the context of radioimmunotherapy and radioimaging, e.g. prolonging the protein's circulating half-life and/or resulting in higher tumour uptake than may be observed with smaller fragments. The Fc domain may be engineered to reduce or eliminate Fc effector function.

One exemplary format comprises a full-length antibody (e.g., an IgG) comprising a first and second antibody heavy chain and a first and second antibody light chain, wherein the first heavy chain and the first light chain assemble to form an antigen binding site for the radiolabelled compound, and wherein the second heavy chain and second light chain assemble to form an antigen binding site for the target antigen.

Correct assembly of the heterodimeric heavy chains can be assisted e.g. by the use of knob into hole mutations and/or other modifications as discussed further below.

Correct assembly of the light chains with their respective heavy chain can be assisted by using cross-mab technology. In this approach, either the first heavy chain and the first light chain, or the second heavy chain and the second light chain, can assemble to form a cross-Fab fragment (while the others assemble to form a conventional Fab). Thus, in one embodiment, the first heavy chain may comprise a VL domain in place of the VH domain (e.g., VL-CH1-hinge-CH2-CH3) and the first light chain may comprise a VH domain exchanged for the VL domain (e.g., VH-CL), or the first heavy chain may comprise a CL domain in place of the HC1 domain (e.g., VH-CL-hinge-CH2-CH3) and the first light chain may comprise a CH1 domain in place of the CL domain (e.g., VL-CH1). In this embodiment, the second heavy chain and the second light chain have the conventional domain structure (e.g., VH-CH1-hinge-CH2-CH3 and VL-CL, respectively). In an alternative embodiment, the second heavy chain may comprise a VL domain in place of the VH domain (e.g., VL-CH1-hinge-CH2-CH3) and the second light chain may comprise a VH domain exchanged for the VL domain (e.g., VH-CL), or the second heavy chain may comprise a CL domain in place of the HC1 domain (e.g., VH-CL-hinge-CH2-CH3) and the second light chain may comprise a CH1 domain in place of the CL domain (e.g., VL-CH1). In this embodiment, the first heavy chain and the first light chain have the conventional domain structure.

In some embodiments, correct assembly of the light chains with their respective heavy chain can additionally or alternatively be assisted by using charge modification, as discussed further below.

In some embodiments of the above format, the format may be bivalent. In another possible embodiment, further antigen binding moieties may be fused e.g., to the first and/or second heavy chain to increase the valency for one or both antigens. For instance, a further antigen binding moiety for the target antigen antigen may be fused to the N-terminus of one or both of the heavy chain molecules. In some embodiments, the antibody may be multivalent, e.g, bivalent, for the tumour associated antigen and monovalent for the radiolabelled compound.

The further antigen binding moiety may for instance be an scFab e.g., comprising an antigen binding site for the first antigen (e.g., the tumour associated antigen). In another embodiment, the further antigen binding moiety is a Fab or a cross-Fab. For instance, the N- or C-terminus of one of the heavy chains may be linked via a polypeptide linker to a first polypeptide consisting of a VH domain and a CH1 domain, which associates with a second polypeptide consisting of a VL and CL domain to form a Fab. In another embodiment, the N- or C-terminus of one of the heavy chains may be linked via a polypeptide linker to a first polypeptide consisting of a VL domain and a CH1 domain, which associates with a second polypeptide consisting of a VH and CL domain. In another embodiment, the N- or C-terminus of one of the heavy chains may be linked via a polypeptide linker to a first polypeptide consisting of a VH domain and a CL domain, which associates with a second polypeptide consisting of a VL and CH1 domain.

In this format, it may be preferred that binding arms of the same antigen specificity are formed by association with the same light chain. Thus, the antigen binding moieties/arms for the target antigen may be cross-Fabs, and the antigen binding moiety(s)/arm(s) for the radiolabelled compound may be conventional Fabs. Alternatively, the antigen binding moieties/arms for the target antigen may be conventional Fabs, and the antigen binding moiety(s)/arm(s) for the radiolabelled compound may be cross-Fabs.

The format may also incorporate charge modification, as discussed further below.

Another exemplary format comprises a full length antibody such as an IgG comprising an antigen binding site for the target antigen (e.g., which may be divalent for the target target antigen), linked to an antigen binding moiety for the radiolabelled compound.

For example, the antigen binding moiety for the radiolabelled compound may be a scFab comprising an antigen binding site for the radiolabelled compound (e.g., the Pb-DOTAM chelate). In some embodiments, the scFab may be fused to the C-terminus of one of the two heavy chains of the full-length antibody, e.g., at the C-terminus of its CH3 domain.

Correct assembly of heterodimeric heavy chains may be assisted e.g. by the use of knob into hole mutations and/or other modifications as discussed further below.

Another exemplary format comprises a full length antibody comprising an antigen binding site for the target antigen (e.g., which may be divalent for the target antigen), wherein the N- or C-terminus of one of the heavy chains is linked via a polypeptide linker to a first polypeptide and wherein the first polypeptide associates with a second polypeptide to form a Fab or a cross-Fab comprising a binding site for the radiolabelled compound. For instance, this format may comprise:
i) a first polypeptide consisting of a VH domain and a CH1 domain, which is associated with a second polypeptide consisting of a VL and CL domain; or
ii) a first polypeptide consisting of a VL domain and a CH1 domain, which is associated with a second polypeptide consisting of a VH and CL domain; or
iii) a first polypeptide consisting of a VH domain and a CL domain, which is associated with a second polypeptide consisting of a VL and CH1 domain;
such that the first and second polypeptide together form an antigen binding site for the radiolabelled compound.

Correct assembly of the heterodimeric heavy chains may be assisted e.g. by the use of knob into hole mutations and/or other modifications as discussed further below, including charge modifications. For instance, the Fab domains of the full-length antibody may include charge modifications.

In another exemplary format the antibody may be a bispecific antibody comprising:
a) a full length antibody specifically binding to the target antigen and consisting of two antibody heavy chains and two antibody light chains;
b) a polypeptide consisting of
   i) an antibody heavy chain variable domain (VH); or ii) an antibody heavy chain variable domain (VH) and an antibody heavy chain constant domain (CH1); or
   iii) an antibody heavy chain variable domain (VH) and an antibody light chain constant domain (CL);
wherein said polypeptide is fused with the N-terminus of the VH domain via a peptide linker to the C-terminus of one of the two heavy chains of said full-length antibody; c) a polypeptide consisting of
   i) an antibody light chain variable domain (VL); or
   ii) an antibody light chain variable domain (VL) and an antibody light chain constant domain (CL); or
   iii) an antibody light chain variable domain (VL) and an antibody heavy chain constant domain (CH1);
wherein said polypeptide is fused with the N-terminus of the VL domain via a peptide linker to the C-terminus of the other of the two heavy chains of said full-length antibody; and wherein the antibody heavy chain variable domain (VL) of the peptide under (b) and the antibody light chain variable domain of the peptide under (c) together form an antigen-binding site for the radiolabelled compound.

In this format, if the first polypeptide is as set out in b(i), then the second polypeptide is as set out in c(i); if the first polypeptide is as set out in b(ii), then the second polypeptide is as set out in c(ii); and if the first polypeptide is as set out in b(iii), then the second polypeptide is as set out in c(iii). Charge modifying substitutions may also be used, e.g., in the Fabs of the full length antibody.

Optionally, the structure may be stabilized, whereby the antibody heavy chain variable region (VH) of the polypeptide under (b) and the antibody light chain variable domain (VL) of the polypeptide under (c) are linked and stabilized via an interchain disulfide bridge, e.g., by introduction of a disulfide bond between the following positions (numbering always according to EU index of Kabat):
i) heavy chain variable domain positon 44 to light chain variable domain position 100,
ii) heavy chain variable domain position 105 to light chain variable domain position 43, or
iii) heavy chain variable domain position 101 to light chain variable domain positon 100.

Examples of the format above in which the antibody of (b) consists of a VH domain and the antibody of (c) consists of a VL domain are PRIT213 (also referred to as PRIT-0213) and PRIT214 (also referred to as PRIT-0214) as described in WO2019/201959. Thus, in a specific embodiment, a multispecific antibody for use in the present invention may comprise:
i) a first heavy chain having the amino acid sequence of SEQ ID NO: 22;
ii) a second heavy chain having the amino acid sequence of SEQ ID NO: 23; and
iii) two antibody light chains having the amino acid sequence of SEQ ID NO: 21, where the sequence numbering is the sequence numbering of WO2019/201959.

In another embodiment, the multispecific antibody for use in the present invention may comprise
i) a first heavy chain having the amino acid sequence of SEQ ID NO: 19;
ii) a second heavy chain having the amino acid sequence of SEQ ID NO: 20; and
iii) two antibody light chains having the amino acid sequence of SEQ ID NO: 21, where the sequence numbering is the sequence numbering of WO2019/201959.

### G. Exemplary Formats for Split Multispecific Antibodies

In other embodiments, an antibody for use in the combination therapy may be a split multispecific antibody. The split multispecific antibody may comprise:
i) a first hemibody that binds to an antigen expressed on the surface of a target cell, and which further comprises a VH domain of an antigen binding site for a radiolabelled compound, but which does not comprise a VL domain of an antigen binding site for the radiolabelled compound; and
ii) a second hemibody that binds to an antigen expressed on the surface of the target cell, and which further comprises a VL domain of an antigen binding site for the radiolabelled compound, but which does not comprise a VH domain of the antigen binding site for the radiolabelled compound,
wherein said VH domain of the first hemibody and said VL domain of the second hemibody are together capable of forming a functional antigen binding site for the radiolabelled compound.

The first and second hemibody may bind to the same target antigen, at the same or a different epitope.

In some embodiments, the first and second hemibody may each comprise an Fc domain. The Fc domain may be engineered to reduce or eliminate Fc effector function.

In some embodiments, as discussed above, where the VH domain of an antigen binding site for a radiolabelled compound is free at its C-terminus (e.g., is not fused to another domain via its C-terminus), then it may be extended by one or more residues to avoid binding of HAVH autoantibodies. For instance, the extension may be by 1-10 residues, e.g., 1,2,3,4,5,6,7,8,9 or 10 residues. In one embodiment, it may be extended by one or more alanine residues, optionally by one alanine residue. The VH sequence may also be extended by an N-terminal portion of the CH1 domain, e.g., by 1-10 residues from the N-terminus of the CH1 domain, e.g., from the human IgG1 CH1 domain. (The first ten residues of the human IgG1 CH1 domain are ASTKGPSVFP, and so in one embodiment, from 1-10 residues may be taken from the N-terminus of this sequence). For instance, in one embodiment, the peptide sequence AST (corresponding to the first 3 residues of the IgG1 CH1 domain) is added to the C-terminus of the VH region. In some embodiments, the first and/or the hemibody may each be multivalent, e.g., bivalent for the target antigen (e.g., the tumour associated antigen). This has the advantage of increasing avidity.

In some embodiments, it may be preferred that when the first and second hemibody are associated, they form an antibody complex which is monovalent for the radiolabelled compound. Thus, the first hemibody may comprise only one VH domain of an antigen binding site for the radiolabelled compound, and the second hemibody may comprise only one VL domain of an antigen binding site for a radiolabelled compound, so that together they form only one complete functional binding site for the radiolabelled compound.

The hemibodies may each comprise i) at least one antigen binding moiety (e.g., antibody fragment) capable of binding to the target antigen, ii) either a VL domain or a VH domain of the antigen binding site for the radiolabelled compound, and iii) optionally a Fc region. The antibody fragment may be for example at least one Fv, scFv, Fab or cross-Fab fragment, comprising an antigen binding site specific for the target antigen. The antigen binding moiety (e.g., antibody fragment) may be fused to a) either a VL domain or a VH domain of the antigen binding site for the radiolabelled compound or b) if the antibodies comprise a Fc region, to a Fc region which is fused to either a VL domain or a VH domain of the antigen binding site for the radiolabelled compound. In some embodiments, the C-terminus of the Fc region is fused to the N-terminus of the VL domain or VH domain.

The fusion may be direct or indirect. In some embodiments, the fusion may be via a linker. For instance, the Fc region may be fused to the antibody fragment via the hinge region or another suitable linker. Similarly, the connection of the VL or VH domain of the antigen binding site for the radiolabelled compound to the rest of the antibody structure may be made via a linker. In one particular embodiment, the first hemibody may comprise or consist of:
a) an scFv fragment, wherein the scFv fragment binds the target antigen; and
b) a polypeptide comprising or consisting of
   i) an antibody heavy chain variable domain (VH); or
   ii) an antibody heavy chain variable domain (VH) and an antibody heavy chain constant domain, wherein the C-terminus of the VH domain is fused to the N terminus of the constant domain;
wherein said polypeptide is fused by the N-terminus of the VH domain, preferably via a peptide linker, to the C-terminus of scFv fragment.

The second hemibody may comprise or consist of:
c) a second scFv binding the target antigen; and
d) a polypeptide comprising or consisting of
   i) an antibody light chain variable domain (VL); or
   ii) an antibody light chain variable domain (VL) and an antibody light chain constant domain, wherein the C-terminus of the VL domain is fused to the N-terminus of the constant domain;
      wherein said polypeptide is fused by the N-terminus of the VL domain, preferably via a peptide linker, to the C-terminus of scFv fragment.

The antibody heavy chain variable domain (VH) of the first hemibody and the antibody light chain variable domain (VL) of the second hemibody together form a functional antigen-binding site for the radiolabelled compound, upon association of the two hemibodies.

Optionally, the polypeptide of part b(i) may additionally comprise one or more residues at the C-terminus of the VH domain, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

The target antigen-recognizing variable domains of the heavy and light chain of an scFv can be connected by a peptide tether. Such a peptide tether may comprise 1 to 25 amino acids, preferably 12 to 20 amino acids, preferably 12 to 16 or 15 to 20 amino acids. The above described tether may comprise one or more (G3S) and/or (G4S) motifs, in particular 1, 2, 3, 4, 5 or 6 (G3S) and/or (G4S) motifs, preferably 3 or 4 (G3S) and/or (G4S) motifs, more preferably 3 or 4 (G4S) motifs.

Optionally, the first hemibody may consist essentially of or consist of the components (a) and (b) listed above and the second hemibody may consist or consist essentially of the components (c) and (d) listed above. In any event, the first hemibody does not comprise an antibody light chain variable domain (VL) capable of forming a functional antigen-binding site for the radiolabelled compound in association with component (b) of the first hemibody; and the second hemibody does not comprise an antibody heavy chain variable (VH) domain capable of forming a functional antigen-binding site for the radiolabelled compound in association with component (d) of the second hemibody.

In another particular embodiment, the first hemibody may comprise or consist of:
a) a Fab fragment binding the target antigen, and
b) a polypeptide comprising or consisting of
   i) an antibody heavy chain variable domain (VH) of an antigen binding site for a radiolabelled compound, or
   ii) an antibody heavy chain variable domain (VH) of an antigen binding site for a radiolabelled compound and an antibody heavy chain constant domain, wherein the C-terminus of VH domain is fused to the N-terminus of the constant domain;
   wherein the polypeptide is fused by the N-terminus of the VH domain, preferably via a peptide linker, to the C terminus of the CL or CH1 domain of the Fab fragment.
      The second hemibody may comprise or consist of:
c) a Fab fragment binding the target antigen, and
d) a polypeptide comprising or consisting of
   iii) an antibody light chain variable domain (VL) of an antigen binding site for a radiolabelled compound, or
   iv) an antibody light chain variable domain (VL) of an antigen binding site for a radiolabelled compound and an antibody light chain constant domain, wherein the C-terminus of the VL domain is fused to the N-terminus of the constant domain;
wherein the polypeptide is fused by the N-terminus of the VL domain, preferably via a peptide linker, to the C-terminus of the CL or CH1 domain of the Fab fragment.

The antibody heavy chain variable domain (VH) of the polypeptide of (b) and antibody light chain variable domain (VL) of polypeptide of (d) together form a functional antigen-binding site for the radiolabelled compound (i.e., upon association of the two hemibodies).

Optionally, the polypeptide of part b(i) may additionally comprise one or more residues at the C-terminus of the VH domain as described above, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

Optionally, the first hemibody may consist essentially of or consist of the components (a) and (b) listed above and the second hemibody may consist or consist essentially of the components (c) and (d) listed above. In any event, the first hemibody does not comprise an antibody light chain variable domain (VL) capable of forming a functional antigen-binding site for the radiolabelled compound in association with component (b) of the first hemibody; and the second hemibody does not comprise an antibody heavy chain variable (VH) domain capable of forming a functional antigen-binding site for the radiolabelled compound in association with component (d) of the second hemibody.

The chain of the Fab fragment which is fused to the polypeptide can be independently selected for the first and for the second hemibody. Thus, in one embodiment, the polypeptide of (b) is fused to the C-terminus of the CH1 domain of the Fab fragment of the first hemibody, and the polypeptide of (d) is fused to the C-terminus of the CH1 domain of the Fab fragment of the second hemibody. In another embodiment, the polypeptide of (b) is fused to the C-terminus of the CL domain of the Fab fragment of the first hemibody, and the polypeptide of (d) is fused to the C-terminus of the CL domain of the Fab fragment of the second hemibody. In another embodiment, the polypeptide of (b) is fused to the C-terminus of the CH1 domain of the Fab fragment of the first hemibody, and polypeptide of (d) is fused to the C-terminus of the CL domain of the Fab fragment of the second hemibody. In a further embodiment, polypeptide of (b) is fused to the C-terminus of the CL domain of the Fab fragment of the first hemibody, and the polypeptide of (d) is fused to the C-terminus of the CH1 domain of the Fab fragment of the second hemibody.

As noted above, in some embodiments, the first and/or the second hemibody may each be multivalent, e.g., bivalent for the target antigen (e.g., the tumour associated antigen). This has the advantage of increasing avidity. The hemibodies may be multivalent, e.g., bivalent, and may each be monospecific for a particular epitope (which may be the same epitope for the first and second hemibody, or may be different for the first and second hemibody). Thus, in some embodiments, the first hemibody may comprise i) two or more antigen binding moieties (e.g., antibody fragments) capable of binding the same epitope of the target antigen, ii) either a VL domain or a VH domain of the antigen binding site for the radiolabelled compound (but not both), and iii) optionally a Fc region. The second hemibody may comprise i) two or more antigen binding moieties (e.g., antibody fragments) capable of binding the same epitope of the target antigen, ii) either a VL domain or a VH domain of the antigen binding site for the radiolabelled compound (but not both), and iii) optionally a Fc region. As stated above, the epitope may be the same for the first and second hemibody, or may be different for the first and second hemibody.

For example, each of the first and the second hemibody may comprise a tandem Fab, i.e., two Fab fragments, which are connected via a peptide linker (Fab-linker-Fab), wherein the first Fab is connected via its C-terminus to the N-terminus of the second Fab.

In one embodiment, the first hemibody comprises
a) a tandem Fab comprising two Fab fragments, wherein the first and the second Fab fragment bind the same target antigen (“target antigen A”) and the epitope bound by the first Fab fragment is the same as the epitope bound by the second Fab fragment, and wherein the first and the second Fab fragment are connected via a peptide linker, wherein the first Fab is connected via its C-terminus to the N-terminus of the second Fab; and
b) a polypeptide comprising or consisting of
   i) an antibody heavy chain variable domain (VH); or
   ii) an antibody heavy chain variable domain (VH) and an antibody constant domain (CH1), wherein the C-terminus of VH domain is fused to the N-terminus of the CH1 domain;
wherein said polypeptide is fused by the N-terminus of the VH domain, preferably via a peptide linker, to the C-terminus of the CL or CH1 domain of the second Fab fragment;
and the second hemibody comprises
c) a tandem Fab comprising two Fab fragments, wherein the first and the second Fab fragment bind target antigen A and the epitope bound by the first Fab fragment is the same as the epitope bound by the second Fab fragment, and wherein the first and the second Fab fragment are connected via a peptide linker, wherein the first Fab is connected via its C-terminus to the N-terminus of the second Fab; and
d) a polypeptide comprising or consisting of
i) an antibody light chain variable domain (VL); or
ii) an antibody light chain variable domain (VL) and an antibody light chain constant domain (CL), wherein the C-terminus of VH domain is fused to the N-terminus of the constant domain;
wherein said polypeptide is fused by the N-terminus of the VL domain, preferably via a peptide linker, to the C-terminus of the CL or CH1 domain of the second Fab fragment.

The antibody heavy chain variable domain (VH) of part b (in the first hemibody) and the antibody light chain variable domain (VL) of part (d) (in the second hemibody) together form a functional antigen-binding site for the radiolabelled compound, i.e., upon association of the two hemibodies.

Optionally, the polypeptide of part b(i) may additionally comprise one or more residues at the C-terminus of the VH domain, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

The chain of the Fab tandem which is fused to the polypeptide (i.e., whether the polypeptide is fused to the CL or the CH1 domain of the second Fab fragment) can be independently selected for the first and for the second hemibody.

As described above, the first Fab fragment of the Fab tandem is connected to the N-terminus of the second Fab fragment. In one embodiment, the C-terminus of the heavy chain fragment of the first Fab fragment is connected to the N terminus of the heavy-chain fragment or light chain fragment of the second Fab fragment. In another embodiment, the C-terminus light chain fragment of the first Fab fragment is connected to the N terminus of the heavy-chain fragment or light chain fragment of the second Fab fragment. Thus, in some embodiments the Fab tandem of the first and/or second hemibody may comprise three chains as follows:
1) the light chain fragment ((VLCL)1) of the first Fab fragment, the heavy chain fragment of the first Fab fragment connected to the heavy chain fragment of the second Fab fragment via a peptide linker ((VHCH1)1-linker-(VHCH1)2) and the light chain fragment of the second Fab fragment ((VLCL)2); or
2) the light chain fragment of the first Fab fragment ((VLCL)1), the heavy chain fragment of the first Fab fragment connected to the light chain fragment of the second Fab fragment via a peptide linker ((VHCH1)1-linker- (VLCL)2) and the heavy chain fragment of the second Fab fragment ((VH-CH1)2); or
3) the heavy chain fragment (VHCH1) of the first Fab fragment, the light chain fragment of the first Fab fragment connected to the light chain fragment of the second Fab fragment via a peptide linker ((VLCL)1-linker-(VLCL)2) and the heavy chain fragment of the second Fab fragment; or
4) the heavy chain fragment (VHCH1) of the first Fab fragment, the light chain fragment of the first Fab fragment connected to the heavy chain fragment of the second Fab fragment via a peptide linker ((VLCL)1-linker-(VHCH1)2) and the light chain fragment of the second Fab fragment ((VLCL)2).

In another embodiment, the first and/or second hemibody may each bind more than one, optionally two, different epitopes of the target antigen. Thus, one or both of the hemibodies may be biparatopic for the target antigen. In some embodiments, the first and second hemibody may each comprise i) an antigen binding moiety (e.g., an antibody fragment) capable of binding a first epitope of the target antigen; ii) an antigen binding moiety (e.g., antibody fragment) capable of binding a second epitope of the target antigen, iii) either a VL domain or a VH domain of the antigen binding site for the radiolabelled compound (but not both), and iv) optionally a Fc region.

In such embodiments, correct assembly of the light chains with their respective heavy chain can be assisted by using cross-mab technology. For instance, in one embodiment, each hemibody may comprise a tandem Fab comprising one Fab and one cross-Fab, in which one fragment selected from the Fab and the cross-Fab is specific for a first epitope, and the other is specific for a second epitope.

In one particular example, the first hemibody may comprise:
a) a tandem Fab comprising a first fragment and a second fragment, wherein the first fragment is connected by its C-terminus via a peptide linker to the N-terminus of the second fragment, wherein the first fragment binds a first epitope of the target antigen and the second fragment binds a second epitope of the target antigen, and wherein one of the fragments selected from the first and second fragments is a Fab and the other is a cross-Fab,
b) a polypeptide comprising or consisting of
   i) an antibody heavy chain variable domain (VH); or
   ii) an antibody heavy chain variable domain (VH) and an antibody heavy chain constant domain (CH1), wherein the C-terminus of VH domain is fused to the N-terminus of the CH1 domain;
wherein said polypeptide is fused by the N-terminus of the VH domain, preferably via a peptide linker, to the C-terminus of one of the chains of the second fragment.

The second hemibody may comprise
c) a tandem Fab comprising a first fragment and a second fragment, wherein the first fragment is connected by its C-terminus to the N-terminus of the second fragment, wherein the first fragment binds a first epitope of the target antigen and the second fragment binds a second epitope of the target antigen, and wherein one of the fragments selected from the first and second fragments is a Fab and the other is a cross-Fab; and
d) a polypeptide comprising or consisting of
i) an antibody light chain variable domain (VL); or
ii) an antibody light chain variable domain (VL) and an antibody light chain constant domain (CL), wherein the C-terminus of VL domain is fused to the N-terminus of the light chain constant domain
wherein said polypeptide is fused by the N-terminus of the VL domain, preferably via a peptide linker, to the C-terminus of one of the chains of the second fragment.

The antibody heavy chain variable domain (VH) of the first hemibody and the antibody light chain variable domain (VL) of the second hemibody together form a functional antigen-binding site for the radiolabelled compound.

Optionally, the polypeptide of part b(i) may additionally comprise one or more residues at the C-terminus of the VH domain, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

Either the first or second fragment can be the cross-Fab, as long as the tandem Fab comprises one conventional Fab and one cross Fab.

In any of the tandem Fab embodiments described above (including those involving cross-Fabs), optionally, the first hemibody may consist essentially of or consist of the components (a) and (b) and the second may consist or consist essentially of the components (c) and (d). In any event, the first hemibody does not comprise an antibody light chain variable domain (VL) capable of forming a functional antigen-binding site for the radiolabelled compound in association with component (b) of the first hemibody; and the second hemibody does not comprise an antibody heavy chain variable (VH) domain capable of forming a functional antigen-binding site for the radiolabelled compound in association with component (d) of the second hemibody.

As noted above, in some embodiments, the first and second hemibody may each comprise an Fc domain, optionally engineered to reduce or eliminate effector function.

In one embodiment, each of the first and second hemibody may comprise i) an Fc domain, ii) at least one antigen binding moiety (e.g., antibody fragment, such as an scFv, Fv, Fab or cross-Fab fragment) capable of binding to the target antigen and iii) either a VL domain or a VH domain of the antigen binding site for the radiolabelled compound (but not both).

Optionally, the hemibodies comprising the Fc domain may be monovalent in respect of binding to the target antigen. In other embodiments, they may be multivalent, e.g., bivalent. The first and second hemibodies may each be multivalent and monospecific for the same epitope of the target antigen. In still other embodiments, the first and second hemibodies may each have binding sites for different epitopes of the target antigen—e.g., they may be biparatopic.

The antibody fragment may be an scFv. Thus, in one embodiment, the first hemibody may comprise or consist of:
a) an scFv fragment, wherein the scFv fragment binds the target antigen;
b) an Fc domain; and
c) a polypeptide comprising or consisting of
i) an antibody heavy chain variable domain (VH); or
ii) an antibody heavy chain variable domain (VH) and an antibody heavy chain constant domain (CH1), wherein the C-terminus of the VH domain is fused to the N-terminus of the constant domain;
wherein the scFv of (a) is fused to the N-terminus of the Fc domain, and wherein the polypeptide of c) is fused by the N-terminus of the VH domain to the C-terminus of the Fc domain, preferably via a peptide linker.

Optionally, the polypeptide of part c(i) may additionally comprise one or more residues at the C-terminus of the VH domain, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

The second hemibody may comprise or consist of:
d) a second scFv binding the target antigen;
e) an Fc domain; and
f) a polypeptide comprising or consisting of
i) an antibody light chain variable domain (VL); or
ii) an antibody light chain variable domain (VL) and an antibody light chain constant domain (CL), wherein the C-terminus of the VL domain is fused to the N-terminus of the constant domain;
wherein the scFv of (d) is fused to the N-terminus of the Fc domain, and wherein the polypeptide of (f) is fused by the N-terminus of the VH domain to the C-terminus of the Fc domain, preferably via a peptide linker.

In another embodiment, the first and second hemibody may each be a one-armed IgG comprising a Fab for the target antigen (e.g., a single Fab for the target antigen) and an Fc domain. Thus, the first hemibody may comprise or consist of:
i) a complete light chain fragment;
ii) a complete heavy chain;
iii) an additional Fc chain lacking Fd; and
iv) a polypeptide comprising or consisting of the VH domain of the antigen binding site for the radiolabeled compound;
wherein the light chain of (i) and the heavy chain of (ii) together provide an antigen binding site for the target antigen; and wherein the polypeptide comprising or consisting of the VH domain of the antigen binding site for the radiolabeled compound is fused by its N-terminus, preferably via a linker, to the C-terminus of either (ii) or (iii).

The second hemibody may comprise or consist of
v) a complete light chain fragment;
vi) a complete heavy chain;
vii) an additional Fc chain lacking Fd; and
viii) a polypeptide comprising or consisting of the VL domain of the antigen binding site for the radiolabeled compound;
wherein the light chain of (v) and the heavy chain of (vi) together provide an antigen binding site for the target antigen; and wherein the polypeptide comprising or consisting of the VL domain of the antigen binding site for the radiolabeled compound is fused by its N-terminus, preferably via a linker, to the C-terminus of either (vi) or (vii).

The polypeptide comprising or consisting of the VH domain of the antigen binding site for the radiolabeled compound may be a polypeptide comprising or consisting of
i) an antibody heavy chain variable domain (VH), in which case the polypeptide may additionally comprise one or more residues at the C-terminus of the VH domain, optionally, one or more alanine residues, optionally a single alanine residue, or optionally an N-terminal portion of the CH1 domain as described above; or
ii) an antibody heavy chain variable domain (VH) and an antibody heavy chain constant domain (CH1), wherein the C-terminus of VH domain is fused to the N-terminus of the CH1 domain.

The polypeptide comprising or consisting of the VL domain of the antigen binding site for the radiolabeled compound may be a polypeptide comprising or consisting of
i) an antibody heavy chain variable domain (VL); or
ii) an antibody heavy chain variable domain (VL) and an antibody light chain constant domain, wherein the C-terminus of VL domain is fused to the N-terminus of the constant domain.

When the first and second hermibodies are heterodimers, e.g., as for one-armed IgGs, their assembly may be assisted by the use of knob-into-hole technology, as described further below.

In another embodiment, the hemibodies may each comprise a tandem Fab as described above (e.g., comprising two Fab fragments, wherein the first and the second Fab fragment both bind the same epitope of target antigen A; or comprising a Fab and a cross Fab wherein one of them binds a first epitope of target antigen A and the other binds a second epitope of target antigen A), wherein the Fab tandem is fused (e.g., via its C-terminus) to the N-terminus of an Fc domain, and wherein peptide comprising or consisting of the VH or VL domain of the antigen binding site for the radiolabelled compound is fused (e.g., via its N-terminus) to the C-terminus of the Fc domain.

Thus, the first hemibody may comprise or consist of:
a) a tandem Fab selected from
   i) a tandem Fab comprising two Fab fragments, wherein the first and the second Fab fragment bind target antigen A and the epitope bound by the first Fab fragment is the same as the epitope bound by the second Fab fragment, and wherein the first and the second Fab fragment are connected via a peptide tether, wherein the first Fab is connected via its C-terminus to the N-terminus of the second Fab; and
   ii) a tandem Fab comprising a first fragment and a second fragment, wherein the first fragment is connected by its C-terminus via a peptide tether to the N-terminus of the second fragment, wherein the first fragment binds a first epitope of target antigen A and the second fragment binds a second epitope of target antigen A, and wherein one of the fragments selected from the first and second fragments is a Fab and the other is a cross-Fab;
b) an Fc domain; and
c) a polypeptide comprising or consisting of:
   i) an antibody heavy chain variable domain (VH); or
   ii) an antibody heavy chain variable domain (VH) and an antibody heavy chain constant domain (CH1), wherein the C-terminus of VH domain is fused to the N-terminus of the CH1 domain,
      wherein the tandem Fab is fused to the N-terminus of one of the chains of the Fc domain, and the polypeptide of c) is fused by the N-terminus of the VH domain to the C-terminus of one of the chains of the Fc domain, preferably via a peptide linker.

Optionally, the polypeptide of part c(i) may additionally comprise one or more residues at the C-terminus of the VH domain, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

The second hemibody may comprise or consist of:
d) a tandem Fab selected from:
   i) a tandem Fab comprising two Fab fragments, wherein the first and the second Fab fragment bind target antigen A and the epitope bound by the first Fab fragment is the same as the epitope bound by the second Fab fragment, and wherein the first and the second Fab fragment are connected via a peptide tether, wherein the first Fab is connected via its C-terminus to the N-terminus of the second Fab; and
   ii) a tandem Fab comprising a first fragment and a second fragment, wherein the first fragment is connected by its C-terminus via a peptide tether to the N-terminus of the second fragment, wherein the first fragment binds a first epitope of target antigen A and the second fragment binds a second epitope of target antigen A, and wherein one of the fragments selected from the first and second fragments is a Fab and the other is a cross-Fab;
e) an Fc domain; and
f) a polypeptide comprising or consisting of:
   i) an antibody heavy chain variable domain (VL); or
   ii) an antibody heavy chain variable domain (VL) and an antibody light chain constant domain, wherein the C-terminus of VL domain is fused to the N-terminus of the light chain constant domain,
wherein the tandem Fab of (d) is fused to the N-terminus one of the chains of the Fe domain, and the polypeptide of (f) is fused by the N-terminus of the VL domain to the C-terminus of one of the chains of the Fe domain, preferably via a peptide linker.

The VH domain of the first hemibody and the VL domain of the second hemibody together form an antigen binding site for the radiolabelled compound, i.e., upon association of the two antibodies.

If the first hemibody comprises a tandem Fab according to (a)(i), then it will generally be the case that the second hemibody will comprise a tandem Fab according to d(i); if the first hemibody comprises a tandem Fab according to (a)(ii), then it will generally be the case that the second hemibody will comprise a tandem Fab according to d(ii).

The tandem Fab may be generally as described above. For instance, the tandem Fab may be composed of any of the sets of chains set out above. Generally, the heavy chain fragment of the second Fab (which may be a cross-Fab) can be linked to the Fc domain.

In a further embodiment, each of the first and second hemibody may comprise a) an Fc domain comprising a first and a second subunit b) at least one antigen binding moiety capable of binding the target antigen (e.g., an antibody fragment, such as an scFv, Fv, Fab or cross-Fab fragment, comprising an antigen binding site for the target antigen) and c) a polypeptide comprising either a VL domain or a VH domain of the antigen binding site for the radiolabelled compound (but not both), wherein the C-terminus of the antigen binding moiety (e.g., antibody fragment) of (b) is fused to the N-terminus of the first subunit of the Fc domain, and the C-terminus of the polypeptide of (c) is fused to the N-terminus of the second subunit of the Fc domain. The fusion of the antibody fragment of (b) is preferably via the hinge region. The fusion of the polypeptide of (c) may be via a linker positioned between the C-terminus of polypeptide and the N-terminus of the Fc region and/or via some or all of the upper hinge region (e.g., the Asp221 and residues C-terminal thereto according to the EU numbering index). In one embodiment, the antibody fragment of (b) may be a Fab fragment. In one embodiment, in the first hemibody, the polypeptide of (c) consists of the VH domain of the antigen binding site for the radiolabelled compound; and in the second hemibody the polypeptide of (c) consists of the VL domain of the antigen binding site for the radiolabelled compound.

Thus, in one embodiment, the first hemibody may comprise or consist of:
i) a complete light chain;
ii) a complete heavy chain;
iii) an additional Fc chain; and
iv) a polypeptide comprising or consisting of the VH domain of the antigen binding site for the radiolabeled compound;
wherein the light chain of (i) and the heavy chain of (ii) together provide an antigen binding site for the target antigen; and wherein the polypeptide comprising or consisting of the VH domain of the antigen binding site for the radiolabeled compound is fused by its C-terminus, preferably via a linker, to the N-terminus of (iii).

The second hemibody may comprise or consist of
v) a complete light chain;
vi) a complete heavy chain;
vii) an additional Fc chain; and
viii) a polypeptide comprising or consisting of the VL domain of the antigen binding site for the radiolabeled compound;
wherein the light chain of (v) and the heavy chain of (vi) together provide an antigen binding site for the target antigen; and wherein the polypeptide comprising or consisting of the VL domain of the antigen binding site for the radiolabeled compound is fused by its c-terminus, preferably via a linker, to the N-terminus of (vii).

The linker may comprise any flexible linker as known to the person skilled in the art or as described herein, e.g., the linker GGGGSGGGGSGGGGSGGSGG (SEQ ID NO.: 152). The linker may further include part of all of the upper hinge region, e.g., may extend from Asp221 to the start of the Fc chain (e.g., at Cys226).

In a still further embodiment, the first and/or second hemibody each comprise a full length antibody having an antigen binding site for the target antigen, and further comprise either a VL domain or a VH domain of the antigen binding site for the radiolabelled compound.

In one particular embodiment, the first hemibody may comprise:
a) a first full length antibody consisting of two antibody heavy chains and two antibody light chains, wherein at least one arm of the full length antibody binds to the target antigen; and
b) a polypeptide comprising or consisting of
i) an further antibody heavy chain variable domain (VH); or
ii) a further antibody heavy chain variable domain (VH) and an further antibody constant domain (CH1), wherein the C-terminus of VH domain is fused to the N-terminus of the CH1 domain,
wherein said polypeptide is fused by the N-terminus of the VH domain, preferably via a peptide linker, to the C-terminus of one of the two heavy chains of said first full-length antibody.

The second hemibody may comprise
c) a second full length antibody consisting of two antibody heavy chains and two antibody light chains, wherein at least one arm of the full length antibody binds to the target antigen; and
d) a polypeptide comprising or consisting of
i) a further antibody light chain variable domain (VL); or
ii) a further antibody light chain variable domain (VL) and a further antibody light chain constant domain (CL), wherein the C-terminus of VL domain is fused to the N-terminus of the CL domain,
wherein said polypeptide is fused by the N-terminus of the VL domain, preferably via a peptide linker, to the C-terminus of one of the two heavy chains of said second full-length antibody.

The antibody heavy chain variable domain (VH) of the first hemibody and the antibody light chain variable domain (VL) of the second hemibody together form a functional antigen-binding site for the radiolabelled compound, i.e., upon association of the two antibodies.

Optionally, the polypeptide of part b(i) may additionally comprise one or more residues at the C-terminus of the VH domain, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

Optionally, the first hemibody may consist essentially of or consist of the components (a) and (b) listed above, and the second hemibody may consist essentially of or consist of the components (c) and (d) listed above. In any event, the first hemibody does not comprise an antibody light chain variable domain (VL) capable of forming a functional antigen-binding site for the radiolabelled compound in association with component (b) of the first hemibody; and the second hemibody does not comprise an antibody heavy chain variable (VH) domain capable of forming a functional antigen-binding site for the radiolabelled compound in association with component (b) of the second hemibody.

It may be preferred that both arms of the full length antibody have binding specificity for the same target antigen. Where the antibody is bivalent for the target antigen, both arms of the full length antibody may bind to the same epitope of the same target antigen.

In another embodiment, the antibody may be biparatopic for the target antigen; e.g., one arm of the full length antibody may bind to a first epitope of the target antigen and one arm may bind to a second epitope of the target antigen. In such embodiments, one arm of the antibody may comprise a Fab and one arm may comprise a cross-Fab, to assist in correct assembly of the light chains with their respective heavy chain. Thus, in one embodiment, the first heavy chain of the full length antibody may comprise a VL domain in place of the VH domain (e.g., VL-CH1-hinge-CH2-CH3) and the first light chain may comprise a VH domain exchanged for the VL domain (e.g., VH-CL), or the first heavy chain may comprise a CL domain in place of the HC1 domain (e.g., VH-CL-hinge-CH2-CH3) and the first light chain may comprise a CH1 domain in place of the CL domain (e.g., VL-CH1). In this embodiment, the second heavy chain and the second light chain have the conventional domain structure (e.g., VH-CH1-hinge-CH2-CH3 and VL-CL, respectively). In an alternative embodiment, the second heavy chain of the full length antibody may comprise a VL domain in place of the VH domain (e.g., VL-CH1-hinge-CH2-CH3) and the second light chain may comprise a VH domain exchanged for the VL domain (e.g., VH-CL), or the second heavy chain may comprise a CL domain in place of the HC1 domain (e.g., VH-CL-hinge-CH2-CH3) and the second light chain may comprise a CH1 domain in place of the CL domain (e.g., VL-CH1).

In this embodiment, the first heavy chain and the first light chain have the conventional domain structure.

In still another possible format for the hemibodies,
i) the first hemibody comprises:
   a) an antigen binding moiety capable of binding an antigen expressed on the surface of a target cell (e.g., an antibody fragment, e.g., a Fab fragment);
   b) a polypeptide comprising or consisting of an antibody heavy chain variable domain (VH) of an antigen binding site for a radiolabelled compound; and
   c) an Fc domain comprising two subunits,
wherein the polypeptide of (b) is fused by its N-terminus to the C-terminus of the antigen binding moiety of (a) (e.g., to the C-terminus of one of the chains of the Fab fragment of (a)) and by its C-terminus to the N-terminus of one of the subunits of the Fe domain of (c);
and wherein the first hemibody does not comprise a VL domain of an antigen binding site for the radiolabelled compound; and
ii) the second hemibody comprises:
   d) an antigen binding moiety capable of binding an antigen expressed on the surface of a target cell (e.g., an antibody fragment, e.g., a Fab);
   e) a polypeptide comprising or consisting of an antibody light chain variable domain (VL) of an antigen binding site for the radiolabelled compound; and
   f) an Fc domain comprising two subunits,
wherein the polypeptide of (e) is fused by its N-terminus to the C-terminus of the antigen binding moiety of (d) (e.g., to the C-terminus of one of the chains of the Fab fragment of (d)) and by its C-terminus to the N-terminus of one of the subunits of the Fc domain of (f);
and wherein the second hemibody does not comprise a VH domain of an antigen binding site for the radiolabelled compound;
wherein said VH domain of the first hemibody and said VL domain of the second hemibody are together capable of forming a functional antigen binding site for the radiolabelled compound.

The fusion may be direct or indirect, e.g., via a peptide linker.

In some embodiments, the first and/or second hemibodies further comprise another antigen binding moiety (e.g., a further antibody fragment) binding to a target antigen, e.g, another Fab fragment binding to a target antigen. Thus, in some embodiments the first and/or second hemibodies (generally both) each comprise two antigen binding moieties capable of binding to a target antigen. The two antigen binding moieties of a hemibody are preferably capable of binding to the same target antigen as each other, at the same or at different epitopes. Optionally, the first and second antibodies each comprise not more than two antigen binding moieties capable of binding to a target antigen. In other embodiments, they may comprise more than two antigen binding moieties capable of binding to a target antigen.

In one embodiment, this further antibody binding moiety (e.g., antibody fragment, e.g., Fab fragment), is fused by the C-terminus (e.g., one of one of its chains, e.g., the heavy chain) to the N-terminus of the other subunit of the Fc domain. Thus, in one embodiment, the first and/or second hemibodies may be a two-armed hemibody, wherein each arm bears a binding moiety for a target antigen.

Thus, in one embodiment, the split antibody comprises:
i) a first hemibody comprising:
a) a first antigen binding moiety (e.g., Fab fragment) wherein the antigen binding moiety (e.g., Fab fragment) binds to an antigen expressed on the surface of a target cell;
b) a polypeptide comprising or consisting of an antibody heavy chain variable domain (VH) of an antigen binding site for a radiolabelled compound; and
c) an Fc domain comprising a first and a second subunit,
wherein the polypeptide of (b) is fused by its N-terminus to the C-terminus of the antigen binding moiety of (a) (e.g., to the C-terminus of one of the chains of the Fab fragment of (a)) and by its C-terminus to the N-terminus of the first subunit of the Fc domain of (c);
and further comprising a second antigen binding moiety (e.g., a second Fab fragment) which binds to an antigen expressed on the surface of a target cell, wherein the second antigen binding moiety (e.g., Fab) is fused by its C-terminus (e.g., by the C-terminus of one of its chains) to the N-terminus of the second subunit of the Fc domain of (c);
wherein the first hemibody does not comprise a VL domain of an antigen binding site for the radiolabelled compound; and
ii) a second hemibody comprising:
d) a first antigen binding moiety (e.g., Fab fragment), wherein the antigen binding moiety (e.g., Fab fragment) binds to an antigen expressed on the surface of a target cell;
e) a polypeptide comprising or consisting of an antibody light chain variable domain (VL) of an antigen binding site for the radiolabelled compound; and
f) an Fc domain comprising a first and a second subunit,
wherein the polypeptide of (e) is fused by its N-terminus to the C-terminus of the antigen binding moiety of (d) (e.g., to the C-terminus of one of the chains of the Fab fragment of (d)) and by its C-terminus to the N-terminus of the first subunit of the Fc domain of (f);
and further comprising a second antigen binding moiety (e.g., a second Fab fragment) which binds to an antigen expressed on the surface of a target cell, wherein the second antigen binding moiety (e.g., Fab) is fused by the C-terminus (e.g., by the C-terminus of one of its chains) to the N-terminus of the second subunit of the Fc domain of (f);
wherein the second hemibody does not comprise a VH domain of an antigen binding site for the radiolabelled compound; and
wherein said VH domain of the first hemibody and said VL domain of the second hemibody are together capable of forming a functional antigen binding site for the radiolabelled compound.

In other embodiments, which may in some instances be preferred, the first and/or the second hemibody each have a single antigen binding moiety capable of specific binding to a target antigen. Thus, the first hemibody and/or second hemibody may be monospecific and monovalent for a target antigen. Preferably the first and second hemibody bind to the same target antigen as each other, at the same or at different epitopes.

In one embodiment, the first and/or second hemibody is a one-armed antibody. In such embodiments, the Fc subunit of the first hemibody which is not fused to the polypeptide of (b) is also not fused to any other antigen binding domain/moiety; and/or the Fc subunit of the second hemibody which is not fused to the polypeptide of (e) is also not fused to any other antigen binding domain/moiety. Thus, the Fc domain may comprise a subunit which is lacking Fd. In some embodiments, one of the polypeptides making up the hemibody may consist or consist essentially of the Fc subunit.

Thus, in some embodiments, the first hemibody may comprise the following polypeptides:
i) a polypeptide comprising from N-terminus to C-terminus: a Fab heavy chain (e.g., VH-CH1); an optional linker; a VH domain of an antigen binding site for a radiolabelled compound; an optional linker; and an Fc subunit (e.g, CH2-CH3);
ii) a Fab light chain polypeptide (e.g., VL-CL); and
iii) an Fc subunit polypeptide (e.g., CH2-CH3);
wherein the Fab heavy chain of (i) and the Fab light chain of (ii) form a Fab fragment capable of binding to a target antigen.

The second hemibody may comprise the following polypeptides:
iv) a polypeptide comprising from N-terminus to C-terminus: a Fab heavy chain (e.g., VH-CH1); an optional linker; a VL domain of an antigen binding site for the radiolabelled compound; an optional linker; and an Fc subunit (e.g, CH2-CH3);
v) a Fab light chain polypeptide (e.g., VL-CL), and
vi) an Fc subunit polypeptide (e.g., CH2-CH3);
wherein the Fab heavy chain of (iv) and the Fab light chain of (v) form a Fab fragment capable of binding to a target antigen.

In some embodiments of these one-armed hemibodies, the Fab heavy chain of (i) and of (iv) may have the same sequence as each other; and the Fab light chain polypeptide of ii) and (v) may have the same sequence as each other.

In some embodiments of any of the above formats, where there are Fabs having different specificities, correct assembly of the light chains with their respective heavy chain can be assisted by using charge modification, as discussed further below.

Correct assembly of heterodimeric heavy chains can be assisted by knob-into-hole technology, as discussed further below.

### H. Exemplary Split Multispecific Split Antibodies

Aspects and embodiments concerning target binding (e.g., CEA-binding) and aspects and embodiments concerning DOTA binding can in some embodiments be combined. In one embodiment, the multispecific antibody may comprise a binding site for CEA, having any of the sequences set out above, and a binding site for a DOTA chelate, having any of the sequences set out above. In another embodiment, the first and second hemibody each comprise a binding site for CEA, e.g., comprising any of the sequences as described above, and associate to form a binding site for a DOTA chelate having any of the sequences as described above. It is also expressly contemplated that aspects and embodiments concerning CEA binding and/or DOTA binding can be combined with preferred formats for the antibody as described above—i.e., in any of the preferred formats, the part that binds the target antigen may be a CEA-binder comprising CDRs or variable regions sequences as described above, and/or the part that binds the radionuclide-labelled compound may be a DOTA binder having CDRs and/or variable region sequences as described above.

In one particular embodiment of a split antibody, the first hemibody may comprise:
a) a first full length antibody specifically binding to CEA and consisting of two antibody heavy chains and two antibody light chains; and
b) a polypeptide comprising or consisting of an antibody heavy chain variable domain (VH) wherein the heavy chain variable domain comprises heavy chain CDRs of SEQ ID NOs 35-37 (or wherein CDR-H1 has the sequence GFSLTDYGVH), and/or wherein the heavy chain variable domain has at least 90, 91, 92, 93, 94, 95, 96, 97, 98, 99 or 100% identity to SEQ ID NO 41;
wherein said polypeptide is fused with the N-terminus of the VH domain, preferably via a peptide linker, to the C-terminus of one of the two heavy chains of said first full-length antibody.

The first hemibody does not comprise a light chain domain which associates with the polypeptide of (b) to form a functional binding domain for a radiolabelled compound.

It may be preferred that the polypeptide of (b) further comprises one or more residues at the C-terminus of the VH domain, e.g., 1-10 residues. Optionally, these may be one or more alanine residues, optionally a single alanine residue. In another embodiment, the additional residues may be an N-terminal portion of the CH1 domain as described above, e.g., 1-10 residues from the N-terminus of the CH1 domain, e.g., from the human IgG1 CH1 domain. For instance, the additional residues may be AST.

In some embodiments, the two antibody heavy chains in part (a) have identical variable domains, optionally identical variable, CH1 and/or CH2 domains. They may optionally differ only in their CH3 domains, e.g., by the creation of knob into hole mutations and other mutations intended to promote the correct association of heterodimers.

The second hemibody may comprise:
c) a second full length antibody specifically binding CEA and consisting of two antibody heavy chains and two antibody light chains; and
d) a polypeptide comprising or consisting of an antibody light chain variable domain (VL) wherein the light chain variable domain comprises CDRs of SEQ ID NO: 38-40 and/or wherein the light chain variable domain has at least 90, 91, 92, 93, 94, 95, 96, 97, 98, 99 or 100% identity to SEQ ID NO 42;
wherein said polypeptide is fused with the N-terminus of the VL domain, preferably via a peptide linker, to the C-terminus of one of the two heavy chains of said second full-length antibody and wherein the second hemibody does not comprise a heavy chain domain which associates with the polypeptide of (d) to form a functional binding domain for a radiolabelled compound.

In some embodiments, the two antibody heavy chains in part (c) have identical variable domains to each other, optionally identical variable, CH1 and/or CH2 domains.

They may optionally differ only in their CH3 domains, e.g., by the creation of knob into hole mutations and other mutations intended to promote the correct association of heterodimers.

The CEA-binding sites/sequences may be any of the CEA-binding sites/sequences described above.

In one particular embodiment, the first hemibody may have CEA binding sequences (i.e., CDRs or VH/VL domains) from the antibody CH1A1A.

For example, the two light chains in (a) may comprise the CDRs of SEQ ID Nos 22-24 and/or may comprise light chains variable domains having at least 90, 91, 92, 93, 94, 95, 96, 97, 98, 99 or 100% identity to SEQ ID NO 26. In some embodiments they may have at least 90, 91, 92, 93, 94, 95, 96, 97, 98, 99 or 100% identity to SEQ ID NO 103. In some embodiments, it may be preferred that the two light chains in (a) are identical to each other.

The two antibody heavy chains in part (a) may comprise the CDRs of SEQ ID NOs: 19-21 and/or the two antibody heavy chains in part (a) comprise a variable domain having at least 90, 91, 92, 93, 94, 95, 96, 97, 98, 99 or 100% identity to SEQ ID NO 25. In one embodiment, one heavy chain in part (a) has the sequence of SEQ ID NO: 100 and the other has the sequence of SEQ ID NO: 102.

In one specific embodiment, the first hemibody may comprise a first heavy chain of SEQ ID NO: 100, and second heavy chain of SEQ ID NO: 101 (wherein the C-terminal AST is optional and may be absent or substituted with anther C-terminal extension as described herein) and a light chain of SEQ ID NO: 103.

The second hemibody may also have CEA binding sequences (i.e., CDRs or VH/VL domains) from the antibody CH1A1A.

For example, the two light chain in (c) may comprise the CDRs of SEQ ID Nos 22-24 and/or may comprise light chains variable domains having at least 90, 91, 92, 93, 94, 95, 96, 97, 98, 99 or 100% identity to SEQ ID NO 26. In some embodiments they may have at least 90, 91, 92, 93, 94, 95, 96, 97, 98, 99 or 100% identity to SEQ ID NO: 103. In some embodiments, it may be preferred that the two light chains in (c) are identical to each other. In some embodiments, it may be preferred that the two light chains in (c) have the same sequence as the light chains in (a) of the first hemibody, e.g., that all said light chains in parts (a) and (c) have the same sequence.

In some embodiments, the two antibody heavy chains in part (c) comprise the CDRs of SEQ ID NOs: 19-21 and/or the two antibody heavy chains in part (c) comprise a variable domain having at least 90, 91, 92, 93, 94, 95, 96, 97, 98, 99 or 100% identity to SEQ ID NO 25. In one embodiment, one heavy chain of part (c) has the sequence of SEQ ID NO: 97 and the other has the sequence of SEQ ID NO: 99.

In one specific embodiment, the second hemibody may comprise a first heavy chain of SEQ ID NO: 97, and second heavy chain of SEQ ID NO: 98 and a light chain of SEQ ID NO: 103.

Similarly, aspects and embodiments concerning target binding (e.g., CEA-binding) and aspects and embodiments concerning Pb-DOTAM binding can in some embodiments be combined. In one embodiment, the multispecific antibody may comprise a binding site for CEA, having any of the sequences set out above, and a binding site for a pb-DOTAM chelate, having any of the sequences set out above. In another embodiment, the first and second hemibody may each comprise a binding site for CEA, e.g., comprising any of the sequences as described above, and associate to form a binding site for a Pb-DOTAM chelate having any of the sequences as described above. It is also expressly contemplated that aspects and embodiments concerning CEA binding and/or Pb-DOTAM binding can be combined with preferred formats for the antibody as described above—i.e., in any of the preferred formats, the part that binds the target antigen may be a CEA-binder comprising CDRs or variable regions sequences as described above, and/or the part that binds the radionuclide-labelled compound may be a Pb-DOTAM binder having CDRs and/or variable region sequences as described above.

In one particular embodiment of a split antibody, the first hemibody may comprise:
a) a first full length antibody specifically binding to CEA and consisting of two antibody heavy chains and two antibody light chains; and
b) a polypeptide comprising or consisting of an antibody heavy chain variable domain (VH) wherein the heavy chain variable domain comprises heavy chain CDRs of SEQ ID NOs 1-3, and/or wherein the heavy chain variable domain has at least 90, 91, 92, 93, 94, 95, 96, 97, 98, 99 or 100% identity to SEQ ID NO 7;
wherein said polypeptide is fused with the N-terminus of the VH domain, preferably via a peptide linker, to the C-terminus of one of the two heavy chains of said first full-length antibody.

The first hemibody does not comprise a light chain domain which associates with the polypeptide of (b) to form a functional binding domain for a radiolabelled compound.

It may be preferred that the polypeptide of (b) further comprises one or more residues at the C-terminus of the VH domain, optionally, one or more alanine residues, optionally a single alanine residue. For example, the polypeptide of (b) may comprise or consists of SEQ ID NO: 7 with a C-terminal alanine extension, e.g., the sequence

VTLKESGPVLVKPTETLTLTCTVSGFSLSTYSMSWIRQPPGKALEWLGF
IGSRGDTYYASWAKGRLTISKDTSKSQVVLTMTNMDPVDTATYYCARER
DPYGGGAYPPHLWGRGTLVTVSSA

In another embodiment, the additional residues may be an N-terminal portion of the CH1 domain as described above, e.g., 1-10 residues from the N-terminus of the CH1 domain, e.g., from the human IgG1 CH1 domain. For instance, the additional residues may be AST.

In some embodiments, the two antibody heavy chains in part (a) have identical variable domains, optionally identical variable, CH1 and/or CH2 domains. They may optionally differ only in their CH3 domains, e.g., by the creation of knob into hole mutations and other mutations intended to promote the correct association of heterodimers.

The second hemibody may comprise:
c) a second full length antibody specifically binding CEA and consisting of two antibody heavy chains and two antibody light chains; and
d) a polypeptide comprising or consisting of an antibody light chain variable domain (VL) wherein the light chain variable domain comprises CDRs of SEQ ID NO: 4-6 and/or wherein the light chain variable domain has at least 90, 91, 92, 93, 94, 95, 96, 97, 98, 99 or 100% identity to SEQ ID NO 8;
wherein said polypeptide is fused with the N-terminus of the VL domain, preferably via a peptide linker, to the C-terminus of one of the two heavy chains of said second full-length antibody and wherein the second hemibody does not comprise a heavy chain domain which associates with the polypeptide of (d) to form a functional binding domain for a radiolabelled compound.

In some embodiments, the two antibody heavy chains in part (c) have identical variable domains to each other, optionally identical variable, CH1 and/or CH2 domains. They may optionally differ only in their CH3 domains, e.g., by the creation of knob into hole mutations and other mutations intended to promote the correct association of heterodimers.

In a particular embodiment, the first hemibody may have CEA binding sequences (i.e., CDRs or VH/VL domains) from the antibody CH1A1A.

For example, the two light chains in (a) may comprise the CDRs of SEQ ID Nos 22-24 and/or may comprise light chains variable domains having at least 90, 91, 92, 93, 94, 95, 96, 97, 98, 99 or 100% identity to SEQ ID NO 26. In some embodiments they may have at least 90, 91, 92, 93, 94, 95, 96, 97, 98, 99 or 100% identity to SEQ ID NO 34. In some embodiments, it may be preferred that the two light chains in (a) are identical to each other.

The two antibody heavy chains in part (a) may comprise the CDRs of SEQ ID NOs: 19-21 and/or the two antibody heavy chains in part (a) comprise a variable domain having at least 90, 91, 92, 93, 94, 95, 96, 97, 98, 99 or 100% identity to SEQ ID NO 25. In one embodiment, one heavy chain in part (a) has the sequence of SEQ ID NO: 27 and the other has the sequence of SEQ ID NO: 28.

In one specific embodiment, the first hemibody may comprise a first heavy chain of SEQ ID NO: 28, and second heavy chain of SEQ ID NO: 32 (or a variant thereof comprising an additional C-terminal alanine or other C-terminal extension as described herein, such as an extension with AST) and a light chain of SEQ ID NO: 34. A variant of SEQ ID NO: 32 with a C-terminal alanine extension is shown below:

QVQLVQSGAEVKKPGASVKVSCKASGYTFTEFGMNWVRQAPGQGLE
WMGWINTKTGEATYVEEFKGRVTFTTDTSTSTAYMELRSLRSDDTAVYY
CARWDFAYYVEAMDYWGQGTTVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPEAAGGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKALGAPIEKTI
SKAKGQPREPQVYTLPPCRDELTKNQVSLWCLVKGFYPSDIAVEWESNG
QPENNYKTTPPVLDSDGSFFLYSKLTVDKSRWQQGNVFSCSVMHEALHN
HYTQKSLSLSPGGGGGSGGGGSGGGGSGGGGSVTLKESGPVLVKPTETL
TLTCTVSGFSLSTYSMSWIRQPPGKALEWLGFIGSRGDTYYASWAKGRL
TISKDTSKSQVVLTMTNMDPVDTATYYCARERDPYGGGAYPPHLWGRGT
LVTVSSA

In another particular embodiment, the first hemibody may have CEA binding sequences (i.e., CDRs or VH/VL domains) from the antibody A5B7 (including a humanized version thereof).

For example, the two light chains in (a) may comprise the CDRs of SEQ ID Nos 46-48 and/or may comprise light chains variable domains having at least 90, 91, 92, 93, 94, 95, 96, 97, 98, 99 or 100% identity to SEQ ID NO 50. In some embodiments they may have at least 90, 91, 92, 93, 94, 95, 96, 97, 98, 99 or 100% identity to SEQ ID NO: 54. In some embodiments, it may be preferred that the two light chains in (a) are identical to each other.

In some embodiments, the two antibody heavy chains in part (a) may comprise the CDRs of SEQ ID NOs: 43-45 and/or the two antibody heavy chains in part (a) comprise a variable domain having at least 90, 91, 92, 93, 94, 95, 96, 97, 98, 99 or 100% identity to SEQ ID NO 49. In one embodiment, one heavy chain in part (a) has the sequence of SEQ ID NO: 51 and the other has the sequence of SEQ ID NO: 53.

In one specific embodiment, the first hemibody may comprise a first heavy chain of SEQ ID NO: 51, and second heavy chain of SEQ ID NO: 52 (or a variant thereof with a C-terminal alanine extension or other C-terminal extension as described herein, such as an extension with AST) and a light chain of SEQ ID NO: 54.

In another particular embodiment, the first hemibody may have CEA binding sequences (i.e., CDRs or VH/VL domains) from the antibody T84.66 (including a humanized version thereof).

For example, the two light chains in (a) may comprise the CDRs of SEQ ID Nos 14-16 and/or may comprise light chains variable domains having at least 90, 91, 92, 93, 94, 95, 96, 97, 98, 99 or 100% identity to SEQ ID NO 18. In some embodiments they may have at least 90, 91, 92, 93, 94, 95, 96, 97, 98, 99 or 100% identity to SEQ ID NO: 89. In some embodiments, it may be preferred that the two light chains in (a) are identical to each other.

In some embodiments, the two antibody heavy chains in part (a) may comprise the CDRs of SEQ ID NOs: 11-13 and/or the two antibody heavy chains in part (a) comprise a variable domain having at least 90, 91, 92, 93, 94, 95, 96, 97, 98, 99 or 100% identity to SEQ ID NO 17. In one embodiment, one heavy chain in part (a) has the sequence of SEQ ID NO: 86 and the other has the sequence of SEQ ID NO: 88.

In one specific embodiment, the first hemibody may comprise a first heavy chain of SEQ ID NO: 86, and second heavy chain of SEQ ID NO: 87 (or a variant thereof in which the C-terminal “AST” is absent or substituted by a different C-terminal extension as disclosed herein) and a light chain of SEQ ID NO: 89.

In another particular embodiment, the first hemibody may have CEA binding sequences (i.e., CDRs or VH/VL domains) from the antibody 28A9 (including a humanized version thereof).

For example, the two light chains in (a) may comprise the CDRs of SEQ ID Nos 62-64 and/or may comprise light chains variable domains having at least 90, 91, 92, 93, 94, 95, 96, 97, 98, 99 or 100% identity to SEQ ID NO: 66. In some embodiments they may have at least 90, 91, 92, 93, 94, 95, 96, 97, 98, 99 or 100% identity to SEQ ID NO: 96. In some embodiments, it may be preferred that the two light chains in (a) are identical to each other.

In some embodiments, the two antibody heavy chains in part (a) may comprise the CDRs of SEQ ID NOs: 59-61 and/or the two antibody heavy chains in part (a) comprise a variable domain having at least 90, 91, 92, 93, 94, 95, 96, 97, 98, 99 or 100% identity to SEQ ID NO 65. In one embodiment, one heavy chain in part (a) has the sequence of SEQ ID NO: 93 and the other has the sequence of SEQ ID NO: 95.

In one specific embodiment, the first hemibody may comprise a first heavy chain of SEQ ID NO: 93, and second heavy chain of SEQ ID NO: 94 (or a variant thereof without the C-terminal “AST” or with a different C-terminal extension as described herein) and a light chain of SEQ ID NO: 96.

In some embodiments, the second hemibody may have CEA binding sequences (i.e., CDRs or VH/VL domains) from the antibody CH1A1A.

For example, the two light chain in (c) may comprise the CDRs of SEQ ID Nos 22-24 and/or may comprise light chains variable domains having at least 90, 91, 92, 93, 94, 95, 96, 97, 98, 99 or 100% identity to SEQ ID NO 26. In some embodiments they may have at least 90, 91, 92, 93, 94, 95, 96, 97, 98, 99 or 100% identity to SEQ ID NO 34. In some embodiments, it may be preferred that the two light chains in (c) are identical to each other. In some embodiments, it may be preferred that the two light chains in (c) have the same sequence as the light chains in (a) of the first hemibody, e.g., that all said light chains in parts (a) and (c) have the same sequence.

In some embodiments, the two antibody heavy chains in part (c) comprise the CDRs of SEQ ID NOs: 19-21 and/or the two antibody heavy chains in part (c) comprise a variable domain having at least 90, 91, 92, 93, 94, 95, 96, 97, 98, 99 or 100% identity to SEQ ID NO 25. In one embodiment, one heavy chain of part (c) has the sequence of SEQ ID NO: 29 and the other has the sequence of SEQ ID NO: 30.

In one specific embodiment, the second hemibody may comprise a first heavy chain of SEQ ID NO: 30, and second heavy chain of SEQ ID NO: 33 and a light chain of SEQ ID NO: 34.

In another particular embodiment, the second hemibody may have CEA binding sequences (i.e., CDRs or VH/VL domains) from A5B7 (including a humanized version thereof).

For example, the two light chain in (c) may comprise the CDRs of SEQ ID Nos 46-48 and/or may comprise light chains variable domains having at least 90, 91, 92, 93, 94, 95, 96, 97, 98, 99 or 100% identity to SEQ ID NO 50. In some embodiments they may have at least 90, 91, 92, 93, 94, 95, 96, 97, 98, 99 or 100% identity to SEQ ID NO 58. In some embodiments, it may be preferred that the two light chains in (c) are identical to each other. In some embodiments, it may be preferred that the two light chains in (c) have the same sequence as the light chains in (a) of the first hemibody, e.g., that all said light chains in parts (a) and (c) have the same sequence.

In some embodiments, the two antibody heavy chains in part (c) comprise the CDRs of SEQ ID NOs: 43-45 and/or the two antibody heavy chains in part (c) comprise a variable domain having at least 90, 91, 92, 93, 94, 95, 96, 97, 98, 99 or 100% identity to SEQ ID NO 49. In one embodiment, one heavy chain of part (c) has the sequence of SEQ ID NO: 55 and the other has the sequence of SEQ ID NO: 57.

In one specific embodiment, the second hemibody may comprise a first heavy chain of SEQ ID NO: 55, and second heavy chain of SEQ ID NO: 56 and a light chain of SEQ ID NO: 58.

In another particular embodiment, the second hemibody may have CEA binding sequences (i.e., CDRs or VH/VL domains) from the antibody T84.66 (including a humanized version thereof).

For example, the two light chains in (c) may comprise the CDRs of SEQ ID Nos 14-16 and/or may comprise light chains variable domains having at least 90, 91, 92, 93, 94, 95, 96, 97, 98, 99 or 100% identity to SEQ ID NO 18. In some embodiments they may have at least 90, 91, 92, 93, 94, 95, 96, 97, 98, 99 or 100% identity to SEQ ID NO: 89. In some embodiments, it may be preferred that the two light chains in (c) are identical to each other.

In some embodiments, the two antibody heavy chains in part (c) may comprise the CDRs of SEQ ID NOs: 11-13 and/or the two antibody heavy chains in part (c) comprise a variable domain having at least 90, 91, 92, 93, 94, 95, 96, 97, 98, 99 or 100% identity to SEQ ID NO 17. In one embodiment, one heavy chain in part (c) has the sequence of SEQ ID NO: 83 and the other has the sequence of SEQ ID NO: 85.

In one specific embodiment, the second hemibody may comprise a first heavy chain of SEQ ID NO: 83, and second heavy chain of SEQ ID NO: 84 and a light chain of SEQ ID NO: 89.

In another particular embodiment, the second hemibody may have CEA binding sequences (i.e., CDRs or VH/VL domains) from the antibody 28A9 (including a humanized version thereof).

For example, the two light chains in (c) may comprise the CDRs of SEQ ID Nos 62-64 and/or may comprise light chains variable domains having at least 90, 91, 92, 93, 94, 95, 96, 97, 98, 99 or 100% identity to SEQ ID NO 66. In some embodiments they may have at least 90, 91, 92, 93, 94, 95, 96, 97, 98, 99 or 100% identity to SEQ ID NO: 96. In some embodiments, it may be preferred that the two light chains in (c) are identical to each other.

In some embodiments, the two antibody heavy chains in part (c) may comprise the CDRs of SEQ ID NOs: 59-61 and/or the two antibody heavy chains in part (a) comprise a variable domain having at least 90, 91, 92, 93, 94, 95, 96, 97, 98, 99 or 100% identity to SEQ ID NO 65. In one embodiment, one heavy chain in part (c) has the sequence of SEQ ID NO: 90 and the other has the sequence of SEQ ID NO: 92.

In one specific embodiment, the second hemibody may comprise a first heavy chain of SEQ ID NO: 90, and second heavy chain of SEQ ID NO: 91 and a light chain of SEQ ID NO: 96.

In some embodiments, the first and the second hemibody bind the same epitope of CEA. Thus, for example, the first and the second hemibody may both have CEA binding sequences from the antibody CH1A1A; or, the first and the second hemibody may both have CEA binding sequences from A5B7 (including a humanized version thereof); or, the first and the second hemibody may both have CEA binding sequences from T84.66 (including a humanized version thereof); or, the first and the second hemibody may both have CEA binding sequences from 28A9 (including a humanized version thereof); or, the first and the second hemibody may both have CEA binding sequences from MFE23 (including a humanized version thereof).

Thus, for example:
i) the first hemibody may comprise a first heavy chain of SEQ ID NO: 28, a second heavy chain of SEQ ID NO: 32 (optionally with a C-terminal extension as described herein, e.g., AST) and a light chain of SEQ ID NO: 34; and the second hemibody may comprise a first heavy chain of SEQ ID NO: 30, a second heavy chain of SEQ ID NO: 33 and a light chain of SEQ ID NO: 34;
ii) the first hemibody may comprise a first heavy chain of SEQ ID NO: 51, a second heavy chain of SEQ ID NO: 52 (optionally with a C-terminal extension as described herein, e.g., AST) and a light chain of SEQ ID NO: 54; and the second hemibody may comprise a first heavy chain of SEQ ID NO: 55, a second heavy chain of SEQ ID NO: 56 and a light chain of SEQ ID NO: 58;
iii) the first hemibody may comprise a first heavy chain of SEQ ID NO: 86, a second heavy chain of SEQ ID NO: 87 (wherein the C-terminal AST residues are optional and may be absent or substituted by an alternative C-terminal extension) and a light chain of SEQ ID NO: 89; and the second hemibody antibody may comprise a first heavy chain of SEQ ID NO: 83, a second heavy chain of SEQ ID NO: 84 and a light chain of SEQ ID NO: 89; or
iv) the first hemibody may comprise a first heavy chain of SEQ ID NO: 93, a second heavy chain of SEQ ID NO: 94 (wherein the C-terminal AST residues are optional and may be absent or substituted by an alternative C-terminal extension) and a light chain of SEQ ID NO: 96; and the second hemibody may comprise a first heavy chain of SEQ ID NO: 90, a second heavy chain of SEQ ID NO: 91 and a light chain of SEQ ID NO: 96.

In other embodiments, the first and the second hemibodies bind to different epitopes of CEA, as discussed above. Thus, for instance, the first hemibody may have CEA binding sequences from the antibody CH1A1A and the second hemibody may have CEA binding sequences from A5B7; or, the first hemibody may have CEA binding sequences from the antibody A5B7 and the second hemibody may have CEA binding sequences from CH1A1A. An example of the use of bi-paratopic (CH1A1A and A5B7) pairs is described in Example 6c.

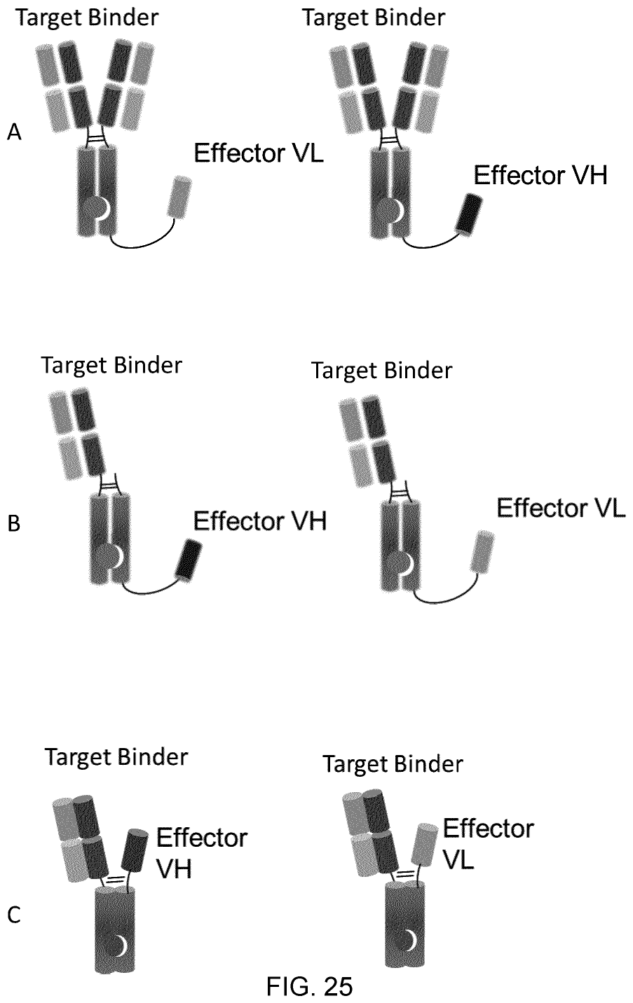

In still further specific embodiments, the target may be CEA, e.g., having CEA binding sequences from the antibody CH1A1A, and the format may be as shown in FIG. 25C. Optionally, the first and second hemibody associate to form a functional antigen binding site for a Pb-DOTAM chelate (Pb-DOTAM).

### I Polypeptide Linkers

In the multispecific or split multispecific antibodies used in the combination therapy, components or domains (e.g., Fc domain, antibody binding moieties, VH, VL) may be fused to other components or domains indirectly via a peptide linker.

The linker (e.g., the linker between the Fab fragment and the VH/VL for the radiolabelled compound and/or between the VH/VL for the radiolabelled compound and the Fc domain) may be a peptide of at least 5 or at least 10 amino acids, preferably 5 to 100, e.g., 5 to 70, 5 to 60, or 5 to 50; or 10 to 100, 10 to 70, 10 to 60 or 10 to 50 amino acids. In some embodiments, it may be preferred that the linker is 15-30 amino acids in length, e.g., 15-25, e.g., 16, 17, 18, 19, 20, 21, 22, 23 or 24 amino acids in length. The linker may be a rigid linker or a flexible linker. In some embodiments, it is a flexible linker comprising or consisting of Thr, Ser, Gly and/or Ala residues. For example, it may comprise or consist of Gly and Ser residues. In some embodiments it may have a repeating motif such as (Gly-Gly-Gly-Gly-Ser)n, where n is for instance 1, 2, 3, 4, 5, 6, 7, 8, 9 or 10. Suitable, non-immunogenic peptide linkers include, for example, (G4S)n, (SG4)n, (G4S)n or G4(SG4)n peptide linkers, where “n” is generally a number between 1 and 10, typically between 2 and 4. In another embodiment said peptide linker is (GxS)n or (GxS)nGm with G=glycine, S=serine, and (x=3, n=3, 4, 5 or 6, and m=0, 1, 2 or 3) or (x=4, n=2, 3, 4 or 5 and m=0, 1, 2 or 3), e.g., x=4 and n=2 or 3, e.g., with x=4, n=2. In some embodiments, the linker may be or may comprise the sequence GGGGSGGGGSGGGGSGGGGS (SEQ ID NO.: 31). In another embodiment the linker may be or comprise GGGGSGGGGSGGGGSGGSGG or GGGGSGGGGSGGGGSGGSGGS or GGGGSGGGGSGGGGSGGSGGG. Another exemplary peptide linker is EPKSC(D)-(G4S)2. Additionally, where an antigen binding moiety is fused to the N-terminus of an Fc domain subunit, it may be fused via an immunoglobulin hinge region or a portion thereof, with or without an additional peptide linker.

The present inventors have determined that in a peptide linker consisting of y amino acids, a Ser in the y position (i.e., a Ser as the last/C-terminal amino acid of the linker) may induce glycosylation of the y+2 amino acid (i.e., of the amino acid positioned 2 residues in the C-terminal direction from the last amino acid in the linker), depending on the nature of this y+2 amino acid. Therefore it may be preferred that the last serine residue of the linker is placed in the y−2 or y−3 position (i.e., that the last serine residue of the linker is at a position 2 or 3 amino acids in the N-terminal direction from the last amino acid in the linker). In some embodiments, the linker may consist of y consecutive amino acid residues selected from the group consisting of Gly and Ser, e.g., wherein y=5 or more; e.g., y=5 to 100, 5 to 70, 5 to 60, 5 to 50; or 10 to 100, 10 to 70, 10 to 60 or 10 to 50; e.g., 15 to 31 or 15 to 30, e.g., 15, 16, 17, 18, 19, 20, 21, 22, 23, 24 or 25, and wherein the last serine is in the y−2 or y−3 position. (Thus, there may be a serine in the y−2 position and a glycine in the y−1 and y position; or there may be a serine in the y−3 position and a glycine in the y−2, y−1 and y positions). In some embodiments it may be preferred that y=20 or 21. In some embodiments, it may be preferred that the linker is (GxS)n(GGSGG) or (GxS)n(GGSGGG) with G=glycine, S=serine, x=4 and n=1 to 20 or 2 to 20 or 1 to 10 or 2 to 10, e.g., n=2, 3, 4, 5, 6, 7, 8 or 9, e.g., n=2 to 5 or 2 to 4. For instance, the linker may be GGGGSGGGGSGGGGSGGSGG or GGGGSGGGGSGGGGSGGSGGG.

### J. CD40 Agonists

The combination therapies of the present invention comprise a CD40 agonist. The human CD40 antigen is a 50 kDa cell surface glycoprotein which belongs to the Tumor Necrosis Factor Receptor (TNF-R) family (Stamenkovic et al., EMBO J. 8:1403-10 (1989)). It is also known as “Tumor necrosis factor receptor superfamily member 5”. Alternative designations include B-cell surface antigen 40, Bp50, CD40L receptor, CDw40, CDW40, MGC9013, p50 or TNFRSF5. It is for example registered under UniProt Entry No. P25942. In one embodiment human CD40 antigen has the sequence shown below (see Table 1).

TABLE 1
Protein sequence of human CD40 antigen

| | |
|---|---|
| Protein | MVRLPLQCVLWGCLLTAVHPEPPTACREKQYLINSQC |
| sequence | CSLCQPGQKLVSDCTEFTETECLPCGESEFLDTWNRE |
| of human | THCHQHKYCDPNLGLRVQQKGTSETDTICTCEEGWH |
| CD40 | CTSEACESCVLHRSCSPGFGVKQIATGVSDTICEPCPV |
| antigen | GFFSNVSSAFEKCHPWTSCETKDLVVQQAGTNKTDV |
| | VCGPQDRLRALVVIPIIFGILFAILLVLVFIKKVAKKP |
| | TNKAPHPKQEPQEINFPDDLPGSNTAAPVQETLHGCQP |
| | VTQEDGKESR ISVQERQ |

CD40 is expressed by antigen-presenting cells (APC) and engagement of its natural ligand on T cells activates APC including dendritic cells and B cells.

The “CD40 agonist” as used herein includes any moiety that agonizes the CD40/CD40L interaction. Typically these moieties will be agonistic CD40 antibodies or agonistic CD40L polypeptides. An “agonist” combines with a receptor on a cell and initiates a reaction or activity that is similar to or the same as that initiated by a natural ligand of said receptor. In one aspect, a “CD40 agonist” induces any or all of, but not limited to, the following responses: B cell proliferation and/or differentiation; upregulation of intercellular adhesion via such molecules as ICAM-1, E-selectin, VC AM, and the like; secretion of pro-inflammatory cytokines such as IL-1, IL-6, IL-8, IL-12, TNF, and the like; signal transduction through the CD40 receptor by such pathways as TRAF {e.g., TRAF2 and/or TRAF3), MAP kinases such as NIK (NF-kB inducing kinase), I-kappa B kinases (IKK/.beta.), transcription factor NF-kB, Ras and the MEK/ERK pathway, the PI3K AKT pathway, the P38 MAPK pathway, and the like; transduction of an anti-apoptotic signal by such molecules as XIAP, mcl-1, bcl-x, and the like; B and/or T cell memory generation; B cell antibody production; B cell isotype switching, up-regulation of cell-surface expression of MHC Class II and CD80/86, and the like.

Exemplary agonists include the CD40 ligand CD40L, including functional variants thereof, or nucleic acids expressing CD40L or functional variants thereof, such as recombinant human CD40L, or adenovirus vector-expressed CD40L.

In other embodiments, the agonist may be an anti-CD40 antibody, e.g., a monoclonal antibody. For example, the antibody may be a human or humanized antibody or a chimeric antibody. The antibody may be an IgG, e.g., IgG1, IgG2, IgG3 or IgG4.

In some embodiments, the antibody may bind CD40 with a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In another embodiment, the antibody may bind to human CD40 with a K_{D} of 4×10⁻¹⁰ M or less.

Exemplary anti-CD40 agonist antibodies are known in the art. Any of these or functional variants thereof may be employed in embodiments of the present invention.

CP-870,893 (Pfizer) (also known as Selicrelumab) is a fully human CD40 agonist IgG₂ mAb that exhibits immune-mediated and non-immune mediated effects on tumor cell death (Vonderheide R H, Flaherty K T, Khalil M, Stumacher M S, Bajor D L, Hutnick N A, et al. Clinical activity and immune modulation in cancer patients treated with CP-870,893, a novel CD40 agonist monoclonal antibody. J Clin Oncol. 2007; 25:876-83).

Dacetuzumab (Seattle Genetics) is a humanized mAb IgG1 against CD40 (Khubchandani S, Czuczman M S, Hernandez-Ilizaliturri F J. Dacetuzumab, a humanized mAb against CD40 for the treatment of hematological malignancies. Curr Opin Investig Drugs. 2009; 10:579-87.).

Chi Lob 7/4 (University of Southampton) is a chimeric IgG1 (Johnson P W, Steven N M, Chowdhury F, Dobbyn J, Hall E, Ashton-Key M, et al. A Cancer Research UK phase I study evaluating safety, tolerability, and biological effects of chimeric anti-CD40 monoclonal antibody (MAb), Chi Lob 7/4. J Clin Oncol. 2010; 28:2507).

APX005M is a humanized rabbit IgG1 (Bjorck P, Filbert E, Zhang Y, Yang X, Trifan O. The CD40 agonistic monoclonal antibody APX005M has potent immune stimulatory capabilities. J Immunother Cancer. 2015; 3:P198. doi: 10.1186/2051-1426-3-S2-P198.) ADC-1013 is a fully human IgG1 (Mangsbo S M, Broos S, Fletcher E, Veitonmaki N, Furebring C, Dahlen E, Norlen P, Lindstedt M, Totterman T H, Ellmark P. The human agonistic CD40 antibody ADC-1013 eradicates bladder tumors and generates T-cell-dependent tumor immunity. Clin Cancer Res. 2015; 21:1115-1126. doi: 10.1158/1078-0432.CCR-14-0913.)

CDX-1140 is a fully human IgG2 (Vitale L A, Thomas L J, He L Z, O'Neill T, Widger J, Crocker A, Sundarapandiyan K, Storey J R, Forsberg E M, Weidlick J, et al. Development of CDX-1140, an agonist CD40 antibody for cancer immunotherapy. Cancer Immunol Immunother. 2019; 68:233-245. doi: 10.1007/s00262-018-2267-0.)

### K. Immune Checkpoint Inhibitors

The combination therapies of the present invention comprise an immune checkpoint inhibitor.

Exemplary immune checkpoint inhibitors include inhibitors of CTLA-4, PDL1, PDL2, PD1, B7-H3, B7-H4, BTLA, HVEM, TIM3, GAL9, LAG3, VISTA, KIR, 2B4, CD160, CGEN-1 5049, CHK1, CHK2, A2aR, B-7 or a combination thereof. In some embodiments, the checkpoint inhibitor may be an inhibitor of PD1, PDL1 or CTLA4.

The human PD-L1 (or PDL1) antigen is also designated as “Programmed cell death 1 ligand 1” or CD274 molecule. Alternative designations comprise B7-H, B7H1, B7-H1, B7 homolog 1, MGC142294, MGC142296, PDCD1L1, PDCD1LG1, PDCD1 ligand 1, PDL1, PD-L1, Programmed death ligand 1. In one embodiment the human PD-L1 antigen has the sequence shown below (Table 2), as for example registered as UniProt Entry No. Q9NZQ7.

TABLE 2

| | |
|---|---|
| Protein | MRIFAVFIFMTYWHLLNAFTVTVPKDLYVVEYGSNM |
| sequence | TIECKFPVEKQLDLAALIVYWEMEDKNIIQFVHGEEDL |
| of human | KVQHSSYRQRARLLKDQLSLGNAALQITDVKLQDAG |
| PD-L1 | VYRCMISYGGADYKRITVKVNAPYNKINQRILVVDPV |
| antigen | TSEHELTCQAEGYPKAEVIWTSSDHQVLSGKTTTTNS |
| | KREEKLFNVTSTLRINTTTNEIFYCTFRRLDPEENHTAE |
| | LVIPELPLAHPPNERTHLVILGAILLCLGVALTFIFRLR |
| | KGRMMDVKKCGIQDTNSKKQSDTHLEET |

In some embodiments, the inhibitor may be a small molecule or peptide, e.g., capable of binding to PD-1 or PD-L1 and blocking the association between PD1 and PD-L1.

In some embodiments, the inhibitor is an antibody against the checkpoint inhibitor, e.g., an anti-PD1, anti-PDL1 or anti-CTLA4 antibody. The antibody may be a monoclonal antibody. In some embodiments, the antibody may be a human or humanized antibody or a chimeric antibody. The antibody may be an IgG, e.g., IgG1, IgG2, IgG3 or IgG4.

In some embodiments, the antibody may bind the checkpoint inhibitor, e.g., PD1, PDL1 or CTLA4 with a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). The antibody may bind to human PD1, PDL1 or CTLA4.

Exemplary antibodies are known in the art. Any of these or functional variants thereof may be employed in embodiments of the present invention. Examples include:
Nivolumab (anti-PD-1 mAb, BMS-936558/ONO-4538, Bristol-Myers Squibb, formerly MDX-1 106);
Pembrolizumab (anti-PD1 mAb, MK-3475, lambrolizumab, Keytruda®, Merck);
Cemiplimab (anti-PD-1, Regeneron);
Atezolizumab (anti-PD-L1 mAb, Tecentriq®, MPDL3280A/RG7446) Roche/Genentech);
Avelumab (Bavencio), a fully human IgG1 anti-PD-L1 antibody developed by Merck Serono and Pfizer;
Durvalumab (Imfinzi), a fully human IgG1 anti-PD-L1 antibody developed by AstraZeneca.

Exemplary PD-1 inhibitors may also be selected from JTX-4014 by Jounce Therapeutics; Spartalizumab (PDR001); Camrelizumab (SHR1210); Sintilimab (IBI308); Tislelizumab (BGB-A317); Toripalimab (JS 001); Dostarlimab (TSR-042, WBP-285); INCMGA00012 (MGA012), a humanized IgG4 monoclonal antibody developed by Incyte and MacroGenics; AMP-224 by AstraZeneca/MedImmune and GlaxoSmithKline; and AMP-514 (MEDI0680) by AstraZeneca.

Exemplary PD-L1 inhibitors may also be selected from KN035; CK-301 by Checkpoint Therapeutics; AUNP12; CA-170; or BMS-986189.

CTLA-4 (cytotoxic T-lymphocyte-associated protein 4), also known as CD152 is another inhibitor member of the CD28 family of receptors, and is expressed on T cells.

Antibodies that bind and inhibit CTLA-4 are known in the art.

In one example, the antibody is ipilimumab (trade name Yervoy®, Bristol-Myers Squibb), a human IgG antibody. In another example, the anti-CTLA-4 antibody is tremelimumab (formerly ticilimumab, CP-675,206), a human IgG2 antibody.

### L. Clearing Agents

Clearing agent may be used in some embodiments of the invention, as discussed above.

Exemplary agent bind to the antibodies and enhance their rate of clearance from the body. They include anti-idiotype antibodies.

Other exemplary agents are those which bind to the antigen binding site for the radiolabelled compound, but which are not themselves radiolabelled. For example, where the radiolabelled compound comprises a chelator loaded with a radioisotope of a certain chemical element (e.g., a metal), the agent may comprise the same chelator loaded with a non-radioactive isotope of the same element (e.g., metal), or may comprise a non-loaded chelator or a chelator loaded with a different non-radioactive moiety (e.g., a non-radioactive isotope of a different element), provided that it can still be bound by the antigen-binding site.

In some cases, the clearing/blocking agent may additionally comprise a moiety which increases the size and/or hydrodynamic radius of the molecule. These hinder the ability of the molecule to access the tumour, without interfering with the ability of the molecule to bind to the antibody in the circulation. Exemplary moieties include hydrophilic polymers. The moiety may be a polymer or co-polymer e.g., of dextran, dextrin, PEG, polysialic acids (PSAs), hyaluronic acid, hydroxyethyl-starch (HES) or poly(2-ethyl 2-oxazoline) (PEOZ). In other embodiments the moiety may be a non-structured peptide or protein such as XTEN polypeptides (unstructured hydrophilic protein polymers), homo-amino acid polymer (HAP), proline-alanine-serine polymer (PAS), elastin-like peptide (ELP), or gelatin-like protein (GLK). Further exemplary moieties include proteins such as albumin e.g., bovine serum albumin, or IgG. Suitable molecular weights for the moieties/polymers may be in the range e.g., of at least 50 kDa, for example between 50 kDa to 2000 kDa. For example, the molecular weight may be 200-800 kDa, optionally greater than 300, 350, 400 or 450 kDa, and optionally less than 700, 650, 600 or 550 kDa, optionally about 500 kDa.

An exemplary clearing agent is described in WO2019/202399, which is incorporated herein by reference. This describes a dextran-based clearing agent comprising dextran or a derivative thereof, such as aminodextran, conjugated to M-DOTAM (where M-DOTAM is DOTAM or a functional variant thereof incorporating a metal ion),where said complex is recognised by the antigen binding site for Pb-DOTAM. The metal present in the clearing agent may be a stable (non-radioactive) isotope of lead, or a stable or essentially stable isotope of another metal ion, provided that the metal ion-DOTAM complex is recognised with high affinity by the antibody.

By “stable isotope” we mean an isotope that does not undergo radioactive decay. By “essentially stable isotope” we mean an isotope that undergoes radioactive decay with a very long half-life, making it safe for use. Preferably, the metal ion is selected from ions of Pb, Ca and Bi. For example, the clearing agent may comprise a stable isotope of Pb complexed with DOTAM or a functional variant thereof, Ca complexed with DOTAM or a functional variant thereof, or ²⁰⁹Bi (an essentially stable isotope with a half-life of 1.9×10¹⁹ years) complexed with DOTAM or a functional variant thereof. The Pb may be naturally occurring lead, which is a mixture of the stable (non-radioactive) isotopes ²⁰⁴Pb, ²⁰⁶Pb, ²⁰⁷Pb and ²⁰⁸Pb.

### M. Therapeutic Methods and Compositions

In certain aspects, the invention provides the combination therapy described herein as a treatment of a proliferative disease or disorder, e.g, tumour or cancer in an individual. An “individual” or “subject” according to any of the above aspects is preferably a mammal, more preferably a human.

The term “cancer” as used herein include both solid and hematologic cancers, such as lymphomas, lymphocytic leukemias, lung cancer, non small cell lung (NSCL) cancer, bronchioloalviolar cell lung cancer, bone cancer, pancreatic cancer including pancreatic ductal adenocarcinoma (PDAC), skin cancer, cancer of the head or neck, cutaneous or intraocular melanoma, uterine cancer, ovarian cancer, cancer of the anal region, stomach cancer, gastric cancer, colorectal cancer, which may be colon cancer and/or rectal cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumours, brain stem glioma, glioblastoma multiforme, astrocytomas, schwanomas, ependymomas, medulloblastomas, meningiomas, squamous cell carcinomas, pituitary adenoma and Ewings sarcoma, including refractory versions of any of the above cancers, checkpoint-inhibitor experienced versions of any of the above cancers, or a combination of one or more of the above cancers. In one embodiment such “cancer” is a solid tumor selected from breast cancer, lung cancer, colon cancer, ovarian cancer, melanoma cancer, bladder cancer, renal cancer, kidney cancer, liver cancer, head and neck cancer, colorectal cancer, pancreatic cancer, gastric carcinoma cancer, esophageal cancer, mesothelioma or prostate cancer. In another embodiment such “cancer” is a hematological tumor such as for example, leukemia (such as AML, CLL), lymphoma, myelomas. In still another embodiment the “cancer” is breast cancer, lung cancer, colon cancer, colorectal cancer, pancreatic cancer, gastric cancer or prostate cancer.

In some embodiments, the cancer may be refractory to the immune checkpoint inhibitor as a monotherapy. Examples may include human melanoma, renal cell carcinoma (RCC), NSCLC, gastrointestinal, breast, pancreatic, prostate, sarcoma, and colorectal cancers e.g., pancreatic ductal adenocarcinoma.

A method of treating the proliferative disorder or cancer may comprise administering to a patient i) a multispecific antibody or split multispecific antibody, said multispecific antibody or split multispecific antibody having a binding site for a radiolabelled compound and a binding site for a target antigen; ii) a radiolabelled compound; iii) a CD40 agonist; and iv) an immune checkpoint inhibitor.

The radiolabelled compound is administered to the patient after the multispecific antibody or the split multispecific antibody. The multispecific antibody or split multispecific antibody binds to the target antigen. The radiolabelled compound then binds to the multispecific antibody or split multispecific antibody, and is thus localised to the target cell.

The anti-CD40 antibody and immune checkpoint inhibitor can be administered simultaneously or sequentially, in either order. They may be administered before or after the administration of the multispecific antibody/split multispecific antibody and the radiolabelled compound. Preferably, they are administered after the multispecific antibody/split multispecific antibody and the radiolabelled compound.

In one embodiment, the treatment comprises a treatment cycle comprising a first step of pre-targeted radioimmunotherapy comprising administering the multispecific antibody or split multispecific antibody and then administering the radiolabelled compound, and a second step of immunotherapy comprising administering a CD40 agonist and an immune checkpoint inhibitor, wherein the anti-CD40 antibody and the immune checkpoint inhibitor are administered simultaneously or sequentially in either order.

In some embodiments, the second step (immunotherapy) may comprise repeated administrations of one or both of the anti-CD40 antibody and the immune checkpoint inhibitor. For instance, the second step may comprise administration of both the anti-CD40 antibody and the immune checkpoint inhibitor (simultaneously or sequentially, in either order), followed by one or more administrations of the immune checkpoint inhibitor alone. The repeated administrations can occur at a suitable interval as can be determined by the skilled practitioner.

The treatment may comprise one cycle, or preferred embodiments may comprise multiple cycles, e.g., 2, 3, 4, 5 or 6 cycles.

In some embodiments, not all cycles of the treatment are the same. In some embodiments:
a first treatment cycle comprises a first step of pre-targeted radioimmunotherapy comprising administering the multispecific antibody or split multispecific antibody and then administering the radiolabelled compound, and a second step of immunotherapy comprising administering a CD40 agonist and an immune checkpoint inhibitor, wherein the anti-CD40 antibody and the immune checkpoint inhibitor are administered simultaneously or sequentially in either order; and
one or more subsequent cycles comprises a first step of pre-targeted radioimmunotherapy comprising administering the multispecific antibody or split multispecific antibody and then administering the radiolabelled compound, and a second step of immunotherapy comprising administering an immune checkpoint inhibitor.

For instance, there may be 1, 2, 3, 4 or 5 subsequent cycles. The radiolabelled compound is labelled with a radioisotope which is cytotoxic to cells. Suitable radioisotopes include alpha and beta emitters as discussed above.

In some embodiments, the radiolabelled compound may be administered to the subject once the multispecific or split multispecific antibody has been given a suitable period of time to localise to the target cells. For instance, in some embodiments, the radiolabelled compound may be administered to the subject immediately after the multispecific or split multispecific antibody or at least 4 hours, 8 hours, 1 day, or 2 days, after the multispecific or split multispecific antibody. Optionally, it may be administered no more than 3 days, 5 days, or 7 days after the multispecific or split multispecific antibody. In one particular embodiment, the radiolabelled compound may be administered to the subject 2 to 7 days after the multispecific or split multispecific antibody.

In some embodiments, the immunotherapy may be administered after the radiolabelled compound.

An exemplary treatment cycle duration is 14 days, in which the multispecific antibody or split multispecific antibody is administered on day 1 of the cycle; the radiolabelled compound is administered during the following 7 days of the cycle, e.g., on day 8 at the latest in this example, and the immunotherapy (e.g., comprising a CD40 agonist and an immune checkpoint inhibitor, wherein the CD40 agonist and the immune checkpoint inhibitor are administered simultaneously or sequentially in either order, or comprising the immune checkpoint inhibitor without anti-CD40) is given at least 1 day after the administration of the radiolabelled compound, e.g., on day 9. In one embodiment, the CD40 agonist is only administered once, at the first treatment cycle. Treatment schedules involved in combination therapy comprising anti-CD40—and anti-PD-L1 antibodies are for example disclosed in WO2016/023875.

In methods of pre-targeted radioimmunotherapy which make use of a bispecific antibody (i.e., not a “split” antibody according to the present invention) it is common practice to administer a clearing agent or a blocking agent, between administration of the antibody and administration of the radiolabelled compound.

In some aspects of the present invention, a clearing agent is administered after the multispecific antibody and before the radiolabelled compound.

In some embodiments, the clearing agent may be administered a matter of hours or days after the treatment with the multispecific antibody. In some embodiments it may be preferred that the clearing agent is administered at least 2, 4, 6, 8, 10, 12, 16, 18, 22 or 24 hours after the multispecific antibody, or at least 1, 2, 3, 4, 5, 6 or 7 days. In some embodiments, it may be preferred that the clearing agent is administered not more than 14 days after the antibody, e.g., not more than 10, 9, 8, 7, 6, 5, 4, 3 or 2 days.

Optionally, the clearing agent is administered in the period between 4 and 10 days, 4 and 7 days, 2 and 7 days, or 2 to 4 days after the multispecific antibody.

In some embodiments, the radionuclide is administered a matter of minutes, hours or days after the clearing agent. In some embodiments it may be preferred that the radionuclide is administered at least 30 minutes after the clearing agent, and optionally within 48 hours, 24 hours, 8 hours or 4 hours of administration of the clearing agent. In some embodiments, the radionuclide may be administered the day after administration of the clearing agent. Thus, for example, if the radiolabelled compound is administered on day 8 of the cycle, the clearing agent may be administered on day 7.

According to other aspects of the present invention, e.g., those involving split multispecific antibodies, there is no step of administering a clearing agent or a blocking agent to the subject. In certain aspects, there is no step of administering any agent which binds to the first or second hemibody or to the split antibody formed from the first and the second hemibody, between the administration of the split antibody and the administration of the radiolabelled compound. In certain aspects, there is no step of administering any agent between the administration of the split antibody and the radiolabelled compound, except optionally a compound selected from a chemotherapeutic agent and a radiosensitizer. In some embodiments, no agent is administered between the administration of the antibody and the administration of the radiolabelled compound. In some embodiments there may be no injection or infusion of any other agent to the subject, between the administration of the antibody and the administration of the radiolabelled compound.

In some embodiments, the antibodies described herein may additionally or alternatively be administered in combination with radiosensitizers. The radiosensitizer and the antibody may be administered simultaneously or sequentially, in either order.

The multispecific antibodies or split multispecific antibodies the radiolabelled compound, the anti-CD40 antibody and the immune checkpoint inhibitor can be administered by any suitable means, including parenteral, intrapulmonary, and intranasal, and, if desired for local treatment, intralesional administration. Parenteral infusions or injections include intramuscular, intravenous, intraarterial, intraperitoneal, or subcutaneous administration. In some embodiments, administration may be by intravenous or subcutaneous injections. In some embodiments, the multispecific antibodies or split multispecific antibodies, and/or the anti-CD40 antibody and/or the immune checkpoint inhibitor may be administered by IV infusion. In some embodiments, the radiolabelled compound may be administered by IV injection and the anti-CD40 antibody and/or the immune checkpoint inhibitor may be administered subcutaneously (s.c.).

In some embodiments, one or more dosimetry cycles may be used prior to one or more treatment cycles as described above. A dosimetry cycle may comprise the steps of i) administering the multispecific antibody or split multispecific antibody and ii) subsequently administering a compound suitable for imaging radiolabelled with a gamma-emitter (wherein said radiolabelled compound binds to functional binding site for the radiolabelled compound). The compound may be the same as the compound used in the subsequent treatment cycles, except that it is labelled with a gamma emitter rather than an alpha or beta emitter. For example, in one embodiment, the radiolabelled compound used in the dosimetry cycle may be ²⁰¹Pb-DOTAM and the radiolabelled compound used in the treatment cycle may be ²¹²Pb-DOTAM. The patient may be subject to imaging to determine the uptake of the compound into the tumour and/or to estimate the absorbed dose of the compound. This information may be used to estimate the expected radiation exposure in subsequent treatment steps and to adjust the dose of the radiolabelled compound used in the treatment steps to a safe level.

### N. Pharmaceutical Formulations

Pharmaceutical formulations of antibodies as described herein may be prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG), poloxamers (e.g. poloxamer 188) and polysorbates (e.g. PS20, PS80, high grade PS80 i.e. PS80 with >98% oleic acid). Pharmaceutical compositions for some of the cancer immunotherapy components used in accordance with the present invention are for example disclosed in WO2003/040170 (for anti-CD40 antibodies) and WO2010/77634 (for PD-L1 antibodies) or are available as commercial pharmaceutical products.

Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO 2006/044908, the latter compositions including a histidine-acetate buffer.

Where the antibody is a split antibody, the first and second hemibodies may be formulated in a single pharmaceutical composition or in separate pharmaceutical compositions.

The formulation herein may also contain more than one active ingredients as necessary for the particular indication being treated, preferably those with complementary activities that do not adversely affect each other. For example, it may be desirable to further provide chemotherapeutic agents and/or radiosensitizers as discussed above. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### O. Antibody Variants

In certain embodiments, amino acid sequence variants of any of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In certain embodiments, antibody variants having one or more amino acid substitutions are provided. Sites of interest for substitutional mutagenesis include the HVRs (CDRs) and FRs. Conservative substitutions are shown in Table 3 under the heading of “preferred substitutions.” More substantial changes are provided in Table 3 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or reduced or eliminated ADCC or CDC.

TABLE 3

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, le;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modelling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighbouring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about 3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

It may be preferred that the antibody is modified to reduce the extent of glycosylation. In some embodiments the antibody may be aglycosylated or deglycosylated. The antibody may include a substitution at N297, e.g., N297D/A.

### Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant with reduced effector function, e.g., reduced or eliminated CDC, ADCC and/or FcγR binding. In certain aspects, the invention contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious.

In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat′l Acad Sci. USA 83:7059-7063 (1986)) and Hellstrom, I et al., Proc. Nat′lAcad Sci. USA 82:1499-1502 (1985); U.S. Pat. No. 5,821,337 (see Bruggemann, M. et al., J. Exp. Med 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat′lAcad Sci. USA 95:652-656 (1998). C₁q binding assays may also be carried out to confirm that the antibody is unable to bind C₁q and hence lacks CDC activity. See, e.g., C₁q and C₃c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056), e.g., P329G. Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG1 Fc region. In one aspect, the substitutions are L234A, L235A and P329G (LALA-PG) in an Fc region derived from a human IgG1 Fc region. (See, e.g., WO 2012/130831). In another aspect, the substitutions are L234A, L235A and D265A (LALA-DA) in an Fc region derived from a human IgG1 Fc region. Alternative substitutions include L234F and/or L235E, optionally in combination with D265A and/or P329G and/or P331S.

In other embodiments, it may be possible to use a IgG subtype with reduced effector function such as IgG4 or IgG2.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, alterations are made in the Fc region that result in altered (i.e., either improved or diminished, preferably diminished) C₁q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).For instance, in some embodiments, normal FcRn binding may be used.

See also Duncan & Winter, Nature 322:738-40 (1988); U.S. Pat. Nos. 5,648,260; 5,624,821; and WO 94/29351 concerning other examples of Fc region variants.

The C-terminus of a heavy chain of the full-length antibody as reported herein can be a complete C-terminus ending with the amino acid residues PGK. The C-terminus of the heavy chain can be a shortened C-terminus in which one or two of the C terminal amino acid residues have been removed. The C-terminus of the heavy chain may be a shortened C-terminus ending PG. In one aspect of all aspects as reported herein, an antibody comprising a heavy chain including a C-terminal CH3 domain, as specified herein, comprises a C-terminal glycine residue (G446, EU index numbering of amino acid positions). This is still explicitly encompassed with the term “full length antibody” or “full length heavy chain” as used herein.

### Variants for Improved Assembly/Stability

Techniques which are known for making multispecific antibodies can be used to make any of the multispecific antibodies or split multispecific antibodies described herein. These include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Other methods include engineering electrostatic steering effects for making antibody Fc-heterodimeric molecule (see, e.g., WO 2009/089004); cross-linking two or more antibodies or fragments (see, e.g., U.S. Pat. No. 4,676,980, and Brennan et al., Science, 229: 81 (1985)); using leucine zippers (see, e.g., Kostelny et al., J. Immunol., 148(5):1547-1553 (1992) and WO 2011/034605); and using the common light chain technology for circumventing the light chain mis-pairing problem (see, e.g., WO 98/50431).

In any of the multispecific or split multispecific antibodies described above, the correct assembly of heavy chain heterodimers may be assisted by various modifications.

The CH3 domains of the Fc region can be altered by the “knob-into-holes” technology which is described in detail with several examples in e.g. WO 96/027011, Ridgway, J. B., et al., Protein Eng 9 (1996) 617-621; and Merchant, A. M., et al., Nat Biotechnol 16 (1998) 677-681. In this method the interaction surfaces of the two CH3 domains are altered to increase the heterodimerisation of both heavy chains containing these two CH3 domains. Each of the two CH3 domains (of the two heavy chains) can be the “knob”, while the other is the “hole”. For instance one comprises called “knob mutations” (e.g., T366W and optionally one of S354C or Y349C, preferably S354C) and the other comprises the so-called “hole mutations” (e.g., T366S, L368A and Y407V and optionally Y349C or S354C, preferably Y349C) (see, e.g., Carter, P. et al., Immunotechnol. 2 (1996) 73) according to EU index numbering.

Thus in some embodiments the antibody or hemibody is further characterized in that: the CH3 domain of one subunit of the Fc domain and the CH3 domain of the other subunit of the Fc domain each meet at an interface which comprises an original interface between the antibody CH3 domains; wherein said interface is altered to promote the formation of the antibody, wherein the alteration is characterized in that:
a) the CH3 domain of one Fc subunit is altered, so that within the original interface the CH3 domain of one subunit that meets the original interface of the CH3 domain of the other Fc subunit, an amino acid residue is replaced with an amino acid residue having a larger side chain volume, thereby generating a protuberance within the interface of the CH3 domain of one Fc subunit which is positionable in a cavity within the interface of the CH3 domain of the other Fc subunit and
b) the CH3 domain of the other Fc subunit is altered, so that within the original interface of the second CH3 domain that meets the original interface of the first CH3 domain within the antibody an amino acid residue is replaced with an amino acid residue having a smaller side chain volume, thereby generating a cavity within the interface of the second CH3 domain within which a protuberance within the interface of the first CH3 domain is positionable.

Said amino acid residue having a larger side chain volume may optionally be selected from the group consisting of arginine (R), phenylalanine (F), tyrosine (Y), tryptophan (W). Said amino acid residue having a smaller side chain volume may optionally be selected from the group consisting of alanine (A), serine (S), threonine (T), valine (V).

The introduction of a disulfide bridge may additionally or alternatively be used to stabilize the heterodimers (Merchant, A. M., et al., Nature Biotech 16 (1998) 677-681; Atwell, S., et al., J. Mol. Biol. 270 (1997) 26-35) and increase the yield. Thus, optionally, in some embodiments, both CH3 domains are further altered by the introduction of cysteine (C) as amino acid in the corresponding positions of each CH3 domain such that a disulfide bridge between both CH3 domains can be formed. Examples include introduction of a disulfide bond between the following positions:
i) heavy chain variable domain positon 44 to light chain variable domain position 100,
ii) heavy chain variable domain position 105 to light chain variable domain position 43, or
iii) heavy chain variable domain position 101 to light chain variable domain positon 100 (numbering always according to EU index of Kabat).

Additionally or alternatively, the antibodies may comprise amino acid substitutions in Fab molecules (including cross-Fab molecules) comprised therein which are particularly efficient in reducing mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety). The ratio of a desired multispecific antibodies compared to undesired side products, in particular Bence Jones-type side products occurring in one of their binding arms, can be improved by the introduction of charged amino acids with opposite charges at specific amino acid positions in the CH1 and CL domains of a Fab molecule (sometimes referred to herein as “charge modifications”).

Therefore, in some embodiments, an antibody comprising Fab molecules comprises at least one Fab with a heavy chain constant domain CH1 domain comprising charge modifications as described herein, and a light chain constant CL domain comprising charge modifications as described herein.

Charge modifications can be made either in conventional Fab molecule(s) comprised in the antibodies, or in crossover Fab molecule(s) comprised in the antibodies (but generally not in both). In particular embodiments, the charge modifications are made in the conventional Fab molecule(s) comprised in the antibodies.

In some embodiments, in a Fab or cross-Fab comprising a light chain constant domain CL comprising charge modifications and a heavy chain constant domain CH1 comprising charge modifications, charge modifications in the light chain constant domain CL are at position 124 and optionally at position 123 (numbering according to Kabat), and charge modifications in the heavy chain constant domain CH1 are at position 147 and/or 213 (numbering according to Kabat EU Index). In some embodiments, in the light chain constant domain CL the amino acid at position 124 is substituted independently by lysine (K), arginine (R) or histidine (H) (numbering according to Kabat) (in one preferred embodiment independently by lysine (K)), and in the heavy chain constant domain CH1 the amino acid at position 147 and/or the amino acid at position 213 is substituted independently by glutamic acid (E) or aspartic acid (D) (numbering according to Kabat EU index).

### Antibody Derivatives

In certain aspects, any antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### P. Assays

Antibodies provided herein may be identified, screened for, or characterized for their physical/chemical properties and/or biological activities by various assays known in the art.

In one aspect, an antibody of the invention is tested for its antigen binding activity, e.g., by known methods such as ELISA, Western blot, etc.

### Antibody Affinity

In certain embodiments, an antibody provided herein has a dissociation constant (K_{D}) for the target antigen of ≤1 μM, ≤100 nM, ≤10 nM, ≤1 nM, ≤0.1 nM, K 0.01 nM, or ≤0.001 nM (e.g., 10⁻⁸ M or less, e.g., from 10⁻¹ M to 10⁻¹³ M, e.g., from 10⁻⁹ M to 10⁻¹³ M), or as otherwise stated herein.

In certain embodiments, an antigen binding site for the radiolabelled compound has a dissociation constant (K_{D}) for the radiolabelled compound of ≤1 μM, 100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In some embodiments, the K_{D} is 1 nM or less, 500 pM or less, 200 pM or less, 100 pM or less, 50 pM or less, 20 pM or less, 10 pM or less, 5 pM or less or 1 pM or less, or as otherwise stated herein. For instance, the functional binding site may bind the radiolabelled compound/metal chelate with a K_{D} of about 1 pM-1 nM, e.g., about 1-10 pM, 1-100 pM, 5-50 pM, 100-500 pM or 500 pM-1 nM.

In one embodiment, K_{D} is measured by a radiolabelled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labelled antigen in the presence of a titration series of unlabelled antigen, then capturing bound antigen with an anti-Fab antibody-coated plate (see, e.g., Chen et al., J. Mol. Biol. 293:865-881(1999)). To establish conditions for the assay, MICROTITER© multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20M) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 106 M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 250C of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO m spectrophotometer (ThermoSpectronic) with a stirred cuvette.

In another embodiment, K_{D} is measured using a SET (solution equilibration titration) assay. According to this assay, test antibodies are typically applied in a constant concentration and mixed with serial dilutions of the test antigen. After incubation to establish an equilibrium, the portion of free antibodies is captured on an antigen coated surface and detected with labelled/tagged anti-species antibody, generally using electochemiluminescence (e.g., as described in Haenel et al Analytical Biochemistry 339 (2005) 182-184).

For example, in one embodiment 384-well streptavidin plates (Nunc, Microcoat #11974998001) are incubated overnight at 4° C. with 25 μl/well of an antigen-Biotin-Isomer Mix in PBS-buffer at a concentration of 20 ng/ml. For equilibration of antibody samples with free antigen: 0.01 nM-1 nM of antibody is titrated with the relevant antigen in 1:3, 1:2 or 1:1.7 dilution steps starting at a concentration of 2500 nM, 500 nM or 100 nM of antigen. The samples are incubated at 4° C. overnight in sealed REMP Storage polypropylene microplates (Brooks). After overnight incubation, streptavidin plates are washed 3× with 90 μl PBST per well. 15 μl of each sample from the equilibration plate is transferred to the assay plate and incubated for 15 min at RT, followed by 3×90 μl washing steps with PBST buffer. Detection is carried out by adding 25 μl of a goat anti-human IgG antibody-POD conjugate (Jackson, 109-036-088, 1:4000 in OSEP), followed by 6×90 μl washing steps with PBST buffer. 25 μl of TMB substrate (Roche Diagnostics GmbH, Cat. No.: 11835033001) are added to each well. Measurement takes place at 370/492 nm on a Safire2 reader (Tecan).

In another embodiment, K_{D} is measured using a KinExA (kinetic exclusion) assay. According to this assay, the antigen is typically titrated into a constant concentration of antibody binding sites, the samples are allowed to equilibrate, and then drawn quickly through a flow cell where free antibody binding sites are captured on antigen-coated beads, while the antigen-saturated antibody complex is washed away. The bead-captured antibody is then detected with a labelled anti-species antibody, e.g., fluorescently labelled (Bee et al PloS One, 2012; 7(4): e36261). For example, in one embodiment, KinExA experiments are performed at room temperature (RT) using PBS pH 7.4 as running buffer. Samples are prepared in running buffer supplemented with 1 mg/ml BSA (“sample buffer”). A flow rate of 0.25 ml/min is used. A constant amount of antibody with 5 pM binding site concentration is titrated with antigen by twofold serial dilution starting at 100 pM (concentration range 0.049 pM-100 pM). One sample of antibody without antigen serves as 100% signal (i.e. without inhibition). Antigen-antibody complexes are incubated at RT for at least 24 h to allow equilibrium to be reached. Equilibrated mixtures are then drawn through a column of antigen-coupled beads in the KinExA system at a volume of 5 ml permitting unbound antibody to be captured by the beads without perturbing the equilibrium state of the solution. Captured antibody is detected using 250 ng/ml Dylight 650©-conjugated anti-human Fc-fragment specific secondary antibody in sample buffer. Each sample is measured in duplicates for all equilibrium experiments. The KD is obtained from non-linear regression analysis of the data using a one-site homogeneous binding model contained within the KinExA software (Version 4.0.11) using the “standard analysis” method.

### Biological Activity

In some embodiments, the combination therapy results in a slower rate of tumour growth in a subject than the pre-targeted radioimmunotherapy alone and/or with the immunotherapy alone. In some embodiments, the combination therapy results in an increased likelihood of subject survival than treatment with the pre-targeted radioimmunotherapy alone and/or with the immunotherapy alone. In some embodiments, the combination therapy results in an increased frequency of activated intratumoral CD8 T cells in the subject (e.g., as measured by upregulation of 41BB expression), and/or an increased frequency of activated plasmacytoid DCs (pDCs) and classical DCs (cDCs) in tumor, spleen and draining lymphnodes (DLNs) of the subject (e.g., as measured by upregulation of CD86 expression), and/or increased frequency of T cells in total immune cells of the subject than treatment with the pre-targeted radioimmunotherapy alone and/or with the immunotherapy alone. In some embodiments, the subject may be a patient, e.g., a human patient. In other embodiments the subject in which the activity is assessed may be a model animal such as a mouse model.

In some embodiments, the combination therapy results in an enhanced immune memory response or reduced likelihood of tumour recurrence in the subject than treatment with the pre-targeted radioimmunotherapy alone and/or with the immunotherapy alone. Enhanced immune memory response can be assessed by greater resistance to tumour rechallenge in a mouse model.

An example of a mouse model may be a mouse inoculated with a tumour cell line expressing the target antigen for the antibody/split antibody. Examples are the Panc02 tumour cell line or MC38 tumour cell line engineered to express the target antigen for the antibody/split antibody, e.g, huCEA. The tumour cell line may also be engineered to express a reporter such as luciferase. The inoculation may be subcutaneous or orthotopic (e.g., intrapancreatic). The inoculated mouse may also be transgenic for the target antigen, e.g., huCEA. An example of a mouse transgenic for human CEA as a model for immunotherapy is discussed in Clarke et al Cancer Research 58, 1469-1477, Apr. 1, 1998.

### III. Sequences

| SEQ | | |
|---|---|---|
| ID | | |
| NO | Description | Sequence |
| 1 | heavy chain | GFSLSTYSMS |
| | CDR1, <Pb- | |
| | Dotam> | |
| 2 | heavy chain | FIGSRGDTYYASWAKG |
| | CDR2 <Pb- | |
| | Dotam> | |
| 3 | heavy chain | ERDPYGGGAYPPHL |
| | CDR3 <Pb- | |
| | Dotam> | |
| 4 | light chain | QSSHSVYSDNDLA |
| | CDR1, | |
| | <Pb-Dotam> | |
| 5 | light chain | QASKLAS |
| | CDR2 | |
| | <Pb-Dotam> | |
| 6 | light chain | LGGYDDESDTYG |
| | CDR3 | |
| | <Pb-Dotam> | |
| 7 | heavy chain | (Q)VTLKESGPVLVKPTETLTLTCTVSGFSLST |
| | variable domain | YSMSWIRQPPGKALEWLGFIGSRGDTYYASWAKGRLTISKDTSKSQVVLT |
| | <Pb-Dotam> | MTNMDPVDTATYYCARERDPYGGGAYPPHLWGRGTLVTVS S |
| | PRIT-0213 | |
| 8 | light chain | (A)IQMTQSPSSLSASVGDRVTI TCQSSHSVYS |
| | variable domain | DNDLAWYQQKPGKAPKLLIYQASKLASGVPSRFSGSGSGTDFTLTISSLQ |
| | <Pb-Dotam> | PEDFATYYCLGGYDDESDTYGFGGGTKVEIK |
| | PRIT-0213 | |
| 9 | heavy chain | (Q)VQLQQWGAGLLKPSETLSLTCAVYGFSLST |
| | variable domain | YSMSWIRQPPGKGLEWIGFIGSRGDTYYASWAKGRVTISRDTSKNQVSLK |
| | <Pb-Dotam> | LSSVTAADTAVYYCARERDPYGGGAYPPHLWGRGTLVTVSS |
| | PRIT-0214 | |
| 10 | light chain | (A)IQMTQSPSSLSASVGDRVTITCQSSHSVYS |
| | variable domain | DNDLAWYQQKPGKAPKLLIYQASKLASGVPSRFSGSGSGTDFTLTISSLQ |
| | <Pb-Dotam> | PEDFATYYCLGGYDDESDTYGFGGGTKVEIK |
| | PRIT-0214 | |
| 11 | heavy chain | GFNIKDTYMH |
| | CDR1 <CEA> | |
| | T84.66 | |
| 12 | heavy chain | RIDPANGNSKYVPKFQG |
| | CDR2 <CEA> | |
| | T84.66 | |
| 13 | heavy chain | FGYYVSDYAMAY |
| | CDR3 <CEA> | |
| | T84.66 | |
| 14 | light chain | RAGESVDIFGVGFLH |
| | CDR1 | |
| | <CEA> T84.66 | |
| 15 | light chain | RASNRAT |
| | CDR2 | |
| | <CEA> T84.66 | |
| 16 | light chain | QQTNEDPYT |
| | CDR3 | |
| | <CEA> T84.66 | |
| 17 | heavy chain | QVQLVQSGAEVKKPGSSVKVSCKASGFNIKDTYMHWVRQAPGQGLEWMGRIDPANGNSKYVPKFQGRV |
| | variable domain | TITADTSTSTAYMELSSLRSEDTAVYYCAPFGYYVSDYAMAYWGQGTLVTVSS |
| | <CEA> T84.66 | |
| 18 | light chain | EIVLTQSPATLSLSPGERATLSCRAGESVDIFGVGFLHWYQQKPGQAPRLLIYRASNRATGIPARFSG |
| | variable domain | SGSGTDFTLTISSLEPEDFAVYYCQQTNEDPYTFGQGTKLEIK |
| | <CEA> T84.66 | |
| 19 | heavy chain | GYTFTEFGMN |
| | CDR1 <CEA> | |
| | CH1A1A | |
| 20 | heavy chain | WINTKTGEATYVEEFKG |
| | CDR2 <CEA> | |
| | CH1A1A | |
| 21 | heavy chain | WDFAYYVEAMDY |
| | CDR3 <CEA> | |
| | CH1A1A | |
| 22 | light chain | KASAAVGTYVA |
| | CDR1 | |
| | <CEA> CH1A1A | |
| 23 | light chain | SASYRKR |
| | CDR2 | |
| | <CEA> CH1A1A | |
| 24 | light chain | HQYYTYPLFT |
| | CDR3 | |
| | <CEA> CH1A1A | |
| 25 | heavy chain | QVQLVQSGAEVKKPGASVKVSCKASGYTFTEFGMNWVRQAPGQGLEWMGWINTKTGEATYVEEFKGRV |
| | variable domain | TFTTDTSTSTAYMELRSLRSDDTAVYYCARWDFAYYVEAMDYWGQGTTVTVSS |
| | <CEA> | |
| | CH1A1A | |
| 26 | light chain | DIQMTQSPSSLSASVGDRVTITCKASAAVGTYVAWYQQKPGKAPKLLIYSASYRKRGVPSRFSGSGSG |
| | variable domain | TDFTLTISSLQPEDFATYYCHQYYTYPLFTFGQGTKLEIK |
| | <CEA> | |
| | CH1A1A | |
| 27 | Heavy chain | QVQLVQSGAEVKKPGASVKVSCKASGYTFTEFGMNWVRQAPGQGLEWMGW |
| | <CEA> of | INTKTGEATYVEEFKGRVTFTTDTSTSTAYMELRSLRSDDTAVYYCARWD |
| | P1AD8749 | FAYYVEAMDYWGQGTTVTVSSASTKGPSVFPLAPSSKSTSGGTAALGCLV |
| | without linker | KDYFPEPVTVSWNSGALTSGVHTFPAVLQSSGLYSLSSVVTVPSSSLGTQ |
| | and <DOTAM- | TYICNVNHKPSNTKVDKKVEPKSCDKTHTCPPCPAPEAAGGPSVFLFPPK |
| | VH>→Same | PKDTLMISRTPEVTCVVVDVSHEDPEVKFNWYVDGVEVHNAKTKPREEQY |
| | Plasmid as | NSTYRVVSVLTVLHQDWLNGKEYKCKVSNKALGAPIEKTISKAKGQPREP |
| | SeqID32, lacking | QVYTLPPCRDELTKNQVSLWCLVKGFYPSDIAVEWESNGQPENNYKTTPP |
| | linker and | VLDSDGSFFLYSKLTVDKSRWQQGNVFSCSVMHEALHNHYTQKSLSLSPG |
| | <DOTAM> | |
| 28 | P1AD8749 Heavy | QVQLVQSGAEVKKPGASVKVSCKASGYTFTEFGMNWVRQAPGQGLEWMGWINTKTGEATYVEEFKGRV |
| | chain hole | TFTTDTSTSTAYMELRSLRSDDTAVYYCARWDFAYYVEAMDYWGQGTTVTVSSASTKGPSVFPLAPSS |
| | <CEA> CH1A1A | KSTSGGTAALGCLVKDYFPEPVTVSWNSGALTSGVHTFPAVLQSSGLYSLSSVVTVPSSSLGTQTYIC |
| | | NVNHKPSNTKVDKKVEPKSCDKTHTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDWLNGKEYKCKVSNKALGAPIEKTI |
| | | SKAKGQPREPQVCTLPPSRDELTKNQVSLSCAVKGFYPSDIAVEWESNGQPENNYKTTPPVLDSDGSF |
| | | FLVSKLTVDKSRWQQGNVFSCSVMHEALHNHYTQKSLSLSPG |
| 29 | Heavy chain | QVQLVQSGAEVKKPGASVKVSCKASGYTFTEFGMNWVRQAPGQGLEWMGW |
| | <CEA> of | INTKTGEATYVEEFKGRVTFTTDTSTSTAYMELRSLRSDDTAVYYCARWD |
| | P1AD8592 | FAYYVEAMDYWGQGTTVTVSSASTKGPSVFPLAPSSKSTSGGTAALGCLV |
| | without linker | KDYFPEPVTVSWNSGALTSGVHTFPAVLQSSGLYSLSSVVTVPSSSLGTQ |
| | and <DOTAM- | TYICNVNHKPSNTKVDKKVEPKSCDKTHTCPPCPAPEAAGGPSVFLFPPK |
| | VL>→Same | PKDTLMISRTPEVTCVVVDVSHEDPEVKFNWYVDGVEVHNAKTKPREEQY |
| | Plasmid as | NSTYRVVSVLTVLHQDWLNGKEYKCKVSNKALGAPIEKTISKAKGQPREP |
| | SeqID33, lacking | QVCTLPPSRDELTKNQVSLSCAVKGFYPSDIAVEWESNGQPENNYKTTPP |
| | linker and | VLDSDGSFFLVSKLTVDKSRWQQGNVFSCSVMHEALHNHYTQKSLSLSPG |
| | <DOTAM> | |
| 30 | P1AD8592 Heavy | QVQLVQSGAEVKKPGASVKVSCKASGYTFTEFGMNWVRQAPGQGLEWMGWINTKTGEATYVEEFKGRV |
| | chain Knob | TFTTDTSTSTAYMELRSLRSDDTAVYYCARWDFAYYVEAMDYWGQGTTVTVSSASTKGPSVFPLAPSS |
| | <CEA> CH1A1A | KSTSGGTAALGCLVKDYFPEPVTVSWNSGALTSGVHTFPAVLQSSGLYSLSSVVTVPSSSLGTQTYIC |
| | | NVNHKPSNTKVDKKVEPKSCDKTHTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDWLNGKEYKCKVSNKALGAPIEKTI |
| | | SKAKGQPREPQVYTLPPCRDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSPG |
| 31 | Linker | GGGGSGGGGSGGGGSGGGGS |
| 32 | P1AD8749 heavy | QVQLVQSGAEVKKPGASVKVSCKASGYTFTEFGMNWVRQAPGQGLEWMGWINTKTGEATYVEEFKGRV |
| | chain knob | TFTTDTSTSTAYMELRSLRSDDTAVYYCARWDFAYYVEAMDYWGQGTTVTVSSASTKGPSVFPLAPSS |
| | <CEA> CH1A1A | KSTSGGTAALGCLVKDYFPEPVTVSWNSGALTSGVHTFPAVLQSSGLYSLSSVVTVPSSSLGTQTYIC |
| | <Dotam-VH> | NVNHKPSNTKVDKKVEPKSCDKTHTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDWLNGKEYKCKVSNKALGAPIEKTI |
| | | SKAKGQPREPQVYTLPPCRDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSPGGGGGSGGGGSGGGGSGGGGSVTLKES |
| | | GPVLVKPTETLTLTCTVSGFSLSTYSMSWIRQPPGKALEWLGFIGSRGDTYYASWAKGRLTISKDTSK |
| | | SQVVLTMTNMDPVDTATYYCARERDPYGGGAYPPHLWGRGTLVTVSS |
| 33 | P1AD8592 heavy | QVQLVQSGAEVKKPGASVKVSCKASGYTFTEFGMNWVRQAPGQGLEWMGWINTKTGEATYVEEFKGRV |
| | chain hole | TFTTDTSTSTAYMELRSLRSDDTAVYYCARWDFAYYVEAMDYWGQGTTVTVSSASTKGPSVFPLAPSS |
| | <CEA> CH1A1A | KSTSGGTAALGCLVKDYFPEPVTVSWNSGALTSGVHTFPAVLQSSGLYSLSSVVTVPSSSLGTQTYIC |
| | <Dotam-VL> | NVNHKPSNTKVDKKVEPKSCDKTHTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDWLNGKEYKCKVSNKALGAPIEKTI |
| | | SKAKGQPREPQVCTLPPSRDELTKNQVSLSCAVKGFYPSDIAVEWESNGQPENNYKTTPPVLDSDGSF |
| | | FLVSKLTVDKSRWQQGNVFSCSVMHEALHNHYTQKSLSLSPGGGGGSGGGGSGGGGSGGGGSIQMTQS |
| | | PSSLSASVGDRVTITCQSSHSVYSDNDLAWYQQKPGKAPKLLIYQASKLASGVPSRFSGSGSGTDFTL |
| | | TISSLQPEDFATYYCLGGYDDESDTYGFGGGTKVEIK |
| 34 | P1AD8749 and | DIQMTQSPSSLSASVGDRVTITCKASAAVGTYVAWYQQKPGKAPKLLIYS |
| | PIAD8592 | ASYRKRGVPSRFSGSGSGTDFTLTISSLQPEDFATYYCHQYYTYPLFTFG |
| | light chain | QGTKLEIKRTVAAPSVFIFPPSDEQLKSGTASVVCLLNNFYPREAKVQWK |
| | <CEA>CH1A1A | VDNALQSGNSQESVTEQDSKDSTYSLSSTLTLSKADYEKHKVYACEVTHQ |
| | | GLSSPVTKSFNRGEC |
| 35 | Heavy chain CDR | DYGVH |
| | 1, <C825> | |
| 36 | Heavy chain CDR | VIWSGGGTAYNTALIS |
| | 2, <C825> | |
| 37 | Heavy chain CDR | RGSYPYNYFDA |
| | 3, <C825> | |
| 38 | Light chain CDR | GSSTGAVTASNYAN |
| | 1,<C825> | |
| 39 | Light chain CDR | GHNNRPP |
| | 2, <C825> | |
| 40 | Light chain CDR | ALWYSDHWV |
| | 3, <C825> | |
| 41 | Heavy chain | HVKLQESGPGLVQPSQSLSLTCTVSGFSLTDYGVHWVRQSPGKGLEWLGVIWSGGGTAYNTALISRLN |
| | variable domain | IYRDNSKNQVFLEMNSLQAEDTAMYYCARRGSYPYNYFDAWGQGTTVTVSS |
| | <C825> | |
| 42 | Light chain | QAVVIQESALTTPPGETVTLTCGSSTGAVTASNYANWVQEKPDHLFTGLIGGHNNRPPGVPARFSGSL |
| | variable domain, | IGDKAALTIAGTQTEDEAIYFCALWYSDHWVIGGGTKLTVL |
| | <C825> | |
| 43 | heavy chain | DYYMN |
| | CDR1 <CEA> | |
| | A5B7 | |
| 44 | heavy chain | FIGNKANAYTTEYSASVKG |
| | CDR2 <CEA> | |
| | A5B7 | |
| 45 | heavy chain | DRGLRFYFDY |
| | CDR3 <CEA> | |
| | A5B7 | |
| 46 | light chain | RASSSVTYIH |
| | CDR1 <CEA> | |
| | A5B7 | |
| 47 | light chain | ATSNLAS |
| | CDR2 <CEA> | |
| | A5B7 | |
| 48 | light chain | QHWSSKPPT |
| | CDR3 <CEA> | |
| | A5B7 | |
| 49 | heavy chain | EVQLLESGGGLVQPGGSLRLSCAASGFTFTDYYMNWVRQAPGKGLEWLGFIGNKANAYTTEYSASVKG |
| | variable domain | RFTISRDKSKNTLYLQMNSLRAEDTATYYCTRDRGLRFYFDYWGQGTTVTVSS |
| | <CEA> A5B7 | |
| 50 | light chain | EIVLTQSPATLSLSPGERATLSCRASSSVTYIHWYQQKPGQAPRSWIYATSNLASGIPARFSGSGSGT |
| | variable domain | DFTLTISSLEPEDFAVYYCQHWSSKPPTFGQGTKLEIK |
| | <CEA> A5B7 | |
| 51 | P1AE4956 heavy | EVQLLESGGGLVQPGGSLRLSCAASGFTFTDYYMNWVRQAPGKGLEWLGFIGNKANAYTTEYSASVKG |
| | chain hole | RFTISRDKSKNTLYLQMNSLRAEDTATYYCTRDRGLRFYFDYWGQGTTVTVSSASTKGPSVFPLAPSS |
| | <CEA> A5B7 | KSTSGGTAALGCLVKDYFPEPVTVSWNSGALTSGVHTFPAVLQSSGLYSLSSVVTVPSSSLGTQTYIC |
| | | NVNHKPSNTKVDKKVEPKSCDKTHTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDWLNGKEYKCKVSNKALGAPIEKTI |
| | | SKAKGQPREPQVCTLPPSRDELTKNQVSLSCAVKGFYPSDIAVEWESNGQPENNYKTTPPVLDSDGSF |
| | | FLVSKLTVDKSRWQQGNVFSCSVMHEALHNHYTQKSLSLSPG |
| 52 | P1AE4956 heavy | EVQLLESGGGLVQPGGSLRLSCAASGFTFTDYYMNWVRQAPGKGLEWLGFIGNKANAYTTEYSASVKG |
| | chain knob | RFTISRDKSKNTLYLQMNSLRAEDTATYYCTRDRGLRFYFDYWGQGTTVTVSSASTKGPSVFPLAPSS |
| | <CEA> A5B7 | KSTSGGTAALGCLVKDYFPEPVTVSWNSGALTSGVHTFPAVLQSSGLYSLSSVVTVPSSSLGTQTYIC |
| | <Dotam-VH> | NVNHKPSNTKVDKKVEPKSCDKTHTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDWLNGKEYKCKVSNKALGAPIEKTI |
| | | SKAKGQPREPQVYTLPPCRDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSPGGGGGSGGGGSGGGGSGGGGSVTLKES |
| | | GPVLVKPTETLTLTCTVSGFSLSTYSMSWIRQPPGKALEWLGFIGSRGDTYYASWAKGRLTISKDTSK |
| | | SQVVLTMTNMDPVDTATYYCARERDPYGGGAYPPHLWGRGTLVTVSS |
| 53 | Heavy chain | EVQLLESGGGLVQPGGSLRLSCAASGFTFTDYYMNWVRQAPGKGLEWLGFIGNKANAYTTEYSASVKG |
| | <CEA> of | RFTISRDKSKNTLYLQMNSLRAEDTATYYCTRDRGLRFYFDYWGQGTTVTVSSASTKGPSVFPLAPSS |
| | PIAE4956 | KSTSGGTAALGCLVKDYFPEPVTVSWNSGALTSGVHTFPAVLQSSGLYSLSSVVTVPSSSLGTQTYIC |
| | without linker | NVNHKPSNTKVDKKVEPKSCDKTHTCPPCPAPEAAGGPSVFLFPPKPKDTLMISRTPEVTCVVVDVSH |
| | and DOTAM-VH> | EDPEVKFNWYVDGVEVHNAKTKPREEQYNSTYRVVSVLTVLHQDWLNGKEYKCKVSNKALGAPIEKTI |
| | →Same Plasmid | SKAKGQPREPQVYTLPPCRDELTKNQVSLWCLVKGFYPSDIAVEWESNGQPENNYKTTPPVLDSDGSF |
| | as SeqID 52, | FLYSKLTVDKSRWQQGNVFSCSVMHEALHNHYTQKSLSLSP |
| | lacking linker | |
| | and <DOTAM> | |
| 54 | P1AE4956 light | EIVLTQSPATLSLSPGERATLSCRASSSVTYIHWYQQKPGQAPRSWIYATSNLASGIPARFSGSGSGT |
| | chain <CEA> | DFTLTISSLEPEDFAVYYCQHWSSKPPTFGQGTKLEIKRTVAAPSVFIFPPSDEQLKSGTASVVCLLN |
| | A5B7 | NFYPREAKVQWKVDNALQSGNSQESVTEQDSKDSTYSLSSTLTLSKADYEKHKVYACEVTHQGLSSPV |
| | | TKSFNRGEC |
| 55 | P1AE4957 | EVQLLESGGGLVQPGGSLRLSCAASGFTFTDYYMNWVRQAPGKGLEWLGFIGNKANAYTTEYSASVKG |
| | heavy chain | RFTISRDKSKNTLYLQMNSLRAEDTATYYCTRDRGLRFYFDYWGQGTTVTVSSASTKGPSVFPLAPSS |
| | knob <CEA> | KSTSGGTAALGCLVKDYFPEPVTVSWNSGALTSGVHTFPAVLQSSGLYSLSSVVTVPSSSLGTQTYIC |
| | A5B7 | NVNHKPSNTKVDKKVEPKSCDKTHTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDWLNGKEYKCKVSNKALGAPIEKTI |
| | | SKAKGQPREPQVYTLPPCRDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSPG |
| 56 | P1AE4957 | EVQLLESGGGLVQPGGSLRLSCAASGFTFTDYYMNWVRQAPGKGLEWLGFIGNKANAYTTEYSASVKG |
| | heavy chain | RFTISRDKSKNTLYLQMNSLRAEDTATYYCTRDRGLRFYFDYWGQGTTVTVSSASTKGPSVFPLAPSS |
| | hole | KSTSGGTAALGCLVKDYFPEPVTVSWNSGALTSGVHTFPAVLQSSGLYSLSSVVTVPSSSLGTQTYIC |
| | <CEA> A5B7 | NVNHKPSNTKVDKKVEPKSCDKTHTCPPCPAPEAAGGPSVFLFPPKPKDTLMISRTPEVTCVVVDVSH |
| | <Dotam-VL> | EDPEVKFNWYVDGVEVHNAKTKPREEQYNSTYRVVSVLTVLHQDWLNGKEYKCKVSNKALGAPIEKTI |
| | | SKAKGQPREPQVCTLPPSRDELTKNQVSLSCAVKGFYPSDIAVEWESNGQPENNYKTTPPVLDSDGSF |
| | | FLVSKLTVDKSRWQQGNVFSCSVMHEALHNHYTQKSLSLSPGGGGGSGGGGSGGGGSGGGGSIQMTQS |
| | | PSSLSASVGDRVTITCQSSHSVYSDNDLAWYQQKPGKAPKLLIYQASKLASGVPSRFSGSGSGTDFTL |
| | | TISSLQPEDFATYYCLGGYDDESDTYGFGGGTKVEIK |
| 57 | Heavy chain | EVQLLESGGGLVQPGGSLRLSCAASGFTFTDYYMNWVRQAPGKGLEWLGFIGNKANAYTTEYSASVKG |
| | <CEA> of | RFTISRDKSKNTLYLQMNSLRAEDTATYYCTRDRGLRFYFDYWGQGTTVTVSSASTKGPSVFPLAPSS |
| | P1AE4957 | KSTSGGTAALGCLVKDYFPEPVTVSWNSGALTSGVHTFPAVLQSSGLYSLSSVVTVPSSSLGTQTYIC |
| | without linker | NVNHKPSNTKVDKKVEPKSCDKTHTCPPCPAPEAAGGPSVFLFPPKPKDTLMISRTPEVTCVVVDVSH |
| | and DOTAM- | EDPEVKFNWYVDGVEVHNAKTKPREEQYNSTYRVVSVLTVLHQDWLNGKEYKCKVSNKALGAPIEKTI |
| | VL>→Same | SKAKGQPREPQVCTLPPSRDELTKNQVSLSCAVKGFYPSDIAVEWESNGQPENNYKTTPPVLDSDGSF |
| | Plasmid as | FLVSKLTVDKSRWQQGNVFSCSVMHEALHNHYTQKSLSLSP |
| | SeqID 56, | |
| | lacking linker | |
| | and <DOTAM> | |
| 58 | P1AE4957 light | EIVLTQSPATLSLSPGERATLSCRASSSVTYIHWYQQKPGQAPRSWIYATSNLASGIPARFSGSGSGT |
| | chain <CEA> | DFTLTISSLEPEDFAVYYCQHWSSKPPTFGQGTKLEIKRTVAAPSVFIFPPSDEQLKSGTASVVCLLN |
| | A5B7 | NFYPREAKVQWKVDNALQSGNSQESVTEQDSKDSTYSLSSTLTLSKADYEKHKVYACEVTHQGLSSPV |
| | | TKSFNRGEC |
| 59 | heavy chain | GGTFSYYAIS |
| | CDR1 <CEA> | |
| | 28A9 | |
| 60 | heavy chain | GILPAFGAANYAQKFQG |
| | CDR2 <CEA> | |
| | 28A9 | |
| 61 | heavy chain | LPPLPGAGLDY |
| | CDR3 <CEA> | |
| | 28A9 | |
| 62 | light chain | RASQSISSWLA |
| | CDR1 <CEA> | |
| | 28A9 | |
| 63 | light chain | DASSLES |
| | CDR2 <CEA> | |
| | 28A9 | |
| 64 | light chain | QQNTQYPMT |
| | CDR3 <CEA> | |
| | 28A9 | |
| 65 | heavy chain | QVQLVQSGAEVKKPGSSVKVSCKASGGTFSYYAISWVRQAPGQGLEWMGGILPAFGAANYAQKFQGRV |
| | variable domain | TITADKSTSTAYMELSSLRSEDTAVYYCARLPPLPGAGLDYWGQGTTVTVSS |
| | <CEA> 28A9 | |
| 66 | light chain | DIQMTQSPSTLSASVGDRVTITCRASQSISSWLAWYQQKPGKAPKLLIYDASSLESGVPSRFSGSGSG |
| | variable domain | TEFTLTISSLQPDDFATYYCQQNTQYPMTFGQGTKVEIK |
| | <CEA> 28A9 | |
| 67 | heavy chain | GFTFSKYAMA |
| | CDR1 | |
| | <GPRC5D> | |
| 68 | heavy chain | SISTGGVNTYYADSVKG |
| | CDR2 | |
| | <GPRC5D> | |
| 69 | heavy chain | HTGDYFDY |
| | CDR3 | |
| | <GPRC5D> | |
| 70 | light chain | RASQSVSISGINLMN |
| | CDR1 | |
| | <GPRC5D> | |
| 71 | light chain | HASILAS |
| | CDR2 | |
| | <GPRC5D> | |
| 72 | light chain | QQTRESPLT |
| | CDR3 | |
| | <GPRC5D> | |
| 73 | Heavy chain | EVQLLESGGGLVQPGGSLRLSCAASGFTFSKYAMAWVRQAPGKGLEWVASISTGGVNTYYADSVKGRF |
| | variable domain | TISRDNSKNTLYLQMNSLRAEDTAVYYCATHTGDYFDYWGQGTMVTVSS |
| | <GPRC5D> | |
| 74 | Light chain | EIVLTQSPGTLSLSPGERATLSCRASQSVSISGINLMNWYQQKPGQQPKLLIYHASILASGIPDRFSG |
| | variable domain | SGSGTDFTLTISRLEPEDFAVYYCQQTRESPLTFGQGTRLEIK |
| | <GPRC5D> | |
| 75 | heavy chain | GFTFSSYAMS |
| | CDR1 <FAP> | |
| | 4B9 | |
| 76 | heavy chain | AIIGSGASTYYADSVKG |
| | CDR2 <FAP> | |
| | 4B9 | |
| 77 | heavy chain | GWFGGFNY |
| | CDR3 <FAP> | |
| | 4B9 | |
| 78 | light chain | RASQSVTSSYLA |
| | CDR1 <FAP> | |
| | 4B9 | |
| 79 | light chain | VGSRRAT |
| | CDR2 <FAP> | |
| | 4B9 | |
| 80 | light chain | QQGIMLPPT |
| | CDR3 <FAP> | |
| | 4B9 | |
| 81 | Heavy chain | EVQLLESGGGLVQPGGSLRLSCAASGFTFSSYAMSWVRQAPGKGLEWVSAIIGSGASTYYADSVKGRF |
| | variable domain | TISRDNSKNTLYLQMNSLRAEDTAVYYCAKGWFGGFNYWGQGTLVTVSS |
| | <FAP> 4B9 | |
| 82 | Light chain | EIVLTQSPGTLSLSPGERATLSCRASQSVTSSYLAWYQQKPGQAPRLLINVGSRRATGIPDRFSGSGS |
| | variable domain | GTDFTLTISRLEPEDFAVYYCQQGIMLPPTFGQGTKVEIK |
| | <FAP> 4B9 | |
| 83 | P1AF0709 | QVQLVQSGAEVKKPGSSVKVSCKASGFNIKDTYMHWVRQAPGQGLEWMGRIDPANGNSKYVPKFQGRV |
| | HCknob <CEA> | TITADTSTSTAYMELSSLRSEDTAVYYCAPFGYYVSDYAMAYWGQGTLVTVSSASTKGPSVFPLAPSS |
| | T84.66 | KSTSGGTAALGCLVKDYFPEPVTVSWNSGALTSGVHTFPAVLQSSGLYSLSSVVTVPSSSLGTQTYIC |
| | (D1AE4688) | NVNHKPSNTKVDKKVEPKSCDKTHTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDWLNGKEYKCKVSNKALGAPIEKTI |
| | | SKAKGQPREPQVYTLPPCRDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSPG |
| 84 | P1AF0709 | QVQLVQSGAEVKKPGSSVKVSCKASGFNIKDTYMHWVRQAPGQGLEWMGRIDPANGNSKYVPKFQGRV |
| | HChole <CEA> | TITADTSTSTAYMELSSLRSEDTAVYYCAPFGYYVSDYAMAYWGQGTLVTVSSASTKGPSVFPLAPSS |
| | T84.66 Dotam- | KSTSGGTAALGCLVKDYFPEPVTVSWNSGALTSGVHTFPAVLQSSGLYSLSSVVTVPSSSLGTQTYIC |
| | VL | NVNHKPSNTKVDKKVEPKSCDKTHTCPPCPAPEAAGGPSVFLFPPKPKDTLMISRTPEVTCVVVDVSH |
| | (D1AA4920) | EDPEVKFNWYVDGVEVHNAKTKPREEQYNSTYRVVSVLTVLHQDWLNGKEYKCKVSNKALGAPIEKTI |
| | | SKAKGQPREPQVCTLPPSRDELTKNQVSLSCAVKGFYPSDIAVEWESNGQPENNYKTTPPVLDSDGSF |
| | | FLVSKLTVDKSRWQQGNVFSCSVMHEALHNHYTQKSLSLSPGGGGGSGGGGSGGGGSGGGGSIQMTQS |
| | | PSSLSASVGDRVTITCQSSHSVYSDNDLAWYQQKPGKAPKLLIYQASKLASGVPSRFSGSGSGTDFTL |
| | | TISSLQPEDFATYYCLGGYDDESDTYGFGGGTKVEIK |
| 85 | P1AF0709 | QVQLVQSGAEVKKPGSSVKVSCKASGFNIKDTYMHWVRQAPGQGLEWMGRIDPANGNSKYVPKFQGRV |
| | HChole <CEA> | TITADTSTSTAYMELSSLRSEDTAVYYCAPFGYYVSDYAMAYWGQGTLVTVSSASTKGPSVFPLAPSS |
| | T84.66 without | KSTSGGTAALGCLVKDYFPEPVTVSWNSGALTSGVHTFPAVLQSSGLYSLSSVVTVPSSSLGTQTYIC |
| | linker and | NVNHKPSNTKVDKKVEPKSCDKTHTCPPCPAPEAAGGPSVFLFPPKPKDTLMISRTPEVTCVVVDVSH |
| | DOTAM | EDPEVKFNWYVDGVEVHNAKTKPREEQYNSTYRVVSVLTVLHQDWLNGKEYKCKVSNKALGAP |
| | | IEKTISKAKGQPREPQVCTLPPSRDELTKNQVSLSCAVKGFYPSDIAVEWESNGQPENNYKTTPPVLD |
| | | SDGSFFLVSKLTVDKSRWQQGNVFSCSVMHEALHNHYTQKSLSLSPG |
| 86 | P1AF0298 | QVQLVQSGAEVKKPGSSVKVSCKASGFNIKDTYMHWVRQAPGQGLEWMGRIDPANGNSKYVPKFQGRV |
| | HCHole <CEA> | TITADTSTSTAYMELSSLRSEDTAVYYCAPFGYYVSDYAMAYWGQGTLVTVSSASTKGPSVFPLAPSS |
| | T84.66 | KSTSGGTAALGCLVKDYFPEPVTVSWNSGALTSGVHTFPAVLQSSGLYSLSSVVTVPSSSLGTQTYIC |
| | (D1AE4687) | NVNHKPSNTKVDKKVEPKSCDKTHTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDWLNGKEYKCKVSNKALGAPIEKTI |
| | | SKAKGQPREPQVCTLPPSRDELTKNQVSLSCAVKGFYPSDIAVEWESNGQPENNYKTTPPVLDSDGSF |
| | | FLVSKLTVDKSRWQQGNVFSCSVMHEALHNHYTQKSLSLSPG |
| 87 | P1AF0298 | QVQLVQSGAEVKKPGSSVKVSCKASGFNIKDTYMHWVRQAPGQGLEWMGRIDPANGNSKYVPKFQGRVTITA |
| | HCknob <CEA> | DTSTSTAYMELSSLRSEDTAVYYCAPFGYYVSDYAMAYWGQGTLVTVSSASTKGPSVFPLAPSSKSTSGGTA |
| | T84.66 Dotam- | ALGCLVKDYFPEPVTVSWNSGALTSGVHTFPAVLQSSGLYSLSSVVTVPSSSLGTQTYICNVNHKPSNTKVD |
| | VH-AST | KKVEPKSCDKTHTCPPCPAPEAAGGPSVFLFPPKPKDTLMISRTPEVTCVVVDVSHEDPEVKFNWYVDGVEV |
| | (D1AE3668) | HNAKTKPREEQYNSTYRVVSVLTVLHQDWLNGKEYKCKVSNKALGAPIEKTISKAKGQPREPQVYTLPPCRD |
| | | ELTKNQVSLWCLVKGFYPSDIAVEWESNGQPENNYKTTPPVLDSDGSFFLYSKLTVDKSRWQQGNVFSCSVM |
| | | HEALHNHYTQKSLSLSPGGGGGSGGGGSGGGGSGGGGSVTLKESGPVLVKPTETLTLTCTVSGFSLSTYSMS |
| | | WIRQPPGKALEWLGFIGSRGDTYYASWAKGRLTISKDTSKSQVVLTMTNMDPVDTATYYCARERDPYGGGAY |
| | | PPHLWGRGTLVTVSSAST |
| 88 | P1AF0298 | QVQLVQSGAEVKKPGSSVKVSCKASGFNIKDTYMHWVRQAPGQGLEWMGRIDPANGNSKYVPKFQGRVTITA |
| | HCknob <CEA> | DTSTSTAYMELSSLRSEDTAVYYCAPFGYYVSDYAMAYWGQGTLVTVSSASTKGPSVFPLAPSSKSTSGGTA |
| | T84.66 without | ALGCLVKDYFPEPVTVSWNSGALTSGVHTFPAVLQSSGLYSLSSVVTVPSSSLGTQTYICNVNHKPSNTKVD |
| | linker and | KKVEPKSCDKTHTCPPCPAPEAAGGPSVFLFPPKPKDTLMISRTPEVTCVVVDVSHEDPEVKFNWYVDGVEV |
| | DOTAM | HNAKTKPREEQYNSTYRVVSVLTVLHQDWLNGKEYKCKVSNKALGAPIEKTISKAKGQPREPQVYTLPPCRD |
| | | ELTKNQVSLWCLVKGFYPSDIAVEWESNGQPENNYKTTPPVLDSDGSFFLYSKLTVDKSRWQQGNVFSCSVM |
| | | HEALHNHYTQKSLSLSPG |
| 89 | P1AF0709 and | EIVLTQSPATLSLSPGERATLSCRAGESVDIFGVGFLHWYQQKPGQAPRLLIYRASNRATGIPARFSGSGSG |
| | P1AF0298 light | TDFTLTISSLEPEDFAVYYCQQTNEDPYTFGQGTKLEIKRTVAAPSVFIFPPSDEQLKSGTASVVCLLNNFY |
| | chain | PREAKVQWKVDNALQSGNSQESVTEQDSKDSTYSLSSTLTLSKADYEKHKVYACEVTHQGLSSPVTKSFNRG |
| | (D1AA4120) | EC |
| 90 | P1AF0710 | QVQLVQSGAEVKKPGSSVKVSCKASGGTFSYYAISWVRQAPGQGLEWMGGILPAFGAANYAQKFQGRVTITAD |
| | HCknob <CEA> | KSTSTAYMELSSLRSEDTAVYYCARLPPLPGAGLDYWGQGTTVTVSSASTKGPSVFPLAPSSKSTSGGTAALG |
| | 28A9 | CLVKDYFPEPVTVSWNSGALTSGVHTFPAVLQSSGLYSLSSVVTVPSSSLGTQTYICNVNHKPSNTKVDKKVE |
| | (D1AE4690) | PKSCDKTHTCPPCPAPEAAGGPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALGAPIEKTISKAKGQPREPQVYTLPPCRDELTKNQ |
| | | VSLWCLVKGFYPSDIAVEWESNGQPENNYKTTPPVLDSDGSFFLYSKLTVDKSRWQQGNVFSCSVMHEALHNH |
| | | YTQKSLSLSPG |
| 91 | P1AF0710 | QVQLVQSGAEVKKPGSSVKVSCKASGGTFSYYAISWVRQAPGQGLEWMGGILPAFGAANYAQKFQGRVTITAD |
| | HChole <CEA> | KSTSTAYMELSSLRSEDTAVYYCARLPPLPGAGLDYWGQGTTVTVSSASTKGPSVFPLAPSSKSTSGGTAALG |
| | 28A9 Dotam- | CLVKDYFPEPVTVSWNSGALTSGVHTFPAVLQSSGLYSLSSVVTVPSSSLGTQTYICNVNHKPSNTKVDKKVE |
| | VL | PKSCDKTHTCPPCPAPEAAGGPSVFLFPPKPKDTLMISRTPEVTCVVVDVSHEDPEVKFNWYVDGVEVHNAKT |
| | (D1AC3172) | KPREEQYNSTYRVVSVLTVLHQDWLNGKEYKCKVSNKALGAPIEKTISKAKGQPREPQVCTLPPSRDELTKNQ |
| | | VSLSCAVKGFYPSDIAVEWESNGQPENNYKTTPPVLDSDGSFFLVSKLTVDKSRWQQGNVFSCSVMHEALHNH |
| | | YTQKSLSLSPGGGGGSGGGGSGGGGSGGGGSIQMTQSPSSLSASVGDRVTITCQSSHSVYSDNDLAWYQQKPG |
| | | KAPKLLIYQASKLASGVPSRFSGSGSGTDFTLTISSLQPEDFATYYCLGGYDDESDTYGFGGGTKVEIK |
| 92 | P1AF0710 | QVQLVQSGAEVKKPGSSVKVSCKASGGTFSYYAISWVRQAPGQGLEWMGGILPAFGAANYAQKFQGRVTITAD |
| | HChole <CEA> | KSTSTAYMELSSLRSEDTAVYYCARLPPLPGAGLDYWGQGTTVTVSSASTKGPSVFPLAPSSKSTSGGTAALG |
| | 28A9 without | CLVKDYFPEPVTVSWNSGALTSGVHTFPAVLQSSGLYSLSSVVTVPSSSLGTQTYICNVNHKPSNTKVDKKVE |
| | linker or | PKSCDKTHTCPPCPAPEAAGGPSVFLFPPKPKDTLMISRTPEVTCVVVDVSHEDPEVKFNWYVDGVEVHNAKT |
| | DOTAM | KPREEQYNSTYRVVSVLTVLHQDWLNGKEYKCKVSNKALGAPIEKTISKAKGQPREPQVCTLPPSRDELTKNQ |
| | | VSLSCAVKGFYPSDIAVEWESNGQPENNYKTTPPVLDSDGSFFLVSKLTVDKSRWQQGNVFSCSVMHEALHNH |
| | | YTQKSLSLSPG |
| 93 | P1AF0711 | QVQLVQSGAEVKKPGSSVKVSCKASGGTFSYYAISWVRQAPGQGLEWMGGILPAFGAANYAQKFQGRVTITAD |
| | HChole <CEA> | KSTSTAYMELSSLRSEDTAVYYCARLPPLPGAGLDYWGQGTTVTVSSASTKGPSVFPLAPSSKSTSGGTAALG |
| | 28A9 | CLVKDYFPEPVTVSWNSGALTSGVHTFPAVLQSSGLYSLSSVVTVPSSSLGTQTYICNVNHKPSNTKVDKKV |
| | (D1AE4689) | EPKSCDKTHTCPPCPAPEAAGGPSVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALGAPIEKTISKAKGQPREPQVCTLPPSRDELT |
| | | KNQVSLSCAVKGFYPSDIAVEWESNGQPENNYKTTPPVLDSDGSFFLVSKLTVDKSRWQQGNVFSCSVMHEA |
| | | LHNHYTQKSLSLSPG |
| 94 | P1AF0711 | QVQLVQSGAEVKKPGSSVKVSCKASGGTFSYYAISWVRQAPGQGLEWMGGILPAFGAANYAQKFQGRVTITAD |
| | HCknob <CEA> | KSTSTAYMELSSLRSEDTAVYYCARLPPLPGAGLDYWGQGTTVTVSSASTKGPSVFPLAPSSKSTSGGTAAL |
| | 28A9 Dotam- | GCLVKDYFPEPVTVSWNSGALTSGVHTFPAVLQSSGLYSLSSVVTVPSSSLGTQTYICNVNHKPSNTKVDKK |
| | VH-AST | VEPKSCDKTHTCPPCPAPEAAGGPSVFLFPPKPKDTLMISRTPEVTCVVVDVSHEDPEVKFNWYVDGVEVHN |
| | (D1AE3671) | AKTKPREEQYNSTYRVVSVLTVLHQDWLNGKEYKCKVSNKALGAPIEKTISKAKGQPREPQVYTLPPCRDEL |
| | | TKNQVSLWCLVKGFYPSDIAVEWESNGQPENNYKTTPPVLDSDGSFFLYSKLTVDKSRWQQGNVFSCSVMHE |
| | | ALHNHYTQKSLSLSPGGGGGSGGGGSGGGGSGGGGSVTLKESGPVLVKPTETLTLTCTVSGFSLSTYSMSWI |
| | | RQPPGKALEWLGFIGSRGDTYYASWAKGRLTISKDTSKSQVVLTMTNMDPVDTATYYCARERDPYGGGAYPP |
| | | HLWGRGTLVTVSSAST |
| 95 | P1AF0711 | QVQLVQSGAEVKKPGSSVKVSCKASGGTFSYYAISWVRQAPGQGLEWMGGILPAFGAANYAQKFQGRVTITAD |
| | HCknob <CEA> | KSTSTAYMELSSLRSEDTAVYYCARLPPLPGAGLDYWGQGTTVTVSSASTKGPSVFPLAPSSKSTSGGTAAL |
| | 28A9 without | GCLVKDYFPEPVTVSWNSGALTSGVHTFPAVLQSSGLYSLSSVVTVPSSSLGTQTYICNVNHKPSNTKVDKK |
| | linker and | VEPKSCDKTHTCPPCPAPEAAGGPSVFLFPPKPKDTLMISRTPEVTCVVVDVSHEDPEVKFNWYVDGVEVHN |
| | DOTAM | AKTKPREEQYNSTYRVVSVLTVLHQDWLNGKEYKCKVSNKALGAPIEKTISKAKGQPREPQVYTLPPCRDEL |
| | | TKNQVSLWCLVKGFYPSDIAVEWESNGQPENNYKTTPPVLDSDGSFFLYSKLTVDKSRWQQGNVFSCSVMHE |
| | | ALHNHYTQKSLSLSPG |
| 96 | P1AF0710 and | DIQMTQSPSTLSASVGDRVTITCRASQSISSWLAWYQQKPGKAPKLLIYDASSLESGVPSRFSGSGSGTEFT |
| | P1AF0711 light | LTISSLQPDDFATYYCQQNTQYPMTFGQGTKVEIKRTVAAPSVFIFPPSDEQLKSGTASVVCLLNNFYPREA |
| | chain | KVQWKVDNALQSGNSQESVTEQDSKDSTYSLSSTLTLSKADYEKHKVYACEVTHQGLSSPVTKSFNRGEC |
| | (D1AA2299) | |
| 97 | P1AF0712 | QVQLVQSGAEVKKPGASVKVSCKASGYTFTEFGMNWVRQAPGQGLEWMGWINTKTGEATYVEEFKGRVTFTT |
| | HCknob <CEA> | DTSTSTAYMELRSLRSDDTAVYYCARWDFAYYVEAMDYWGQGTTVTVSSASTKGPSVFPLAPSSKSTSGGTA |
| | CH1A1A | ALGCLVKDYFPEPVTVSWNSGALTSGVHTFPAVLQSSGLYSLSSVVTVPSSSLGTQTYICNVNHKPSNTKVD |
| | (D1AC4023) | KKVEPKSCDKTHTCPPCPAPEAAGGPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALGAPIEKTISKAKGQPREPQVYTLPPCRD |
| | | ELTKNQVSLWCLVKGFYPSDIAVEWESNGQPENNYKTTPPVLDSDGSFFLYSKLTVDKSRWQQGNVFSCSVM |
| | | HEALHNHYTQKSLSLSPG |
| 98 | P1AF0712 | QVQLVQSGAEVKKPGASVKVSCKASGYTFTEFGMNWVRQAPGQGLEWMGWINTKTGEATYVEEFKGRVTFTT |
| | HChole <CEA> | DTSTSTAYMELRSLRSDDTAVYYCARWDFAYYVEAMDYWGQGTTVTVSSASTKGPSVFPLAPSSKSTSGGTAA |
| | CH1A1A | LGCLVKDYFPEPVTVSWNSGALTSGVHTFPAVLQSSGLYSLSSVVTVPSSSLGTQTYICNVNHKPSNTKVDK |
| | DOTA-VL | KVEPKSCDKTHTCPPCPAPEAAGGPSVFLFPPKPKDTLMISRTPEVTCVVVDVSHEDPEVKFNWYVDGVEVH |
| | (D1AE4684) | NAKTKPREEQYNSTYRVVSVLTVLHQDWLNGKEYKCKVSNKALGAPIEKTISKAKGQPREPQVCTLPPSRD |
| | | ELTKNQVSLSCAVKGFYPSDIAVEWESNGQPENNYKTTPPVLDSDGSFFLVSKLTVDKSRWQQGNVFSCSVM |
| | | HEALHNHYTQKSLSLSPGGGGGSGGGGSGGGGSGGGGSQAVVIQESALTTPPGETVTLTCGSSTGAVTASNY |
| | | ANWVQEKPDHLFTGLIGGHNNRPPGVPARFSGSLIGDKAALTIAGTQTEDEAIYFCALWYSDHWVIGGGTKL |
| | | TVL |
| 99 | P1AF0712 | QVQLVQSGAEVKKPGASVKVSCKASGYTFTEFGMNWVRQAPGQGLEWMGWINTKTGEATYVEEFKGRVTFTT |
| | HChole <CEA> | DTSTSTAYMELRSLRSDDTAVYYCARWDFAYYVEAMDYWGQGTTVTVSSASTKGPSVFPLAPSSKSTSGGTAA |
| | without linker | LGCLVKDYFPEPVTVSWNSGALTSGVHTFPAVLQSSGLYSLSSVVTVPSSSLGTQTYICNVNHKPSNTKVDK |
| | or | KVEPKSCDKTHTCPPCPAPEAAGGPSVFLFPPKPKDTLMISRTPEVTCVVVDVSHEDPEVKFNWYVDGVEVH |
| | DOTA | NAKTKPREEQYNSTYRVVSVLTVLHQDWLNGKEYKCKVSNKALGAPIEKTISKAKGQPREPQVCTLPPSRDE |
| | | LTKNQVSLSCAVKGFYPSDIAVEWESNGQPENNYKTTPPVLDSDGSFFLVSKLTVDKSRWQQGNVFSCSVM |
| | | HEALHNHYTQKSLSLSPG |
| 100 | P1AF0713 | QVQLVQSGAEVKKPGASVKVSCKASGYTFTEFGMNWVRQAPGQGLEWMGWINTKTGEATYVEEFKGRVTFTT |
| | HCHole <CEA> | DTSTSTAYMELRSLRSDDTAVYYCARWDFAYYVEAMDYWGQGTTVTVSSASTKGPSVFPLAPSSKSTSGGTAA |
| | CH1A1A | LGCLVKDYFPEPVTVSWNSGALTSGVHTFPAVLQSSGLYSLSSVVTVPSSSLGTQTYICNVNHKPSNTKVDK |
| | (D1AC4022) | KVEPKSCDKTHTCPPCPAPEAAGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALGAPIEKTISKAKGQPREPQVCTLPPSRDE |
| | | LTKNQVSLSCAVKGFYPSDIAVEWESNGQPENNYKTTPPVLDSDGSFFLVSKLTVDKSRWQQGNVFSCSVMH |
| | | EALHNHYTQKSLSLSPG |
| 101 | P1AF0713 | QVQLVQSGAEVKKPGASVKVSCKASGYTFTEFGMNWVRQAPGQGLEWMGWINTKTGEATYVEEFKGRVTFTT |
| | HCknob <CEA> | DTSTSTAYMELRSLRSDDTAVYYCARWDFAYYVEAMDYWGQGTTVTVSSASTKGPSVFPLAPSSKSTSGGTAA |
| | CH1A1A | LGCLVKDYFPEPVTVSWNSGALTSGVHTFPAVLQSSGLYSLSSVVTVPSSSLGTQTYICNVNHKPSNTKVDK |
| | DOTA-VH-AST | KVEPKSCDKTHTCPPCPAPEAAGGPSVFLFPPKPKDTLMISRTPEVTCVVVDVSHEDPEVKFNWYVDGVEVH |
| | (D1AE3670) | NAKTKPREEQYNSTYRVVSVLTVLHQDWLNGKEYKCKVSNKALGAPIEKTISKAKGQPREPQVYTLPPCRDE |
| | | LTKNQVSLWCLVKGFYPSDIAVEWESNGQPENNYKTTPPVLDSDGSFFLYSKLTVDKSRWQQGNVFSCSVMH |
| | | EALHNHYTQKSLSLSPGGGGGSGGGGSGGGGSGGGGSHVKLQESGPGLVQPSQSLSLTCTVSGFSLTDYGVH |
| | | WVRQSPGKGLEWLGVIWSGGGTAYNTALISRLNIYRDNSKNQVFLEMNSLQAEDTAMYYCARRGSYPYNYFD |
| | | AWGQGTTVTVSSAST |
| 102 | P1AF0713 | QVQLVQSGAEVKKPGASVKVSCKASGYTFTEFGMNWVRQAPGQGLEWMGWINTKTGEATYVEEFKGRVTFTT |
| | HCknob <CEA> | DTSTSTAYMELRSLRSDDTAVYYCARWDFAYYVEAMDYWGQGTTVTVSSASTKGPSVFPLAPSSKSTSGGTAA |
| | CH1A1A | LGCLVKDYFPEPVTVSWNSGALTSGVHTFPAVLQSSGLYSLSSVVTVPSSSLGTQTYICNVNHKPSNTKVDK |
| | without linker | KVEPKSCDKTHTCPPCPAPEAAGGPSVFLFPPKPKDTLMISRTPEVTCVVVDVSHEDPEVKFNWYVDGVEVH |
| | and DOTA | NAKTKPREEQYNSTYRVVSVLTVLHQDWLNGKEYKCKVSNKALGAPIEKTISKAKGQPREPQVYTLPPCRDE |
| | | LTKNQVSLWCLVKGFYPSDIAVEWESNGQPENNYKTTPPVLDSDGSFFLYSKLTVDKSRWQQGNVFSCSVMH |
| | | EALHNHYTQKSLSLSPG |
| 103 | P1AF0712 and | DIQMTQSPSSLSASVGDRVTITCKASAAVGTYVAWYQQKPGKAPKLLIYSASYRKRGVPSRFSGSGSGTDFT |
| | P1AF0713 light | LTISSLQPEDFATYYCHQYYTYPLFTFGQGTKLEIKRTVAAPSVFIFPPSDEQLKSGTASVVCLLNNFYPRE |
| | chain | AKVQWKVDNALQSGNSQESVTEQDSKDSTYSLSSTLTLSKADYEKHKVYACEVTHQGLSSPVTKSFNRGEC |
| | (D1AA3384) | |
| 104 | P1AF8284 and | EVQLLESGGGLVQPGGSLRLSCAASGFTFSKYAMAWVRQAPGKGLEWVASISTGGVNTYYADSVKGRFTISR |
| | P1AF8285 | DNSKNTLYLQMNSLRAEDTAVYYCATHTGDYFDYWGQGTMVTVSSASTKGPSVFPLAPSSKSTSGGTAALG |
| | HCknob | CLVKDYFPEPVTVSWNSGALTSGVHTFPAVLQSSGLYSLSSVVTVPSSSLGTQTYICNVNHKPSNTKVDKKV |
| | <GPRC5D> | EPKSCDKTHTCPPCPAPEAAGGPSVFLFPPKPKDTLMISRTPEVTCVVVDVSHEDPEVKFNWYVDGVEVHN |
| | (D1AF6517) | AKTKPREEQYNSTYRVVSVLTVLHQDWLNGKEYKCKVSNKALGAPIEKTISKAKGQPREPQVYTLPPCRDEL |
| | | TKNQVSLWCLVKGFYPSDIAVEWESNGQPENNYKTTPPVLDSDGSFFLYSKLTVDKSRWQQGNVFSCSVMH |
| | | EALHNHYTQKSLSLSPGK |
| 105 | P1AF8284 | DKTHTCPPCPAPEAAGGPSVFLFPPKPKDTLMISRTPEVTCVVVDVSHEDPEVKFNWYVDGVEVHNAKTKPR |
| | HChole Dotam- | EEQYNSTYRVVSVLTVLHQDWLNGKEYKCKVSNKALGAPIEKTISKAKGQPREPQVCTLPPSRDELTKNQV |
| | VL | SLSCAVKGFYPSDIAVEWESNGQPENNYKTTPPVLDSDGSFFLVSKLTVDKSRWQQGNVFSCSVMHEALHN |
| | (D1AG3592) | HYTQKSLSLSPGKGGGGSGGGGSGGGGSGGGGSSIQMTQSPSSLSASVGDRVTITCQSSHSVYSDNDLAWY |
| | | QQKPGKAPKLLIYQASKLASGVPSRFSGSGSGTDFTLTISSLQPEDFATYYCLGGYDDESDTYGFGGGTKV |
| | | EIK |
| 106 | P1AF8285 | DKTHTCPPCPAPEAAGGPSVFLFPPKPKDTLMISRTPEVTCVVVDVSHEDPEVKFNWYVDGVEVHNAKTKPR |
| | Hhole Dotam- | EEQYNSTYRVVSVLTVLHQDWLNGKEYKCKVSNKALGAPIEKTISKAKGQPREPQVCTLPPSRDELTKNQVS |
| | VHA | LSCAVKGFYPSDIAVEWESNGQPENNYKTTPPVLDSDGSFFLVSKLTVDKSRWQQGNVFSCSVMHEALHNH |
| | (D1AG3591) | YTQKSLSLSPGKGGGGSGGGGSGGGGSGGGGSVTLKESGPVLVKPTETLTLTCTVSGFSLSTYSMSWIRQPP |
| | | GKALEWLGFIGSRGDTYYASWAKGRLTISKDTSKSQVVLTMTNMDPVDTATYYCARERDPYGGGAYPPHLWG |
| 107 | P1AF8284 and | RGTLVTVSSAEIVLTQSPGTLSLSPGERATLSCRASQSVSISGINLMNWYQQKPGQQPKLLIYHASILASGI |
| | P1AF8285 light | PDRFSGSGSGTDFTLTISRLEPEDFAVYYCQQTRESPLTFGQGTRLEIKRTVAAPSVFIFPPSDEQLKSGTA |
| | chain | SVVCLLNNFYPREAKVQWKVDNALQSGNSQESVTEQDSKDSTYSLSSTLTLSKADYEKHKVYACEVTHQGL |
| | (D1AF6469) | SSPVTKSFNRGEC |
| 108 | P1AF8286 and | EVQLLESGGGLVQPGGSLRLSCAASGFTFSSYAMSWVRQAPGKGLEWVSAIIGSGASTYYADSVKGRFTISR |
| | P1AF8287 | DNSKNTLYLQMNSLRAEDTAVYYCAKGWFGGFNYWGQGTLVTVSSASTKGPSVFPLAPSSKSTSGGTAAL |
| | HCknob <FAP> | GCLVKDYFPEPVTVSWNSGALTSGVHTFPAVLQSSGLYSLSSVVTVPSSSLGTQTYICNVNHKPSNTKVDK |
| | 4B9 | KVEPKSCDKTHTCPPCPAPEAAGGPSVFLFPPKPKDTLMISRTPEVTCVVVDVSHEDPEVKFNWYVDGVEVH |
| | (D1AF6515) | NAKTKPREEQYNSTYRVVSVLTVLHQDWLNGKEYKCKVSNKALGAPIEKTISKAKGQPREPQVYTLPPCRD |
| | | ELTKNQVSLWCLVKGFYPSDIAVEWESNGQPENNYKTTPPVLDSDGSFFLYSKLTVDKSRWQQGNVFSCSVM |
| | | HEALHNHYTQKSLSLSPGK |
| 109 | P1AF8286 | DKTHTCPPCPAPEAAGGPSVFLFPPKPKDTLMISRTPEVTCVVVDVSHEDPEVKFNWYVDGVEVHNAKTKPR |
| | HChole Dotam- | EEQYNSTYRVVSVLTVLHQDWLNGKEYKCKVSNKALGAPIEKTISKAKGQPREPQVCTLPPSRDELTKNQVS |
| | VL | LSCAVKGFYPSDIAVEWESNGQPENNYKTTPPVLDSDGSFFLVSKLTVDKSRWQQGNVFSCSVMHEALHNH |
| | (D1AG3592) | YTQKSLSLSPGKGGGGSGGGGSGGGGSGGGGSSIQMTQSPSSLSASVGDRVTITCQSSHSVYSDNDLAWYQ |
| | | QKPGKAPKLLIYQASKLASGVPSRFSGSGSGTDFTLTISSLQPEDFATYYCLGGYDDESDTYGFGGGTKVEIK |
| 110 | P1AF8287 | DKTHTCPPCPAPEAAGGPSVFLFPPKPKDTLMISRTPEVTCVVVDVSHEDPEVKFNWYVDGVEVHNAKTKPR |
| | HChole Dotam- | EEQYNSTYRVVSVLTVLHQDWLNGKEYKCKVSNKALGAPIEKTISKAKGQPREPQVCTLPPSRDELTKNQV |
| | VHA | SLSCAVKGFYPSDIAVEWESNGQPENNYKTTPPVLDSDGSFFLVSKLTVDKSRWQQGNVFSCSVMHEALHNH |
| | (D1AG3591) | YTQKSLSLSPGKGGGGSGGGGSGGGGSGGGGSVTLKESGPVLVKPTETLTLTCTVSGFSLSTYSMSWIRQPP |
| | | GKALEWLGFIGSRGDTYYASWAKGRLTISKDTSKSQVVLTMTNMDPVDTATYYCARERDPYGGGAYPPHLWG |
| | | RGTLVTVSSA |
| 111 | P1AF8286 and | EIVLTQSPGTLSLSPGERATLSCRASQSVTSSYLAWYQQKPGQAPRLLINVGSRRATGIPDRFSGSGSGTD |
| | P1AF8287 light | FTLTISRLEPEDFAVYYCQQGIMLPPTFGQGTKVEIKRTVAAPSVFIFPPSDEQLKSGTASVVCLLNNFYP |
| | chain | REAKVQWKVDNALQSGNSQESVTEQDSKDSTYSLSSTLTLSKADYEKHKVYACEVTHQGLSSPVTKSFNRGEC |
| | (D1AB9974) | |
| 112 | P1AF7782 and | QVQLVQSGAEVKKPGASVKVSCKASGYTFTEFGMNWVRQAPGQGLEWMGWINTKTGEATYVEEFKGRVTFTT |
| | P1AF7784 | DTSTSTAYMELRSLRSDDTAVYYCARWDFAYYVEAMDYWGQGTTVTVSSASTKGPSVFPLAPSSKSTSGGTAA |
| | HCknob <CEA> | LGCLVKDYFPEPVTVSWNSGALTSGVHTFPAVLQSSGLYSLSSVVTVPSSSLGTQTYICNVNHKPSNTKVDK |
| | CH1A1A | KVEPKSCDKTHTCPPCPAPEAAGGPSVFLFPPKPKDTLMISRTPEVTCVVVDVSHEDPEVKFNWYVDGVEVH |
| | (D1AD3419) | NAKTKPREEQYNSTYRVVSVLTVLHQDWLNGKEYKCKVSNKALGAPIEKTISKAKGQPREPQVYTLPPCRDE |
| | | LTKNQVSLWCLVKGFYPSDIAVEWESNGQPENNYKTTPPVLDSDGSFFLYSKLTVDKSRWQQGNVFSCSVM |
| | | HEALHNHYTQKSLSLSPGK |
| 113 | P1AF7782 | SIQMTQSPSSLSASVGDRVTITCQSSHSVYSDNDLAWYQQKPGKAPKLLIYQASKLASGVPSRFSGSGSGT |
| | HChole Dotam- | DFTLTISSLQPEDFATYYCLGGYDDESDTYGFGGGTKVEIKGGGGSGGGGSGGGGSGGSGGDKTHTCPPCP |
| | VL | APEAAGGPSVFLFPPKPKDTLMISRTPEVTCVVVDVSHEDPEVKFNWYVDGVEVHNAKTKPREEQYNSTYR |
| | (D1AG2237) | VVSVLTVLHQDWLNGKEYKCKVSNKALGAPIEKTISKAKGQPREPQVCTLPPSRDELTKNQVSLSCAVKGF |
| | | YPSDIAVEWESNGQPENNYKTTPPVLDSDGSFFLVSKLTVDKSRWQQGNVFSCSVMHEALHNHYTQKSLSL |
| | | SPGK |
| 114 | P1AF7784 | GVTLKESGPVLVKPTETLTLTCTVSGFSLSTYSMSWIRQPPGKALEWLGFIGSRGDTYYASWAKGRLTISK |
| | HChole Dotam- | DTSKSQVVLTMTNMDPVDTATYYCARERDPYGGGAYPPHLWGRGTLVTVSSGGGGSGGGGSGGGGSGGSG |
| | VH | GDKTHTCPPCPAPEAAGGPSVFLFPPKPKDTLMISRTPEVTCVVVDVSHEDPEVKFNWYVDGVEVHNAKTK |
| | (D1AG2236) | PREEQYNSTYRVVSVLTVLHQDWLNGKEYKCKVSNKALGAPIEKTISKAKGQPREPQVCTLPPSRDELTK |
| | | NQVSLSCAVKGFYPSDIAVEWESNGQPENNYKTTPPVLDSDGSFFLVSKLTVDKSRWQQGNVFSCSVMHEA |
| | | LHNHYTQKSLSLSPGK |
| 115 | P1AF7782 and | DIQMTQSPSSLSASVGDRVTITCKASAAVGTYVAWYQQKPGKAPKLLIYSASYRKRGVPSRFSGSGSGTD |
| | P1AF7784 light | FTLTISSLQPEDFATYYCHQYYTYPLFTFGQGTKLEIKRTVAAPSVFIFPPSDEQLKSGTASVVCLLNNFY |
| | chain | PREAKVQWKVDNALQSGNSQESVTEQDSKDSTYSLSSTLTLSKADYEKHKVYACEVTHQGLSSPVTKSFNR |
| | (D1AD3421) | GEC |
| 116 | heavy chain | DSYMH |
| | CDR1 <CEA> | |
| | MFE23 | |
| 117 | heavy chain | WIDPENGDTEYAPKFQG |
| | CDR2 <CEA> | |
| | MFE23 | |
| 118 | heavy chain | WIDPENGGTNYAQKFQG |
| | CDR2 <CEA> | |
| | MFE23-H26 | |
| 119 | heavy chain | GTPTGPYYFDY |
| | CDR3 <CEA> | |
| | MFE23 | |
| 120 | light chain | SASSSVSYMH |
| | CDR1 <CEA> | |
| | MFE23 | |
| 121 | light chain | RASSSVSYMH |
| | CDR1 <CEA> | |
| | MFE23-L24, | |
| | L25 | |
| 122 | light chain | RASQSISSYM |
| | CDR1 <CEA> | |
| | MFE23-L26 | |
| 123 | light chain | STSNLAS |
| | CDR2 <CEA> | |
| | MFE23 | |
| 124 | Light chain | YTSNLAS |
| | CDR2 <CEA> | |
| | MFE23-L26 | |
| 125 | Light chain | STSSLQS |
| | CDR2 <CEA> | |
| | MFE23-L29 | |
| 126 | light chain | QQRSSYPLT |
| | CDR3 <CEA> | |
| | MFE23 | |
| 127 | Heavy chain | QVKLQQSGAELVRSGTSVKLSCTASGFNIKDSYMHWLRQGPEQGLEWIGWIDPENGDTEYAPKFQGKATFT |
| | variable domain | TDTSSNTAYLQLSSLTSEDTAVYYCNEGTPTGPYYFDYWGQGTTVTVSS |
| | <CEA> MFE23 | |
| 128 | Light chain | ENVLTQSPAIMSASPGEKVTITCSASSSVSYMHWFQQKPGTSPKLWIYSTSNLASGVPARFSGSGSGTSYS |
| | variable domain | LTISRMEAEDAATYYCQQRSSYPLTFGAGTKLELK |
| | <CEA> MFE23 | |
| 129 | MFE-H24 | QVQLVQSGAEVKKPGASVKVSCKASGFNIKDSYMHWVRQAPGQGLEWMGWIDPENGDTEYAPKFQGRVTMT |
| | | TDTSISTAYMELSRLRSDDTAVYYCNEGTPTGPYYFDYWGQGTLVTVSS |
| 130 | MFE-H25 | QVQLVQSGAEVKKPGASVKVSCKASGYTFKDSYMHWVRQAPGQGLEWMGWIDPENGDTEYAPKFQGRVTMT |
| | | TDTSISTAYMELSRLRSDDTAVYYCNEGTPTGPYYFDYWGQGTLVTVSS |
| 131 | MFE-H26 | QVQLVQSGAEVKKPGASVKVSCKASGFNIKDSYMHWVRQAPGQGLEWMGWIDPENGGTNYAQKFQGRVTMT |
| | | TDTSISTAYMELSRLRSDDTAVYYCNEGTPTGPYYFDYWGQGTLVTVSS |
| 132 | MFE-H27 | QVQLVQSGAEVKKPGASVKVSCKASGFNIKDSYMHWVRQAPGQGLEWMGWIDPENGDTEYAPKFQGRVTMT |
| | | TDTSISTAYMELSRLRSDDTAVYYCARGTPTGPYYFDYWGQGTLVTVSS |
| 133 | MFE-H28 | QVQLVQSGAEVKKPGASVKVSCKASGFNIKDSYMHWVRQAPGQGLEWMGWIDPENGDTEYAPKFQGRVTMT |
| | | RDTSISTAYMELSRLRSDDTAVYYCNEGTPTGPYYFDYWGQGTLVTVSS |
| 134 | MFE-H29 | QVQLVQSGAEVKKPGSSVKVSCKASGFNIKDSYMHWVRQAPGQGLEWMGWIDPENGDTEYAPKFQGRVTITT |
| | | DESTSTAYMELSSLRSEDTAVYYCNEGTPTGPYYFDYWGQGTLVTVSS |
| 135 | MFE-L24 | DIQMTQSPSSLSASVGDRVTITCRASSSVSYMHWYQQKPGKAPKLLIYSTSNLASGVPSRFSGSGSGTDFT |
| | | LTISSLQPEDFATYYCQQRSSYPLTFGGGTKLEIK |
| 136 | MFE-L25 | EIQMTQSPSSLSASVGDRVTITCRASSSVSYMHWYQQKPGKAPKLLIYSTSNLASGVPSRFSGSGSGTDFT |
| | | LTISSLQPEDFATYYCQQRSSYPLTFGGGTKLEIK |
| 137 | MFE-L26 | EIQMTQSPSSLSASVGDRVTITCRASQSISSYMHWYQQKPGKAPKLLIYSTSNLASGVPSRFSGSGSGTDF |
| | | TLTISSLQPEDFATYYCQQRSSYPLTFGGGTKLEIK |
| 138 | MFE-L27 | EIQMTQSPSSLSASVGDRVTITCRASSSVPYMHWYQQKPGKAPKLLIYSTSNLASGVPSRFSGSGSGTDFT |
| | | LTISSVQPEDFATYYCQQRSSYPLTFGGGTKLEIK |
| 139 | MFE-L28 | EIQMTQSPSSLSASVGDRVTITCRASSSVPYMHWLQQKPGKAPKLLIYSTSNLASGVPSRFSGSGSGTDFTL |
| | | TISSVQPEDFATYYCQQRSSYPLTFGGGTKLEIK |
| 140 | MFE-L29 | EIQMTQSPSSLSASVGDRVTITCRASSSVPYMHWLQQKPGKAPKLLIYSTSSLQSGVPSRFSGSGSGTDFT |
| | | LTISSVQPEDFATYYCQQRSSYPLTFGGGTKLEIK |
| 141 | A2 domain of | PKPFITSNNSNPVEDEDAVALTCEPEIQNTTYLWWVNNQSLPVSPRLQLSNDNRTLTLLSVTRNDVGP |
| | CEA | YECGIQNKLSVDHSDPVILN |
| 142 | A1 domain of | PKPSISSNNSKPVEDKDAVAFTCEPETQDATYLWWVNNQSLPVSPRLQLSNGNRTLTLFNVTRNDTAS |
| | CEA | YKCETQNPVSARRSDSVILN |

### IV. EXAMPLES

The following are examples of methods and compositions of the invention. It is understood that various other embodiments may be practiced, given the general description provided above.

### Glossary of Abbreviations

ADA Anti-drug antibody
AST Alanine, serine, threonine
BLI Bioluminescence imaging
BsAb Bispecific antibody
BW Body weight
CA Clearing agent
cDC Classical dendritic cell
CEA Carcinoembryonic antigen
CIT Cancer immunotherapy
DC Dendritic cell
DLN Draining lymph node
DOTAM 1,4,7,10-tetrakis(carbamoylmethyl)-1,4,7,10-tetraazacyclododecane
ID Injected dose
ELISA Enzyme-linked immunosorbent assay
FACS Fluorescence-activated cell sorting
FAP Fibroblast activation protein
GPRC5D G-protein coupled receptor family C group 5 member D
huCEA Human carcinoembryonic antigen
ID Injected dose
IFN Interferon
IL Interleukin
IP Intraperitoneal
IV Intravenous
Luc Luciferase
MCH Major histocompatibility complex
MFI Mean fluorescence intensity
MW Molecular weight
NBF Neutral buffered formalin
PBS Phosphate-buffered saline
PD Pharmacodynamic
pDC Plasmacytoid dendritic cell
p.i. Post injection
PMA Phorbol 12-myristate-13-acetate
PK Pharmacokinetic
PRIT Pretargeted radioimmunotherapy
RIT Radioimmunotherapy
RCF Relative centrifugal force (G-force)
ROI Region of interest
RT Room temperature
SC Subcutaneous
SCID Severe combined immunodeficiency
SD Standard deviation
SEM Standard error of the mean
SOPF Specific and opportunistic pathogen-free
SPLIT SeParated v-domains LInkage Technology
TA Target antigen
TGI Tumor growth inhibition
TR Tumor regression
Treg Regulatory T cell

### Example 1: Generation of CEA-Split-DOTAM VH/VL Antibodies

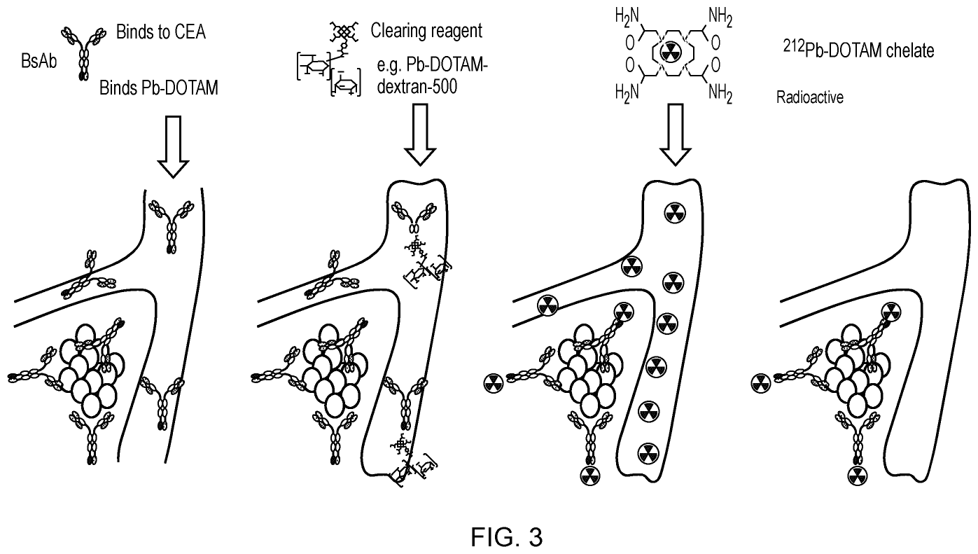

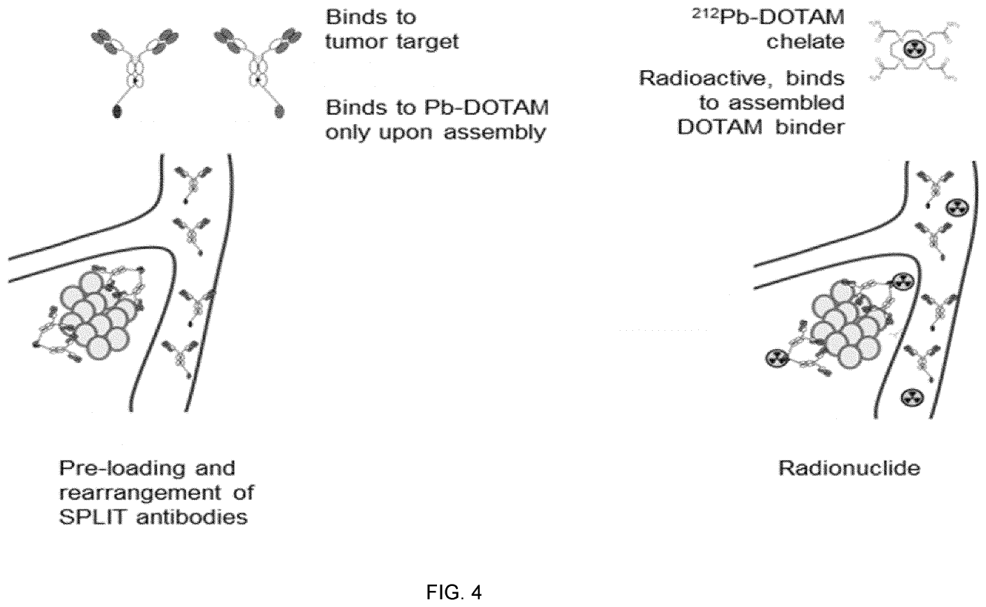

Methods of PRIT (Pretargeted radioimmunotherapy) using bispecific antibodies having a binding site for the target antigen and a binding site for the radiolabelled compound commonly use a clearing agent (CA) between the administrations of antibody and radioligand, to ensure effective targeting and high tumour-to-normal tissue absorbed dose ratios (see FIG. 3). In an example of one such method, injected BsAb is allowed sufficient time for penetrating into the tumours, generally 4-10 days, after which circulating BsAb is neutralized using a Pb-DOTAM-dextran-500 CA. The CA blocks ²¹²Pb-DOTAM binding to nontargeted BsAb without penetrating into the tumour, which would block the pretargeted sites. This pretargeting regimen allows efficient tumour accumulation of the subsequently administered radiolabelled chelate, ²¹²Pb-DOTAM.

However, in methods involving a clearing agent, the use of a CA introduces a further step to the method which is inefficient. Moreover, it can be important to choose the timing and dosing of the CA administration with care, which is a complicating factor.

To address the problems associated with use of a clearing agent, the present inventors have proposed a strategy of splitting the DOTAM VL and VH domains, such that they are found on separate antibodies.

The generation of exemplary split DOTAM VH/VL antibodies is discussed further below

### Generation of Plasmids for the Recombinant Expression of Antibody Heavy or Light Chains

Desired proteins were expressed by transient transfection of human embryonic kidney cells (HEK 293). For the expression of a desired gene/protein (e.g. full length antibody heavy chain, full length antibody light chain, or a full length antibody heavy chain containing an additional domain (e.g. an immunoglobulin heavy or light chain variable domain at its C-terminus) a transcription unit comprising the following functional elements was used:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence (SS),
a gene/protein to be expressed, and
the bovine growth hormone polyadenylation sequence (BGH pA).

In addition to the expression unit/cassette including the desired gene to be expressed the basic/standard mammalian expression plasmid contained
an origin of replication from the vector pUC18 which allows replication of this plasmid in E. coli, and
a beta-lactamase gene which confers ampicillin resistance in E. coli.

### a) Expression Plasmid for Antibody Heavy Chains

Antibody heavy chain encoding genes including C-terminal fusion genes comprising a complete and functional antibody heavy chain, followed by an additional antibody V-heavy or V-light domain was assembled by fusing a DNA fragment coding for the respective sequence elements (V-heavy or V-light) separated each by a G4Sx4 linker to the C-terminus of the CH3 domain of a human IgG molecule (VH-CH1-hinge-CH2-CH3-linker-VH or VH-CH1-hinge-CH2-CH3-linker-VL). Recombinant antibody molecules bearing one VH and one VL domain at the C-termini of the two CH3 domains, respectively, were expressed using the knob-into-hole technology.

The expression plasmids for the transient expression of an antibody heavy chain with a C-terminal VH or VL domain in HEK293 cells comprised besides the antibody heavy chain fragment with C-terminal VH or VL domain expression cassette, an origin of replication from the vector pUC18, which allows replication of this plasmid in E. coli, and a beta-lactamase gene which confers ampicillin resistance in E. coli. The transcription unit of the antibody heavy chain fragment with C-terminal VH or VL domain fusion gene comprises the following functional elements:
the immediate early enhancer and promoter from the human cytomegalovirus (P-CMV) including intron A,
   a human heavy chain immunoglobulin 5′-untranslated region (5′UTR),
   a murine immunoglobulin heavy chain signal sequence,
   an antibody heavy chain (VH-CH1-hinge-CH2-CH3-linker-VH or VH-CH1-hinge-CH2-CH3-linker-VL) encoding nucleic acid, and
   the bovine growth hormone polyadenylation sequence (BGH pA).

### b) Expression Plasmid for Antibody Light Chains

Antibody light chain encoding genes comprising a complete and functional antibody light chain was assembled by fusing a DNA fragment coding for the respective sequence elements.

The expression plasmid for the transient expression of an antibody light chain comprised besides the antibody light chain fragment an origin of replication from the vector pUC18, which allows replication of this plasmid in E. coli, and a beta-lactamase gene which confers ampicillin resistance in E. coli. The transcription unit of the antibody light chain fragment comprises the following functional elements:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
an antibody light chain (VL-CL) encoding nucleic acid, and
the bovine growth hormone polyadenylation sequence (BGH pA).

### Transient Expression of the Antibody Molecules

The antibody molecules were generated in transiently transfected HEK293 cells (human embryonic kidney cell line 293-derived) cultivated in F17 Medium (Invitrogen Corp.). For transfection “293-Free” Transfection Reagent (Novagen) was used. The respective antibody heavy- and light chain molecules as described above were expressed from individual expression plasmids. Transfections were performed as specified in the manufacturer's instructions. Immunoglobulin-containing cell culture supernatants were harvested three to seven (3-7) days after transfection. Supernatants were stored at reduced temperature (e.g. −80° C.) until purification.

General information regarding the recombinant expression of human immunoglobulins in e.g. HEK293 cells is given in: Meissner, P. et al., Biotechnol. Bioeng. 75 (2001) 197-203.

The PRIT Hemibodies (split antibodies) were purified by a MabSelect Sure (Affinity Chromatography) and followed by Superdex 200 (Size Exclusion Chromatography).

Sequences of exemplary antibodies are summarised below.

| Antibody | First heavy | Second heavy | Light |
|---|---|---|---|
| name | chain | chain | chain |
| P1AD8592 | SEQ ID | SEQ ID | SEQ ID |
| | NO: 30 | NO: 33 | NO: 34 |
| P1AD8749 | SEQ ID | SEQ ID | SEQ ID |
| | NO: 28 | NO: 32 | NO: 34 |
| P1AE4956 | SEQ ID | SEQ ID | SEQ ID |
| | NO: 51 | NO: 52 | NO: 54 |
| P1AE4957 | SEQ ID | SEQ ID | SEQ ID |
| | NO 55 | NO 56 | NO: 58 |

For the PRIT Split Antibody with DOTAM-VL-P1AD8592 5 mg with a concentration of 1.372 mg/mL and a purity >96% based on analytical SEC and CE-SDS were produced. For the PRIT Split Antibody with DOTAM-VH-P1AD8749 14 mg with a concentration of 2.03 mg/mL and a purity >91% based on analytical SEC and CE-SDS were produced.

Antibodies P1AE4956 and P1AE4957 were also generated. For the PRIT Split Antibody with DOTAM-VL-P1AE4957, 19 mg with a concentration of 2.6 mg/mL and a purity >81.6% based on analytical SEC and CE-SDS were produced. For the PRIT Split Antibody with DOTAM-VH-P1AE4956, 6.9 mg with a concentration of 1.5 mg/mL and a purity >90% based on analytical SEC and CE-SDS were produced. ESI-MS was used too confirm the identity of the PRIT hemibodies.

### Example 2: FACS Analysis of Split Antibody Functionality

To assess the functionality of the split antibodies or hemibodies, MKN-45 cells were detached from the culture vessel using accutase at 37° C. for 10 minutes. Subsequently, the cells were washed twice in PBS, and seeded into 96 well v-bottom plates to a final density of 4×10⁶ cells/well.

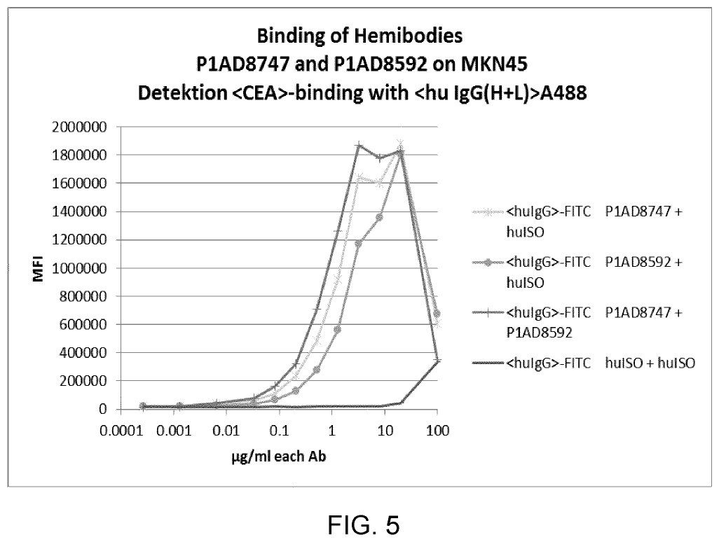

The hemibodies P1AD8749 and P1AD8592 and a human ISO control were mixed 1:1 added to the cells in concentrations as indicated in FIG. 5. Subsequently, the cells were incubated for 1 h on ice and washed twice in PBS. The cell pellet was resuspended and 40 μl/well of detection reagent was added, either <human IgG(H+L)>FITC, (10 μg/ml) or Pb_Dotam_FITC 1:100=>(10 μg/ml) in PBS/5% FCS. After 60 min incubation on ice, the cells were washed twice in PBS and resuspended in 200 μl PBS/5% FCS for measurement of FITC fluorescence using a FACS canto.

To assess the binding capability of the hemibodies to CEA on MKN-45 cells, they were detected using of antibodies using human IgG specific secondary antibodies (FIG. 5). As expected, no significant binding of the human ISO control is observed on these cells. When adjusted to the same IgG concentration, both hemibodies as well as the combination of both shows a dose dependent binding to MKN-45 cells, with a pronounced hook effect at very high concentrations as expected. This experiment demonstrates that the CEA binding is functional in the hemibodies.

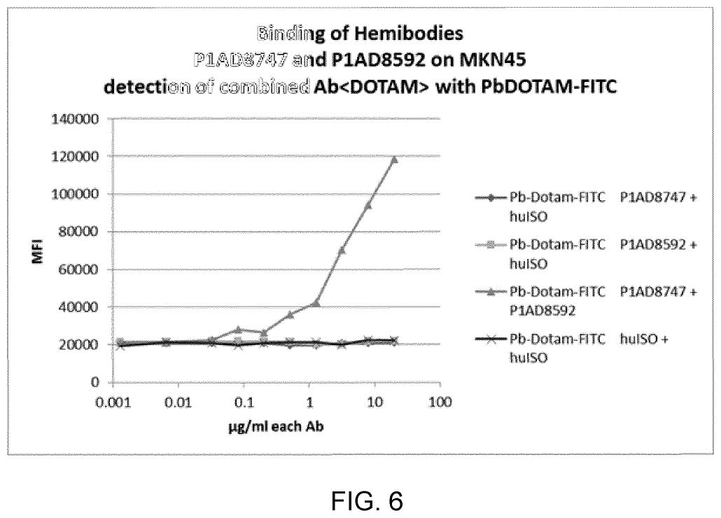

To assess the binding capability of the hemibodies to DOTAM, they were bound to the cells either in the presence of a human ISO control or their respective split antibody partner in a 1:1 ratio. After their binding to MKN-45 cells, the cells were washed to remove unbound antibody. Subsequently, Pb-DOTAM-FITC (fluorescently labelled Pb-DOTAM) was added to detect DOTAM binding competent cell bound antibodies (FIG. 6). As expected, no significant FITC is observed on these cells when one of the split antibody partners is combined with the of the human ISO control. Only a combination of both hemibodies in a 1:1 ratio shows a dose dependent FITC signal. This experiment shows that the DOTAM binding site becomes functional when both hemibodies come together on one cell.

### Example 3: In Vivo Studies

### Example 3a: Materials and Methods—General

All experimental protocols were reviewed and approved by the local authorities (Comité Regional d'Ethique de l'Experimentation Animale du Limousin [CREEAL], Laboratoire Départemental d'Analyses et de Recherches de la Haute-Vienne). Female severe combined immunodeficiency (SCID) mice (Charles River) were maintained under specific and opportunistic pathogen free (SOPF) conditions with daily cycles of light and darkness (12 h/12 h), in line with ethical guidelines. No manipulations were performed during the first 5 days after arrival, to allow the animals to acclimatize to the new environment. Animals were controlled daily for clinical symptoms and detection of adverse events.

Solid xenografts were established by subcutaneous (SC) injection of CEA-expressing tumor cells in cell culture media mixed 1:1 with Corning® Matrigel® basement membrane matrix (growth factor reduced; cat No. 354230). Tumor volumes were estimated through manual calipering 3 times per week, calculated according to the formula: volume 0.5× length×width². Additional tumor measurements were made as needed depending on the tumor growth rate.

Mice were euthanized before the scheduled endpoint if they showed signs of unamenable distress or pain due to tumor burden, side effects of the injections, or other causes. Indications of pain, distress, or discomfort include, but are not limited to, acute body weight (BW) loss, scruffy fur, diarrhea, hunched posture, and lethargy. The BW of treated animals was measured 3 times per week, with additional measurements as needed depending on the health status. Wet food was provided to all mice starting the day after the radioactive injection, for 7 days or until all individuals had recovered sufficiently from any acute BW loss. Mice whose BW loss exceeded 20% of their initial BW or whose tumor volume reached 3000 mm³ were euthanized immediately. Other factors taken into account for euthanasia for ethical reasons were tumor status (e.g. necrotic areas, blood/liquid leaking out, signs of automutilation) and general appearance of the animal (e.g. fur, posture, movement).

To minimize re-ingestion of radioactive urine/feces, all efficacy study mice were placed in cages with grilled floors for 4 hours after ²¹²Pb-DOTAM administration, before being transferred to new cages with standard bedding. All cages were then changed at 24 hours post injection (p.i.). This procedure was not performed for mice sacrificed for biodistribution purposes within 24 hours after the radioactive injection.

Blood was collected at the time of euthanasia from the venous sinus using retro-orbital bleeding on anesthetized mice, before termination through cervical dislocation followed by additional tissue harvest for radioactive measurements and/or histological analysis, as mandated by the protocols. Unexpected or abnormal conditions were documented. Tissues collected for formalin fixation were immediately put in 10% neutral buffered formalin (4° C.) and then transferred to phosphate-buffered saline (PBS; 4° C.) after 5 days. Organs and tissues collected for biodistribution purposes were weighed and measured for radioactivity using a 2470 WIZARD² automatic gamma counter (PerkinElmer), and the percent injected dose per gram of tissue (% ID/g) subsequently calculated, including corrections for decay and background.

Statistical analysis was performed using GraphPad Prism 7 (GraphPad Software, Inc.) and JMP 12 (SAS Institute Inc.). Curve analysis of tumor growth inhibition (TGI) was performed based on mean tumor volumes using the formula:

$$TGI = 100 - \frac{\overline{{v}_{treatment , d} - {v}_{treatment , 0}}}{\overline{{v}_{ref , d} - {v}_{ref , 0}}} \times 100$$

where d indicates study day and 0 the baseline value. Vehicle was selected as the reference group. Tumor regression (TR) was calculated according to:

$$TR = \frac{\overline{{v}_{treatment , 0} - {v}_{treatment , d}}}{\overline{{v}_{treatment , 0}}}$$

where positive values indicated tumor regression, and values below −1 growth beyond the double baseline value.

### Test Compounds

The compounds utilized in the described studies are presented in the tables below, respectively for bispecific antibodies, clearing agents, and radiolabeled chelates.

CEA-DOTAM (RO7198427, PRIT-0213) is a fully humanized BsAb targeting the T84.66 epitope of CEA (see also WO2019/201959). PRIT-0213 is composed of
i) a first heavy chain as shown below;
ii) a second heavy chain as shown below; and
iii) two antibody light chains as shown below.

| Description | SEQUENCE | |
|---|---|---|
| light chain | 1 | eivltqspat lslspgerat lscragesvd |
| of | | ifgvgflhwy qqkpgqaprl |
| PRIT-00213 | 51 | liyrasnrat giparfsgsg sgtdftltis |
| | | slepedfavy ycqqtnedpy |
| | 101 | tfgqgtklei krtvaapsvf ifppsdeqlk |
| | | sgtasvvell nnfypreakv |
| | 151 | qwkvdnalqs gnsqesvteq dskdstysls |
| | | stltlskady ekhkvyacev |
| | 201 | thqglsspvt ksfnrgec |
| heavy chain | 1 | qvqlvqsgae vkkpgssvkv sckasgfnik |
| 1 PRIT-0213 | | dtymhwvrqa pgqglewmgr |
| | 51 | idpangnsky vpkfqgrvti tadtststay |
| | | melsslrsed tavyycapfg |
| | 101 | yyvsdyamay wgqgtlvtvs sastkgpsvf |
| | | plapssksts ggtaalgclv |
| | 151 | kdyfpepvtv swnsgaltsg vhtfpavlqs |
| | | sglyslssvv tvpssslgtq |
| | 201 | tyicnvnhkp sntkvdkkve pkscdkthtc |
| | | ppcpapeaag gpsvflfppk |
| | 251 | pkdtlmisrt pevtcvvvdv shedpevkfn |
| | | wyvdgvevhn aktkpreeqy |
| | 301 | nstyrvvsvl tvlhqdwing keykckvsnk |
| | | algapiekti skakgqprep |
| | 351 | qvytlppcrd eltknqvslw clvkgfypsd |
| | | iavewesngq pennykttpp |
| | 401 | vldsdgsffl yskltvdksr wqqgnvfscs |
| | | vmhealhnhy tqkslslspg |
| | 451 | ggggsggggs ggggsggggs vtlkesgpvl |
| | | vkptetltlt ctvsgfslst |
| | 501 | ysmswirqpp gkalewlgfi gsrgdtyyas |
| | | wakgrltisk dtsksqvvlt |
| | 551 | mtnmdpvdta tyycarerdp ygggaypphl |
| | | wgrgtlvtvs s |
| heavy chain | 1 | qvqlvqsgae vkkpgssvkv sckasgfnik |
| 2 of | | dtymhwvrqa pgqglewmgr |
| PRIT-0213 | 51 | idpangnsky vpkfqgrvti tadtststay |
| | | melsslrsed tavyycapfg |
| | 101 | yyvsdyamay wgqgtlvtvs sastkgpsvf |
| | | plapssksts ggtaalgclv |
| | 151 | kdyfpepvtv swnsgaltsg vhtfpavlqs |
| | | sglyslssvv tvpssslgtq |
| | 201 | tyicnvnhkp sntkvdkkve pkscdkthtc |
| | | ppcpapeaag gpsvflfppk |
| | 251 | pkdtlmisrt pevtcvvvdv shedpevkfn |
| | | wyvdgvevhn aktkpreeqy |
| | 301 | nstyrvvsvl tvlhqdwlng keykckvsnk |
| | | algapiekti skakgqprep |
| | 351 | qvctlppsrd eltknqvsls cavkgfypsd |
| | | iavewesngq pennykttpp |
| | 401 | vldsdgsffl vskltvdksr wqqgnvfscs |
| | | vmhealhnhy tqkslslspg |
| | 451 | ggggsggggs ggggsggggs iqmtqspssl |
| | | sasvgdrvti tcqsshsvys |
| | 501 | dndlawyqqk pgkapklliy qasklasgvp |
| | | srfsgsgsgt dftltisslq |
| | 551 | pedfatyycl ggyddesdty gfgggtkvei |
| | | k |

DIG-DOTAM (RO7204012) is a non-CEA-binding BsAb used as a negative control.

P1AD8749, P1AD8592, P1AE4956, and P1AE4957 are CEA-split-DOTAM-VH/VL antibodies targeting the CH1A1A or A5B7 epitopes of CEA. Their sequences are described above. All antibody constructs were stored at −80° C. until the day of injection when they were thawed and diluted in standard vehicle buffer (20 mM Histidine, 140 mM NaCl; pH 6.0) or 0.9% NaCl to their final respective concentrations for intravenous (IV) or intraperitoneal (IP) administration.

The Pb-DOTAM-dextran-500 CA (RO7201869) was stored at −20° C. until the day of injection when it was thawed and diluted in PBS for IV or IP administration.

The DOTAM chelate for radiolabeling was provided by Macrocyclics and maintained at −20° C. before radiolabeling, performed by Orano Med (Razes, France). ²¹²Pb-DOTAM (RO7205834) was generated by elution with DOTAM from a thorium generator, and subsequently quenched with Ca after labeling. The ²¹²Pb-DOTAM solution was diluted with 0.9% NaCl to obtain the desired ²¹²Pb activity concentration for IV injection. Mice in vehicle control groups received multiple injections of vehicle buffer instead of BsAb, CA, and ²¹²Pb-DOTAM.

Bispecific antibodies

| Compound | Target | Protocols |
|---|---|---|
| CEA-DOTAM | T84.66 | 144, 158, 160 |
| (RO7198427, | | |
| PRIT-0213) | | |
| DIG-DOTAM | Digoxigenin | 160 |
| (RO7204012) | | |
| CEA-split-DOTAM-VH | CH1A1A | 144, 158 |
| P1AD8749 | | |
| CEA-split-DOTAM- | CH1A1A | 175, 185, 189 |
| VH-AST | | |
| P1AF0171 | | |
| CEA-split-DOTAM-VL | CH1A1A | 144, 158, |
| P1AD8592 | | 175, 185, 189 |
| CEA-split-DOTAM-VH | A5B7 | 158 |
| P1AE4956 | | |
| CEA-split-DOTAM-VL | A5B7 | 158 |
| P1AE4957 | | |
| CEA-split-DOTAM- | T84.66 | 185, 189 |
| VH-AST | | |
| P1AF0298 | | |
| CEA-split-DOTAM-VL | T84.66 | 185, 189 |
| P1AF0709 | | |

Clearing agents

| Compound | Protocols |
|---|---|
| Ca-DOTAM-dextran-500 | 144, 158, 160 |
| (RO7201869) | |

Radiolabeled chelates

| Compound | Quenching | Protocols |
|---|---|---|
| ²¹²Pb-DOTAM | Ca | 144, 158, |
| (RO7205834) | | 160, 175, |
| | | 185, 189 |
| ²¹²Pb-DOTAM-CEA- | Ca | 160 |
| DOTAM | | |

### Tumor Models

The tumor cell line used and the injected amount for inoculation in mice is described in the table below. BxPC3 is a human primary pancreatic adenocarcinoma cell line, naturally expressing CEA. Cells were cultured in RPMI 1640 Medium, GlutaMAX™ Supplement, HEPES (Gibco, ref. No. 72400-021) enriched with 10% fetal bovine serum (GE Healthcare Hyclone SH30088.03). Solid xenografts were established in each SCID mouse on study day 0 by subcutaneous injection of cells in RPMI media mixed 1:1 with Corning® Matrigel® basement membrane matrix (growth factor reduced; cat No. 354230), into the right flank.

Tumor cell lines

| | Cells per | Injected | | |
|---|---|---|---|---|
| Cell line | mouse | volume | Protocols | Supplier |
| BxPC3 | 5 × 10⁶ | 100 μL | 144, 158, 160, | ECACC* |
| | | | 175, 185, 189 | |

*European Collection of Authenticated Cell Cultures (Salisbury, UK)

### Example 3b: Protocol 144

The aim of protocol 144 was to provide PK and in vivo distribution data of pretargeted ²¹²Pb-DOTAM in SCID mice carrying SC BxPC3 tumors after 2-step PRIT using CEA-split-DOTAM-VH/VL BsAbs.

Two-step PRIT was performed by injection of the CEA-split-DOTAM-VH and CEA-split-DOTAM-VL (P1AD8749 and P1AD8592), separately or together, followed 7 days later by ²¹²Pb-DOTAM. Mice were sacrificed 6 hours after the radioactive injection, and blood and organs harvested for radioactive measurement. The 2-step scheme was compared with 3-step PRIT using the standard CEA-DOTAM bispecific antibody, followed 7 days later by Ca-DOTAM-dextran-500 CA, and ²¹²Pb-DOTAM 24 hours after the CA.

PK data of CEA-split-DOTAM-VH/VL clearance was collected by repeated blood sampling from 1 hour to 7 days after the antibody injection, and subsequently analyzed by an ELISA.

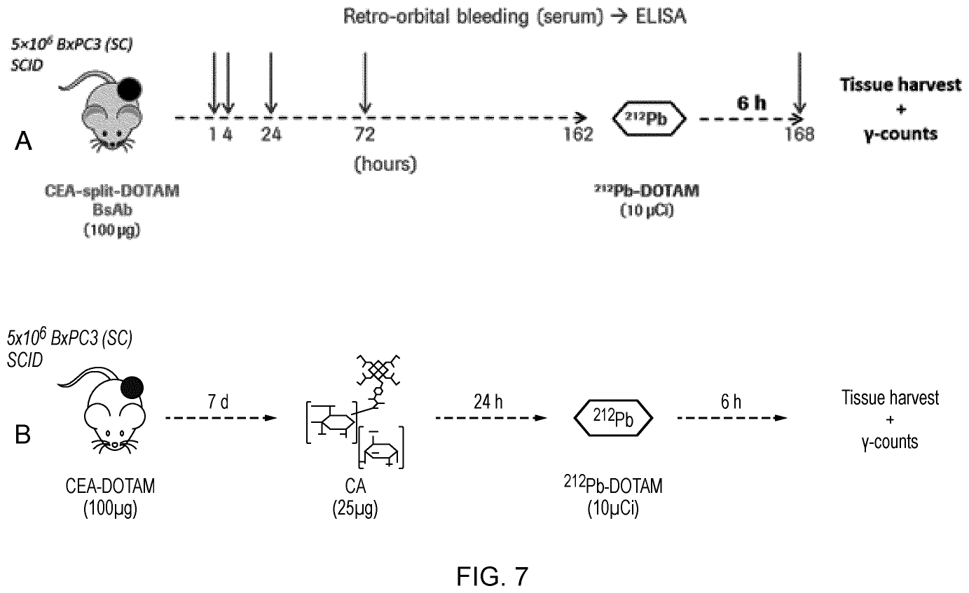

The study outline is shown in FIG. 7. FIG. 7a shows the outline of the 2-step PRIT regimen, including blood sampling for CEA-split-DOTAM-VH/VL PK, in SCID mice carrying SC BxPC3 tumors. FIG. 7b shows the outline of the 3-step PRIT regimen, performed in SCID mice carrying SC BxPC3 tumors (h=hours, d=days).

### Study Design

The time course and design of protocol 144 is shown in the tables below.

Time course of protocol 144

| Study day | Date | Experimental procedure |
|---|---|---|
| 0 | 2018 May 2 | Preparation of BxPC3 cells and |
| | | filling of syringes |
| 0 | 2018 May 2 | SC injection of BxPC3 cells |
| 14 | 2018 May 16 | IV injection of CEA-DOTAM BsAb (group D) |
| 15 | 2018 May 17 | IV injection of CEA-split-DOTAM-VH/VL |
| | | BsAbs (groups Aa, Ab, Ba, Bb, Ca, Cb) |
| 15 | 2018 May 17 | Retro-orbital bleeding (1 and 4 h p.i.; |
| | | groups Aa, Ba, Ca, and Ab, Bb, Cb, |
| | | respectively) |
| 16 | 2018 May 18 | Retro-orbital bleeding (24 h p.i.; |
| | | groups Aa, Ba, Ca) |
| 18 | 2018 May 20 | Retro-orbital bleeding (72 h p.i.; |
| | | groups Ab, Bb, Cb) |
| 21 | 2018 May 23 | IV injection of CA (group D) |
| 21 | 2018 May 23 | Elution of ²¹²Pb-DOTAM |
| | | and filling of syringes |
| 22 | 2018 May 24 | IV injection of ²¹²Pb- |
| | | DOTAM (groups Aa, Ba, Ca, D) |
| 22 | 2018 May 24 | Retro-orbital bleeding (168 h p.i.) and |
| | | euthanasia (groups Ab, Bb, Cb) |
| 22 | 2018 May 24 | Euthanasia and tissue harvest, incl. |
| | | retro-orbital bleeding (6 h p.i.) + gamma |
| | | counting (groups Aa, Ba, Ca, D) |

Study groups in protocol 144

| | P1AD8749 | P1AD8592 | | | | | | |
|---|---|---|---|---|---|---|---|---|
| | (VH) | (VL) | CEA-DOTAM | | | | | |
| | CH1A1A | CH1A1A | BsAb | PK | CA | ²¹²Pb | BD | n |
| Group | (μg) | (μg) | (μg) | (h p.i.) | (μg) | (μCi) | (h p.i.) | (mice) |
| Aa | 100 | 0 | 0 | 1, 24, 168 | 0 | 10 | 6 | 4 |
| Ab | 100 | 0 | 0 | 4, 72, 168 | 0 | 0 | — | 4 |
| Ba | 0 | 100 | 0 | 1, 24, 168 | 0 | 10 | 6 | 4 |
| Bb | 0 | 100 | 0 | 4, 72, 168 | 0 | 0 | — | 4 |
| Ca | 100 | 100 | 0 | 1, 24, 168 | 0 | 10 | 6 | 4 |
| Cb | 100 | 100 | 0 | 4, 72, 168 | 0 | 0 | — | 4 |
| D | 0 | 0 | 100 | — | 25 | 10 | 6 | 4 |

Solid xenografts were established in each SCID mouse on study day 0 by SC injection of 5×10⁶ cells (passage 26) in RPMI/Matrigel into the right flank. Fourteen days after tumor cell injection, mice were sorted into experimental groups with an average tumor volume of 116 mm³. The ²¹²Pb-DOTAM was injected on day 22 after inoculation; the average tumor volume was 140 mm³ on day 21.

Blood from mice in groups Aa, Ba, and Ca was collected through retro-orbital bleeding under anesthesia 1 h (right eye), 24 h (left eye), and 168 h (right eye, at termination) after CEA-split-DOTAM-VH/VL injection. Similarly, samples were taken from mice in groups Ab, Bb, and Cb 4 h (right eye), 72 h (left eye), and 168 h (right eye, at termination) after CEA-split-DOTAM-VH/VL injection.

Mice in groups Aa, Ba, Ca, and D were sacrificed and necropsied 6 hours after injection of ²¹²Pb-DOTAM, and the following organs and tissues harvested for measurement of radioactive content: blood, skin, bladder, stomach, small intestine, colon, spleen, pancreas, kidneys, liver, lung, heart, femoral bone, muscle, brain, tail, ears, and tumor.

### Results

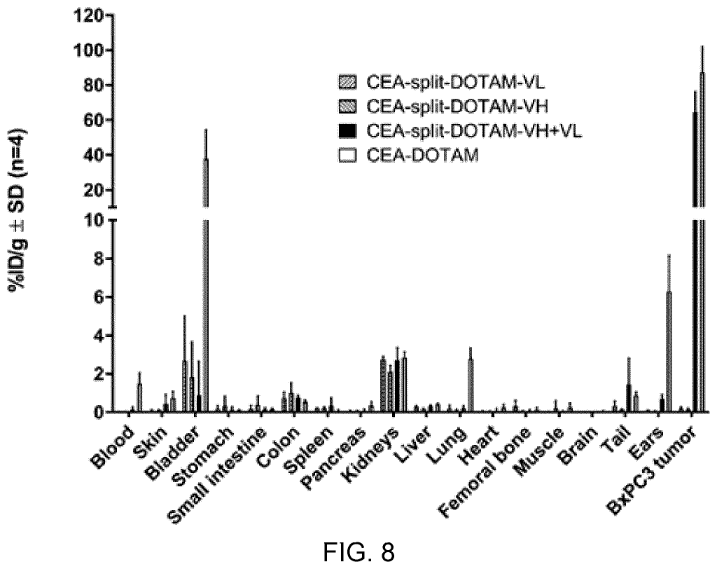

The average ²¹²Pb accumulation and clearance in all collected tissues 6 hours after injection is displayed in FIG. 8. Pretargeting with either CEA-split-DOTAM-VH or CEA-split-DOTAM-VL alone resulted in no accumulation of radioactivity in tumors. Combined, the two complimentary antibodies resulted in a tumor uptake after 2-step PRIT of 65±12% ID/g, to be compared with 87±15% ID/g for the standard 3-step PRIT regimen. Two-way analysis of variance (ANOVA) with Tukey's multiple comparisons test showed that the difference in tumor uptake between the two PRIT treatments was significant, as was the difference in bladder (1±2% ID/g and 38±17% ID/g for 2- and 3-step PRIT, respectively); no other differences in tissue accumulation were statistically significant using this test (p=0.05).

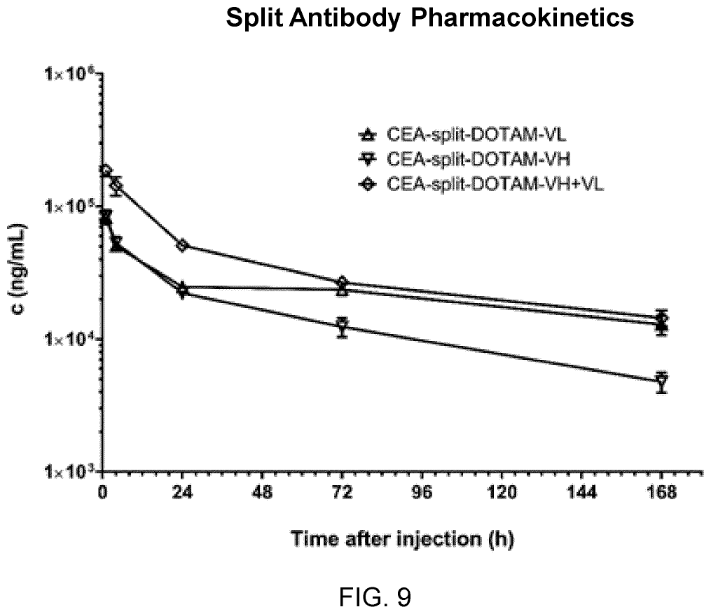

The clearance of IV injected CEA-split-DOTAM-VH/VL constructs as analyzed by an enzyme-linked immunosorbent assay (ELISA) is shown in FIG. 9.

### Adverse Events and Toxicity

There were no adverse events or toxicity associated with this study.

### Conclusion

The results of the study demonstrated proof-of-concept of CA-independent 2-step pretargeting using complimentary CEA-split-DOTAM-VH/VL antibodies. High and specific tumor uptake of ²¹²Pb-DOTAM was achieved using 2-step PRIT and standard 3-step PRIT, with very little accumulation of radioactivity in normal tissues using the complimentary CEA-split-DOTAM-VH/VL antibodies.

### Example 3c: Protocol 158

The aim of protocol 158 was to assess the association of ²¹²Pb-DOTAM to subcutaneous BxPC3 tumors in mice pretargeted by bi-paratopic (CH1A1A and A5B7) pairs of CEA-split-DOTAM-VH/VL antibodies for clearing agent-independent 2-step CEA-PRIT. The tumor uptake was compared with that of standard 3-step CEA-PRIT.

Mice carrying subcutaneous BxPC3 tumors were injected with either
CEA-split-DOTAM-VH/VL antibodies followed 7 days later by the radiolabeled ²¹²Pb-DOTAM (2-step PRIT), or
CEA-DOTAM BsAb followed 7 days later by the CA, and finally the radiolabeled ²¹²Pb-DOTAM 24 hours later (3-step PRIT).

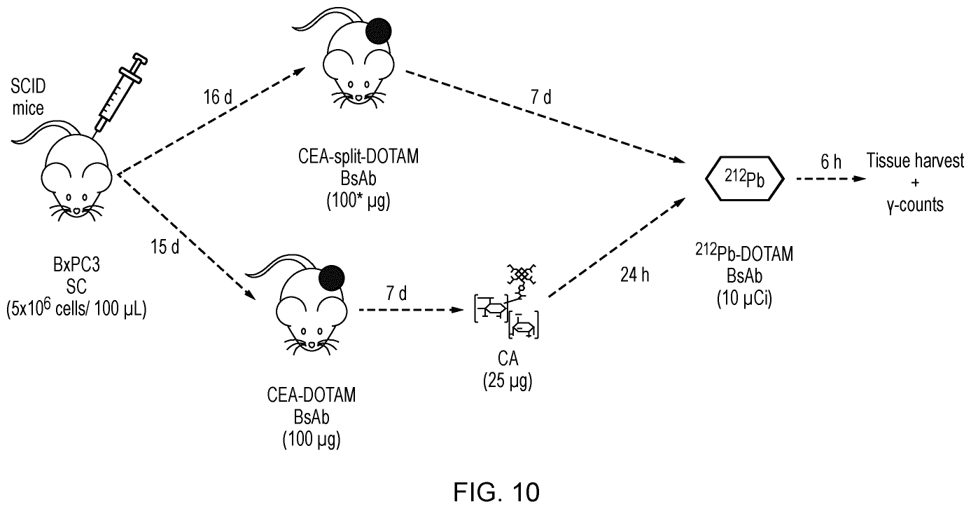

The in vivo distribution of ²¹²Pb-DOTAM was assessed 6 hours after the radioactive injection. The study outline is shown in FIG. 10.

### Study Design

The time course and design of protocol 158 is shown in the tables below.

Time course of protocol 158

| Study day | Date | Experimental procedure |
|---|---|---|
| 0 | 2018 Nov. 26 | Preparation of BxPC3 cells and |
| | | filling of syringes |
| 0 | 2018 Nov. 26 | SC injection of BxPC3 cells |
| 15 | 2018 Dec. 11 | IV injection of CEA-DOTAM |
| | | BsAb (group C) |
| 16 | 2018 Dec. 12 | IV injection of CEA-split-DOTAM-VH/VL |
| | | BsAbs (groups A, B) |
| 22 | 2018 Dec. 18 | IV injection of CA (group C) |
| 22 | 2018 Dec. 18 | Elution of ²¹²Pb-DOTAM |
| | | and filling of syringes |
| 23 | 2018 Dec. 19 | IV injection of ²¹²Pb-DOTAM (all) |
| 23 | 2018 Dec. 19 | Euthanasia and tissue harvest, incl. |
| | | retro-orbital bleeding (6 h p.i.) + gamma |
| | | counting (all) |

Study groups in protocol 158

| | P1AD8749 | P1AD8592 | P1AE4956 | P1AE4957 | CEA- | | | |
|---|---|---|---|---|---|---|---|---|
| | (VH) | (VL) | (VH) | (VL) | DOTAM | | | |
| | CH1A1A | CH1A1A | A5B7 | A5B7 | BsAb | CA | ²¹²Pb | n |
| Group | (μg) | (μg) | (μg) | (μg) | (μg) | (μg) | (μCi) | (mice) |
| A | 154* | 0 | 0 | 100 | 0 | 0 | 10 | 4 |
| B | 0 | 100 | 167** | 0 | 0 | 0 | 10 | 4 |
| C | 0 | 0 | 0 | 0 | 100 | 25 | 10 | 4 |

*P1AD8749 dose adjusted to 154 μg to compensate for a 35% hole/hole impurity;
**P1AD8592 dose adjusted to 167 μg to compensate for a 40% hole/hole impurity.

Solid xenografts were established in each SCID mouse on study day 0 by SC injection of 5×10⁶ cells (passage 27) in RPMI/Matrigel into the right flank. Fourteen days after tumor cell injection, mice were sorted into experimental groups with an average tumor volume of 177 mm³. The ²¹²Pb-DOTAM was injected on day 20 after inoculation; the average tumor volume was 243 mm³ on day 21.

Mice in all groups were sacrificed and necropsied 6 hours after injection of ²¹²Pb-DOTAM, and the following organs and tissues harvested for measurement of radioactive content: blood, skin, bladder, stomach, small intestine, colon, spleen, pancreas, kidneys, liver, lung, heart, femoral bone, muscle, brain, tail, and tumor.

### Results

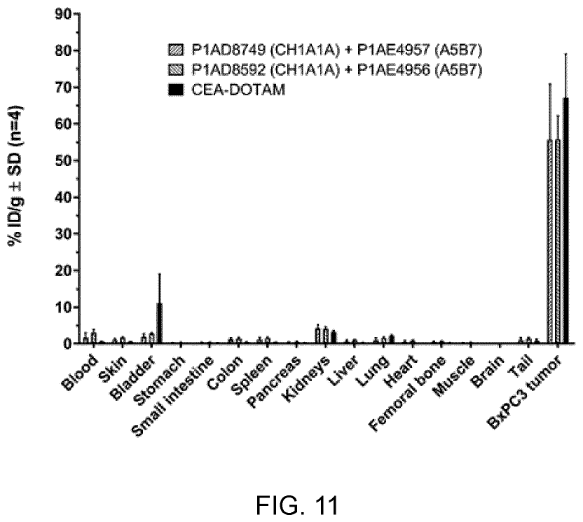

The average ²¹²Pb distribution in all collected tissues 6 hours after injection is shown in FIG. 11. Two-way ANOVA with Tukey's multiple comparisons test showed that there was no significant difference in normal tissue uptake of ²¹²Pb between the three treatments, except for bladder, where both bi-paratopic CEA-split-DOTAM-VH/VL pairs yielded lower accumulation than the standard 3-step PRIT. The kidney uptake was 3-4% ID/g for all three treatments. Either bi-paratopic combination resulted in tumor accumulation of approximately 56% ID/g, compared with 67% ID/g for 3-step PRIT; the difference between 2- and 3-step PRIT was statistically significant (p<0.0001).

### Adverse Events and Toxicity

There were no adverse events or toxicity associated with this study.

### Conclusion

This study assessed the association of ²¹²Pb-DOTAM to SC BxPC3 tumors in mice pretargeted by bi-paratopic pairs of CEA-split-DOTAM-VH/VL antibodies for CA-independent 2-step CEA-PRIT, compared with standard 3-step PRIT. The distribution of ²¹²Pb 6 hours after injection was comparable for 2- and 3-step PRIT, with high accumulation in tumor and very little radioactivity in healthy tissues. This demonstrated proof of concept of bi-paratopic pretargeting of CEA-expressing tumors for 2-step CEA-PRIT using CEA-split-DOTAM-VH/VL antibodies.

### Example 3d: Protocol 160

The aim of protocol 160 was to compare the therapeutic efficacy after 3 cycles of CA-independent 2-step CEA-PRIT using complimentary CEA-split-DOTAM-VH/VL antibodies, with that of standard 3-step CEA-PRIT in mice bearing SC BxPC3 tumors. A comparison was also made with 1-step CEA-RIT, using BsAbs that were pre-incubated with ²¹²Pb-DOTAM before injection.

Mice carrying SC BxPC3 tumors were injected with either
CEA-DOTAM BsAb followed 7 days later by the CA, and finally the radiolabeled ²¹²Pb-DOTAM 24 hours later (3-step PRIT),
CEA-split-DOTAM-VH/VL antibodies followed 7 days later by the radiolabeled ²¹²Pb-DOTAM (2-step PRIT), or
²¹²Pb-DOTAM-CEA-DOTAM BsAb (pre-incubated; 1-step PIT).

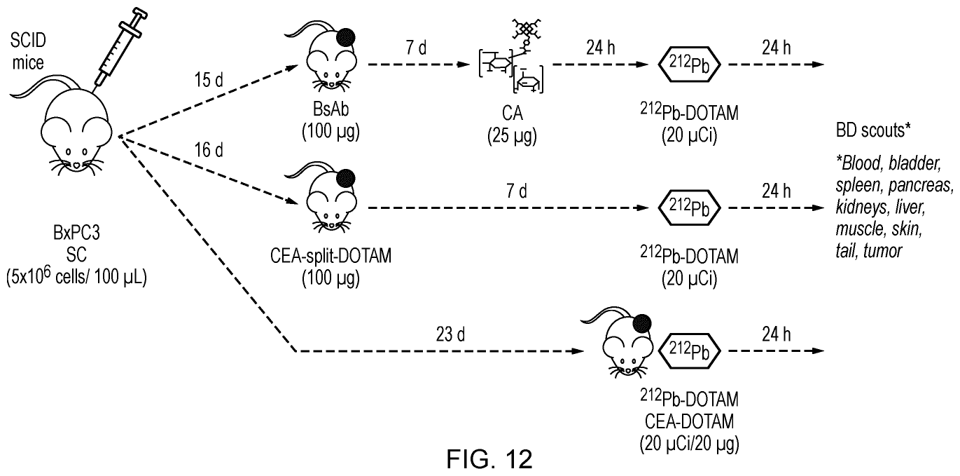

The therapy was administered in 3 repeated cycles of 20 μCi of ²¹²Pb-DOTAM, also including comparison with a non-CEA binding control antibody (DIG-DOTAM), and no treatment (vehicle). Dedicated mice were sacrificed for biodistribution purposes to confirm ²¹²Pb-DOTAM targeting and clearance at each treatment cycle. The treatment efficacy was assessed in terms of TGI and TR, and the mice were carefully monitored for the duration of the study to assess the tolerability of the treatment. The study outline is shown in FIG. 12.

The time course and design of protocol 160 are shown in the tables below.

Time course of protocol 160

| Study day | Date | Experimental procedure |
|---|---|---|
| 0 | 2019 Jan. 29 | Preparation of BxPC3 cells and filling of syringes |
| 0 | 2019 Jan. 29 | SC injection of BxPC3 cells |
| 15 | 2019 Feb. 13 | IP injection of BsAb or histidine buffer (groups A, B, C, |
| | | F, G, H, I) |
| 16 | 2019 Feb. 14 | IP injection of CEA-split-DOTAM-VH/VL or histidine |
| | | buffer (groups D, J, K, L) |
| 22 | 2019 Feb. 20 | IP injection of CA or PBS (groups A, B, C, F, G, H, I) |
| 23 | 2019 Feb. 21 | Elution of ²¹²Pb-DOTAM and filling of syringes |
| 23 | 2019 Feb. 21 | IV injection of ²¹²Pb-DOTAM-CEA-DOTAM or histidine |
| | | buffer (groups E, M) |
| 23 | 2019 Feb. 21 | IV injection of ²¹²Pb-DOTAM or 0.9% NaCl (groups B, |
| | | C, D, F, G, H, I, J, K, L) |
| 24 | 2019 Feb. 22 | Euthanasia and tissue harvest (24 h p.i.) + gamma |
| | | counting (groups F, G, J, M) |
| 29 | 2019 Feb. 27 | IP injection of PRIT BsAb or PBS (groups A, B, C, H, I) |
| 30 | 2019 Feb. 28 | IP injection of CEA-split-DOTAM-VH/VL or histidine |
| | | buffer (groups D, K, L) |
| 36 | 2019 Mar. 6 | IP injection of CA or PBS (groups A, B, C, H, I) |
| 37 | 2019 Mar. 7 | Elution of ²¹²Pb-DOTAM and filling of syringes |
| 37 | 2019 Mar. 7 | IV injection of ²¹²Pb-DOTAM or 0.9% NaCl (groups B, |
| | | C, D, H, I, K, L) |
| 38 | 2019 Mar. 8 | Euthanasia and tissue harvest (24 h p.i.) + gamma |
| | | counting (groups H, K) |
| 43 | 2019 Mar. 13 | IP injection of PRIT BsAb or PBS (groups A, B, C, I) |
| 44 | 2019 Mar. 14 | IP injection of CEA-split-DOTAM-VH/VL or histidine |
| | | buffer (groups D, L) |
| 50 | 2019 Mar. 20 | IP injection of CA or PBS (groups A, B, C, I) |
| 51 | 2019 Mar. 21 | Elution of ²¹²Pb-DOTAM and filling of syringes |
| 51 | 2019 Mar. 21 | IV injection of ²¹²Pb-DOTAM or 0.9% NaCl (groups B, |
| | | C, D, I, L) |
| 52 | 2019 Mar. 22 | Euthanasia and tissue harvest (24 h p.i.) + gamma |
| | | counting (groups I, L) |

Study groups in protocol 160

| | | | CA | ²¹²Pb- | | |
|---|---|---|---|---|---|---|
| | | BsAb | per | DOTAM | | |
| | | per cycle | cycle | per cycle | Cycles | n |
| Group | BsAb | (μg) | (μg) | (μCi) | (#) | (mice) |
| A | — | 0 | 0 | 0 | 3 | 10 |
| B | DIG-DOTAM | 100 | 25 | 20 | 3 | 10 |
| C | CEA-DOTAM | 100 | 25 | 20 | 3 | 10 |
| D | CEA-split- | 154* + 100** | 0 | 20 | 3 | 10 |
| | DOTAM | | | | | |
| E | ²¹²Pb-DOTAM- | 100 | 0 | 20 | 1*** | 10 |
| | CEA-DOTAM | | | | | |
| F | DIG-DOTAM | 100 | 25 | 20 | 1 | 3 |
| G | CEA-DOTAM | 100 | 25 | 20 | 1 | 3 |
| H | CEA-DOTAM | 100 | 25 | 20 | 2 | 3 |
| I | CEA-DOTAM | 100 | 25 | 20 | 3 | 3 |
| J | CEA-split- | 154* + 100** | 0 | 20 | 1 | 3 |
| | DOTAM | | | | | |
| K | CEA-split- | 154* + 100** | 0 | 20 | 2 | 3 |
| | DOTAM | | | | | |
| L | CEA-split- | 154* + 100** | 0 | 20 | 3 | 3 |
| | DOTAM | | | | | |
| M | ²¹²Pb-DOTAM- | 100 | 0 | 20 | 1 | 3 |
| | CEA-DOTAM | | | | | |

*P1AD8749: dose adjusted to 154 μg to compensate for a 35% hole/hole impurity in the stock solution;
**P1AD8592;
***Adjusted from 3 cycles to 1 cycle due to acute radiation-induced toxicity at the first treatment cycle.

Solid xenografts were established in SCID mice on study day 0 by SC injection of 5×10⁶ cells (passage 24) in RPMI/Matrigel into the right flank. Fifteen days after tumor cell injection, mice were sorted into experimental groups with an average tumor volume of 122 mm³. The ²¹²Pb-DOTAM was injected on day 23 after inoculation; the average tumor volume was 155 mm³ on day 22.

The CEA-DOTAM and DIG-DOTAM antibodies were diluted in vehicle buffer to a final concentration of 100 jig per 200 μL for IP administration according to the table above (Study groups in protocol 160). The CEA-split-DOTAM-VH/VL antibodies were mixed together into one single injection solution for IP administration, containing 100 μg of each construct per 200 μL. For P1AD8749, the dosing was adjusted to 154 μg to compensate for a 35% hole/hole impurity in the stock solution (the side of the molecule that does not carry the VH/VL). The Ca-DOTAM-dextran-500 CA was administered IP (25 μg per 200 μL of PBS) 7 days after the BsAb injection, followed 24 hours later by ²¹²Pb-DOTAM (RO7205834) according to the experimental schedule in FIG. 12. PRIT-treated mice (2- and 3-step) were injected IV with 100 μL of the Ca-quenched ²¹²Pb-DOTAM solution (20 μCi in 100 μL 0.9% NaCl).

Mice treated with 1-step RIT received only one injection: pre-bound ²¹²Pb-DOTAM-CEA-DOTAM (20 μCi/20 μg BsAb in 100 μL 0.9% NaCl for IV injection). The direct-labeled antibody was prepared by incubating the ²¹²Pb-DOTAM with the CEA-DOTAM BsAb for 10 minutes at 37° C.

The following organs and tissues were harvested from mice in groups A-E at the time of euthanasia: serum, liver, spleen, kidneys, pancreas, and tumor. Before euthanasia, the live mouse was anesthetized for retro-orbital blood collection. The collected blood samples were centrifuged at 10 000 rcf during 5 minutes and the resulting serum fractions isolated, frozen, and stored at −20° C. The excised tissues were immediately put in 10% neutral buffered formalin (4° C.) and then transferred to 1×PBS (4° C.) after 24 hours. The formalin-fixed samples were shipped to Roche Pharma Research and Early Development, Roche Innovation Center Basel, for further processing and analysis.

Mice in groups F, G, J, and M were sacrificed and necropsied 24 hours after their first and only injection of ²¹²Pb-DOTAM or ²¹²Pb-DOTAM-BsAb; groups H and K were sacrificed and necropsied 24 hours after their second ²¹²Pb-DOTAM injection; groups I and L were sacrificed and necropsied 24 hours after their third ²¹²Pb-DOTAM injection. Blood was collected at the time of euthanasia from the venous sinus using retro-orbital bleeding on anesthetized mice, before termination through cervical dislocation. The following organs and tissues were also harvested for biodistribution purposes: bladder, spleen, kidneys, liver, lung, muscle, tail, skin, and tumor.

### Results

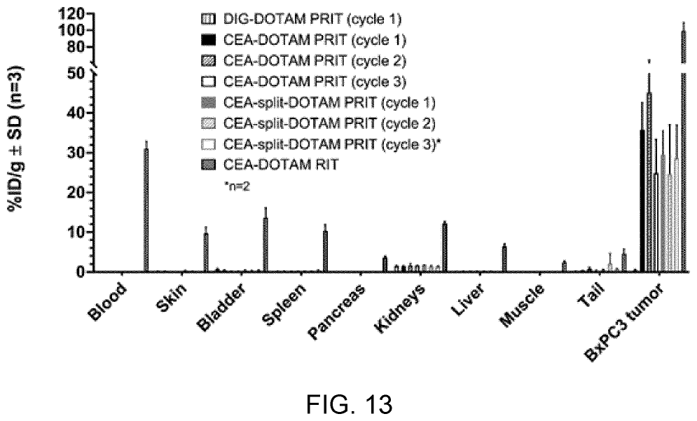

The average ²¹²Pb accumulation and clearance in all collected tissues 24 hours after injection is shown for each therapy and treatment cycle in FIG. 13. The negative control resulted in no uptake (0.4% ID/g) in tumor. Two-way analysis of variance (ANOVA) with Tukey's multiple comparisons test showed that the distributions were not significantly different at any cycle for the 2-step and 3-step PRIT; however, the differences were at all cycles statistically significant compared with the negative control and the 1-step RIT (p<0.05). The tumor uptake was 25-45% ID/g for 3-step PRIT and 25-30% ID/g for 2-step PRIT, without any statistically significant difference between either treatment or cycle. For 1-step RIT, the tumor uptake was 99% at the one and only treatment cycle. The uptake in normal tissues was very low for both PRIT regimens, but significantly higher in all organs and tissues after 1-step RIT, due to the much longer circulating time of the pre-incubated antibody compared with the small, radiolabeled DOTAM chelate.

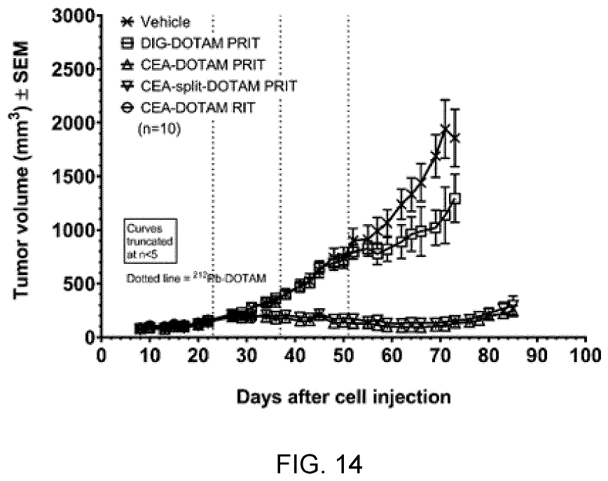

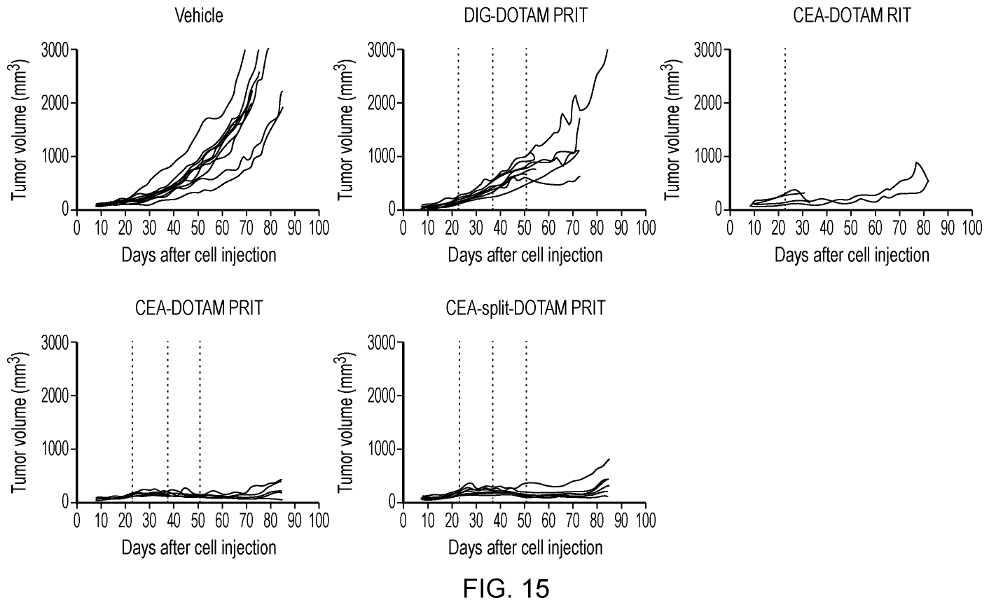

The average tumor development and the individual tumor growth curves are shown in FIG. 14 and FIG. 15, respectively. Tumors in the non-treated vehicle group and the DIG-DOTAM group grew steadily, albeit with slightly lower doubling rate in the latter after the third treatment. In contrast, tumors in the PRIT and RIT groups decreased in size after the first treatment cycle, and maintained tumor control until approximately 10 weeks after inoculation, when the tumors started to increase in size. The 2-step and 3-step PRIT treatments resulted in near identical tumor control. No tumors regressed completely.

On study day 83, the last day on which all treatment groups could be analyzed based on means, the TGI was 91.7% and 88.4% for PRIT using CEA-DOTAM (3-step) and CEA-split-DOTAM-VH/VL (2-step), respectively, compared with the vehicle control. The corresponding number for 1-step RIT was 72.6%, whereas the TGI was −59.7% for the non-specific DIG-DOTAM control. On the same day, the TR based on means was −1.9 for 3-step CEA-DOTAM PRIT, −2.9 for 2-step CEA-split-DOTAM-VH/VL PRIT, −4.7 for 1-step RIT, −28.8 for DIG-DOTAM PRIT, and −39.3 for the vehicle control.

Due to the adverse events described below, survival analysis was not considered statistically relevant.

### Adverse Events and Toxicity

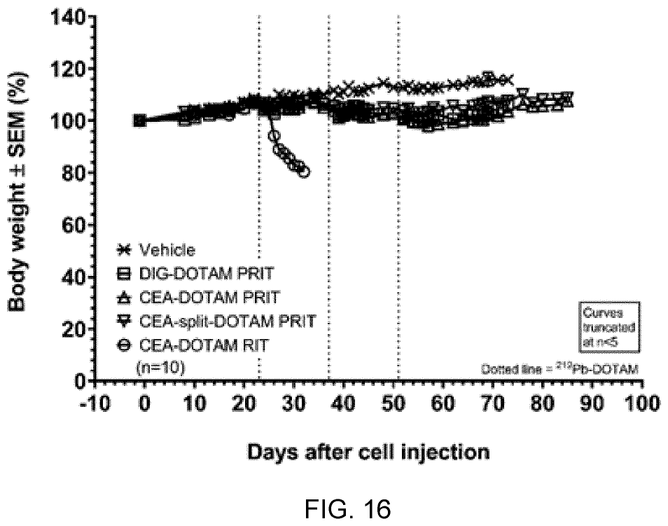

The BW development in all therapy groups is shown in FIG. 16. The multiple cycles of 2- and 3-step PRIT with 20 μCi of ²¹²Pb-DOTAM were well tolerated, but acute BW loss occurred in mice receiving 1-step RIT, with 8/10 mice in group E euthanized after the first RIT cycle (6-11 days after ²¹²Pb irradiation) due to a drop in BW of 20% or more. The remaining 2 RIT mice were not given any further ²¹²Pb-DOTAM-CEA-DOTAM injections but were continuously followed up for tumor growth assessment.

In addition, a number of mice were sacrificed for ethical reasons due to declining tumor status, i.e. tumors opening up or leaking. In the DIG-DOTAM group, 9/10 mice were euthanized before reaching a tumor volume of 3000 mm³ for this reason; for the non-treated vehicle control, the corresponding number was 5/10. The problem was less pronounced in the PRIT and RIT groups, with 1/10, 2/10, and 2/10 mice euthanized for this reason in the 3-step PRIT, 2-step PRIT, and 1-step RIT groups, respectively. This is reflected in the individual tumor growth curves in FIG. 15.

Finally, 1 mouse in group C was euthanized due to a degrading wound under the anus. All adverse events are listed in the table below.

Adverse events in protocol 160

| | Mice | | |
|---|---|---|---|
| | sacrificed | | |
| | (n per | Study | Reason for |
| Group | group) | day | termination |
| A: Vehicle | 5(10) | 53, 71, 71, | Declining |
| | | 73, 75 | tumor |
| | | | status |
| B: DIG-DOTAM | 9(10) | 55, 55, 55, | Declining |
| | | 55, 61, 73, | tumor |
| | | 73, 73, 74 | status |
| C: CEA -DOTAM | 1(10) | 83 | Wound under |
| | | | the anus |
| C: CEA -DOTAM | 1(10) | 85 | Declining |
| | | | tumor |
| | | | status |
| D: CEA-split- | 2(10) | 83, 83 | Declining |
| DOTAM | | | tumor |
| | | | status |
| E: ²¹²Pb-DOTAM-CEA- | 8(10) | 29, 29, 30, 31, | BW |
| DOTAM | | 31, 32, 32, 34 | loss ≥20% |
| E: ²¹²Pb-DOTAM-CEA- | 2(10) | 83, 83 | Declining |
| DOTAM | | | tumor |
| | | | status |

²¹²Pb irradiation was performed on study day 23 (cycle 1), 37 (cycle 2), and 51 (cycle 3).

### Conclusion

No difference was seen between CEA-PRIT using the 3-step scheme (CEA-DOTAM BsAb, CA, and ²¹²Pb-DOTAM) and the 2-step scheme (CEA-split-DOTAM-VH/VL antibodies and ²¹²Pb-DOTAM); the TGI was significant and near identical for the two treatments, and 3 cycles of 20 μCi could be safely administered in both cases. Contrastingly, 20 μCi of ²¹²Pb-DOTAM pre-bound to CEA-DOTAM before injection (1-step RIT) was not tolerated by a large majority of the treated mice.

The study thus demonstrated tolerability and therapeutic efficacy of CA-independent 2-step PRIT using the developed CEA-split-DOTAM-VH/VL constructs.

### Example 4: Protocol 175

The aim of protocol 175 was to assess the impact of increased injected pretargeting antibody amount on the subsequent ²¹²Pb accumulation in tumor and healthy tissues. Two different doses of CEA-split-DOTAM-VH/VL antibodies were compared: the standard amount (100 μg) and 2.5 times higher dose (250 μg). Moreover, a modification was made to the CEA-split-DOTAM-VH construct to extend its VH to avoid anti-drug antibody (ADA) formation (this was used together with a previously tested CEA-split-DOTAM-VL construct). The VH was extended to comprise the first three amino acids from the antibody CH1 domain: alanine, serine, and threonine (AST), and the construct hereafter referred to as CEA-split-DOTAM-VH-AST.

Antibody P1AD8592 has already been described above, in example 1. P1AF0171 is the same as P1AD8749 except that the fusion HC is extended by the residues AST—thus, antibody P1AD0171 consists of the light chain D1AA3384 as described above (SEQ ID NO: 34), the first heavy chain D1AC4022 as described above (SEQ ID NO: 28), and a second heavy chain D1AE3669 as shown below:

D1AE3669 (HCknob <CEA> CH1A1A Dotam-VH-AST)
QVQLVQSGAEVKKPGASVKVSCKASGYTFTEFGMNWVRQAPGQGLEWMGW
INTKTGEATYVEEFKGRVTFTTDTSTSTAYMELRSLRSDDTAVYYCARWD
FAYYVEAMDYWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALGAPIEKTISKAKGQPREP
QVYTLPPCRDELTKNQVSLWCLVKGFYPSDIAVEWESNGQPENNYKTTPP
VLDSDGSFFLYSKLTVDKSRWQQGNVFSCSVMHEALHNHYTQKSLSLSPG
GGGGSGGGGSGGGGSGGGGSVTLKESGPVLVKPTETLTLTCTVSGFSLST
YSMSWIRQPPGKALEWLGFIGSRGDTYYASWAKGRLTISKDTSKSQVVLT
MTNMDPVDTATYYCARERDPYGGGAYPPHLWGRGTLVTVSSAST

Mice Carrying SC BxPC3 Tumors were Injected with Either
1× the standard dose of CEA-split-DOTAM-VH/VL BsAb followed 7 days later by the radiolabeled ²¹²Pb-DOTAM, or
2.5× the standard dose of CEA-split-DOTAM-VH/VL BsAb followed 7 days later by the radiolabeled ²¹²Pb-DOTAM.

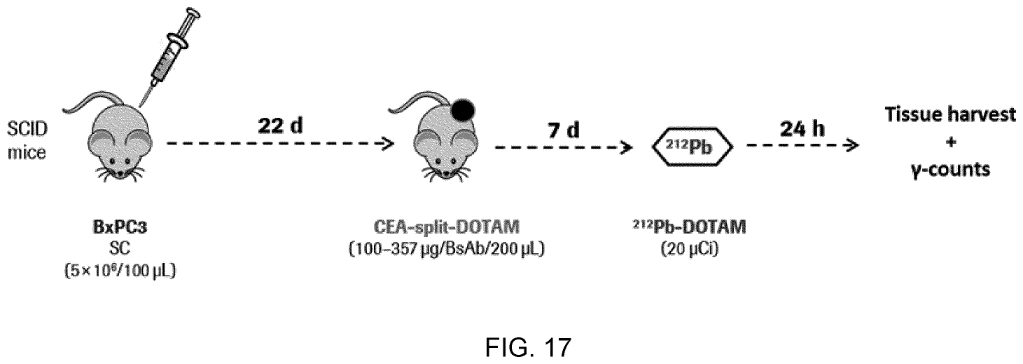

The in vivo distribution of ²¹²Pb-DOTAM was assessed 24 hours after the radioactive injection. The study outline is shown in FIG. 17.

Study Design

The time course and design of protocol 175 are shown below.

Time course of protocol 175

| Study day | Experimental procedure |
|---|---|
| 0 | Preparation of BxPC3 cells and filling of syringes |
| 0 | SC injection of BxPC3 cells |
| 22 | IP* injection of CEA-split-DOTAM-VH/VL BsAbs |
| 29 | Elution of ²¹²Pb-DOTAM and filling of syringes |
| 29 | IV injection of ²¹²Pb-DOTAM |
| 30 | Euthanasia and tissue harvest (24 h p.i.) + gamma |
| | counting |

*IP injection required due to low compound concentration (200 μL per construct = 400 μL in total)

Study groups in protocol 175

| | P1AF0171 | P1AD85 | | | |
|---|---|---|---|---|---|
| | (VH-AST) | 92 (VL) | ²¹²Pb | BD | n |
| Group | (μg) | (μg) | (μCi) | (h p.i.) | (mice) |
| A | 143* | 100 | 10 | 24 | 4 |
| B | 357* | 250 | 10 | 24 | 4 |

*P1AF0171 dose adjusted to 143 and 357 μg to compensate for a ~30% hole/hole impurity.

Solid xenografts were established in each SCID mouse on study day 0 by SC injection of 5×10⁶ cells (passage 24) in RPMI/Matrigel into the right flank. Twenty-one days after tumor cell injection, mice were sorted into experimental groups with an average tumor volume of 310 mm³. The ²¹²Pb-DOTAM was injected on day 29 after inoculation; the average tumor volume was 462 mm³ on day 30.
All mice were sacrificed and necropsied 24 hours after injection of ²¹²Pb-DOTAM, and the following organs and tissues harvested for measurement of radioactive content: blood, skin, spleen, pancreas, kidneys, liver, muscle, tail, and tumor.

Results

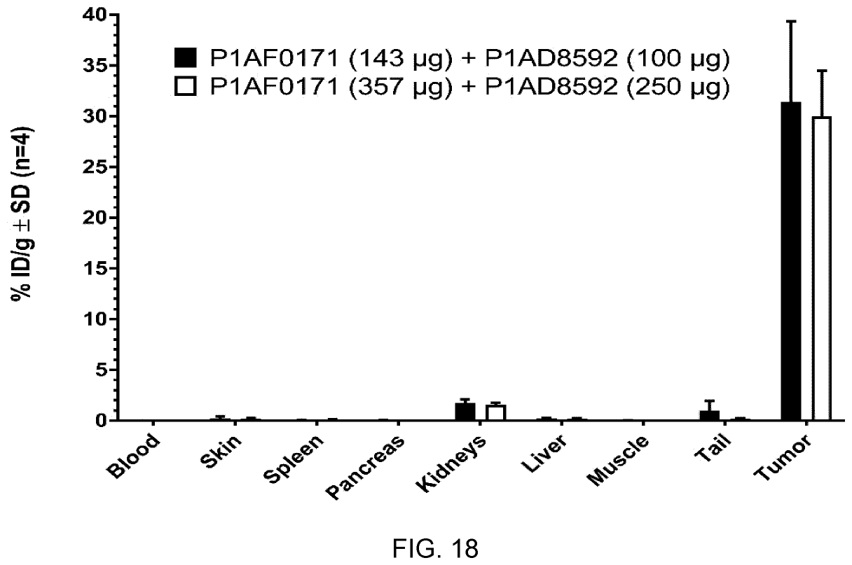

The average ²¹²Pb distribution in all collected tissues 24 hours after injection is shown in FIG. 18. There was no significant difference in tumor or normal tissue uptake of ²¹²Pb between the two dose levels. The tumor accumulation was 30-31% ID/g for both treatment groups, with a kidney uptake of ≤2% ID/g at this time point. One mouse had ˜1% ID/g in the tail due to ²¹²Pb-DOTAM injection issues, but no other collected healthy tissues showed any appreciable ²¹²Pb accumulation.

Adverse Events and Toxicity

There were no adverse events or toxicity associated with this study.

Conclusion

Increasing the dose of the pretargeting CEA-split-DOTAM-VH/VL antibodies by 2.5-fold did not improve the tumor accumulation of subsequently administered ²¹²Pb-DOTAM in this in vivo model. However, it also did not increase the accumulation of radioactivity in normal tissues, highlighting the strong specificity achieved using this 2-step pretargeting regimen. Finally, the results verified the function of the extended-VH CEA-split-DOTAM-VH-AST construct.

### Example 5: Protocol 185

The aim of protocol 185 was to assess a CEA-split-DOTAM-VH/VL targeting the T84.66 epitope. Sequences of P1AF0709 and P1AF0298 are provided herein. P1AF0709 has a first heavy chain of D1AE4688 (SEQ ID NO: 83) and a second heavy chain of D1AA4920 (SEQ ID NO: 84). P1AF0298 has a first heavy chain of D1AE4687 (SEQ ID NO: 86) and a second heavy chain of D1AE3668 (SEQ ID NO: 87). Both have the light chain of D1AA4120 (SEQ ID NO: 89).

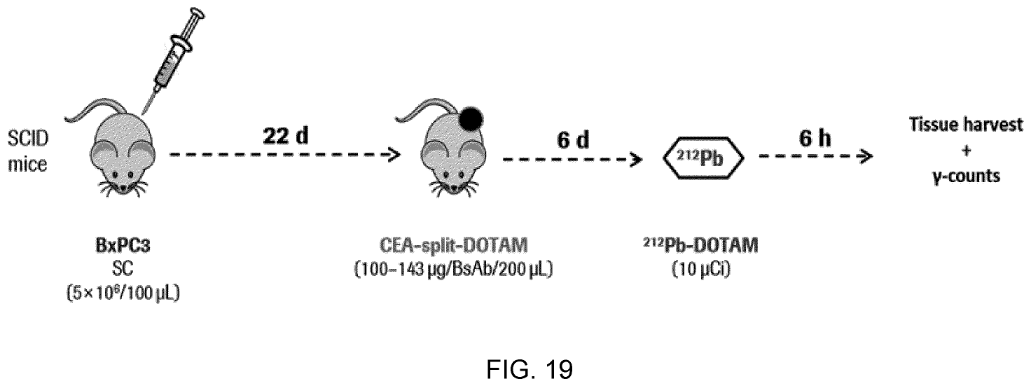

Mice carrying SC BxPC3 tumors were injected with the standard dose of CEA-split-DOTAM-VH/VL BsAb (100 μg per antibody) followed 6 days later by the radiolabeled ²¹²Pb-DOTAM. The in vivo distribution of ²¹²Pb-DOTAM was assessed 6 hours after the radioactive injection. The study outline is shown in FIG. 19.

Study Design

The time course and design of protocol 185 is shown below.

Time course of protocol 185

| Study day | Date | Experimental procedure |
|---|---|---|
| 0 | 2020 Mar. 4 | Preparation of BxPC3 cells |
| | | and filling of syringes |
| 0 | 2020 Mar. 4 | SC injection of BxPC3 cells |
| 22 | 2020 Mar. 26 | IV injection of CEA-split- |
| | | DOTAM-VH/VL BsAbs |
| 27 | 2020 Mar. 31 | Elution of ²¹²Pb-DOTAM |
| | | and filling of syringes |
| 28 | 2020 Apr. 1 | IV injection of ²¹²Pb-DOTAM |
| 28 | 2020 Apr. 1 | Euthanasia and tissue harvest |
| | | (6 h p.i.) + gamma counting |

Study groups in protocol 185

| | P1AF0298 | P1AF0709 | P1AF0171 | P1AD8592 | | | |
|---|---|---|---|---|---|---|---|
| | T84.66 | T84.66 | CH1A1A | CH1A1A | | | |
| | (VH-AST) | (VL) | (VH-AST) | (VL) | ²¹²Pb | BD | n |
| Group | (μg) | (μg) | (μg) | (μg) | (μCi) | (h p.i.) | (mice) |
| A | 100 | 100 | 0 | 0 | 10 | 6 | 5 |
| B | 0 | 0 | 143* | 100 | 10 | 6 | 5 |

*P1AF0171 dose adjusted to 143 μg to compensate for a ~30% hole/hole impurity.

Solid xenografts were established in each SCID mouse on study day 0 by SC injection of 5×10⁶ cells (passage 27) in RPMI/Matrigel into the right flank. Twenty-two days after tumor cell injection, mice were sorted into experimental groups with an average tumor volume of 224 mm³. The ²¹²Pb-DOTAM was injected on day 28 after inoculation, at which point the average tumor volume had reached 385 mm³.

All mice were sacrificed and necropsied 6 hours after injection of ²¹²Pb-DOTAM, and the following organs and tissues harvested for measurement of radioactive content: blood, skin, spleen, pancreas, kidneys, liver, muscle, tail, and tumor. Collected tumors were split in two pieces: one was measured for radioactive content, and the other put in a cryomold containing Tissue-Tek® optimum cutting temperature (OCT) embedding medium, and put on dry ice for rapid freezing. Frozen samples in OCT were maintained at −80° C. before cryosectioning, immunofluorescence staining, and analysis using a Zeiss Axio Scope.A1 modular microscope.

Results

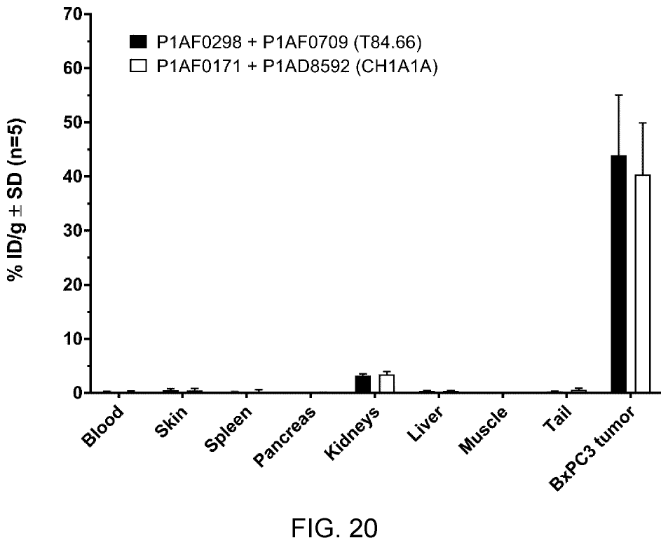

The average ²¹²Pb distribution in all collected tissues 6 hours after injection is shown in FIG. 20. The tumor accumulation was 40% ID/g (CH1A1A) or 44% ID/g (T84.66). The only other appreciable accumulation of radioactivity was found in kidneys: 3-5% ID/g at 6 h p.i. for the two groups.

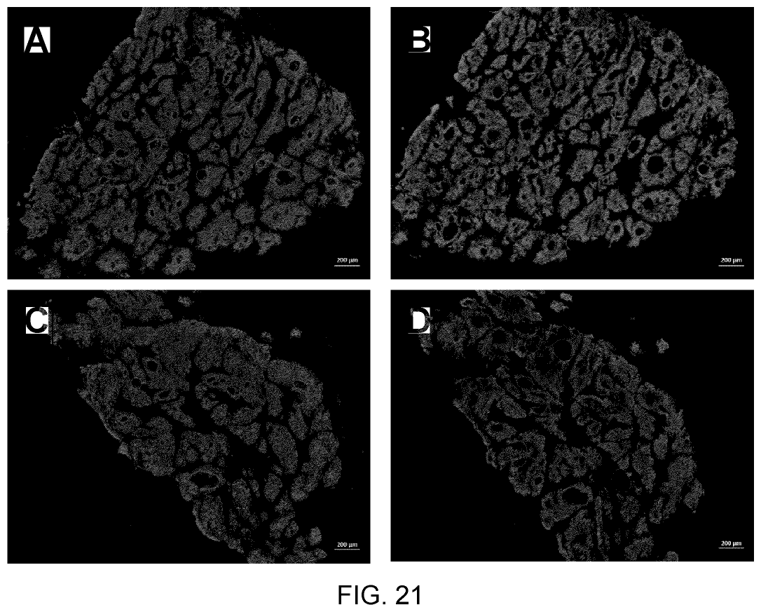

Examples of the intratumoral distribution of CEA-split-DOTAM-VH/VL pairs targeting either T84.66 (group A) or CH1A1A (group B) are shown in FIG. 21. Panels A and C show that the CEA expression is high and homogeneous in BxPC3 tumors, and panels B and D demonstrate that the antibody distribution 7 days after injection is distributed similarly. However, the samples from group A displayed a stronger signal overall, compared with tumor samples from group B, providing evidence that T84.66 is a stronger binder than CH1A1A.

Adverse Events and Toxicity

There were no adverse events or toxicity associated with this study.

Conclusion

The results verified the function of CEA-split-DOTAM-VH/VL constructs targeting the T84.66 epitope of CEA. The resulting accumulation of 212Pb in pretargeted CEA-expressing tumors was high and specific, and CEA-split-DOTAM-VH/VL pairs targeting either the CH1A1A or T84.66 epitope were homogeneously distributed inside the CEA-expressing tumors.

### Example 6: Protocol 189

The aim of protocol 189 was to assess bi-paratopic CEA-split-DOTAM-VH/VL antibody pairs targeting T84.66 VH-AST/CH1A1A VL and T84.66 VL/CH1A1 VH-AST, compared with the positive control pair targeting CH1A1A VH-AST/VL. This bi-paratopic combination precludes formation of the full Pb-DOTAM binder on soluble CEA that only expresses one of the two epitopes (e.g. T84.66), thereby mitigating potential adverse effects thereof, such as increased circulating radioactivity and associated radiation-induced toxicity, and decreased efficacy from competition with off-tumor targets.

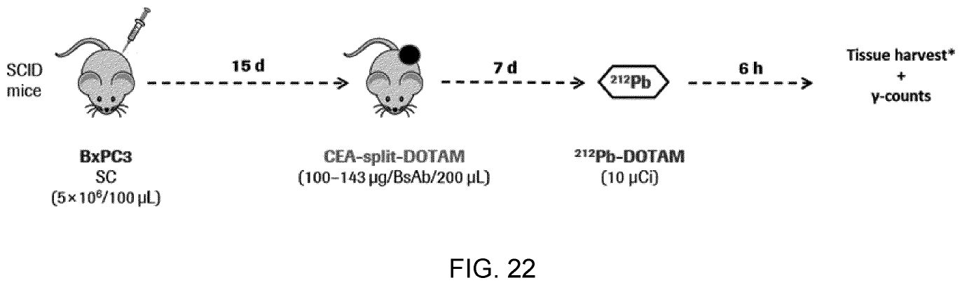

Mice carrying SC BxPC3 tumors were injected with the standard dose of CEA-split-DOTAM-VH/VL BsAb (100 μg per antibody) followed 7 days later by the radiolabeled ²¹²Pb-DOTAM. The in vivo distribution of ²¹²Pb-DOTAM was assessed 6 hours after the radioactive injection. The study outline is shown in FIG. 22.

Study Design

The time course and design of protocol 189 is shown below.

Study groups in protocol 189

| | P1AF0298 | P1AF0709 | P1AF0171 | P1AD8592 | | | |
|---|---|---|---|---|---|---|---|
| | T84.66 | T84.66 | CH1A1A | CH1A1A | | | |
| | (VH-AST) | (VL) | (VH-AST) | (VL) | ²¹²Pb | BD | n |
| Group | (μg) | (μg) | (μg) | (μg) | (μCi) | (h p.i.) | (mice) |
| A | 100 | 0 | 0 | 100 | 10 | 6 | 5 |
| B | 0 | 100 | 143* | 0 | 10 | 6 | 5 |
| C | 0 | 0 | 143* | 100 | 10 | 6 | 3 |

*P1AF0171 dose adjusted to 143 μg to compensate for a ~30% hole/hole impurity.

Time course of protocol 189

| Study day | Experimental procedure |
|---|---|
| 0 | Preparation of BxPC3 cells and filling of syringes |
| 0 | SC injection of BxPC3 cells |
| 15 | IV injection of CEA-split-DOTAM-VH/VL BsAbs |
| 21 | Elution of ²¹²Pb-DOTAM and filling of syringes |
| 22 | IV injection of ²¹²Pb-DOTAM |
| 22 | Euthanasia and tissue harvest (6 h p.i.) + gamma counting |

Solid xenografts were established in each SCID mouse on study day 0 by SC injection of 5×10⁶ cells (passage 31) in RPMI/Matrigel into the right flank. Fourteen days after tumor cell injection, mice were sorted into experimental groups with an average tumor volume of 343 mm³. The ²¹²Pb-DOTAM was injected on day 22 after inoculation; the average tumor volume had reached 557 mm³ on day 21.

All mice were sacrificed and necropsied 6 hours after injection of ²¹²Pb-DOTAM, and the following organs and tissues harvested for measurement of radioactive content: blood, skin, spleen, pancreas, kidneys, liver, muscle, tail, and tumor.

Results

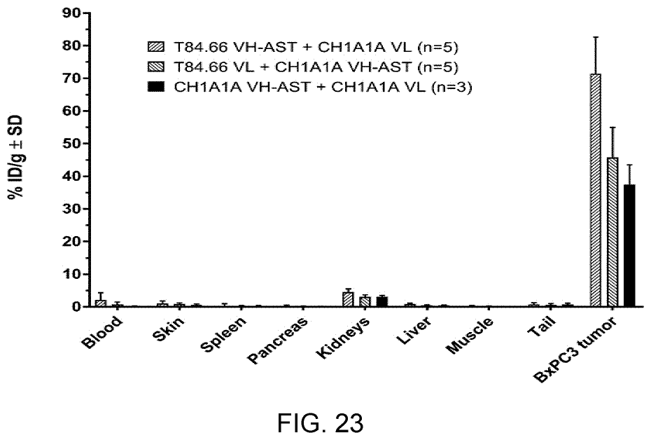

The average ²¹²Pb distribution in all collected tissues 6 hours after injection is shown in FIG. 23. The tumor accumulation of the bi-paratopic variations was 71% ID/g and 46% ID/g for T84.66 VH-AST+CH1A1A VL and T84.66 VL+CH1A1A VH-AST, respectively. The positive CH1A1A control resulted in 37% ID/g. Two-way ANOVA with Tukey's multiple comparisons test showed that all three groups were significantly different from each other in terms of tumor uptake (p<0.0001 for T84.66 VH-AST+CH1A1A VL versus the two other groups; p=0.0020 for T84.66 VL+CH1A1A VH-AST versus CH1A1A only). No other organs showed statistically significant differences between groups, although a slightly higher retention in blood was indicated for the T84.66 VH-AST+CH1A1A VL combination compared with the two other groups: 2% ID/g compared with <1% ID/g. The kidney uptake was similarly slightly higher, although not statistically significantly so: 4.5% ID/g for T84.66 VH-AST+CH1A1A compared with 3% ID/g for the other two.

Adverse Events and Toxicity

There were no adverse events or toxicity associated with this study. However, the BxPC3 tumor growth was significantly faster, and with greater variability, in this study compared with the standard growth rate. On necropsy, it was concluded that the big tumors (a majority) were filled with liquid, which was emptied when tumors were cut in half before radioactive measurement; this liquid likely caused the accelerated growth rate, but did not affect the % IA/g to any great extent as the tumors were weighed and measured after being opened.

Conclusion

The results verified the function of bi-paratopic targeting of the T84.66 and CH1A1A epitopes of CEA using the tested CEA-split-DOTAM-VH/VL constructs and demonstrated surprisingly high efficacy for this combination as compared to the positive CH1A1A control. The resulting accumulation of ²¹²Pb in pretargeted CEA-expressing tumors was high and specific, with indications of a particular advantage for the T84.66 VH-AST+CH1A1A VL pair.

### Example 7

These examples investigate recruitment of Pb-DOTA to cells by split antibodies as described herein.

P1AF0712 has a first heavy chain of SEQ ID NO:97, a second heavy chain of SEQ ID NO: 98 and a light chain of SEQ ID NO: 103. P1AF0713 has a first heavy chain of SEQ ID NO: 100, a second heavy chain of SEQ ID NO: 101 and a light chain of SEQ ID NO: 103.

MKN-45 cells were detached from the culture bottle using Trypsin and were counted using a Casy cell counter. After pelleting at 4° C., 300 g the cells were resuspended in FACS Buffer (2.5% FCS in PBS), adjusted to 2.0E+06 cells/mL dispensed to 96-well PP V-bottom-Platte (25 μL/well=5.0E+04Zellen/well).

FACS Staining Using DOTA-FITC

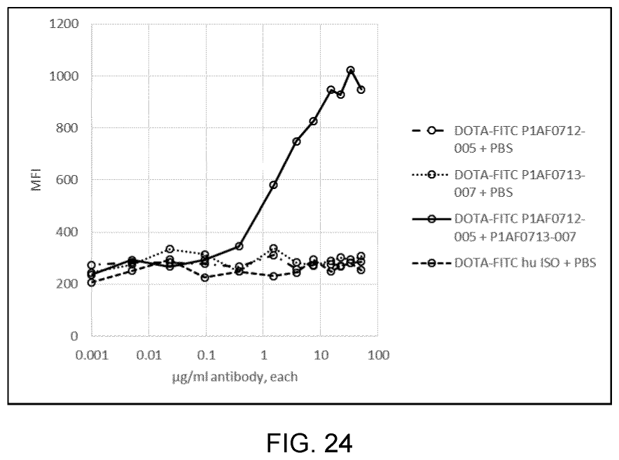

The CEA specific SPLIT antibodies (P1AF0712 or P1AF0713respectively) were adjusted to 40 μg/mL in FACS buffer, resulting in a final concentration of 10 μg/mL. Both antibodies were added to the cells either combined or separated and followed by buffer and incubated at 4° C. for 1 h. Subsequently, Pb-DOTA labeled with FITC was added to the cells in equimolar ratio to the antibodies and incubated for 1 h at 4° C. The cells were then washed twice in FACS buffer and resuspended in 70 μl/well FACS buffer for measurement using a FACS Canto (BD, Pharmingen). It was shown (FIG. 24) that neither of the SPLIT halves was giving rise to a fluorescence signal, indicating a lack of Pb-DOTA binding capability. Only a combination of both SPLIT halves was able to recruit Pb-DOTAM-FITC to the target cells (FIG. 24).

FACS Staining Using <huIgG(H+L)A488>

The CEA specific SPLIT antibodies (P1AF0712 or P1AF0713 respectively) were adjusted to 40 μg/mL in FACS buffer, resulting in a final concentration of 10 μg/mL. Both antibodies were added to the cells either separated followed by buffer or combined and incubated at 4° C. for 1 h. The cells were then washed twice in FACS buffer. After washing, the cells were resuspended in 50 μL FACS-buffer containing secondary antibody (<huIgG(H+L)>-Alexa488, c=10 μg/mL) and incubated 1h at 4° C. The cells were then washed twice in FACS buffer and resuspended in 70 μl/well FACS buffer for measurement using a FACS Canto (BD, Pharmingen). EC50 for both SPLIT antibodies was comparable, indicating CEA specific cell binding of both SPLIT antibodies. Due to the higher amount of antibody in the mixture, a lower EC50 was obtained under these circumstances, as shown in the table below.

EC50 Determination of SPLIT Antibodies

| | | EC50 |
|---|---|---|
| | | μg/ml |
| | | absolute |
| <hu>A488 | P1AF0712 + PBS | 2.7 |
| | P1AF0713 + PBS | 2.3 |
| | P1AF0712 + P1AF0713 | 0.9 |
| | hu ISO + PBS | |
| DOTA-FITC | P1AF0712 + PBS | na |
| | P1AF0713 + PBS | na |
| | P1AF0712 + P1AF0713 | 2.4 |
| | hu ISO + PBS | |

EC50 was determined for the SPLIT antibodies using either secondary antibody based detection, (<hu>488, top panel) or Pb-DOTA-FITC (DOTA-FITC, bottom panel)

### Example 8: Biacore Binding Experiments

This example tests binding of TA-split-DOTAM-VH and TA-split-DOTAM-VL individually to DOTAM, as compared to the reference antibody CEA-DOTAM (RO7198427, PRIT-0213). It further tests binding of DOTAM to the TA-split-DOTAM-VH/VL pairs, as compared to the reference antibody.

The correspondence between the coding used in these examples and the protein numbers used elsewhere in this application is shown below. Sequences are also provided. The reference antibody is coded as “PRIT_RS” in this example.

| | | | | | | | Fusion |
|---|---|---|---|---|---|---|---|
| | | | | | LC | HC | HC |
| | SPR Code | | | | (SEQ | (SEQ | (SEQ |
| | (Prodrug | SPR | | | ID | ID | ID |
| Target binder | A + B) | Code | DOTAM | Protein | NO) | NO) | NO) |
| <CEA> CH1A1A | P1_AB | P1_A | VL | P1AD8592 | 34 | 30 | 33 |
| | | P1_B | VH | P1AD8749 | 34 | 28 | 32 |
| <CEA> CH1A1A | P2_AB | P2_A | VL | P1AD8592 | 34 | 30 | 33 |
| | | P2_B | VH | P1AF0171 | 34 | 28 | 147 |
| <CEA> T84.66 | P3_AB | P3_A | VL | P1AF0709 | 89 | 83 | 84 |
| | | P3_B | VH | P1AF0298 | 89 | 86 | 89 |
| <CEA> 28A9 | P4_AB | P4_A | VL | P1AF0710 | 96 | 90 | 91 |
| | | P4_B | VH | P1AF0711 | 96 | 93 | 94 |
| <CEA> | P5_AB | P5_A | VL | P1AE4957 | 58 | 55 | 56 |
| A5H1EL1(G54A) | | P5_B | VH | P1AE4956 | 54 | 51 | 52 |
| <CEA> CH1A1A | P6_AB | P6_A | VL | P1AF0712 | 103 | 97 | 98 |
| | | P6_B | VH | P1AF0713 | 103 | 100 | 101 |
| <GPRC5D> | P7_AB | P7_A | VL | P1AF8284 | 107 | 104 | 105 |
| | | P7_B | VH | P1AF8285 | 107 | 104 | 106 |

For these experiments, the PRIT SPLIT antibodies were purified by a first step of MabSelect Sure (Affinity Chromatography) and a second step of ion exchange chromatography (e.g. POROS XS), and then polished by Superdex 200 (Size Exclusion Chromatography).

The experiments were performed with Biacore 71200 at 25° C. measuring temperature. All Biacore T200 experiments were carried out in BS-P+ (GE Healthcare, Br-1008-27) pH 7,4 running buffer. Two experiments were performed for each test antibody/antibody pair, using different DOTAM fractions.

1. In a first experiment, the binding of individual TA-split-DOTAM-VHF and TA-split-DOTAM-VL antibodies to biotinylated l)TAM captured on a chip was assessed, relative to the reference antibody.

DOTAM (120 nM solution in HBS-P+) was captured in high density on CAP Chip Surface (10 μl/min, 60Sec). Then the 600 nM solutions in H7BS-P+ of Prodrug_A or Prodrug_B were injected over the DOTAM surface (10 μl/min, 90 sec). The dissociation was monitored for 240 sec at a flow rate of 10 μl/min. The relative maximum response determination was evaluated using T200 evaluation software.

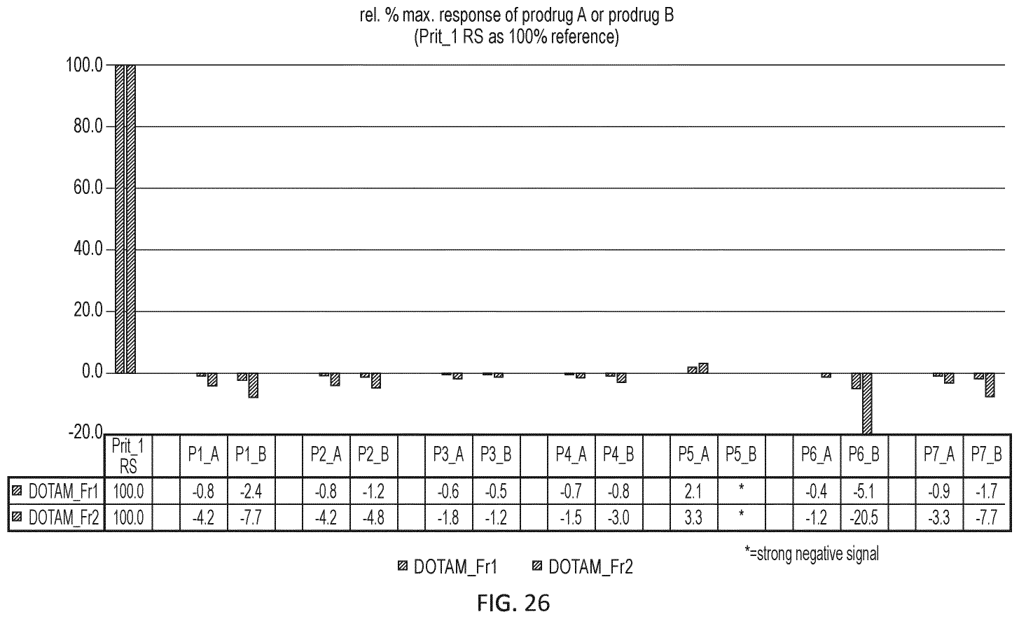

The results are shown in FIG. 26. None of the individual antibodies showed binding to the captured DOTAM.

2. In a second experiment, individual TA-split-DOTAM-VH and TA-split-DOTAM-VL antibodies were first captured in a chip using an immobilized anti-hFab, and then binding of a DOTAM-monoStreptavidin complex (DOTAM+monoSteptavidin coupling 600 nM, 1:1 mol, 1h at RT) was assessed.

The 600 nM solution in 11BS-P+ of Prodrug_A or Prodrug_B was injected over the anti hFab (GE Healthcare, BR-1008-27) CM5 Chip surface (10 μl/min, 120 sec). After the high density capturing of Prodrug A or B solution the DOTAM-monoStreptavidin complex was injected (20 μl/min, 90 sec). The dissociation was monitored for 180 sec at a flow rate of 20 μl/min. For new cycle the surface was regenerated by using of Glycin 2.1 and 75 sec regeneration time with 10 i/min. The relative maximum response determination was evaluated using T200 evaluation software.

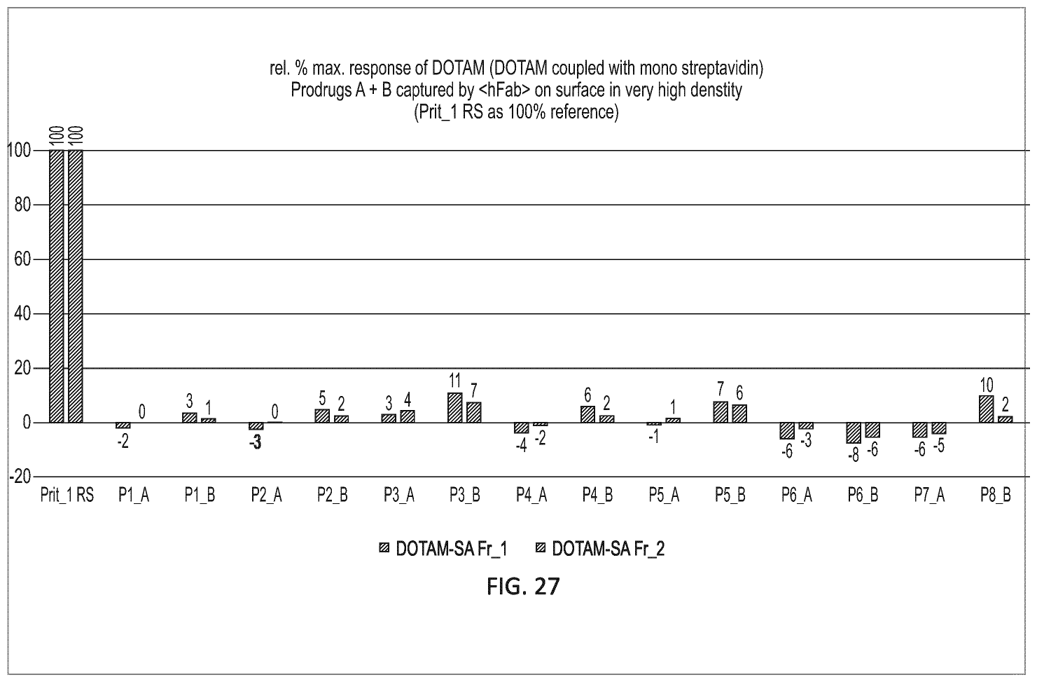

The results are shown in FIG. 27. Low percentage max. responses (as marked with * in the figure) are believed to be “traces” or unspecific interactions with DOTAM-SA, and reflect a need to optimize the assay.

3. In a third experiment, binding of the TA-split-DOTAM-VH/VL pairs to DOTAM is assessed, as compared to the reference antibody. Antibodies were first captured in a chip using an immobilized anti-hFab, and then binding of a DOTAM-monoStreptavidin complex (DOTAM+monoSteptavidin coupling 600 nM, 1:1 mol, 1h at RT) was assessed.

The 300 nM solution in HBS-P+ of Prodrug_A and Prodrug B was injected over the anti hFab (GE Healthcare, BR-1008-27) CM5 Chip surface (101/min, 120 sec). After the high density capturing of Prodrug A and B solution the DOTAM-monoStreptavidin complex was injected (20 μl/min, 90 sec). The dissociation was monitored for 180 sec at a flow rate of 20 μl/min. For new cycle the surface was regenerated by using of Glycin 2.1 and 75 sec regeneration time with 10 μl/min. The relative maximum response determination was evaluated using T200 evaluation software.

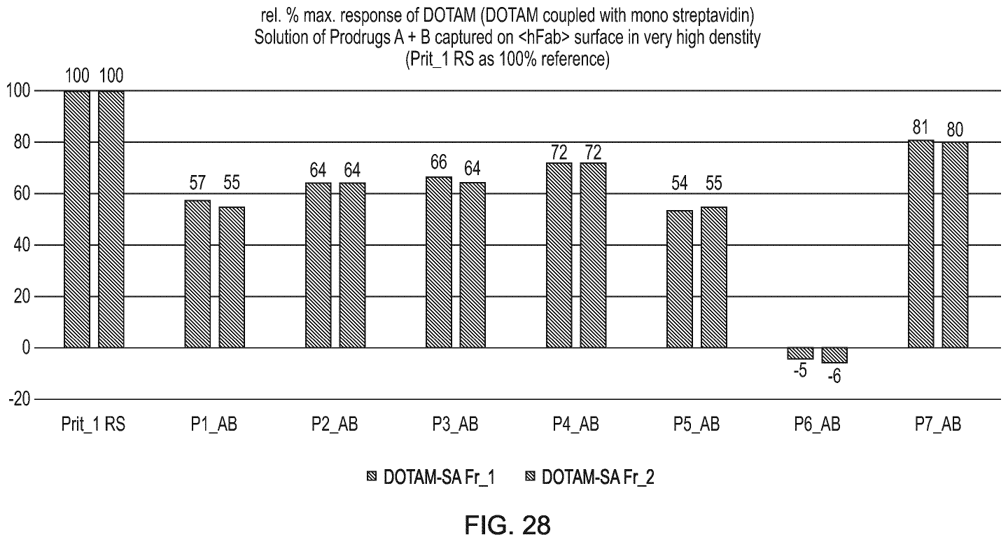

The results are shown in FIG. 28. All TA-split-DOTAM-VH/VL pairs showed a significant amount of binding for DOTAM, except for the P6_AB (P1AF0712/P1 AF0713) pair, which are DOTA binders.

Similar results showing a significant amount of DOTAM binding for the TA-split-DOTAM-VH/VL pair but not for the individual members of the pair have also been obtained for the FAP-binders P1AF8286 and P1AF8287. P1AF8286 is composed of a first heavy chain of SEQ ID NO: 108, a second heavy chain of SEQ ID NO: 109 and a light chain of SEQ ID NO: 111, and P1AF8287 is composed of a first heavy chain of SEQ ID NO: 108, a second heavy chain of SEQ ID NO: 110 and a light chain of SEQ ID NO: 111. However, this assay still needs to be optimised.

### Example 9: Combination Therapies

### Example 9a: Materials and Methods, General

Health Monitoring and Termination Criteria

All experimental protocols were reviewed and approved by the local authorities (Comite Regional d'Ethique de l'Experimentation Animale du Limousin [CREEAL], Laboratoire Départemental d'Analyses et de Recherches de la Haute-Vienne). Female transgenic C₅₇BL/6J-TgN(CEAGe)18FJP [Clarke P, Mann J, Simpson J F, Rickard-Dickson K, Primus F J. Mice transgenic for human carcinoembryonic antigen as a model for immunotherapy. Cancer Res. 1998; 58(7):1469-77] (“B6-huCEA”) and C₅₇BL/6J-Tg(CEACAM5)2682Wzm [Eades-Perner A M, van der Putten H, Hirth A, Thompson J, Neumaier M, von Kleist S, Zimmermann W. Mice transgenic for the human carcinoembryonic antigen gene maintain its spatiotemporal expression pattern. Cancer Res. 1994; 54(15):4169-76] (“huCEACAM5”) mice from Charles River were maintained under specific and opportunistic pathogen free (SOPF) conditions with daily cycles of light and darkness (12 h/12 h), in line with ethical guidelines. No manipulations were performed during the first 5 days after arrival, to allow the animals to acclimatize to the new environment.

Primary solid xenografts were established in each B6-huCEA or huCEACAM5 mouse on study day 0 through subcutaneous (SC) or intrapancreatic injection of carcinoembryonic antigen (CEA)-expressing tumor cells. Animals were controlled daily for clinical symptoms and detection of adverse events, and euthanized before the scheduled endpoint if they showed signs of unamenable distress or pain due to tumor burden, side effects of the injections or surgery, or other causes. Indications of pain, distress, or discomfort include, but are not limited to, acute body weight (BW) loss, scruffy fur, diarrhea, hunched posture, pale skin, and reluctance to move. Poor SC tumor status (e.g. ulceration, teeth marks, or open wounds) may also prompt euthanasia; in the orthotopic model, abdominal swelling indicates increasing tumor burden, which may prompt euthanasia.

SC tumor volumes were estimated through manual calipering 3 times per week, calculated according to the formula: volume 0.5× length×width². Additional tumor measurements were made as needed depending on the tumor growth rate. In the orthotopic model, the tumor progress was assessed regularly through bioluminescence imaging (BLI). The BW of the animals was measured at least 3 times per week, with additional measurements as needed depending on the health status. Mice whose BW loss exceeded 20% of their initial BW or whose tumor volume reached 3000 mm³ (Protocols 119, 136, 150) or 2000 mm³ (Protocol 195) were euthanized immediately.

To minimize re-ingestion of radioactive urine/feces, mice were placed in cages with grilled floors for 4 hours after ²¹²Pb-1,4,7,10-tetrakis(carbamoylmethyl)-1,4,7,10-tetraazacyclododecane (DOTAM) administration, before being transferred to new cages with standard bedding. All cages were then changed 24 hours post injection (p.i.). Wet food was provided to all mice from the day after the radioactive injection, for 7 days or until all individuals had recovered sufficiently from any acute BW loss.

Bioluminescence Imaging

The tumor progress was assessed through repeated BLI using a Bruker In-Vivo FX PRO system. To limit interference with the signal, the fur on and around the injection area was removed as much as possible before imaging using an electrical razor. For imaging, mice were SC injected with 100 μL of luciferin (D-Luciferin; Thermo Scientific, reference No. 88294), on the back. The solution was diluted in phosphate-buffered saline (PBS) to 15 mg/mL, passed through a 0.2-μm filter, and stored at −20° C. until use. During imaging, mice were placed side by side with the injection site facing down. An optical photo of the setup was taken, followed by a 1-minute BLI acquisition. For maximization of the signal, the acquisition was started 10 minutes after the luciferin injection. The images were then overlaid for visual assessment. Rectangular regions of interest (ROI) were drawn and the background-corrected signal within the ROIs compared for each mouse to assess the tumor progression.

Tissue Harvest

Blood was sampled from mice after the immunotherapy administration to validate the antibody injections by measuring their respective serum concentrations. Samples were centrifuged at 10 000 RCF during 5 minutes, and the resulting serum fractions isolated, frozen, and stored at −20° C. for subsequent analysis by enzyme-linked immunosorbent assay (ELISA) performed by Discovery Pharmacology, Roche Innovation Center Zurich.

Blood was also collected at the time of euthanasia from the venous sinus using retro-orbital bleeding on anesthetized mice, before termination through cervical dislocation. This was followed by additional tissue harvest for radioactive measurements and/or histological analysis, as mandated by the protocols. Unexpected or abnormal conditions were documented. Tissues collected for formalin fixation were immediately put in 10% neutral buffered formalin (NBF; 4° C.) and then transferred to PBS (4° C.) after 24 h. Organs and tissues collected for biodistribution purposes were weighed and measured for radioactivity using a 2470 WIZARD² automatic gamma counter (PerkinElmer), and the percent injected dose per gram of tissue (% ID/g) subsequently calculated, including corrections for decay and background.

Statistical Analysis

Statistical analysis was performed using GraphPad Prism 7 (GraphPad Software, Inc.), JMP 12 (SAS Institute Inc.), and DOPsa (in-house application). Curve analysis of tumor growth inhibition (TGI) was performed based on mean tumor volumes using the formula:

$$TGI = 100 - \frac{\overline{{v}_{treatment , d} - {v}_{treatment , 0}}}{\overline{{v}_{ref , d} - {v}_{ref , 0}}} \times 100$$

where d indicates study day and 0 the baseline value. “Vehicle” was selected as the reference group.

1.1 Test Compounds

CEA-DOTAM (mu) (P1AD8758) is a murinized BsAb targeting the CH1A1A epitope of CEA. It is composed of the following polypeptide chains:

>LC
DIQMTQSPSSLSASVGDRVTITCKASAAVGTYVAWYQQKPGKAPKLLIYS
ASYRKRGVPSRFSGSGSGTDFTLTISSLQPEDFATYYCHQYYTYPLFTFG
QGTKLEIKRADAAPTVSIFPPSSEQLTSGGASVVCFLNNFYPKDINVKWK
IDGSERQNGVLNSWTDQDSKDSTYSMSSTLTLTKDEYERHNSYTCEATHK
TSTSPIVKSFNRNEC
>HC 1
QVQLVQSGAEVKKPGASVKVSCKASGYTFTEFGMNWVRQAPGQGLEWMGW
INTKTGEATYVEEFKGRVTFTTDTSTSTAYMELRSLRSDDTAVYYCARWD
FAYYVEAMDYWGQGTTVTVSSAKTTPPSVYPLAPGSAAQTNSMVTLGCLV
KGYFPEPVTVTWNSGSLSSGVHTFPAVLQSDLYTLSSSVTVPSSTWPSET
VTCNVAHPASSTKVDKKIVPRDCGCKPCICTVPEVSSVFIFPPKPKDVLT
ITLTPKVTCVVVAISKDDPEVQFSWFVDDVEVHTAQTQPREEQFNSTFRS
VSELPIMHQDWLNGKEFKCRVNSAAFGAPIEKTISKTKGRPKAPQVYTIP
PPKEQMAKDKVSLTCMITNFFPEDITVEWQWNGQPAENYDNTQPIMDTDG
SYFVYSDLNVQKSNWEAGNTFTCSVLHEGLHNHHTEKSLSHSPGGGGGSG
GGGSGGGGSGGGGSQSVEESGGRLVTPGTPLTLTCTVSGFSLSTYSMSWV
RQAPGKGLEWIGFIGSRGDTYYASWAKGRFTVSRTSTTVDLKITSPTTED
TATYFCARERDPYGGGAYPPHLWGPGTLVTVSS
>HC 2C
QVQLVQSGAEVKKPGASVKVSCKASGYTFTEFGMNWVRQAPGQGLEWMGW
INTKTGEATYVEEFKGRVTFTTDTSTSTAYMELRSLRSDDTAVYYCARWD
FAYYVEAMDYWGQGTTVTVSSAKTTPPSVYPLAPGSAAQTNSMVTLGCLV
KGYFPEPVTVTWNSGSLSSGVHTFPAVLQSDLYTLSSSVTVPSSTWPSET
VTCNVAHPASSTKVDKKIVPRDCGCKPCICTVPEVSSVFIFPPKPKDVLT
ITLTPKVTCVVVAISKDDPEVQFSWFVDDVEVHTAQTQPREEQFNSTFRS
VSELPIMHQDWLNGKEFKCRVNSAAFGAPIEKTISKTKGRPKAPQVYTIP
PPKKQMAKDKVSLTCMITNFFPEDITVEWQWNGQPAENYKNTQPIMKTDG
SYFVYSKLNVQKSNWEAGNTFTCSVLHEGLHNHHTEKSLSHSPGGGGGSG
GGGSGGGGSGGGGSAVLTQTPSPVSPAVGGTVTISCQSSHSVYSDNDLAW
YQQKLGQPPKLLIYQASKLASGVSSRFSGSGSGTQFTLTISGVQSDDAAT
YYCLGGYDDESDTYGFGGGTEVVVK

CEA-split-DOTAM-VL as used in protocol 195 is P1AD8592, described elsewhere in this application (see for instance example 1). CEA-split-DOTAM-VH-AST as used in protocol 195 is P1AF0171, described elsewhere in this application (see for instance example 4).

DIG-DOTAM (RO7204012) is a non-CEA-binding BsAb (target=digoxigenin), used as a negative control.

The anti-CD40 antibody is muIgG1 CD40 FGK4.5 B6 CHO W(9). It has the heavy chain of SEQ ID NO: 61 and the light chain of SEQ ID NO: 62 as taught in WO2018/189220, using the sequence numbering of that document. The anti-PD-L1 used in protocol 119 is 6E11 muIgG1 GNE w(1) (also termed “murine IgG1, clone 6E11, Genentech”). See for instance WO2018/055145. The anti-PD-L1 used in protocol 136 and 195 is 6E11.mIgG2a.LALAPG.

All antibody constructs were stored at −80° C. until the day of injection when they were thawed and diluted in standard vehicle buffer (20 mM Histidine, 140 mM NaCl; pH 6.0) or 0.9% NaCl to their final respective concentrations for intravenous (IV) or intraperitoneal (IP) administration. Likewise, the anti-CD40 and anti-PD-L1 antibodies were stored at −80° C. and diluted in histidine buffer to 200 μg per 200 μL on the day of IP injection.

The Pb-DOTAM-dextran-500 and Ca-DOTAM-dextran-500 CAs (RO7201869) were stored at −20° C. until the day of injection when they were thawed and diluted in PBS for IV or IP administration.

The DOTAM chelate for radiolabeling was provided by Macrocyclics and maintained at −20° C. before radiolabeling, performed by Orano Med (Razes, France). ²¹²Pb-DOTAM (RO7205834) was generated by elution with DOTAM from a thorium generator, and subsequently quenched with Cu or Ca after labeling. The ²¹²Pb-DOTAM solution was diluted with PBS or 0.9% NaCl to obtain the desired ²¹²Pb activity concentration for IV injection.

Mice in vehicle control groups received multiple injections of vehicle buffer instead of BsAb, CA, and ²¹²Pb-DOTAM.

Tumor Models

The tumor cell lines used for inoculation in mice are Panc02-huCEA-luc or MC38-huCEA.

Panc02 is a cell line derived from mouse pancreatic ductal adenocarcinoma cells, acquired from The University of Texas MD Anderson Cancer Center (Houston, TX) and engineered by Roche to express human CEA (huCEA) and luciferase (luc), producing Panc02-huCEA-luc. Cells were cultured in RPMI-1640 medium enriched with 1% GlutaMAX (Gibco, cat No. 72400-021), 10% fetal bovine serum (GE Healthcare, cat No. SH30088.03), 4 μg/mL of puromycin (VWR, cat No. J593), and 50 μg/mL of hygromycin (Cayman Chemicals, cat No. 14291).

MC38-huCEA is a murine colon adenocarcinoma cell line engineered to express huCEA, acquired from City of Hope, CA, USA. Cells were cultured in DMEM medium enriched with GlutaMAX (Gibco, cat No. 61965-026), 10% fetal bovine serum (GE Healthcare, cat No. SH30088.03) and 500 μg/mL of geneticin (Gibco, cat No. 10131-027).

SC xenografts were established in each mouse on study day 0 by SC injection of cells in media mixed 1:1 with Corning® Matrigel® basement membrane matrix (growth factor reduced; cat No. 354230), into the right flank. In the orthotopic model, primary solid intrapancreatic xenografts were established in each mouse through injection of cells in media directly into the pancreas.

### Example 9b: Protocol 119

The aim of protocol 119 was to assess the efficacy following three cycles of CEA-pretargeted radioimmunotherapy (PRIT), alone and in combination with cancer immunotherapy (CIT), for treatment in a syngeneic orthotopic murine model of pancreatic adenocarcinoma.

Immunocompetent transgenic B6-huCEA mice were injected in the pancreas with Panc02-huCEA-luc tumor cells (0.2×10⁶ cells in 10 μL), and the tumor development followed by BLI. The CEA-PRIT regimen comprised IV injection of CEA-DOTAM (mu) BsAb (100 μg in 100 μL) followed 4 days later by IV administration of a Pb-DOTAM-dextran-500 CA (25 μg in 100 μL), followed in turn by the Cu-quenched ²¹²Pb-DOTAM effector molecule (20 μCi in 100 μL), IV injected 2 hours after the CA. The CIT treatment was administered IP 24 hours after the radioactive injection, consisting of a one-time administration of anti-CD40 antibody and multiple injections of anti-PD-L1 antibody (200 μg of each antibody in 200 μL). Scout mice were taken for biodistribution assessment to confirm ²¹²Pb-DOTAM targeting and clearance during the first treatment cycle. The treatment efficacy was assessed in terms of TGI (based on BLI) and survival.

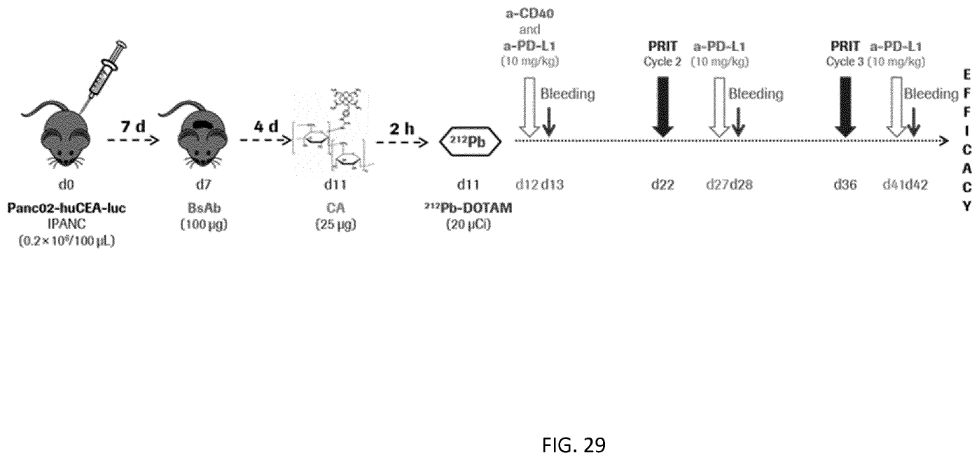

The study outline is shown in FIG. 29.

The time course and design of protocol 119 are shown in the tables below.

Time course of protocol 119

| Study | | |
|---|---|---|
| day | Date | Experimental procedure |
| 0 | 2017 Jun. 8 | Preparation of Panc02-huCEA-luc cells and |
| | | filling of syringes |
| 0 | 2017 Jun. 8 | SC injection of Panc02-huCEA-luc cells |
| 6 | 2017 Jun. 14 | SC injection of D-Luciferin + imaging (BLI) |
| 7 | 2017 Jun. 15 | IV injection of BsAb |
| 11 | 2017 Jun. 19 | SC injection of D-Luciferin + imaging (BLI) |
| 11 | 2017 Jun. 19 | IV injection of CA |
| 11 | 2017 Jun. 19 | Elution of ²¹²Pb-DOTAM and filling of syringes |
| 11 | 2017 Jun. 19 | IV injection of ²¹²Pb-DOTAM |
| 12 | 2017 Jun. 20 | Euthanasia and tissue harvest (24 h p.i.) + |
| | | gamma counting |
| 12 | 2017 Jun. 20 | IP injection of anti-CD40 and anti-PD-L1 |
| 13 | 2017 Jun. 21 | Retro-orbital bleeding |
| 14 | 2017 Jun. 22 | SC injection of D-Luciferin + imaging (BLI) |
| 18 | 2017 Jun. 26 | SC injection of D-Luciferin + imaging (BLI) |
| 21 | 2017 Jun. 29 | SC injection of D-Luciferin + imaging (BLI) |
| 22 | 2017 Jun. 30 | IV injection of BsAb |
| 25 | 2017 Jul. 3 | SC injection of D-Luciferin + imaging (BLI) |
| 26 | 2017 Jul. 4 | IV injection of CA |
| 26 | 2017 Jul. 4 | Elution of ²¹²Pb-DOTAM and filling of syringes |
| 26 | 2017 Jul. 4 | IV injection of ²¹²Pb-DOTAM |
| 27 | 2017 Jul. 5 | IP injection of anti-PD-L1 |
| 28 | 2017 Jul. 6 | Retro-orbital bleeding |
| 28 | 2017 Jul. 6 | SC injection of D-Luciferin + imaging (BLI) |
| 32 | 2017 Jul. 10 | SC injection of D-Luciferin + imaging (BLI) |
| 35 | 2017 Jul. 13 | SC injection of D-Luciferin + imaging (BLI) |
| 36 | 2017 Jul. 14 | IV injection of BsAb |
| 39 | 2017 Jul. 17 | SC injection of D-Luciferin + imaging (BLI) |
| 40 | 2017 Jul. 18 | IV injection of CA |
| 40 | 2017 Jul. 18 | Elution of ²¹²Pb-DOTAM and filling of syringes |
| 40 | 2017 Jul. 18 | IV injection of ²¹²Pb-DOTAM |
| 41 | 2017 Jul. 19 | IP injection of anti-PD-L1 |
| 42 | 2017 Jul. 20 | Retro-orbital bleeding |
| 43 | 2017 Jul. 21 | SC injection of D-Luciferin + imaging (BLI) |
| 46 | 2017 Jul. 24 | SC injection of D-Luciferin + imaging (BLI) |
| 49 | 2017 Jul. 27 | SC injection of D-Luciferin + imaging (BLI) |
| 53 | 2017 Jul. 31 | SC injection of D-Luciferin + imaging (BLI) |
| 53 | 2017 Aug. 3 | SC injection of D-Luciferin + imaging (BLI) |
| 60 | 2017 Aug. 7 | SC injection of D-Luciferin + imaging (BLI) |
| 63 | 2017 Aug. 10 | SC injection of D-Luciferin + imaging (BLI) |

Study groups in protocol 119

| | | BsAb | CA | ²¹²Pb | anti-CD40* | anti- | | n |
|---|---|---|---|---|---|---|---|---|
| Group | BsAb | (μg) | (μg) | (μCi) | (μg) | PD-L1 (μg) | Cycles | (mice) |
| A | — | 0 | 0 | 0 | 0 | 0 | 3 | 8 |
| B | — | 0 | 0 | 0 | 200 | 200 | 3 | 8 |
| C | CEA-DOTAM (mu) | 100 | 25 | 20 | 0 | 0 | 3 | 8 |
| D | CEA-DOTAM (mu) | 100 | 25 | 20 | 200 | 200 | 3 | 8 |
| E | CEA-DOTAM (mu) | 100 | 25 | 20 | 0 | 0 | 1 | 3 |
| F | DIG-DOTAM | 100 | 25 | 20 | 0 | 0 | 1 | 3 |

*Anti-CD40 only administered once, at the first treatment cycle

Primary solid xenografts were established in each B6-huCEA mouse (age 10 weeks) on study day 0 through injection of Panc02-huCEA-luc cells (passage 19) in RPMI-1640 media (0.2×10⁶ cells in 10 μL) directly into the pancreas. The tumor progress was assessed in all mice through BLI with measurements on days 6, 11, 14, 18, 21, and then twice weekly until day 63 after inoculation.

Mice in groups A-D were followed to assess therapeutic efficacy until the end of the study or until one or several of the termination criteria were reached. Blood was sampled from mice in groups B and D 24 hours after administration of the immunotherapy, to validate the anti-CD40 and anti-PD-L1 injections by analysis of serum fractions through ELISA. Serum was also isolated before euthanasia through retro-orbital blood collection, and then frozen and stored at −20° C. The following tissues were collected for histological processing and analysis and immediately put in 10% NBF for 24 hours, before being transferred to 1×PBS solution: serum, liver, spleen, kidneys, and pancreas with tumor.

Mice in groups E and F were sacrificed and necropsied 24 hours after ²¹²Pb-DOTAM injection to confirm tumor uptake and clearance from normal tissues. The following organs and tissues were thus harvested and measured for radioactivity: blood, bladder, spleen, pancreas (without tumor), kidneys, liver, muscle, skin, tail, and tumor.

Results

Biodistribution and ELISA

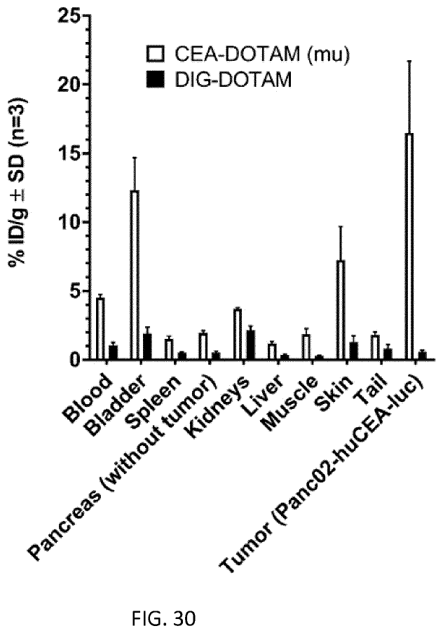

The average ²¹²Pb accumulation and clearance in all collected tissues 24 hours after the first ²¹²Pb-DOTAM injection is displayed in FIG. 30. The tumor uptake was specific, with 16.5% ID/g in the tumor after pretargeting with CEA-DOTAM (mu), compared with 0.6% ID/g with DIG-DOTAM. In pancreas tissue without tumor, the ²¹²Pb accumulation was 1.9% ID/g.

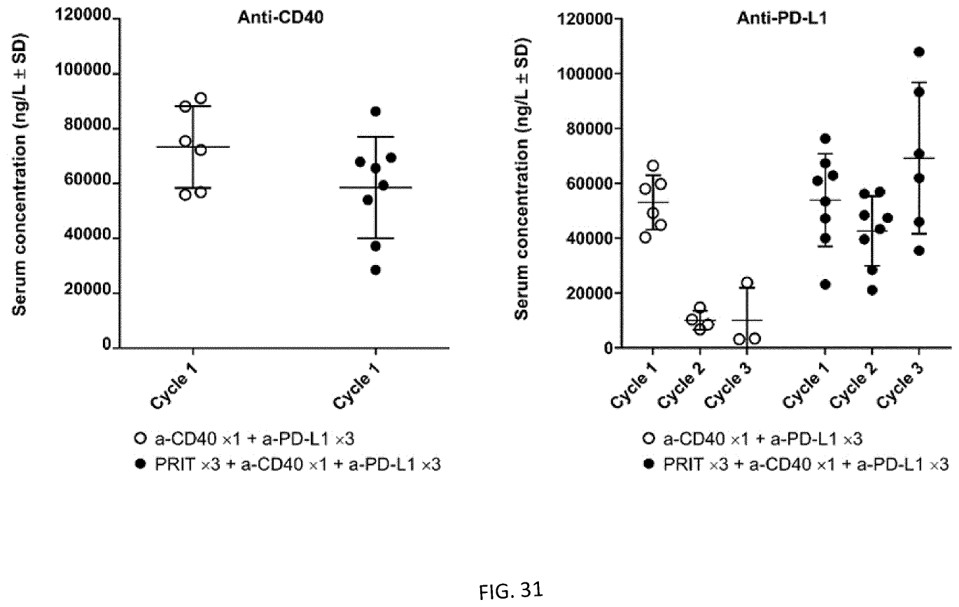

The serum concentration of anti-CD40 and anti-PD-L1 antibodies 24 hours after immunotherapy administration is shown in FIG. 31.

Tumor Development and Survival

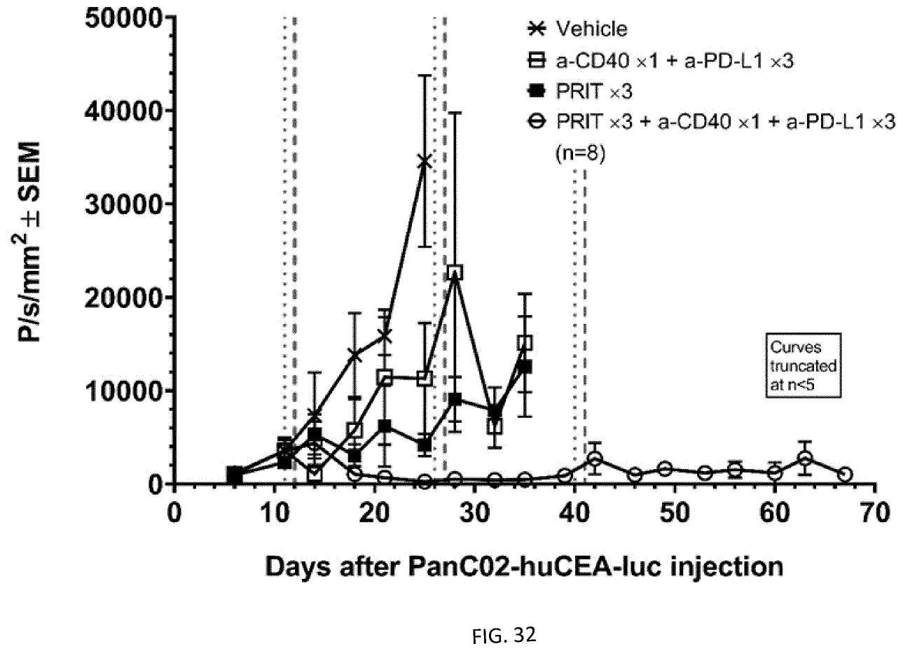

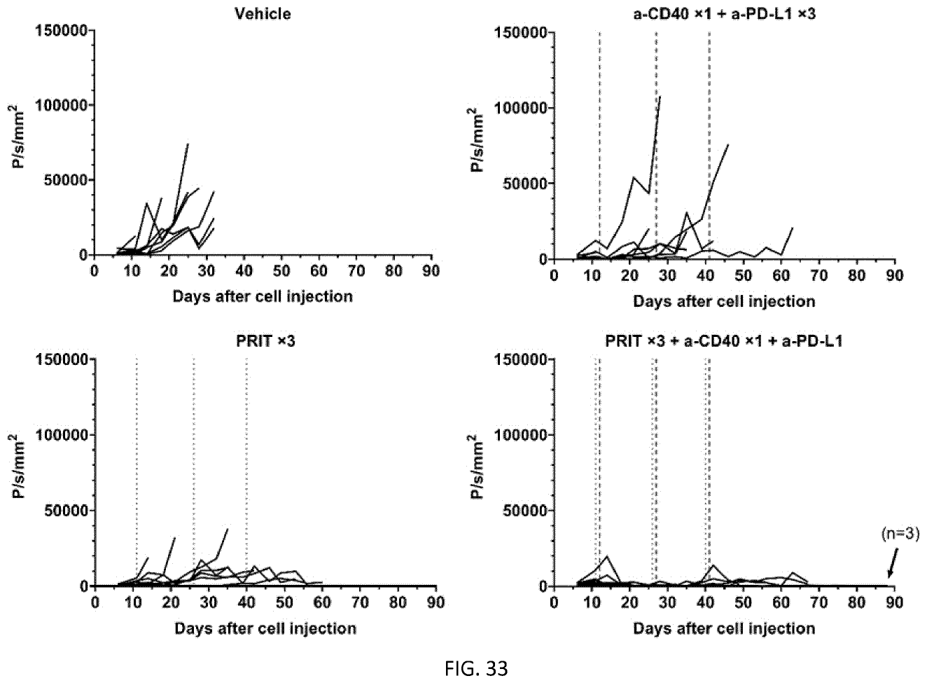

The average background-subtracted BLI signal after CEA-PRIT and control treatments is shown in FIG. 32 expressed as photons per second per mm². The corresponding individual curves are shown in FIG. 33. The average signal intensified exponentially in untreated control mice, whereas the increase was slower and with a bigger individual variation among mice treated with either monotherapy (immunotherapy or CEA-PRIT). In the CEA-PRIT/immunotherapy combination group, the BLI signal either increased at a slower rate compared with the other groups or diminished to background level. On day 88, the last day of imaging, there was no signal distinguishable from background noise in ⅜ CEA-PRIT/immunotherapy-treated mice.

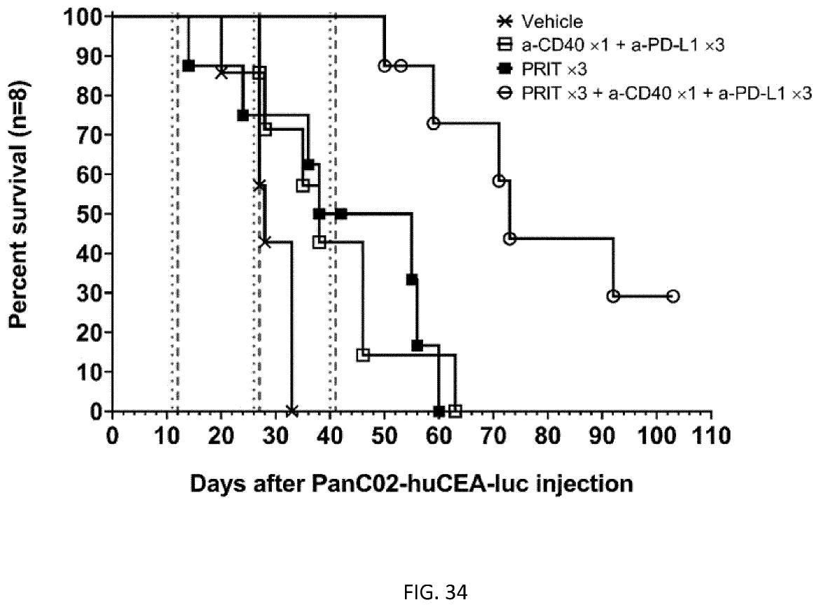

The survival curves are shown in FIG. 34. The study was terminated on day 103 after cell injection, at which point 2/8 mice in the CEA-PRIT/immunotherapy combination group were alive and tumor-free. No other groups had tumor-free or surviving mice. Time-to-event statistics (event=euthanasia/death) for the individual treatment groups are shown in the table below, showing the median survival time with upper and lower 9500 confidence limits, together with the quartile survival times (2500 and 750%).

Quantiles with time-to-event* statistics (days)

| | Median | Lower | Upper | 25% | 75% |
|---|---|---|---|---|---|
| Group | time | 95% | 95% | failures | failures |
| Vehicle | 27.5 | 11 | 33 | 23.5 | 33 |
| anti-CD40 + | 36.5 | 11 | 46 | 27.5 | 46 |
| anti-PD-L1 | | | | | |
| CEA-PRIT | 46.5 | 14 | 60 | 30 | 56 |
| CEA-PRIT + | 73 | 50 | — | 59 | — |
| anti-CD40 + | | | | | |
| anti-PD-L1 | | | | | |

*Event = euthanasia/death due to tumor burden.

Pairwise tests were performed to specify which groups were significantly different in terms of survival: the Log-Rank test (more weight on later survival events), and the Wilcoxon test (more weight on early survival times), both using Bonferroni correction for multiple testing. The results are shown in the tables below. The CEA-PRIT/immunotherapy combination significantly increased the survival compared with either monotherapy and the vehicle group.

Pairwise Log-Rank test (multiple test level = 0.00833)

| | | | | CEA-PRIT + |
|---|---|---|---|---|
| | | anti-CD40 + | | anti-CD40 + |
| Group | Vehicle | anti-PD-L1 | CEA-PRIT | anti-PD-L1 |
| Vehicle | 1.0000 | 0.0341 | 0.0131 | <0.0001* |
| anti-CD40 + | 0.0341 | 1.0000 | 0.7100 | 0.0004* |
| anti-PD-L1 | | | | |
| CEA-PRIT | 0.0131 | 0.7100 | 1.0000 | 0.0029* |
| CEA-PRIT + | <0.0001* | 0.0004* | 0.0029* | 1.0000 |
| anti-CD40 + | | | | |
| anti-PD-L1 | | | | |

Pairwise Wilcoxon test (multiple test level = 0.00833)

| | | | | CEA-PRIT + |
|---|---|---|---|---|
| | | anti-CD40 + | | anti-CD40 + |
| Group | Vehicle | anti-PD-L1 | CEA-PRIT | anti-PD-L1 |
| Vehicle | 1.0000 | 0.0864 | 0.0396 | 0.0002* |
| anti-CD40 + | 0.0864 | 1.0000 | 0.5250 | 0.0007* |
| anti-PD-L1 | | | | |
| CEA-PRIT | 0.0396 | 0.5250 | 1.0000 | 0.0065* |
| CEA-PRIT + | 0.0002* | 0.0007* | 0.0065* | 1.0000 |
| anti-CD40 + | | | | |
| anti-PD-L1 | | | | |

Adverse Events and Toxicity

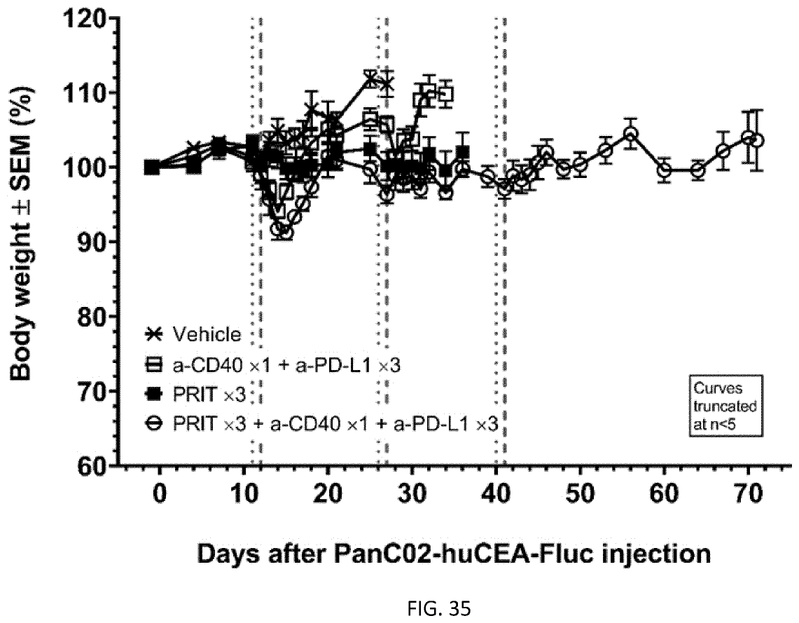

The average BW development in all therapy groups is shown in FIG. 35. Administration of anti-CD40 triggered an expected acute weight loss in injected mice, which was resolved within approximately 1 week after injection. Injection of ²¹²Pb-DOTAM caused transient weight loss in irradiated mice, which was less severe than that following the anti-CD40 injection. No mice were euthanized due to acute post-injection BW loss.

In the CEA-PRIT monotherapy group, 1 mouse was excluded from the protocol during the third treatment cycle due to a failed ²¹²Pb-DOTAM injection; 1 mouse in the same group died while under anesthesia during BLI acquisition. In the CEA-PRIT/immunotherapy combination group, 1 mouse was euthanized due to necrosis on the tail.

Adverse events in protocol 119

| | Mice sacrificed | | Reason for |
|---|---|---|---|
| Group | (n per group) | Study day | termination |
| C: CEA-PRIT | 1(8) | 42 | Failed ²¹²Pb- |
| | | | DOTAM injection |
| C: CEA-PRIT | 1(8) | 56 | Mouse dead during |
| | | | anesthesia/BLI |
| | | | acquisition |
| D: CEA-PRIT + | 1(8) | 53 | Necrotic tail |
| anti-CD40 + anti- | | | |
| PD-L1 | | | |

²¹²Pb irradiation (20 μCi) was performed on study days 11, 26, and 40. Immunotherapy was administered on study days 12 (anti-CD-40 + anti-PD-L1), 27 (anti-PD-L1), and 41 (anti-PD-L1).

Histopathological Examination

In all mice were examined the following tissues: kidneys, liver, lungs, spleen, and primary tumor (intrapancreatic). Tissue sections were stained with hematoxylin and eosin (H&E) or Periodic acid-Schiff (PAS, kidneys only) and examined by light microscopy on a Leica Diaplan microscope. Histopathological findings were graded in severity using a five-point system of minimal (grade 1), slight (grade 2), moderate (grade 3), marked (grade 4) or severe (grade 5).

Tumor

All mice (8/8) in groups A, B, and C had presence of implanted tumor within the pancreas, whereas the corresponding number was 5/8 for group D. Metastatic implantation was recorded in the liver of 1/8 mice of groups B, C and D, and in the spleen of 1 mouse of group B. Size, necrosis and hemorrhage were overall similar among groups (table below). The number of mitotic figures was as follows: group A>group B>group C>group D, while the apoptotic figures were lower in groups A and B when compared to groups C and D.

Size and mean score* of hemorrhage, mitotic figures
and apoptotic figures in implanted pancreatic tumor

| | | | | D: |
|---|---|---|---|---|
| | | B: | | CEA-PRIT + |
| | A: | anti-CD40 + | C: | anti-CD40 + |
| Group | Vehicle | anti-PD-L1 | CEA-PRIT | anti-PD-L1 |
| n examined | 8 | 8 | 8 | 5 |
| Mean size in | 11.13 ± 1.89 | 14.13 ± 5.69 | 10.63 ± 4.14 | 14.00 ± 3.39 |
| cm ± SD | | | | |
| Necrosis | 2.5 | 2.5 | 2.1 | 2.6 |
| Hemorrhage | 1.9 | 0.9 | 1.3 | 1.2 |
| Mitotic figures | 4 | 2.9 | 2.5 | 0.8 |
| Apoptotic | 1.5 | 1.4 | 2.5 | 2.4 |
| figures | | | | |

*Mean score = Σ number of animals × severity/number of tumors in the group

Organs

The main treatment-related effects were present in the kidneys of mice from groups C and D, and consisted of degeneration/regeneration and anisokaryosis involving mainly the proximal tubules (S3 segment) within the outer stripe of the outer medulla.

Degeneration/regeneration and anisokaryosis, proximal
tubules in the kidneys of groups C and D

| | | D: |
|---|---|---|
| | | CEA-PRIT + |
| | C: | anti-CD40 + |
| Group | CEA-PRIT | anti-PD-L1 |
| n examined | 8 | 5 |

Degeneration/regeneration, proximal tubules

| | | |
|---|---|---|
| Minimal | 4 | 5 |
| Slight | 0 | 1 |
| Moderate | 0 | 2 |
| Mean score* | 0.5 | 1.6 |

Anisokaryosis, proximal tubules

| | | |
|---|---|---|
| Minimal | 3 | 0 |
| Slight | 3 | 5 |
| Moderate | 1 | 2 |
| Marked | 0 | 1 |
| Mean score* | 1.5 | 2.5 |

*Mean score = Σ number of animals × severity/number of tumors in the group

Conclusion

The combination of CEA-PRIT with CIT (anti-CD40+anti-PD-L1) significantly increased the survival compared with vehicle and either monotherapy, and 2/8 CEA-PRIT/immunotherapy-treated mice that initially had established orthotopic tumors were tumor-free at the end of the study. BLI confirmed the improved tumor control using the combination treatment.

No mice in either group were euthanized due to BW loss, indicating good tolerance of the treatments. However, a number of animals suffered from the isoflurane-mediated anesthesia during the repeated imaging sessions, requiring prolonged time to wake up/recover and seemingly aging faster (greying fur). One mouse did not wake up from anesthesia, and died after image acquisition.

Treatment with CEA-PRIT induced tubular degeneration/regeneration and anisokaryosis within the kidney (proximal tubules), the severity of the kidney findings being slightly higher after combination with immunotherapy. The main effects on tumor were reduction of incidence with CEA-PRIT/immunotherapy combination treatment, decreased mitotic figures with CEA-PRIT and/or immunotherapy treatment, and increased apoptotic figures with CEA-PRIT.

### Example 9c: Protocol 136

The aim of protocol 136 was to assess the efficacy following three cycles of CEA-PRIT, alone and in combination with CIT, for treatment of SC Panc02-huCEA-luc tumors in immunocompetent transgenic mice.

The PRIT regimen was administered in 3 repeated cycles comprising IP injection of CEA-DOTAM (mu) BsAb (100 μg in 200 μL) followed 7 days later by IP administration of a Pb-DOTAM-dextran-500 CA (25 μg in 200 μL), followed in turn 24 hours later by the effector molecule ²¹²Pb-DOTAM (20 μCi).

The immunotherapy treatment was administered IP 24 hours after the radioactive injection, consisting of a one-time administration of anti-CD40 antibody and multiple injections of anti-PD-L1 antibody (200 μg of each antibody in 200 μL). Scout mice were taken for biodistribution assessment to confirm ²¹²Pb-DOTAM targeting and clearance during the first treatment cycle, in addition to mice sacrificed for flow cytometric analysis of immunopharmacodynamic (immuno-PD) effects after the second cycle. Comparisons were made between the CEA-PRIT/immunotherapy combination, CEA-PRIT alone, immunotherapy alone, and no treatment. The treatment efficacy was assessed in terms of TGI, survival, and immune memory. In addition to calipering, the tumor growth was followed through BLI.

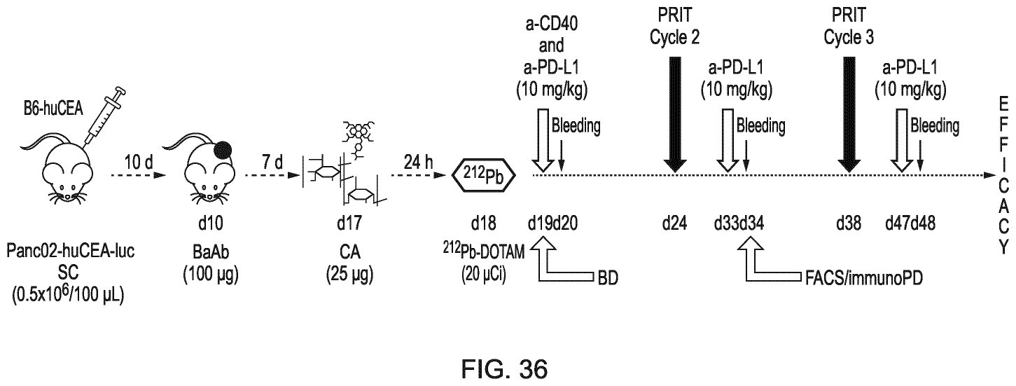

The study outline is shown in FIG. 36.

The time course and design of protocol 136 are shown in the tables below.

Time course of protocol 136

| Study | | |
|---|---|---|
| day | Date | Experimental procedure |
| 0 | 2018 Jan. 12 | Preparation of Panc02-huCEA-luc cells and |
| | | filling of syringes |
| 0 | 2018 Jan. 12 | SC injection of Panc02-huCEA-luc cells |
| 10 | 2018 Jan. 22 | IP injection of BsAb |
| 11 | 2018 Jan. 23 | SC injection of D-Luciferin + imaging (BLI) |
| 14 | 2018 Jan. 26 | SC injection of D-Luciferin + imaging (BLI) |
| 17 | 2018 Jan. 29 | SC injection of D-Luciferin + imaging (BLI) |
| 17 | 2018 Jan. 29 | IP injection of CA |
| 18 | 2018 Jan. 30 | Elution of ²¹²Pb-DOTAM and filling of syringes |
| 18 | 2018 Jan. 30 | IV injection of ²¹²Pb-DOTAM |
| 19 | 2018 Jan. 31 | Euthanasia and tissue harvest (24 h p.i.) + |
| | | gamma counting |
| 19 | 2018 Jan. 31 | IP injection of anti-CD40 and anti-PD-L1 |
| 20 | 2018 Feb. 1 | Retro-orbital bleeding |
| 20 | 2018 Feb. 1 | SC injection of D-Luciferin + imaging (BLI) |
| 24 | 2018 Feb. 5 | IP injection of BsAb |
| 24 | 2018 Feb. 5 | SC injection of D-Luciferin + imaging (BLI) |
| 27 | 2018 Feb. 8 | SC injection of D-Luciferin + imaging (BLI) |
| 31 | 2018 Feb. 12 | IP injection of CA |
| 31 | 2018 Feb. 12 | SC injection of D-Luciferin + imaging (BLI) |
| 31 | 2018 Feb. 12 | Elution of ²¹²Pb-DOTAM and filling of syringes |
| 32 | 2018 Feb. 13 | IV injection of ²¹²Pb-DOTAM |
| 33 | 2018 Feb. 14 | IP injection of anti-PD-L1 |
| 34 | 2018 Feb. 15 | Retro-orbital bleeding |
| 34 | 2018 Feb. 15 | Euthanasia and tissue harvest for immuno-PD |
| | | (FACS) |
| 35 | 2018 Feb. 16 | SC injection of D-Luciferin + imaging (BLI) |
| 38 | 2018 Feb. 19 | IP injection of BsAb |
| 38 | 2018 Feb. 19 | SC injection of D-Luciferin + imaging (BLI) |
| 41 | 2018 Feb. 22 | SC injection of D-Luciferin + imaging (BLI) |
| 45 | 2018 Feb. 26 | IP injection of CA |
| 45 | 2018 Feb. 26 | SC injection of D-Luciferin + imaging (BLI) |
| 46 | 2018 Feb. 27 | Elution of ²¹²Pb-DOTAM and filling of syringes |
| 46 | 2018 Feb. 27 | IV injection of ²¹²Pb-DOTAM |
| 47 | 2018 Feb. 28 | IP injection of anti-PD-L1 |
| 48 | 2018 Mar. 1 | Retro-orbital bleeding |

Study groups in protocol 136

| | | BsAb | CA | ²¹²Pb | anti-CD40* | anti- | | n |
|---|---|---|---|---|---|---|---|---|
| Group | BsAb | (μg) | (μg) | (μCi) | (μg) | PD-L1 (μg) | Cycles | (mice) |
| A | — | 0 | 0 | 0 | 0 | 0 | 3 | 9 |
| B | — | 0 | 0 | 0 | 200 | 200 | 3 | 9 |
| C | CEA-DOTAM (mu) | 100 | 25 | 20 | 0 | 0 | 3 | 9 |
| D | CEA-DOTAM (mu) | 100 | 25 | 20 | 200 | 200 | 3 | 9 |
| E | CEA-DOTAM (mu) | 100 | 25 | 20 | 0 | 0 | 1 | 3 |
| F | DIG-DOTAM | 100 | 25 | 20 | 0 | 0 | 1 | 3 |
| G | — | 0 | 0 | 0 | 0 | 0 | 2 | 4 |
| H | — | 0 | 0 | 0 | 200 | 200 | 2 | 4 |
| I | CEA-DOTAM (mu) | 100 | 25 | 20 | 0 | 0 | 2 | 4 |
| J | CEA-DOTAM (mu) | 100 | 25 | 20 | 200 | 200 | 2 | 4 |

*Anti-CD40 only administered once, at the first treatment cycle

Primary solid xenografts were established in each B6-huCEA mouse (age 11-12 weeks) on study day 0 by SC injection of 0.5×10⁵ cells (passage 18) in RPMI/Matrigel, into the right flank. Ten days after tumor cell injection, mice were sorted into experimental groups with an average tumor volume of 114 mm³. The CA was injected on day 17 after inoculation, at which point the average tumor volume was 233 mm³. On day 19, the day after the ²¹²Pb-DOTAM injection, the average tumor volume was 326 mm³.

Mice in groups A-D were followed to assess therapeutic efficacy until the end of the study or until one or several of the termination criteria were reached. Blood was sampled from mice in groups B and D 24 hours after administration of the immunotherapy, to validate the anti-CD40 and anti-PD-L1 injections by analysis of serum fractions through ELISA. Serum was also isolated from all mice before euthanasia through retro-orbital blood collection, and then frozen and stored at −20° C. The following tissues were collected for histological processing and analysis and immediately put in 10% NBF for 24 hours, before being transferred to 1×PBS solution: serum, liver, spleen, kidneys, pancreas, and tumor.

Mice in groups E and F were sacrificed and necropsied 24 hours after ²¹²Pb-DOTAM injection to confirm tumor uptake and clearance from normal tissues. The following organs and tissues were thus harvested and measured for radioactivity: blood, bladder, spleen, kidneys, liver, lung, muscle, tail, skin, and tumor.

Groups G-J comprised immuno-PD scout mice that were sacrificed and necropsied after retro-orbital bleeding, 24 hours after the second anti-PD-L1 injection, to assess the generation of anti-tumor T cell and dendritic cell (DC) responses by functional and phenotypical characterization of T cells and DCs from different compartments. From all immuno-PD mice was harvested: tumor, spleen, and draining lymph nodes (DLN; from the tumor side).

An ex vivo PMA/ionomycin (Thermo Fisher, cat No. 00-4970-03, 00-4980-03) restimulation assay was performed on spleen samples to assess T cell effector and memory generation. In addition, flow cytometry (fluorescence-activated cell sorting [FACS]) was performed using a MACSQuant Analyzer 10 (Miltenyi Biotec) and analysis of results was performed using the FlowJo 10.5.3 software. The staining panel design is shown in the table below.

FACS panel design for protocol 136

| | | | | PMA/ |
|---|---|---|---|---|
| T cells | DCs | Pentamer | Tregs | ionomycin |
| Marker | Marker | Marker | Marker | Marker |
| live/dead | live/dead | live/dead | live/dead | live/dead |
| CD45 | CD45 | Pentamer | CD45 | CD45 |
| | | p15E | | |
| CD3 | Negative | CD45 | CD3 | CD3 |
| | selection (CD19, | | | |
| | Gr1, F4/80) | | | |
| CD4 | CD11c | CD3 | CD4 | CD4 |
| CD8a | MHC class II | CD8 | CD8 | CD8 |
| 41BB | CD86 | CD4 | CD25 | CD44 |
| Lag3 | CD11b | CD11b | CD11b | IFNγ |
| PD-1 | CD317 | B220 | FoxP3 | IL-2 |

IFNγ = interferon gamma;
IL-2 = interleukin-2;
MCH = major histocompatibility complex;
PMA = phorbol 12-myristate-13-acetate;
Treg = regulatory T cell

Results

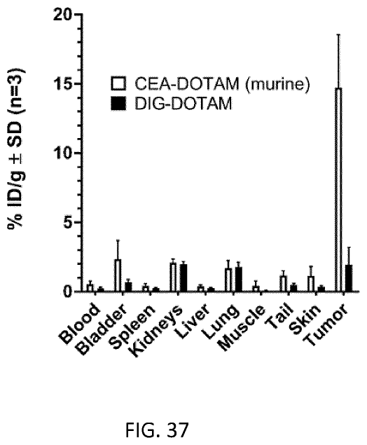

Biodistribution and ELISA The average ²¹²Pb accumulation and clearance in all collected tissues 24 hours after ²¹²Pb-DOTAM injection (cycle 1) is displayed in FIG. 37. The tumor uptake was specific, with 14.7% ID/g in the tumor after pretargeting with CEA-DOTAM (mu), compared with <2.5% ID/g for all collected normal tissues. Using DIG-DOTAM, the resulting tumor accumulation was 1.9% ID/g.

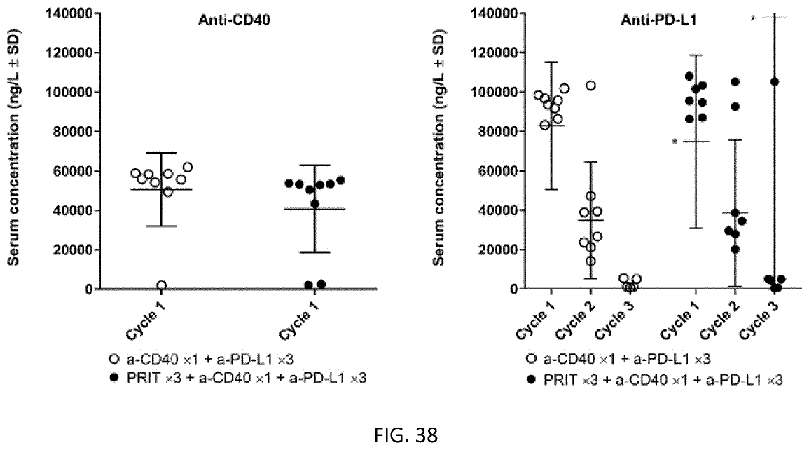

The serum concentration of anti-CD40 and anti-PD-L1 antibodies 24 hours after immunotherapy administration is shown in FIG. 38.

Tumor Development and Survival

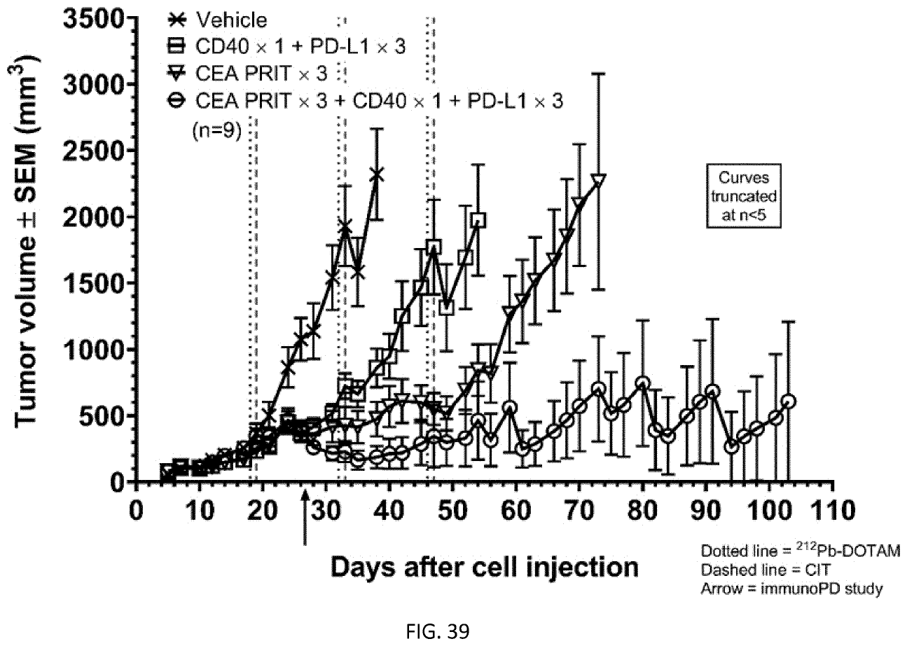

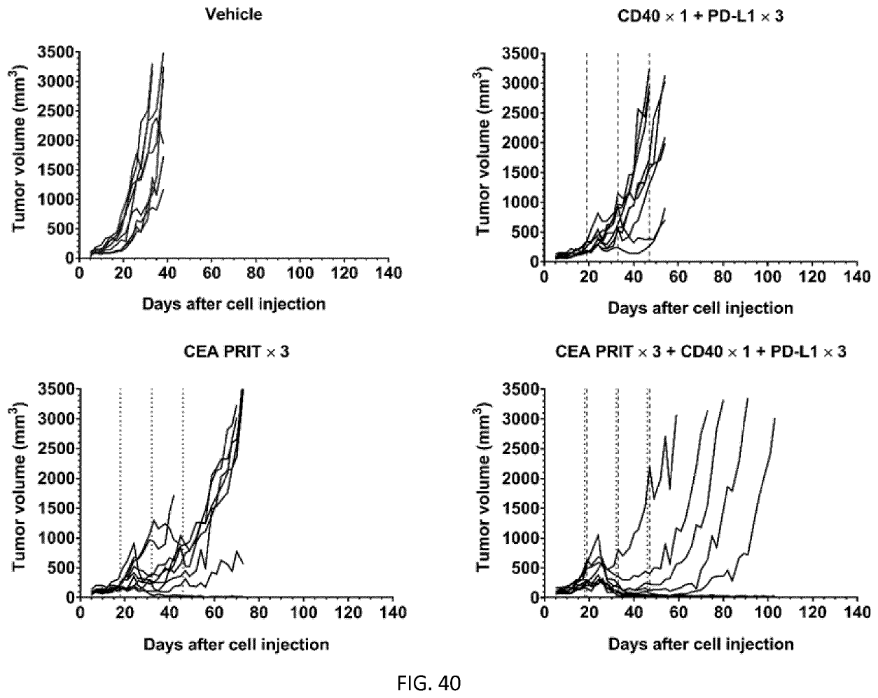

The average Panc02-huCEA-luc tumor development after CEA-PRIT and control treatments is shown in FIG. 39, with individual tumor growth curves for all treatment groups displayed in FIG. 40. Tumors in mice treated with either monotherapy (CIT or CEA-PRIT) grew steadily, albeit with a delay compared with the vehicle control; CEA-PRIT had a stronger effect than the immunotherapy. In the CEA-PRIT/immunotherapy combination group, 8/9 mice exhibited an initial response to the treatment in terms of decreasing tumor volume; no correlation was seen between the strength/duration of the response and the tumor size at the onset of therapy.

On day 47, the last day on which all treatment groups could be analyzed based on means, the TGI was 34.0%, 81.4%, and 89.7% for immunotherapy, CEA-PRIT, and the combination of CEA-PRIT and immunotherapy, respectively, compared with the vehicle control. The primary study was terminated on day 140 after cell injection, at which point 4/9 mice were alive and tumor-free in the CEA-PRIT/immunotherapy combination group. In the PRIT monotherapy group, 1/9 mice had a tumor that regressed completely, but the mouse was euthanized on day 117 due to BW loss.

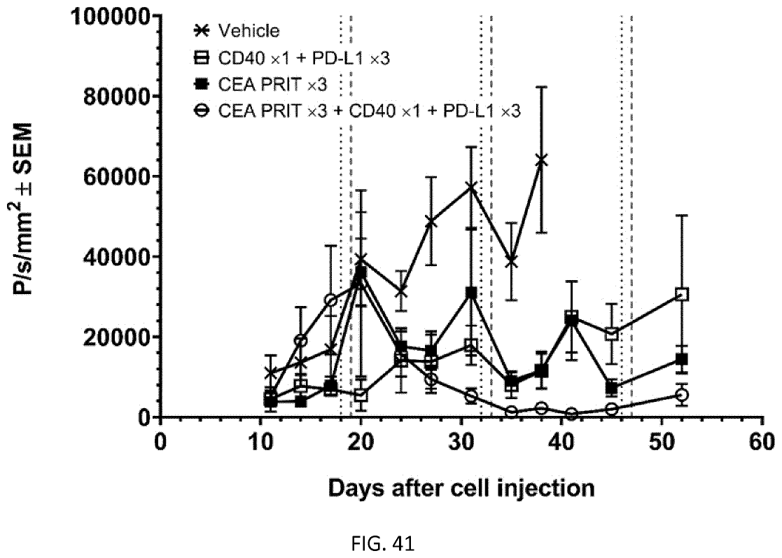

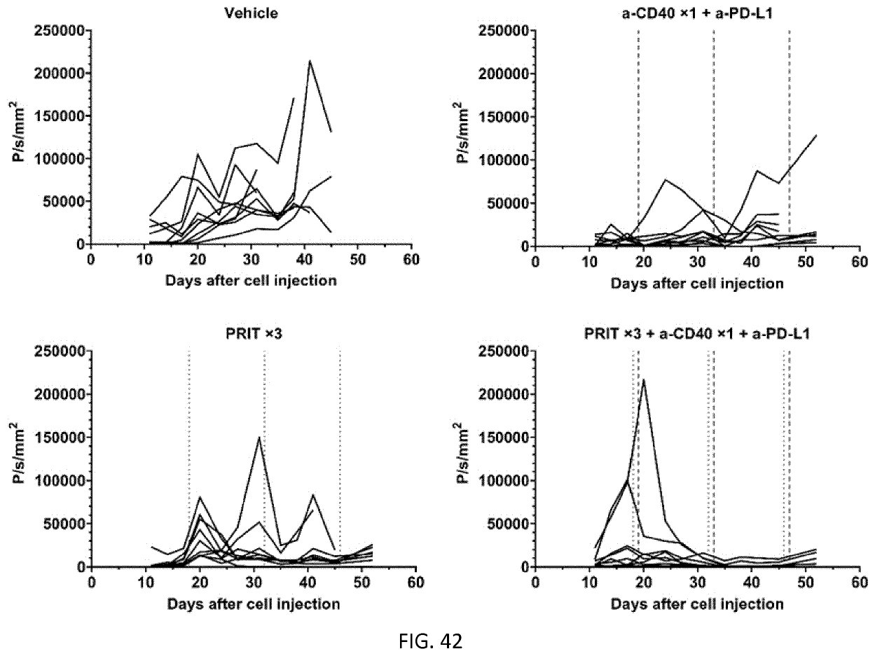

The average background-subtracted BLI signal after CEA-PRIT and control treatments is shown in FIG. 41, expressed as photons per second per mm². The corresponding individual curves are shown in FIG. 42. Compared with the corresponding orthotopic tumor model (protocol 119), the results were more variable; CEA-PRIT with and without immunotherapy showed a strong decrease in BLI signal in individual mice, whereas the immunotherapy mice had lower initial signal, making any decrease hard to distinguish.

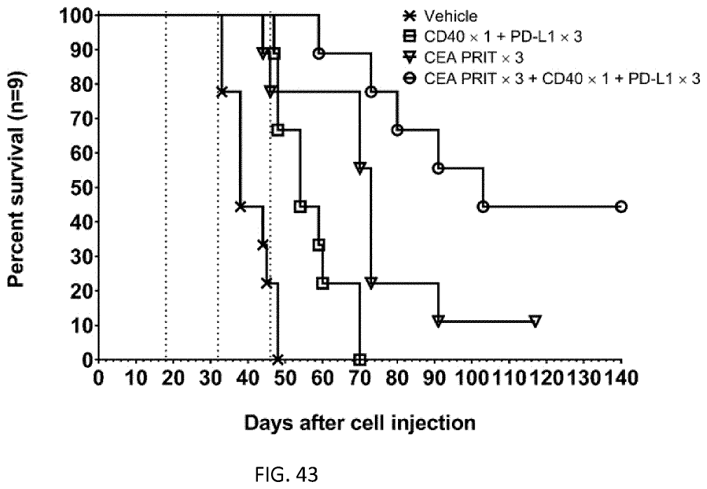

The overall survival is shown in FIG. 43, based on the termination criteria of tumor volume ≥3000 mm³. Time-to-event (tumor volume exceeding 3000 mm³) statistics for the individual treatment groups are shown in the table below, showing the median survival time with upper and lower 95% confidence limits, together with the quartile survival times (25% and 75%).

Quantiles with time-to-event* statistics (days)

| | Median | Lower | Upper | 25% | 75% |
|---|---|---|---|---|---|
| Group | time | 95% | 95% | failures | failures |
| Vehicle | 38 | 33 | 48 | 38 | 45 |
| anti-CD40 + anti-PD-L1 | 54 | 47 | 70 | 48 | 60 |
| CEA-PRIT | 73 | 70 | 91 | 70 | 91 |
| CEA-PRIT + | 103 | 59 | — | 80 | — |
| anti-CD40 + anti-PD-L1 | | | | | |

*Event = tumor volume ≥ 3000 mm³.

Pairwise tests were performed to specify which groups were significantly different in terms of survival: the Log-Rank test (more weight on later survival events), and the Wilcoxon test (more weight on early survival times), both using Bonferroni correction for multiple testing. The results are shown in the tables below. All treatments significantly increased the survival compared with the vehicle group.

Pairwise Log-Rank test (multiple test level = 0.00833)

| | | | | CEA-PRIT + |
|---|---|---|---|---|
| | | anti-CD40 + | | anti-CD40 + |
| Group | Vehicle | anti-PD-L1 | CEA-PRIT | anti-PD-L1 |
| Vehicle | 1.0000 | 0.0023* | 0.0002* | 0.0001* |
| anti-CD40 + | 0.0023* | 1.0000 | 0.0006* | 0.0001* |
| anti-PD-L1 | | | | |
| CEA-PRIT | 0.0002* | 0.0006* | 1.0000 | 0.1242 |
| CEA-PRIT + | 0.0001* | 0.0001* | 0.1242 | 1.0000 |
| anti-CD40 + | | | | |
| anti-PD-L1 | | | | |

Pairwise Wilcoxon test (multiple test level = 0.00833)

| | | | | CEA-PRIT + |
|---|---|---|---|---|
| | | anti-CD40 + | | anti-CD40 + |
| Group | Vehicle | anti-PD-L1 | CEA-PRIT | anti-PD-L1 |
| Vehicle | 1.0000 | 0.0018* | 0.0005* | 0.0003* |
| anti-CD40 + | 0.0018* | 1.0000 | 0.0011* | 0.0004* |
| anti-PD-L1 | | | | |
| CEA-PRIT | 0.0005* | 0.0011* | 1.0000 | 0.1085 |
| CEA-PRIT + | 0.0003* | 0.0004* | 0.1085 | 1.0000 |
| anti-CD40 + | | | | |
| anti-PD-L1 | | | | |

Immuno-Pharmacodynamics

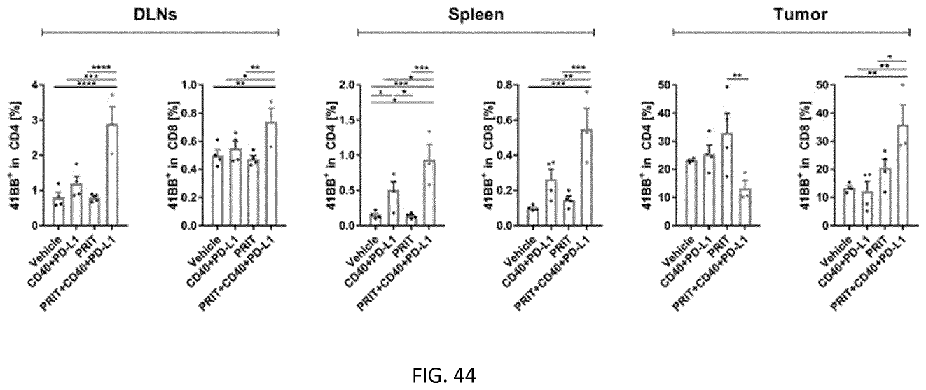

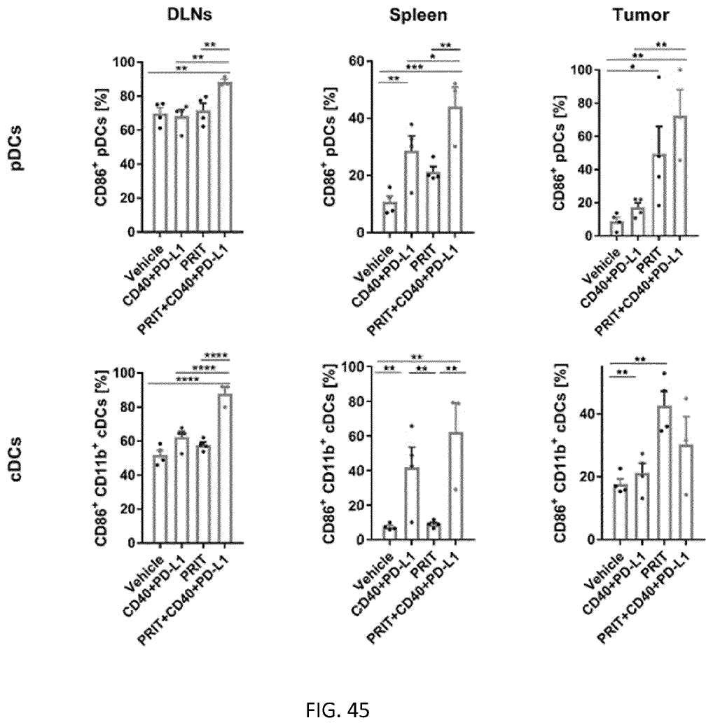

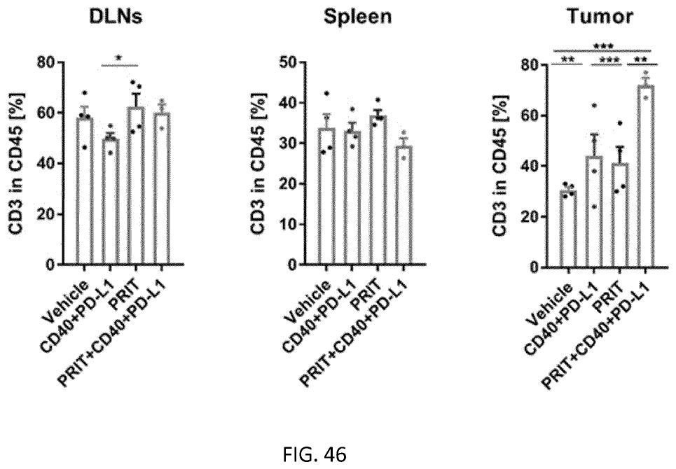

No significant results were achieved using the regulatory T cell (Treg) or pentamer staining. The CEA-PRIT+CIT combination correlated with significantly increased frequency of activated intratumoral CD8 T cells (as measured by upregulation of 41BB expression), activated plasmacytoid DCs (pDCs) and classical DCs (cDCs) in tumor, spleen and draining lymphnodes (DLNs) (as measured by upregulation of CD86 expression), and increased frequency of T cells in total immune cells, as compared to all other treatments. Key results are shown in FIG. 44, FIG. 45 and FIG. 46.

Rechallenge

To assess the development of anti-tumor immune memory response to the primary tumor, tumor-free mice after treatment were rechallenged with Panc02-huCEA-luc cells in the opposite flank to the primary injection. Two untreated control groups were inoculated: one with B6-huCEA mice aged 11 weeks, and the other with B6-huCEA mice age-matched with the rechallenged mice (33 weeks). The rechallenge was performed on day 140 after the first tumor cell injection.

For rechallenge of 5 treated (tumor-free) mice (age 31-32 weeks) and 5 control mice (age 11 weeks), tumor grafts were established by SC injection of 0.5×10⁵ cells (passage 18) into the left flank. At a later time point, 5 age-matched (age 33 weeks) non-treated control mice were injected SC with 0.5×10⁵ cells (passage 22) into the left flank. The study groups and time course of the rechallenge/controls are shown in the tables below.

Study groups in protocol 136 (rechallenge)

| | Treatment | Age | | Cells/ | n |
|---|---|---|---|---|---|
| Group | received | (weeks) | Cell line | mouse | (mice) |
| D* | CEA-PRIT × 3 + | 31-32 | Panc02- | 0.5 × 10⁶ | 4 + 1** |
| | anti-CD40 × 1 + | | huCEA-luc | | |
| | anti-PD-L1 × 3 | | | | |
| K | Untreated control | 11 | Panc02- | 0.5 × 10⁶ | 5 |
| | | | huCEA-luc | | |
| L | Untreated age- | 33 | Panc02- | 0.5 × 10⁶ | 5 |
| | matched control | | huCEA-luc | | |

*Retained group designation from protocol 136;
**Additional mouse treated like the mice in group D, but not included in the primary efficacy study.

Time course of protocol 136 (rechallenge)

| Study | | |
|---|---|---|
| day | Date | Experimental procedure |
| 140 | 2018 Jun. 1 | Preparation of Panc02-huCEA-luc cells and filling |
| | | of syringes |
| 140 | 2018 Jun. 1 | SC injection of Panc02-huCEA-luc cells (group D* |
| | | and K) |
| 172 | 2018 Jul. 3 | Euthanasia and tissue sampling (group D* and K) |
| — | 2018 Dec. 4 | Preparation of Panc02-huCEA-luc cells and filling |
| | | of syringes |
| — | 2018 Dec. 4 | SC injection of Panc02-huCEA-luc cells (group L) |
| — | 2019 Jan. 3 | Euthanasia (group L) |

Blood, spleen, and DLNs were collected from groups D and K for flow cytometry analysis upon termination. Characterization of T cells was performed on blood, spleen and DLNs, and an ex vivo PMA/ionomycin restimulation assay was performed on spleen.

Results Rechallenge

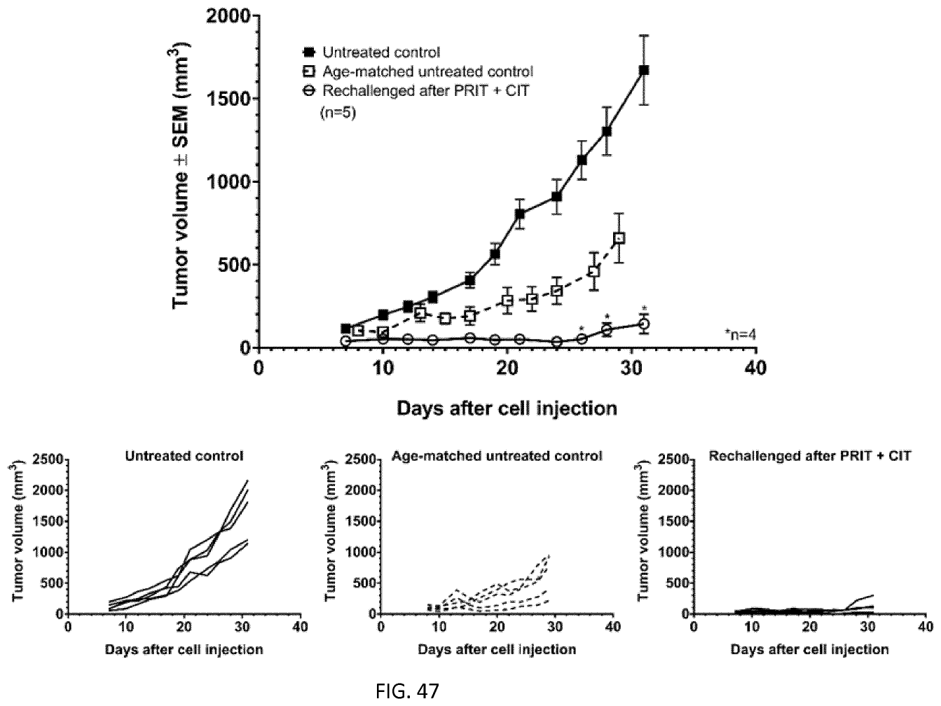

The tumor growth in rechallenged and naïve mice is shown in FIG. 47. All non-treated mice (5/5+5/5) developed tumors, although the growth kinetics were slower in the age-matched control mice. Of the rechallenged mice, 4/5 remained tumor-free until termination of the experiment, 32 days after the second inoculation (172 days after the initial inoculation); 1/5 had a tumor that started growing 28 days after rechallenge. One tumor-free rechallenged mouse was euthanized on day 24 after the second inoculation (164 days after the initial inoculation) due to a sudden drop in BW.

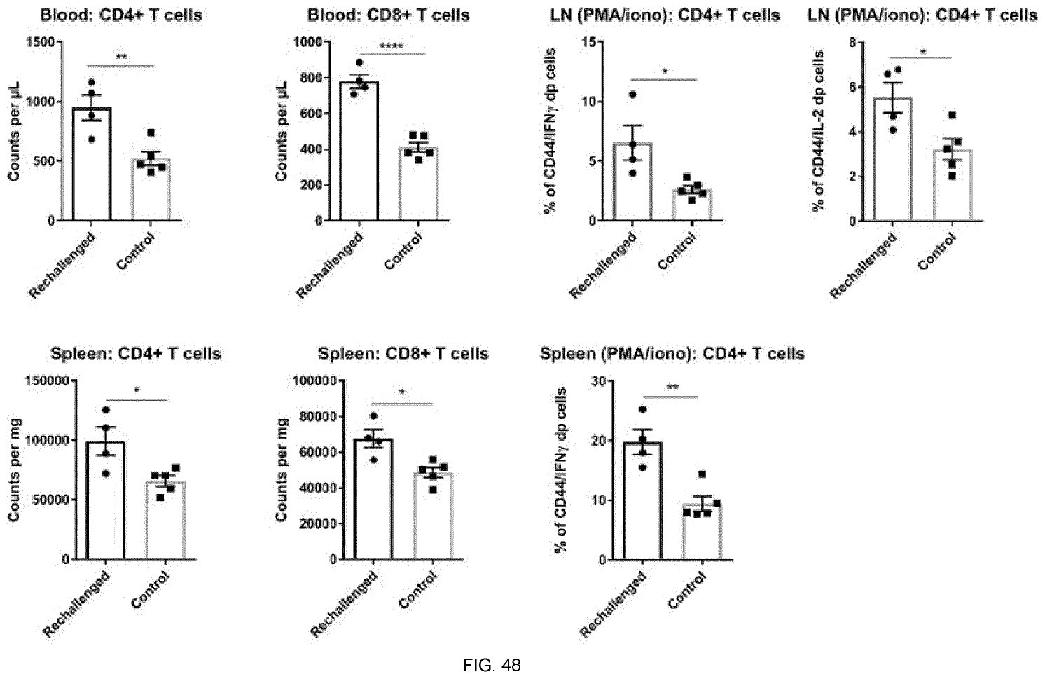

FACS analysis of samples from rechallenged and naïve (not age-matched) mice revealed an increased population of CD4+ and CD8+ T cells in blood and spleen in the rechallenged group compared to the control. Other findings included an increased CD4+ effector memory cell population (CD44+) in spleen, producing interleukin-2 (IL-2) upon ex vivo PMA/ionomycin stimulation, and an increased CD4+ effector memory cell population (CD44+) in DLNs producing IL-2 and interferon gamma (IFNγ) upon ex vivo PMA/ionomycin stimulation. Key results are shown in FIG. 48.

Adverse Events and Toxicity

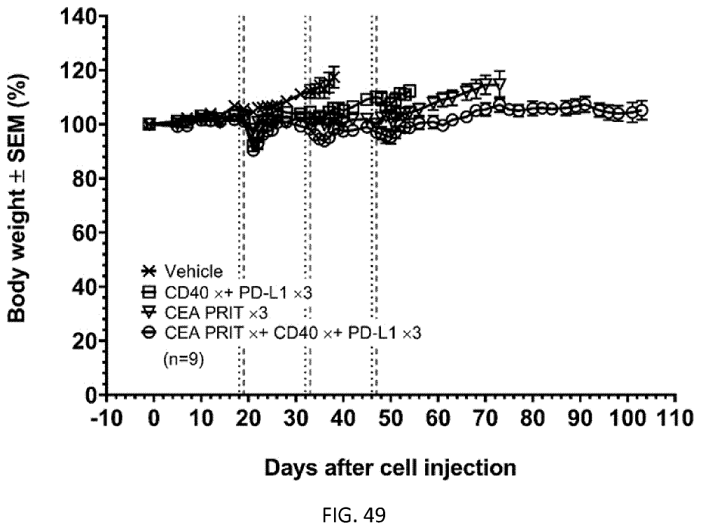

The average BW development in all therapy groups is shown in FIG. 49. Administration of anti-CD40 triggered an expected acute weight loss in injected mice, which was resolved within a week after injection. Injection of ²¹²Pb-DOTAM caused transient weight loss in irradiated mice, which was less severe than that from the anti-CD40. No mice were euthanized due to acute post-injection BW loss; 1 tumor-free CEA-PRIT mouse was euthanized on day 117 after a sudden decline in overall status.

From all groups, a total of 2 mice were euthanized due to declining tumor status (open tumor with risk of degradation); 1 additional mouse was euthanized due to a degrading tumor in combination with declining overall status. All adverse events in the primary and rechallenge parts of the study are described in the table below.

Adverse events in protocols 136 and 136b

| | Mice sacrificed | Study | Reason for |
|---|---|---|---|
| Group | (n per group) | day | termination |
| A: Vehicle | 1(9) | 48 | Declining tumor/ |
| | | | overall status |
| C: CEA-PRIT | 2(9) | 44, 46 | Declining tumor |
| | | | status |
| C: CEA-PRIT | 1(9) | 117 | BW loss ≥ 20% |
| D*: CEA-PRIT + | 1(5) | 164 | BW loss ≥ 20% |
| anti-CD40 + | | | |
| anti-PD-L1 | | | |

²¹²Pb irradiation (20 μCi) was performed on study days 18, 32, and 46. Immunotherapy was administered on study days 19 (anti-CD-40 + anti-PD-L1), 33 (anti-PD-L1), and 47 (anti-PD-L1).
*Rechallenged on day 140.

Conclusion

The combination of CEA-PRIT with CIT (anti-CD40+anti-PD-L1) resulted in significantly improved survival compared with control mice and mice treated with immunotherapy alone. The combination treatment resulted in several tumor-free mice, and strong indications of immune memory, demonstrated by immuno-PD and the diminished tumor development in rechallenged mice.

### Example 9d: Protocol 150

The aim of protocol 150 was to assess the efficacy following three cycles of CEA-PRIT, alone and in combination with CIT, for treatment of SC MC38-huCEA tumors in immunocompetent transgenic mice.

The PRIT regimen was administered in 3 repeated cycles comprising IP injection of CEA-DOTAM (mu) BsAb (100 μg in 200 μL) followed 7 days later by IP administration of a Ca-DOTAM-dextran-500 CA (25 μg in 200 μL), followed in turn 24 hours later by the effector molecule ²¹²Pb-DOTAM (20 μCi).

The immunotherapy treatment was administered IP 24 hours after the radioactive injection, consisting of a one-time administration of anti-CD4c antibody, or a one-time administration of anti-CD40 antibody followed by multiple injections of anti-PD-L1 antibody (200 g of each antibody in 200 μL). Scout mice were taken for biodistribution assessment to confirm ²¹²Pb-DOTAM targeting and clearance during the first treatment cycle, in addition to mice sacrificed for flow cytometric analysis of immuno-PD effects after the second cycle. Comparisons were made between the CEA-PRIT/immunotherapy combination, CEA-PRIT alone, immunotherapy alone, and no treatment. The treatment efficacy was assessed in terms of TGI, survival, and immune memory.

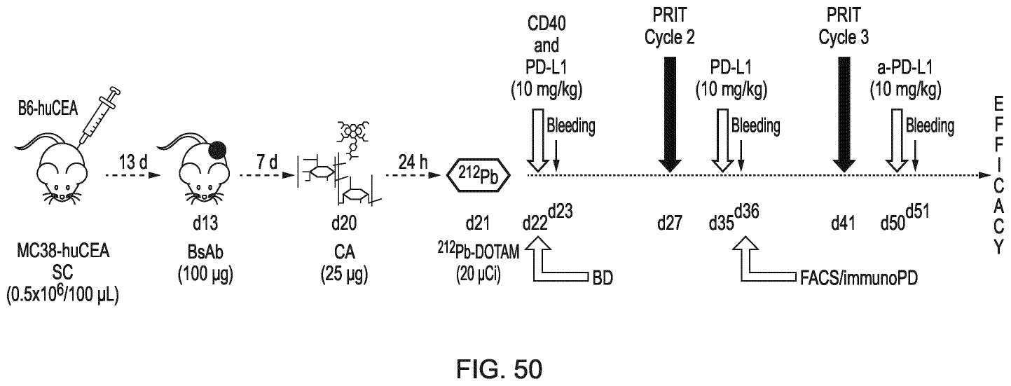

The study outline is shown in FIG. 50.

The time course and design of protocol 150 are shown in the tables below.

Time course of protocol 150

| Study day | Date | Experimental procedure |
|---|---|---|
| 0 | 2018 Sep. 5 | Preparation of MC38-huCEA cells and filling of syringes |
| 0 | 2018 Sep. 5 | SC injection of MC38-huCEA cells |
| 13 | 2018 Sep. 18 | IP injection of BsAb |
| 20 | 2018 Sep. 25 | IP injection of CA |
| 21 | 2018 Sep. 26 | Elution of ²¹²Pb-DOTAM and filling of syringes |
| 21 | 2018 Sep. 26 | IV injection of ²¹²Pb-DOTAM |
| 22 | 2018 Sep. 27 | Euthanasia and tissue harvest (24 h p.i.) + gamma counting |
| 22 | 2018 Sep. 27 | IP injection of anti-CD40 and anti-PD-L1 |
| 23 | 2018 Sep. 28 | Sublingual bleeding |
| 27 | 2018 Oct. 2 | IP injection of BsAb |
| 33 | 2018 Oct. 8 | IP injection of CA |
| 33 | 2018 Oct. 8 | Elution of ²¹²Pb-DOTAM and filling of syringes |
| 34 | 2018 Oct. 9 | IV injection of ²¹²Pb-DOTAM |
| 35 | 2018 Oct. 10 | IP injection of anti-PD-L1 |
| 36 | 2018 Oct. 11 | Sublingual bleeding |
| 36 | 2018 Oct. 11 | Euthanasia and tissue harvest for immuno-PD (FACS) |
| 41 | 2018 Oct. 16 | IP injection of BsAb |
| 48 | 2018 Oct. 23 | IP injection of CA |
| 48 | 2018 Oct. 23 | Elution of ²¹²Pb-DOTAM and filling of syringes |
| 49 | 2018 Oct. 24 | IV injection of ²¹²Pb-DOTAM |
| 50 | 2018 Oct. 25 | IP injection of anti-PD-L1 |
| 51 | 2018 Oct. 26 | Retro-orbital bleeding |

Study groups in protocol 150

| | | BsAb | CA | ²¹²Pb | anti-CD40* | anti- | | n |
|---|---|---|---|---|---|---|---|---|
| Group | BsAb | (μg) | (μg) | (μCi) | (μg) | PD-L1 (μg) | Cycles | (mice) |
| A | — | 0 | 0 | 0 | 0 | 0 | 3 | 9 |
| B | — | 0 | 0 | 0 | 200 | 0 | 3 | 9 |
| C | — | 0 | 0 | 0 | 200 | 200 | 3 | 9 |
| D | CEA-DOTAM (mu) | 100 | 25 | 20 | 0 | 0 | 3 | 9 |
| E | CEA-DOTAM (mu) | 100 | 25 | 20 | 200 | 200 | 3 | 9 |
| F | CEA-DOTAM (mu) | 100 | 25 | 20 | 0 | 0 | 1 | 4 |
| G | DIG-DOTAM | 100 | 25 | 20 | 0 | 0 | 1 | 4 |
| H | — | 0 | 0 | 0 | 0 | 0 | 2 | 4 |
| I | — | 0 | 0 | 0 | 200 | 0 | 2 | 4 |
| J | — | 0 | 0 | 0 | 200 | 200 | 2 | 4 |
| K | CEA-DOTAM (mu) | 100 | 25 | 20 | 0 | 0 | 2 | 4 |
| L | CEA-DOTAM (mu) | 100 | 25 | 20 | 200 | 200 | 2 | 4 |

*Anti-CD40 only administered once, at the first treatment cycle

Primary solid xenografts were established in each B6-huCEA mouse (age 10⁻¹² weeks) on study day 0 by SC injection of 0.5×10⁶ cells (passage 17) in DMEM/Matrigel, into the right flank. Twelve days after tumor cell injection, mice were sorted into experimental groups with an average tumor volume of 103 mm³. The CA was injected one week later; the average tumor volume on day 19 being 284 mm³ in the efficacy groups (A-E), 303 mm³ in the biodistribution scout groups (F, G), and 262 mm³ in the immuno-PD scout groups (H-L).

Mice in groups A-E were mice followed to assess therapeutic efficacy until the end of the study or until one or several of the termination criteria were reached. Blood was sampled from mice in groups B, C and E 24 hours after administration of the immunotherapy, to validate the anti-CD40 and anti-PD-L1 injections by analysis of serum fractions through ELISA. Serum was also isolated from all mice before euthanasia through retro-orbital blood collection, and then frozen and stored at −20° C. The following tissues were collected for histological processing and analysis and immediately put in 10% NBF for 24 hours, before being transferred to 1×PBS solution: serum, liver, spleen, kidneys, pancreas, and tumor.

Mice in groups F and G were sacrificed and necropsied 24 hours after ²¹²Pb-DOTAM injection to confirm tumor uptake and clearance from normal tissues. The following organs and tissues were harvested and measured for radioactivity: blood, skin, bladder, spleen, pancreas, kidneys, liver, muscle, tail, and tumor.

Groups H-L comprised immuno-PD scout mice that were sacrificed and necropsied after retro-orbital bleeding, 24 hours after the second anti-PD-L1 injection, to assess the generation of anti-tumor T cell and DC responses by functional and phenotypical characterization of T cells and DCs from different compartments. From all immuno-PD mice were harvested: tumor, spleen, and DLNs (from the tumor side).

An ex vivo PMA/ionomycin (Thermo Fisher, cat No. 00-4970-03, 00-4980-03) restimulation assay was performed on spleen samples to assess T cell memory. In addition, FACS was performed using a MACSQuant Analyzer 10 (Miltenyi Biotec) and analysis of results was performed using the FlowJo 10.5.3 software. The staining panel design is shown in the Table below

FACS panel design for protocol 150

| | | | PMA/ | |
|---|---|---|---|---|
| T cells | DCs | Tregs | ionomycin | Differentiation |
| Marker | Marker | Marker | Marker | Marker |
| live/dead | live/dead | live/dead | live/dead | live/dead |
| CD45 | CD45 | CD45 | CD45 | CD45 |
| CD3 | Negative | CD3 | CD3 | CD3 |
| | selection | | | |
| | (CD19, Grl, F4/80) | | | |
| CD4 | CD11c | CD4 | CD4 | CD4 |
| CD8a | MHC class II | CD8 | CD8 | CD8a |
| 41BB | CD86 | CD25 | CD44 | CD62L |
| Lag3 | CD11b | CD11b | IFNγ | CD127 |
| PD-1 | CD317 | FoxP3 | IL-2 | CD44 |

IFNγ = interferon gamma; IL-2 = interleukin-2; MCH = major histocompatibility complex; PMA = phorbol 12-myristate-13-acetate; Treg = regulatory T cell

Results

Biodistribution and ELISA

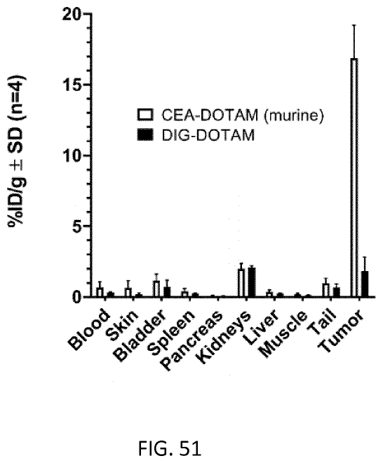

The average ²¹²Pb accumulation and clearance in all collected tissues 24 hours after ²¹²Pb-DOTAM injection (cycle 1) is displayed in FIG. 51. The tumor uptake was specific, with 16.9% ID/g in the tumor after pretargeting with CEA-DOTAM (mu), compared with <2.0% ID/g for all collected normal tissues. Using DIG-DOTAM, the resulting tumor accumulation was 1.8% ID/g.

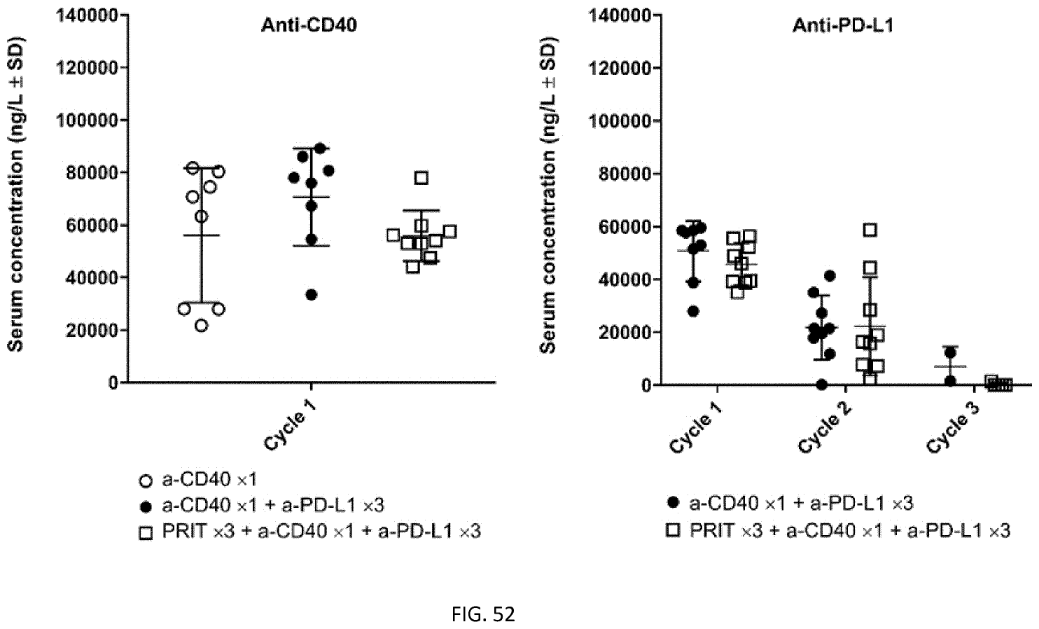

The serum concentration of anti-CD40 and anti-PD-L1 antibodies 24 hours after immunotherapy administration is shown in FIG. 52.

Tumor Development and Survival

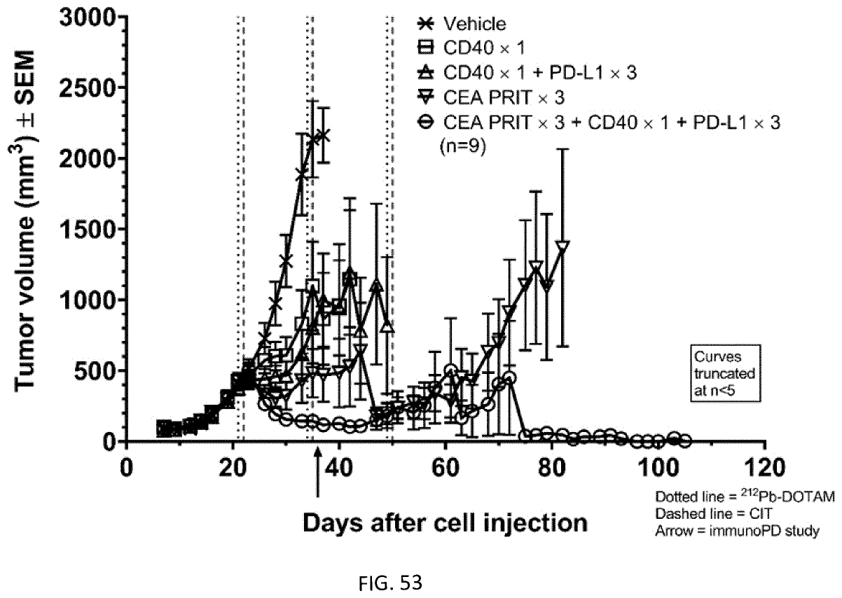

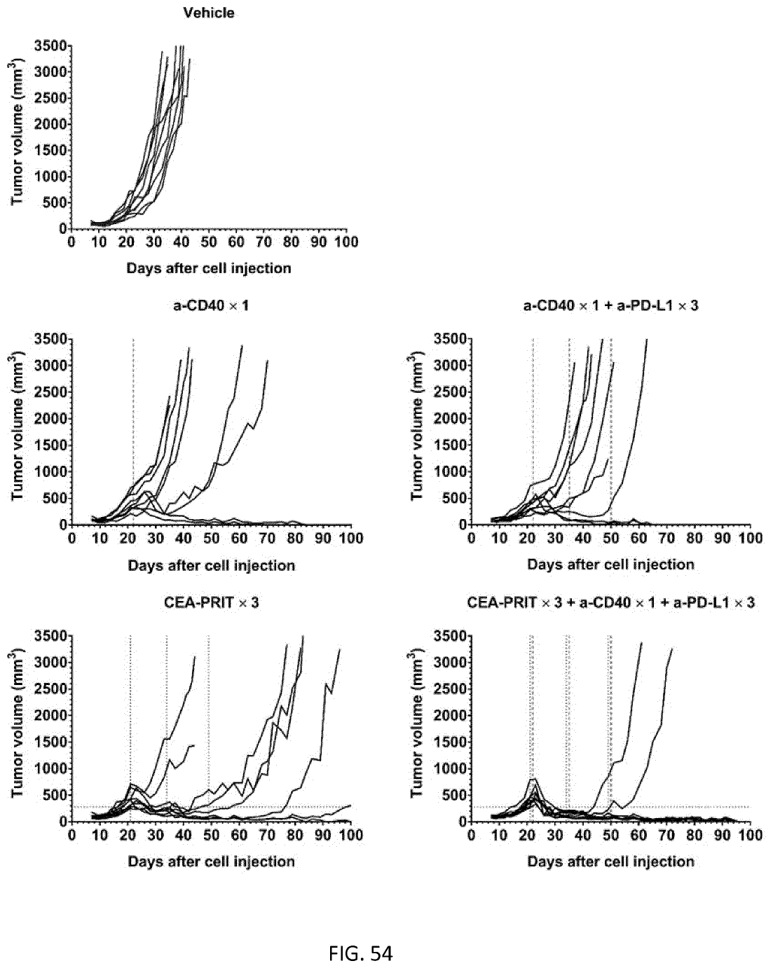

The average MC38-huCEA tumor development after CEA-PRIT and control treatments is shown in FIG. 53, with individual tumor growth curves for all treatment groups displayed in FIG. 54. On day 42, the last day on which all treatment groups could be analyzed based on means, the TGI was 57.8%, 54.5%, 82.8% and 99.6% for anti-CD40, anti-CD40+anti-PD-L1, CEA-PRIT, and the combination of CEA-PRIT and anti-CD40+anti-PD-L1, respectively, compared with the vehicle control. The primary study was terminated on day 103 after cell injection, at which point 2/9 mice were alive and tumor free (or with minuscule tumor) in the anti-CD40 group; the corresponding numbers were 2/9 and 1/9 in the anti-CD40+anti-PD-L1 and the CEA-PRIT groups, respectively. The combination of CEA-PRIT and anti-CD40+anti-PD-L1 resulted in a corresponding number of 7/9 mice.

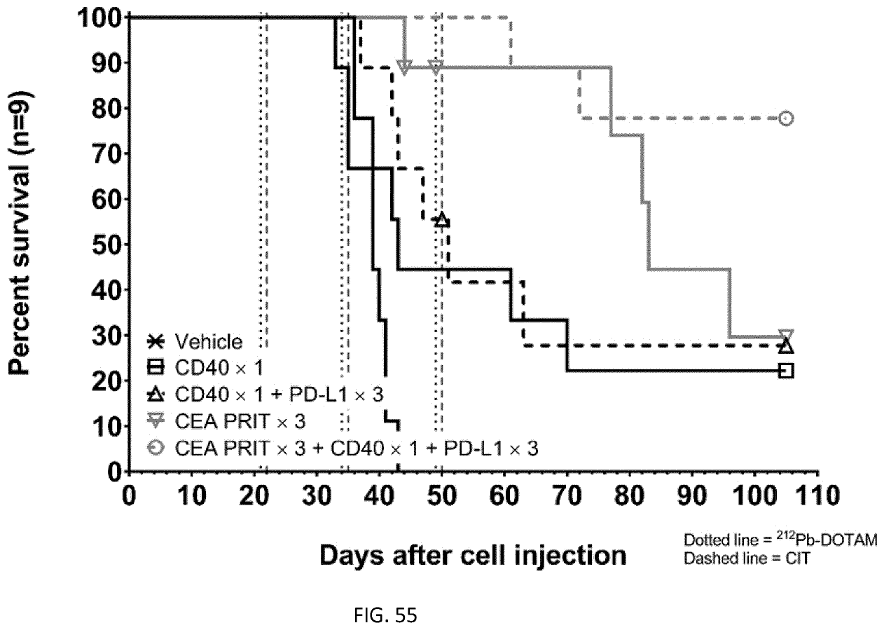

The overall survival is shown in FIG. 55, based on the termination criteria of tumor volume ≥3000 mm³. Time-to-event (tumor volume exceeding 3000 mm³) statistics for the individual treatment groups are shown in the table below, showing the median survival time with upper and lower 95% confidence limits, together with the quartile survival times (25% and 75%).

Quantiles with time-to-event* statistics (days)

| Group | Median time | Lower 95% | Upper 95% | 25% failures | 75% failures |
|---|---|---|---|---|---|
| Vehicle | 39 | 33 | 41 | 35 | 41 |
| anti-CD40 | 43 | 36 | — | 39 | 70 |
| anti-CD40 + PD-L1 | 51 | 37 | — | 43 | — |
| CEA-PRIT | 83 | 44 | — | 77 | — |
| CEA-PRIT + | — | 61 | — | — | — |
| anti-CD40 + anti-PD-L1 | | | | | |

*Event = tumor volume ≥3000 mm³.

Pairwise tests were performed to specify which groups were significantly different in terms of survival: the Log-Rank test (more weight on later survival events), and the Wilcoxon test (more weight on early survival times), both using Bonferroni correction for multiple testing. The results are shown in the tables below. Looking at the later survival times, all treatments except anti-CD40 alone significantly increased the survival compared with the vehicle group.

Pairwise Log-Rank test (multiple test level = 0.005)

| | | | | | CEA-PRIT + |
|---|---|---|---|---|---|
| | | | anti-CD40 + | | anti-CD40 + |
| Group | Vehicle | anti-CD40 | anti-PD-L1 | CEA-PRIT | anti-PD-L1 |
| Vehicle | 1.0000 | 0.0210 | 0.0008* | <0.0001* | <0.0001* |
| anti-CD40 | 0.0210 | 1.0000 | 0.6349 | 0.1300 | 0.0075 |
| anti-CD40 + anti-PD-L1 | 0.0008* | 0.6349 | 1.0000 | 0.2792 | 0.0167 |
| CEA-PRIT | <0.0001* | 0.1300 | 0.2792 | 1.0000 | 0.0992 |
| CEA-PRIT + | <0.0001* | 0.0075 | 0.0167 | 0.0992 | 1.0000 |
| anti-CD40 + anti-PD-L1 | | | | | |

Pairwise Wilcoxon test (multiple test level = 0.005)

| | | | | | CEA-PRIT + |
|---|---|---|---|---|---|
| | | | anti-CD40 + | | anti-CD40 + |
| Group | Vehicle | anti-CD40 | anti-PD-L1 | CEA-PRIT | anti-PD-L1 |
| Vehicle | 1.0000 | 0.0470 | 0.0019* | <0.0001* | <0.0001* |
| anti-CD40 | 0.0470 | 1.0000 | 0.4428 | 0.0297 | 0.0063 |
| anti-CD40 + anti-PD-L1 | 0.0019* | 0.4428 | 1.0000 | 0.1052 | 0.0129 |
| CEA-PRIT | <0.0001* | 0.0297 | 0.1052 | 1.0000 | 0.1550 |
| CEA-PRIT + | <0.0001* | 0.0063 | 0.0129 | 0.1550 | 1.0000 |
| anti-CD40 + anti-PD-L1 | | | | | |

Immuno-Pharmacoldynamics

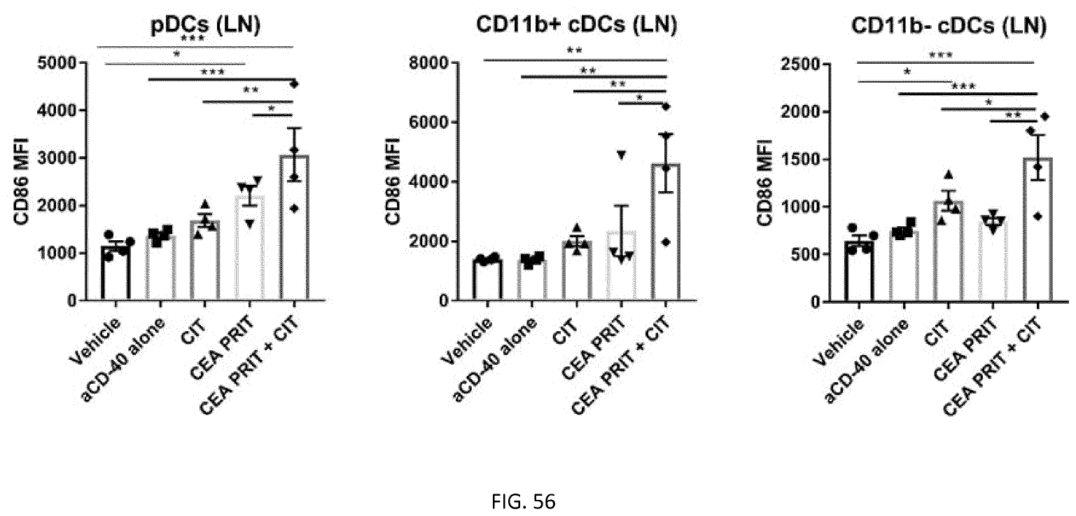

The CEA-PRIT/immunotherapy combination correlated with an increase in pDCs, activated pDCs, and cDCs (as measured by the increase of CD86 surface expression) in lymph nodes, in line with findings from protocol 136 (Panc02-huCEA-luc). This is shown in FIG. 56.

Rechallenge

To assess the development of anti-tumor immune memory response to the primary tumor, tumor-free mice after treatment were rechallenged with MC38-huCEA cells in the opposite flank to the primary injection. As control, untreated age-matched B6-huCEA mice were injected at the same time.

A total of 12 treated (tumor-free) mice were rechallenged on day 98 after the initial start of protocol 150: 2 treated with anti-CD40, 2 treated with anti-CD40+anti-PD-L1, 1 treated with CEA-PRIT alone, and 7 treated with the CEA-PRIT/immunotherapy combination. Xenografts were established by SC injection of 0.5×10⁵ cells (passage 17) into the left flank. The study groups and time course of the rechallenge/controls are shown in the tables below.

Study groups in protocol 150 (rechallenge)

| | | Age | | | n |
|---|---|---|---|---|---|
| Group | Treatment received | (weeks) | Cell line | Cells/mouse | (mice) |
| B* | anti-CD40 × 1 | 24-26 | MC38-huCEA | 0.5 × 10⁶ | 2 |
| C* | anti-CD40 × 1 + anti-PD-L1 × 3 | 24-26 | MC38-huCEA | 0.5 × 10⁶ | 2 |
| D* | CEA-PRIT × 3 | 24-26 | MC38-huCEA | 0.5 × 10⁶ | 1 |
| E* | CEA-PRIT × 3 + | 24-26 | MC38-huCEA | 0.5 × 10⁶ | 7 |
| | anti-CD40 × 1 + anti-PD-L1 × 3 | | | | |
| M | Untreated age-matched control | 24-26 | MC38-huCEA | 0.5 × 10⁶ | 5 |

*Retained group designation from protocol 150

Time course of protocol 150 (rechallenge)

| Study day | Date | Experimental procedure |
|---|---|---|
| 98 | 2018 Dec. 12 | Preparation of MC38-huCEA cells and filling of syringes |
| 98 | 2018 Dec. 12 | SC injection of MC38-huCEA cells (group B*, C*, D*, E* and M) |
| 125 | 2019 Jan. 8 | Euthanasia and tissue harvest for immuno-PD (FACS) (group B*, |
| | | C*, and M) |
| 127 | 2019 Jan. 10 | Euthanasia and tissue harvest for immuno-PD (FACS) (group D* and |
| | | E*) |

Blood, spleen, and DLNs were collected from all mice for flow cytometry analysis upon termination. Characterization of T cells was performed on blood and DLNs and an ex vivo PMA/ionomycin restimulation assay was performed on spleen and DLNs.

Results Rechallenge

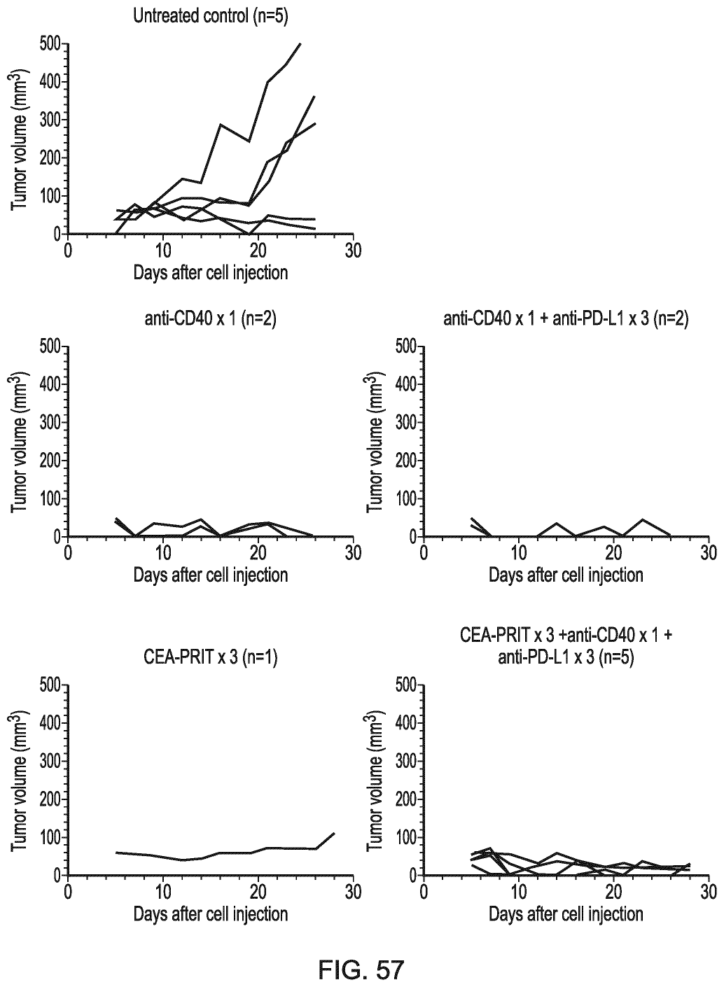

The tumor growth in rechallenged and naïve mice is shown in FIG. 57. Of the naïve control mice, only 3/5 developed tumors (known variability in this in vivo model). Of the rechallenged mice, only 1 mouse in the CEA-PRIT group had a tumor whose volume surpassed 100 mm³ when the experiment was terminated 28 days after the second inoculation; all other rechallenged mice remained essentially tumor-free.

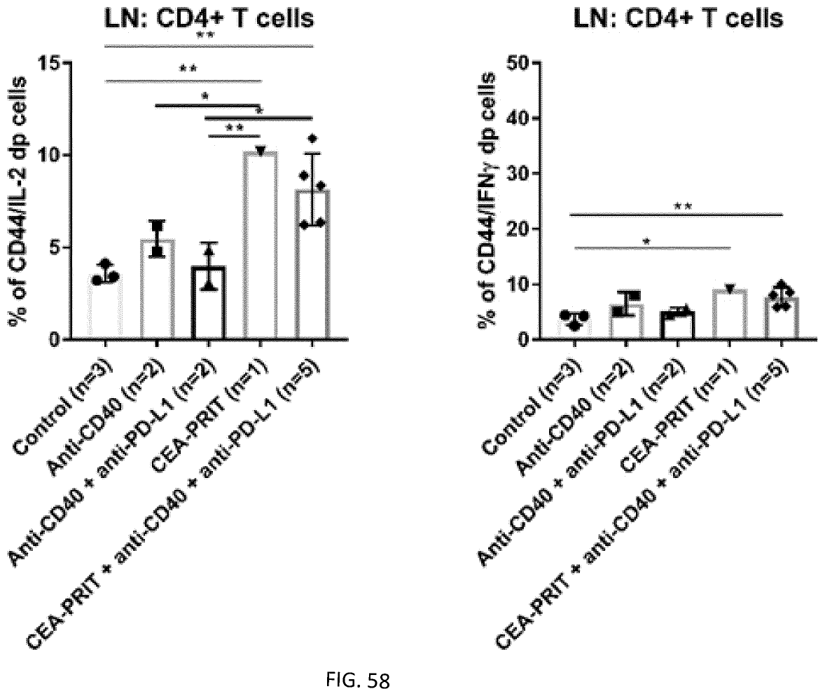

FACS analysis of samples from rechallenged and naïve mice revealed an increase of CD44+/IL-2 and CD44+/IFNγ CD4+cells in the DLNs of mice treated with the CEA-PRIT/immunotherapy combination, indicating the generation of effector memory T cell responses, in line with findings from protocol 136 (Panc02-huCEA-luc), shown in FIG. 58.

Adverse Events and Toxicity

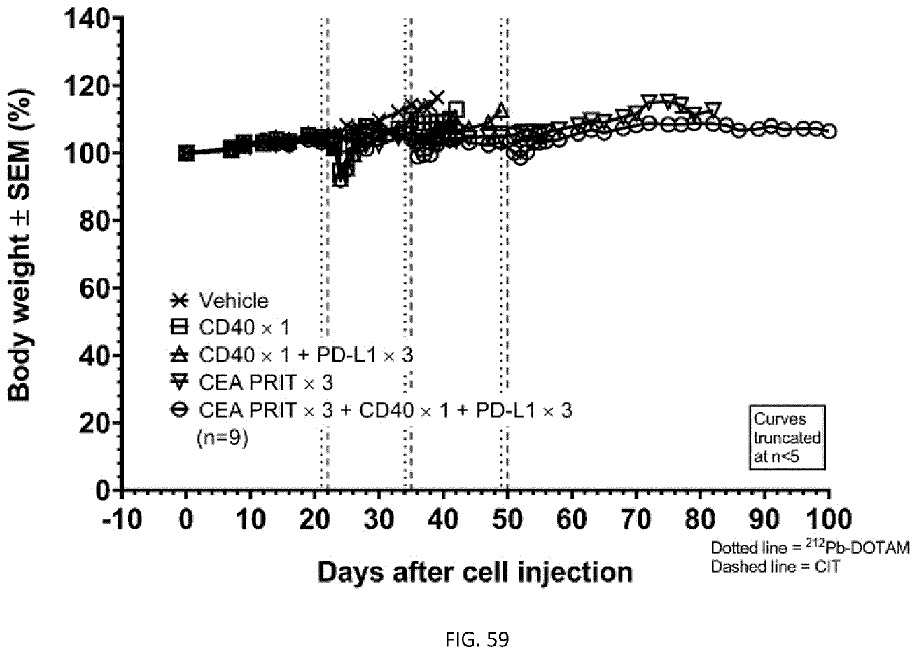

The average BW development in all therapy groups is shown in FIG. 59. Administration of anti-CD40 triggered an expected acute weight loss in injected mice, which was resolved within a week after injection. Injection of ²¹²Pb-DOTAM caused transient weight loss in irradiated mice, which was less severe than that from the anti-CD40. No mice were euthanized due to acute post-injection BW loss.

All animals (14/14) receiving a third cycle of anti-PD-L1, alone or after CEA-PRIT, exhibited a strong reaction within minutes after the injection, not previously seen in the Panc02-huCEA-luc models (protocols 119 and 136). Reactions included sluggish behaviour, somnolence, and death in 1/14 mice. Recovery followed approximately 15-40 minutes after the anti-PD-L1 injection; the single death occurred 20-30 minutes after injection.

From all groups, 1 mouse in the PRIT group was euthanized due to declining tumor status (open tumor with risk of degradation); another mouse in the PRIT group was excluded from the study due to a tail problem, preventing further IV injections. All adverse events are described in the table below.

Adverse events in protocol 150

| | Mice affected | | |
|---|---|---|---|
| Group | (n per group) | Study day | Observation(s) |
| C: anti-CD40 + anti-PD-L1 | 5(5) | 50 | Somnolence, lethargy, death |
| | | | (1 mouse) after 3^{rd} anti-PD- |
| | | | L1 injection |
| D: CEA-PRIT | 1(9) | 44 | Declining tumor status → |
| | | | euthanasia |
| D: CEA-PRIT | 1(9) | 49 | Injured tail → euthanasia |
| E: CEA-PRIT + anti-CD40 + anti- | 9(9) | 50 | Somnolence, lethargy, after |
| PD-L1 | | | 3^{rd} anti-PD-L1 injection |

²¹²Pb irradiation (20 μCi) was performed on study days 21, 34, and 49. Immunotherapy was administered on study days 22 (anti-CD-40 + anti-PD-L1), 35 (anti-PD-L1), and 50 (anti-PD-L1).

Conclusion

Compared with Panc02-huCEA-luc, immunotherapy alone was more efficient in the MC38-huCEA model. Either monotherapy (immunotherapies or CEA-PRIT) resulted in a number of tumor-free mice, but there was a clear benefit in terms of tumor control from combining anti-CD40 and anti-PD-L1 with the radiation treatment.

The results of the rechallenge experiment were more difficult to interpret compared with the Panc02-huCEA-luc equivalent, due to the more variable tumor take of MC38-huCEA. Additionally, the low sample number from monotherapy-treated groups made subsequent comparisons with the CEA-PRIT/immunotherapy combination group difficult to interpret.

### Example 9e: Protocol 195

The aim of protocol 195 was to assess the efficacy following CA (clearing agent)-independent PRIT using SeParated v-domains LInkage Technology (SPLIT) pretargeting antibodies (“SPLIT PRIT”), alone and in combination with CIT, for treatment of SC Panc02-huCEA-Fluc tumors in immunocompetent transgenic mice.

The PRIT treatment comprised IP injection of complementary SPLIT BsAbs (100 μg each in a total of 200 μL) followed 7 days later by IV administration of the effector molecule ²¹²Pb-DOTAM (20 μCi).

The immunotherapy treatment was administered IP 24 hours after the radioactive injection, consisting of a one-time administration of anti-CD40 antibody and multiple injections of anti-PD-L1 antibody (200 μg of each antibody in 200 μL). Scout mice were taken for biodistribution assessment to confirm ²¹²Pb-DOTAM targeting after PRIT treatment. Comparisons were made between the SPLIT PRIT/immunotherapy combination, SPLIT PRIT alone, immunotherapy alone, and no treatment. The treatment efficacy was assessed in terms of TGI, survival, and immune memory (rechallenge).

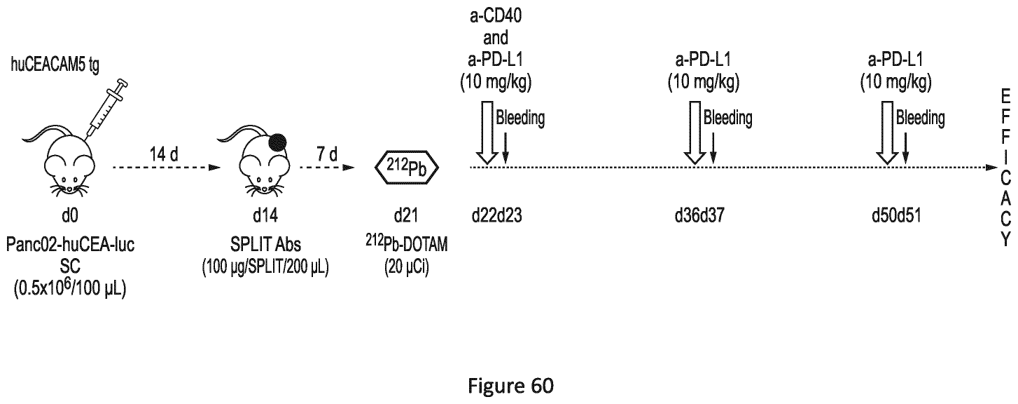

The study outline is shown in FIG. 60.

The time course and design of protocol 195 are shown in the tables below.

Time course of protocol 195

| Study day | Experimental procedure |
|---|---|
| 0 | Preparation of Panc02-huCEA-Fluc cells and filling of |
| | syringes |
| 0 | SC injection of Panc02-huCEA-Fluc cells |
| 14 | IP injection of SPLIT antibodies |
| 21 | Elution of ²¹²Pb-DOTAM and filling of syringes |
| 21 | IV injection of ²¹²Pb-DOTAM |
| 22 | Euthanasia and tissue harvest (24 h p.i.) + gamma |
| | counting |
| 22 | IP injection of anti-CD40 and anti-PD-L1 |
| 23 | Submandibular bleeding for ELISA |
| 36 | IP injection of anti-PD-L1 |
| 37 | Submandibular bleeding for ELISA |
| 50 | IP injection of anti-PD-L1 |
| 51 | Submandibular bleeding for ELISA |

Study groups in protocol 195

| | P1AD8592 | P1AF0171 | | | anti- | anti- | | |
|---|---|---|---|---|---|---|---|---|
| | (VL) | (VH-AST) | ²¹²Pb | PRIT | CD40* | PD-L1 | CIT | n |
| Group | (μg) | (μg) | (μCi) | cycles | (μg) | (μg) | cycles | (mice) |
| A | 0 | 0 | 0 | 0 | 0 | 0 | 0 | 10 |
| B | 0 | 0 | 0 | 0 | 200 | 200 | 3 | 10 |
| C | 100 | 143 | 20 | 1 | 0 | 0 | 0 | 10 |
| D | 100 | 143 | 20 | 1 | 200 | 200 | 3 | 10 |
| E | 100 | 143 | 20 | 1 | 0 | 0 | 0 | 3 |

*Anti-CD40 only administered once, at the first CIT cycle

Primary solid xenografts were established in each huCEACAM5 mouse (age 9-12 weeks) on study day 0 by SC injection of 0.5×10⁶ cells (passage 28) in RPMI/Matrigel, into the right flank. Fourteen days after tumor cell injection, mice were sorted into experimental groups with an average tumor volume of 102 mm³. The ²¹²Pb-DOTAM was injected seven days later; the average tumor volume on day 20 being 196 mm³ in the efficacy groups (A-D) and 187 mm³ in the biodistribution scout group (E).

Mice in groups A-D were mice followed to assess therapeutic efficacy until the end of the study or until one or several of the termination criteria were reached. Blood was sampled from mice in groups B and D 24 hours after administration of the immunotherapy, to validate the anti-CD40 and anti-PD-L1 injections by analysis of serum fractions through ELISA.

Mice in group E were sacrificed and necropsied 24 hours after the ²¹²Pb-DOTAM injection to confirm tumor uptake and clearance from normal tissues. The following organs and tissues were harvested and measured for radioactivity: blood, spleen, stomach, small intestine, colon, pancreas, kidneys, liver, lung, muscle, tail, and tumor.

Results

Biodistribution

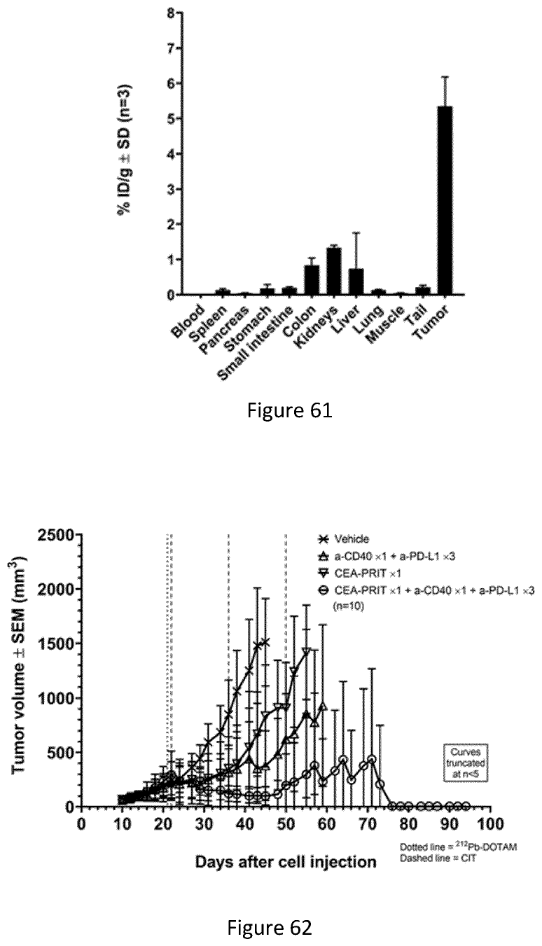

The average ²¹²Pb accumulation and clearance in all collected tissues 24 hours after ²¹²Pb-DOTAM injection is displayed in FIG. 61. The tumor uptake was specific, with 5.4% ID/g in the tumor after SPLIT PRIT, compared with <1.4% ID/g for all collected normal tissues.

Tumor Development and Survival

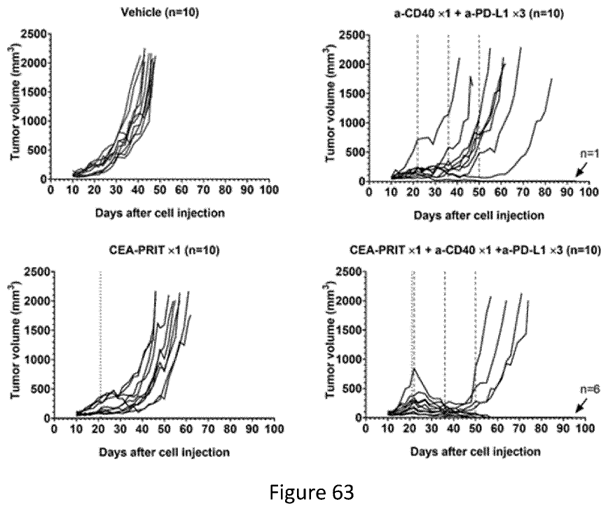

The average Panc02-huCEA-Fluc tumor development for all treatment groups is shown in FIG. 62, with individual tumor growth curves displayed in FIG. 63. On day 48, the last day on which all treatment groups could be analyzed based on means, the TGI was 78.9%, 58.6% and 100% for anti-CD40+anti-PD-L1, SPLIT CEA-PRIT, and the combination of SPLIT CEA-PRIT and anti-CD40+anti-PD-L1, respectively, compared with the vehicle control. The primary study was terminated on day 94 after cell injection, at which point 1/10 and 6/10 mice were alive and tumor free (or with minuscule tumor) in the anti-CD40+anti-PD-L1 and the SPLIT CEA-PRIT+anti-CD40+anti-PD-L1 groups, respectively. The vehicle and SPLIT CEA-PRIT groups had no tumor-free mice.

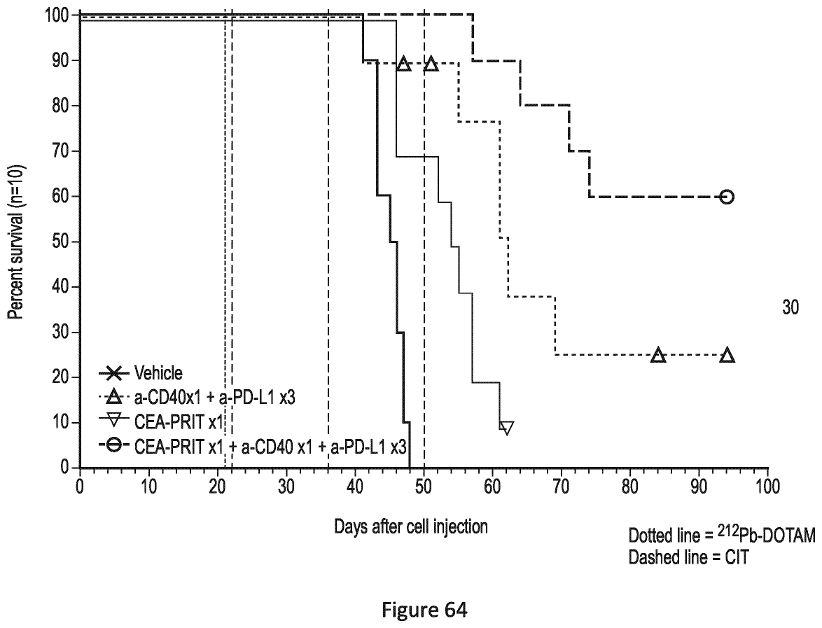

The overall survival is shown in FIG. 64, based on the termination criteria of tumor volume ≥2000 mm³.

Time-to-event (tumor volume exceeding 2000 mm³) statistics for the individual treatment groups are shown in the table below showing the median survival time with upper and lower 95% confidence limits, together with the quartile survival times (25% and 75%).

Time-to-event* statistics (days)

| | | | Median | | |
|---|---|---|---|---|---|
| Group | n | Events | time | Lower 95% | Upper 95% |
| Vehicle | 10 | 10 | 45.5 | 43 | — |
| anti-CD40 + PD-L1 | 10 | 7 | 62 | 61 | — |
| SPLIT CEA-PRIT | 10 | 9 | 54.5 | 46 | — |
| SPLIT CEA-PRIT + | 10 | 4 | — | 71 | — |
| anti-CD40 + | | | | | |
| anti-PD-L1 | | | | | |

*Event = tumor volume ≥2000 mm³.

Pairwise tests were performed to specify which groups were significantly different in terms of survival: the Log-Rank test (more weight on later survival events), and the Wilcoxon test (more weight on early survival times), both using Bonferroni correction for multiple testing. The results are shown in the two tables below. Looking at the later survival times, all treatments except anti-CD40 alone significantly increased the survival compared with the vehicle group.

Pairwise Log-Rank test (multiple test level = 0.005)

| | | | | CEA-PRIT + |
|---|---|---|---|---|
| | | | | anti-CD40 + |
| | | anti-CD40 + | | anti-PD- |
| Group | Vehicle | anti-PD-L1 | CEA-PRIT | L1 |
| Vehicle | 1.0000 | 0.0003* | 0.001* | 0* |
| anti-CD40 + anti- | 0.0003* | 1.0000 | 0.0453 | 0.0291 |
| PD-L1 | | | | |
| CEA-PRIT | 0.001* | 0.0453 | 1.0000 | 0.0004* |
| CEA-PRIT + | 0* | 0.0291 | 0.0004* | 1.0000 |
| anti-CD40 + anti- | | | | |
| PD-L1 | | | | |

Pairwise Wilcoxon test (multiple test level = 0.005)

| | | | | CEA-PRIT + |
|---|---|---|---|---|
| | | | | anti-CD40 + |
| | | anti-CD40 + | | anti-PD- |
| Group | Vehicle | anti-PD-L1 | CEA-PRIT | L1 |
| Vehicle | 1.0000 | 0.0019* | 0.0035* | 0.0001* |
| anti-CD40 + anti- | 0.0019* | 1.0000 | 0.0469 | 0.0301 |
| PD-L1 | | | | |
| CEA-PRIT | 0.0035* | 0.0469 | 1.0000 | 0.0011 |
| CEA-PRIT + | 0.0001* | 0.0301 | 0.0011* | 1.0000 |
| anti-CD40 + anti- | | | | |
| PD-L1 | | | | |

Rechallenge

To assess the development of anti-tumor immune memory response to the primary tumor, tumor-free mice after treatment were rechallenged with Panc02-huCEA-Fluc cells in the opposite flank to the primary injection. As control, untreated age-matched huCEACAM5 mice were injected at the same time.

A total of 6 treated (tumor-free) mice were rechallenged on day 94 after the initial start of protocol 195: all treated with the SPLIT PRIT/immunotherapy combination. Xenografts were established by SC injection of 0.5×10⁵ cells (passage 30) into the left flank. The study groups and time course of the rechallenge/controls are shown in the two tables below. Thirteen days after rechallenge, 3 mice per group were euthanized and samples taken for immuno-PD analysis (data not shown). Therefore, only 3 mice per group were followed up until the end of the study.

Study groups in protocol 195 (rechallenge)

| | | Age | | | |
|---|---|---|---|---|---|
| Group | Treatment received | (weeks) | Cell line | Cells/mouse | n (mice) |
| D* | SPLIT CEA-PRIT × 1 + | 22-25 | Panc02-huCEA- | 0.5 × 10⁶ | 6 |
| | anti-CD40 × 1 + anti-PD- | | Fluc | | |
| | L1 × 3 | | | | |
| F | Untreated age-matched | 26-27 | Panc02-huCEA- | 0.5 × 10⁶ | 6 |
| | control | | Fluc | | |

*Retained group designation from previously in protocol 195

Time course of protocol 195 (rechallenge)

| Study day | Experimental procedure |
|---|---|
| 94 | Preparation of Panc02-huCEA-Fluc cells and filling of |
| | syringes |
| 94 | SC injection of Panc02-huCEA-Fluc cells (opposite flank |
| | to primary injection) |
| 107 | Euthanasia and tissue harvest of 3 mice per group for |
| | immuno-PD analysis (data not shown) |
| 157 | Termination of study |

Results Rechallenge

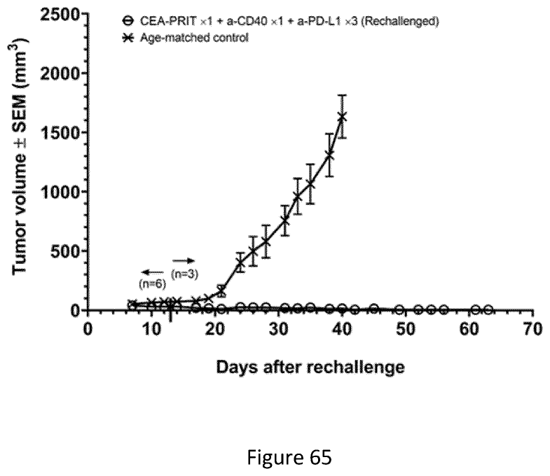

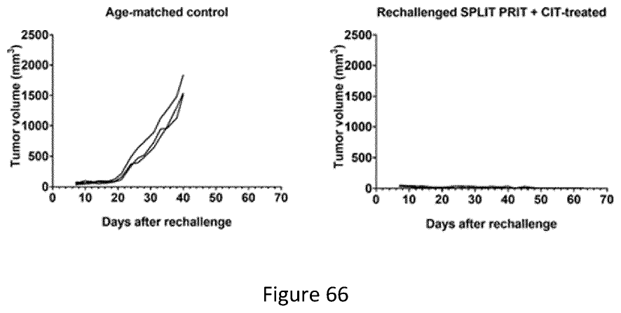

The tumor growth in rechallenged and naïve mice is shown FIGS. 65 and 66. Of the naïve control mice, 6/6 developed tumors. Of the rechallenged mice, 6/6 mice remained essentially tumor-free.

Adverse Events and Toxicity

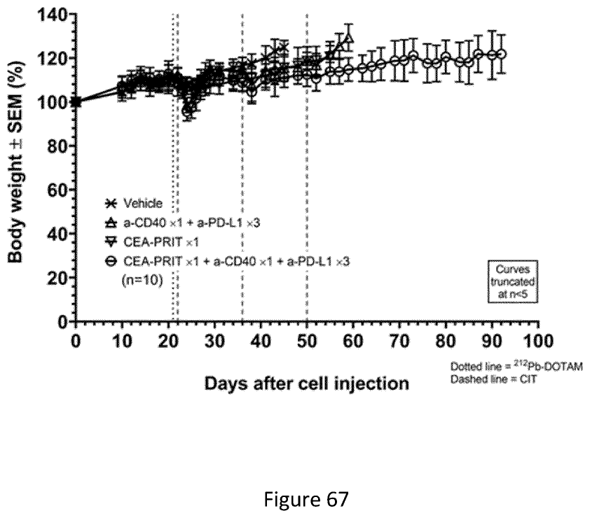

The average BW development in all therapy groups is shown in FIG. 67. Administration of anti-CD40 triggered an expected acute weight loss in injected mice, which was resolved within a week after injection. Injection of ²¹²Pb-DOTAM did not cause any significant weight loss by itself. No mice were euthanized due to acute post-injection BW loss.

Mice receiving multiple administrations of anti-PD-L1, alone or after CEA-PRIT, exhibited a reaction within approximately 10 minutes after the injection: ca 40% and 80% after the second and third administrations, respectively; no specific reactions were seen after the first anti-PD-L1 injection. Reactions included skin irritation, redness, decreased activity, spasms, ruffled fur, arched back. All mice recovered 1-2 hours after the anti-PD-L1 injection.

From all groups, 2 mice were euthanized due to declining tumor status (open tumor with risk of degradation). In addition, 1 mouse in the anti-CD40+anti-PD-L1 group was found dead 1 day after the third anti-PD-L1 injection, for unknown reasons. All adverse events are described in the table below.

Adverse events in protocol 195

| | Mice affected | | |
|---|---|---|---|
| Group | (n per group) | Study day | Observation(s) |
| B: anti-CD40 + anti-PD-L1 | 1(10) | 47 | Declining tumor status |
| B: anti-CD40 + anti-PD-L1 | ~40-80% | 36, 51 | Reaction after |
| | | | repeated anti-PD-L1 |
| | | | injection |
| B: anti-CD40 + anti-PD-L1 | 1(10) | 51 | Mouse found dead; no |
| | | | apparent reason |
| B: anti-CD40 + anti-PD-L1 | | 36, 50 | |
| C: SPLIT CEA-PRIT | 1(10) | 62 | Declining tumor status |
| D: SPLIT CEA-PRIT + | ~40-80% | 36, 51 | Reaction after |
| anti-CD40 + anti-PD-L1 | | | repeated anti-PD-L1 |
| | | | injection |

²¹²Pb irradiation (20 μCi) was performed on study day 21. Immunotherapy was administered on study days 22 (anti-CD-40 + anti-PD-L1), 36 (anti-PD-L1), and 50 (anti-PD-L1).

### CONCLUSION

The combination of one cycle of two-step SPLIT CEA-PRIT with three cycles of immunotherapy was as efficient as three full cycles of three-step CEA-PRIT combined with immunotherapy in the SC Panc02-huCEA-luc model (see Protocol 136), and resulted in 60% (6/10) cured mice. The presence of immune memory was strongly indicated by the rechallenge experiment, in which none of the pre-treated, rechallenged mice developed tumors, compared with 100% of the naïve control mice.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of treating a proliferative disorder in a subject, comprising treating the subject with:
i) pre-targeted radioimmunotherapy comprising administering to the subject a multispecific antibody or split multispecific antibody, said antibody or split antibody having a binding site for a radiolabelled compound and a binding site for a target antigen, and further comprising administering to the subject the radiolabelled compound; and
ii) immunotherapy comprising administering to the subject a CD40 agonist and an immune checkpoint inhibitor.

2. The method of claim 1, wherein the method comprises a treatment cycle comprising a first step of pre-targeted radioimmunotherapy comprising administering the multispecific antibody or split multispecific antibody and then administering the radiolabelled compound, and a second step of immunotherapy comprising administering a CD40 agonist and an immune checkpoint inhibitor, wherein the anti-CD40 antibody and the immune checkpoint inhibitor are administered simultaneously or sequentially in either order.

3. The method of claim 2, wherein the method comprises one or more additional cycles of treatment, wherein each additional cycle comprises a first step of pre-targeted radioimmunotherapy comprising administering the multispecific antibody or split multispecific antibody and then administering the radiolabelled compound, and a second step of immunotherapy comprising administering an immune checkpoint inhibitor.

4. The method of claim 3, wherein the method comprises 2, 3, 4 or 5 additional cycles.

5. The method of any one of claims 1 to 5, wherein the radiolabelled compound is DOTAM chelated with ²¹²Pb, ²¹²Bi or ²¹³Bi.

6. The method of any one of claims 1 to 5, wherein the target antigen is CEA.

7. The method of any one of the preceding claims, wherein the multispecific antibody comprises an Fc domain.

8. The method of claim 7, wherein the Fe domain is modified to reduce or eliminate effector function.

9. The method according to any one of claims 1 to 6, wherein the method comprises administering a split multispecific antibody, wherein the split antibody comprises
i) a first hemibody that binds to a target antigen, and which further comprises a VH domain of an antigen binding site for a radiolabelled compound, but which does not comprise a VL domain of an antigen binding site for the radiolabelled compound; and
ii) a second hemibody that binds to a target antigen, and which further comprises a VL domain of an antigen binding site for the radiolabelled compound, but which does not comprise a VH domain of the antigen binding site for the radiolabelled compound, wherein said VH domain of the first hemibody and said VL domain of the second hemibody are together capable of forming a functional antigen binding site for the radiolabelled compound.

10. The method of claim 9, wherein the first and second hemibodies each comprise an Fc domain.

11. The method of claim 10, wherein the Fc domain is modified to reduce or eliminate effector function.

12. The method of any one of the preceding claims, wherein the CD40 agonist is an agonistic anti-CD40 antibody.

13. The method according to any one of the preceding claims, wherein the immune checkpoint inhibitor is selected from an inhibitor of PD1, PDL1 or CTLA4.

14. The method according to claim 13, wherein the immune checkpoint inhibitor is an antibody selected from an antibody against PD1, an antibody against PDL1 and an antibody against CTLA4.

15. The method of claim 14, wherein the immune checkpoint inhibitor is an antibody against PDL1.

16. The method of any one of the preceding claims, wherein the proliferative disorder is cancer.

17. The method according to any one of the preceding claims, wherein the subject is human.

18. The method according to any one of the preceding claims, wherein the method results in a slower rate of tumour growth than treatment with the pre-targeted radioimmunotherapy and/or the immunotherapy alone.

19. The method according to any one of the preceding claims, wherein the method results in an increased likelihood of subject survival than treatment with the pre-targeted radioimmunotherapy and/or the immunotherapy alone.

20. The method according to any one of the preceding claims, wherein the method results in an increased frequency of activated intratumoral CD8 T cells and/or an increased frequency of activated plasmacytoid DCs (pDCs) and classical DCs (cDCs) in tumor, spleen and draining lymph nodes (DLNs) than treatment with the pre-targeted radioimmunotherapy and/or the immunotherapy alone.

21. The method according to any one of the preceding claims, wherein the method results in an enhanced immune memory response than treatment with the pre-targeted radioimmunotherapy and/or the immunotherapy alone.

22. A multispecific antibody or a split multispecific antibody, said multispecific antibody or a split multispecific antibody having a binding site for a radiolabelled compound and a binding site for a target antigen, for use in a method of treating a proliferative disorder, wherein the treatment comprises administering the multispecific antibody or split multispecific antibody, and wherein the treatment further comprises administering i) the radiolabelled compound, ii) an CD40 agonist and iii) an immune checkpoint inhibitor.

23. A multispecific antibody or a split multispecific antibody for use according to claim 22, wherein the method is a method according to any one of claims 1 to 21.

24. A CD40 agonist for use in a method of treating a proliferative disorder, wherein the treatment further comprises administering i) a multispecific antibody or split multispecific antibody having a binding site for a radiolabelled compound and a binding site for a target antigen; ii) the radiolabelled compound, and iii) an immune checkpoint inhibitor.

25. The CD40 agonist for use according to claim 24, wherein the method is a method according to any one of claims 1 to 21.

26. An immune checkpoint inhibitor for use in a method of treating a proliferative disorder, wherein the treatment further comprises administering i) a multispecific antibody or split multispecific antibody having a binding site for a radiolabelled compound and a binding site for a target antigen; ii) the radiolabelled compound and iii) a CD40 agonist.

27. The immune checkpoint inhibitor according to claim 26, wherein the method is a method according to any one of claims 1 to 21.

28. A multispecific antibody or a split multispecific antibody having a binding site for a radiolabelled compound and a binding site for a target antigen, a radiolabelled compound; a CD40 agonist and an immune checkpoint inhibitor for use in combination in a method of treating a proliferative disorder.

29. The multispecific antibody or a split multispecific antibody having a binding site for a radiolabelled compound and a binding site for a target antigen, a radiolabelled compound; a CD40 agonist and an immune checkpoint inhibitor for use according to claim 28, wherein the method is a method according to any one of claims 1 to 21.

30. A pharmaceutical product comprising A) as a first component a composition comprising as an active ingredient a multispecific antibody or a split multispecific antibody having a binding site for a radiolabelled compound and a binding site for a target antigen; B) as a second component a composition comprising as an active ingredient a CD40 agonist; and C) as a third component a composition comprising as an active ingredient an immune checkpoint inhibitor, preferably a PD-L1 inhibitor, for the combined, simultaneous or sequential, treatment of a proliferative disease.

31. The pharmaceutical product of claim 30, wherein the proliferative disease is cancer.

32. The pharmaceutical product of claim 30 or claim 31, further comprising D) as a fourth component a composition comprising as an active ingredient the radiolabelled compound.
